(19) United States
(12) Patent Application Publication
(10) Pub. No.: US 2026/0176274 A1
(43) Pub. Date: Jun. 25, 2026

(54) ENAMINE N-OXIDE TOOLS FOR TARGET IDENTIFICATION APPLICATIONS

(71) Applicant: DANA-FARBER CANCER INSTITUTE, INC., Boston, MA (US)

(72) Inventors: Justin KIM, Boston, MA (US); Surached SIRIWONGSUP, Boston, MA (US)

(73) Assignee: DANA-FARBER CANCER INSTITUTE, INC., Boston, MA (US)

(21) Appl. No.: 19/127,526

(22) PCT Filed: Nov. 21, 2023
(86) PCT No.: PCT/US2023/080688

§ 371 Date: May 6, 2025

Related U.S. Application Data
(60) Provisional application No. 63/427,334, filed on Nov. 22, 2022.

(51) Int. Cl. C07D 487/04

(52) U.S. Cl. CPC C07D 487/04

(57) ABSTRACT

Disclosed are compounds and pharmaceutically acceptable salts and stereoisomers thereof that are suitable for cellular labeling. Also disclosed are pharmaceutical compositions containing same, and methods of making and using the compounds.

### RELATED APPLICATIONS

This application claims the benefit of priority under 35 U.S.C. § 119(e) to U.S. Provisional Application No. 63/427,334, filed Nov. 22, 2022, which is incorporated herein by reference in its entirety.

### GOVERNMENT SUPPORT

This invention was made with government support under grant number DP2 ES030448 awarded by The National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Nov. 21, 2023, is named 52095_775001WO_ST.xml and is 12 KB bytes in size.

### BACKGROUND OF THE DISCLOSURE

Target identification (ID) refers to the important process of identifying the molecular target responsible for a drug's pharmacological activity, and it is considered the rate-limiting step in phenotype-based drug discovery programs (Schenone et al., Nat. Chem. Biol., 9(4):232-240 (2013); Williams, M., Curr. Opin. Pharmacol., 3(5):571-577 (2003); Chan et al., Trends Pharmacol. Sci., 31(2):82-88 (2010); Ha et al., Cell Chem. Biol., 28(3):394-423 (2021); Morgan et al., Nat. Rev. Drug Discov., 17:167-181 (2018); Hart, C., Drug Discovery Today, 10(7):513-519 (2005); Park et al., Mol. BioSyst., 9:544-550 (2013); Tamura et al., J. Am. Chem. Soc., 141(7):2782-2799 (2019)). Similarly, off-target identification is important in inventorying and elucidating the role of proteins that interact with a lead compound and is essential in building an accurate risk profile for lead drug candidates. In canonical target ID studies, bead-immobilized small molecules are used to pull down protein targets. This method works suitably well for covalent and strong single-digit nanomolar binding compounds. However, non-covalent small molecules identified from primary phenotypic screens seldom display this level of potency. For these weakly binding compounds, affinity handles such as azides, alkynes, or biotin are first transferred to the protein target for subsequent pull-down experiments. This is typically accomplished with a reactive species.

Notwithstanding developments in the space of electrophilic label transfer using ligand-directed chemistry by Hamachi and others (Tamura et al., J. Am. Chem. Soc., 141(7):2782-2799 (2019); Tamura et al., J. Am. Chem. Soc., 139(40):14181-14191 (2017); Tsukiji et al., Curr. Opin. Chem. Biol., 21:136-143 (2014); Hayashi et al., Acc. Chem. Res., 45(9):1460-1469 (2012)), the most widely adopted method of label transfer is photoaffinity labeling (PAL) (Ha et al., Cell Chem. Biol., 28(3):394-423 (2021); Smith et al., Future Med. Chem., 7(2):159-183 (2015); Murale et al., Proteome Sci., 15:14 (2016); Das, J., Chem. Rev., 111(8):4405-4417 (2011)). This method employs trivalent compounds consisting of a ligand, an affinity handle, and a photoreactive group. Because highly reactive species with short half-lives and labeling radii are necessary for covalently modifying ligand-associated target proteins with great specificity, a triggered activation mechanism is an essential component of this technology. Diazirines, aryl azides, and benzophenones have proven useful for this purpose.

While photoaffinity labeling (PAL) is state-of-the-art in target identification (ID) applications, it has a few key limitations: labeling efficiency and linker dependence. Poor labeling efficiency plagues diazirine-based probes as greater than 99% of carbenes generated by photoirradiation results in quenching by water (Park et al., ACS Chem. Biol., 11(1):44-52 (2016)). Dialkyldiazirine probes, which have become more popular than the trifluoromethylaryldiazirines due to their smaller size, additionally face unproductive carbene to alkene isomerization as well as a reactivity altering diazirine to diazo rearrangement (West et al., J. Am. Chem. Soc., 143(17):6691 (2021)). Diazo compounds neither have the degree nor scope of reactivity of singlet carbenes, favoring reaction with acidic residues in specific cellular contexts. The challenges of target ID arising from the poor labeling efficiency of diazirine probes are well-documented (Park et al., ACS Chem. Biol., 11(1):44-52 (2016); Wright et al., Nat. Prod. Rep., 33(5):681-708 (2016)). Furthermore, a linker connects diazirine to ligand in PAL reagents. The linker must adequately position the reactive species near a suitable amino acid on the target protein for label transfer to take place. Consequently, PAL involves extensive optimization of linker length and composition. Linker dependence is a core limitation of nearly all affinity-based target ID tools including other photoactivatable systems like 2-aryl-5-carboxytetrazoles (Herner et al., J. Am. Chem. Soc., 138(44):14609-14615 (2016)) as well as non-photochemical ligand-directed (Tamura et al., J. Am. Chem. Soc., 141(7):2782-2799 (2019); Tamura et al., J. Am. Chem. Soc., 139(40):14181-14191 (2017); Tsukiji et al., Curr. Opin. Chem. Biol., 21:136-143 (2014); Hayashi et al., Acc. Chem. Res., 45(9):1460-1469 (2012)) and electroaffinity methods (Kawamata et al., ChemRxiv®, Feb. 7, 2022).

Recently, MacMillan and co-workers introduced a μMap system that employs an iridium photocatalyst, which undergoes Dexter energy transfer to a biotin-trifluoromethylalkyldiazirine upon photoirradiation and generates a carbene for proximity labeling (Geri et al., Science, 367(6482):1091-1097 (2020)). This method circumvents the issue of linker dependence and seeks to improve labeling efficiency through catalytic turnover. While useful for antibody-based proximity labeling, application of this system to target identification importantly requires the attachment of a huge, hydrophobic cation onto a small molecule of interest (Trowbridge et al., Proc. Natl. Acad. Sci. USA, 119(34):e2208077119 (2022)). The system still relies on a diazirine precursor with low labeling efficiency, and it is unclear if Dexter energy transfer ultimately results in significantly more diazirine activation even with catalytic turnover.

Currently, the triggered activation of reactive species for affinity labeling can only be achieved photochemically. Comparable chemically induced processes do not yet exist. Chemically activated systems require the use of a bioorthogonal transformation, but the current compendium of bioorthogonal reactions is still inadequate, especially in non-ligation reactions.

### SUMMARY OF DISCLOSURE

A first aspect of the present disclosure is directed to a compound represented by any one of formulas I-VI:

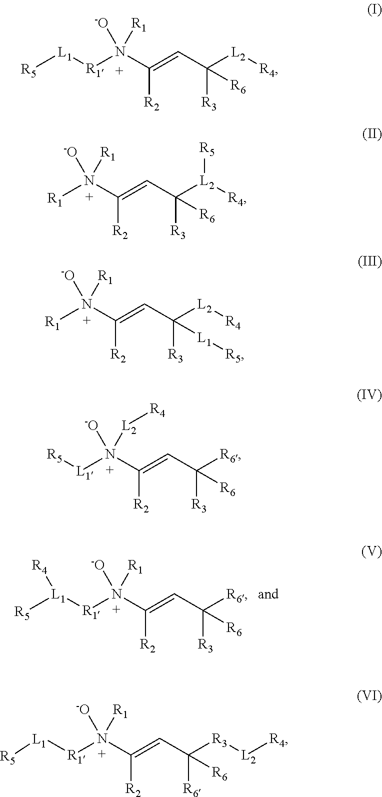

or a pharmaceutically acceptable salt or stereoisomer thereof,
wherein:
each of L₁ and L₂ is independently absent or a linking group, which may be the same or different;
R_{1′} is absent, or
R_{1′} and R₁, together with the nitrogen atom to which they are bound, form an optionally substituted 4- or 10-membered heterocyclyl comprising 1 to 3 heteroatoms selected from O, N, and S;
each R₁ is independently (C₁-C₈) alkyl, (C₃-C₁₀) carbocyclyl, or 4- or 10-membered heterocyclyl comprising 1 to 3 heteroatoms selected from O, N, and S, wherein said alkyl, carbocyclyl or heterocyclyl is further optionally substituted, or
two R₁ groups, together with the nitrogen atom to which they are bound, form an optionally substituted 4- or 10-membered heterocyclyl comprising 1 to 3 heteroatoms selected from O, N, and S;
R₂ is halo or alkoxy;
R₃ is a leaving group;
R₄ is a ligand that binds a cellular protein or a nucleic acid;
R₅ is a bioorthogonal handle, an affinity handle, or a reporter; and
R₆ is hydrogen, (C₁-C₆) alkyl, or optionally substituted aryl,
provided that R₃ is not halo for the compound of formula VI.

Other aspects of the present disclosure are directed to compounds represented by formula VII:

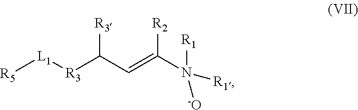

or a pharmaceutically acceptable salt or stereoisomer thereof,
wherein:
L is a linking group;
R₁ and R_{1′} are independently (C₁-C₈) alkyl, (C₃-C₁₀) carbocyclyl, or 4- or 10-membered heterocyclyl comprising 1 to 3 heteroatoms selected from O, N, and S, wherein said alkyl, carbocyclyl or heterocyclyl is further optionally substituted, or
R₁ and R_{1′} together with the nitrogen atom to which they are attached, form a 4- to 7-membered heterocyclyl comprising 1 to 3 heteroatoms selected from O, N, and S;
R₂ is halo or alkoxy;
R₃ is absent, OC(O), C(O)O, OC(O)O, OC(O)NR′, NR′C(O)O, S(O), S(O)₂, OS(O)₂, S(O)₂O, OP(OR′)O₂, or a leaving group, wherein each R′ is independently hydrogen, (C₁-C₆) alkyl, (C₃-C₁₀) carbocyclyl, 4- or 7-membered heterocyclyl, and wherein said alkyl, carbocyclyl, or heterocyclyl is optionally substituted;
R_{3′} is hydrogen, (C₁-C₆) alkyl, (C₃-C₁₀) carbocyclyl, or 4- or 10-membered heterocyclyl comprising 1 to 3 heteroatoms selected from O, N, and S, or a leaving group, wherein said alkyl, carbocyclyl or heterocyclyl is further optionally substituted; and
R₅ is a bioorthogonal handle, an affinity handle, a reporter, or a ligand that binds a cellular protein or a nucleic acid, provided that one of R₃ or R_{3′} is a leaving group.

Another aspect of the present disclosure is directed to a diagnostic composition that includes a diagnostically effective amount of a compound of formula (I-VII) or a pharmaceutically acceptable salt or stereoisomer thereof, and a pharmaceutically acceptable carrier.

Further aspects of the present disclosure are directed to processes of preparing a compound of formula (I-VI). A process of preparing a compound of formula I:

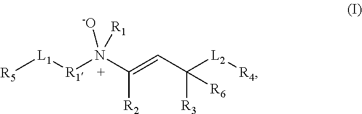

comprising reacting a compound of formula VIII:

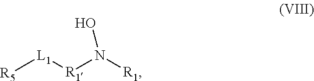

with a compound of formula IX:

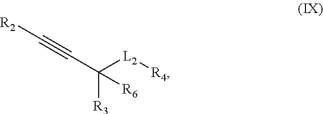

or
a compound of formula II:

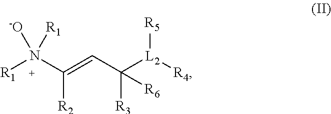

comprising reacting a compound of formula X:

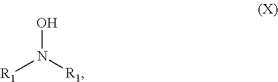

with a compound of formula XI:

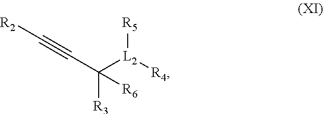

a compound of formula III:

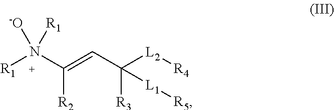

comprising reacting a compound of formula X:

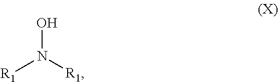

with a compound of formula XII:

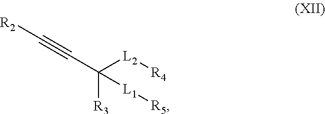

or
a compound of formula IV:

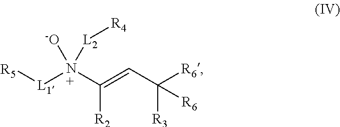

comprising reacting a compound of formula XIII:

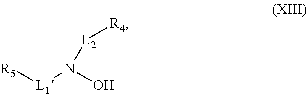

with a compound of formula XIV:

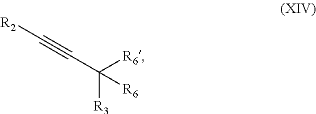

or
a compound of formula V:

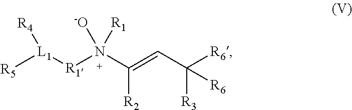

comprising reacting a compound of formula XV:

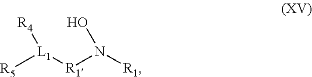

with a compound of formula XIV:

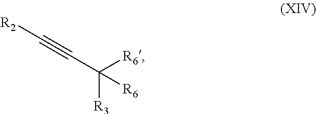

or
a compound of formula VI:

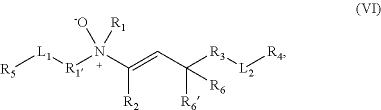

comprising reacting a compound of formula VIII:

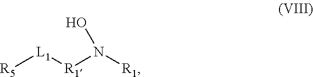

with a compound of formula XV:

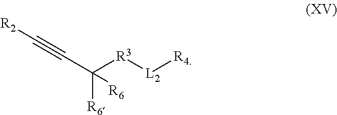

Bioorthogonal activation of reactive species described herein are suitable for labeling a range of amino acid residues on proteins in a 1,2- or 1,4-fashion. Affinity labeling reagents bearing the electrophilic α,β-unsaturated moiety described herein enable ligand-directed protein modification and afford highly sensitive and selective target identification.

Yet further aspects of the present disclosure are directed to methods of protein and nucleic acid labeling using the inventive compounds. The methods may entail contacting a cell or lysate thereof with a compound of formula I-VII or pharmaceutically acceptable salt or stereoisomer, and a diboron reagent. The methods may be performed in vitro as well as on live cells (e.g. in vivo and ex vivo).

### BRIEF DESCRIPTION OF THE DRAWINGS

FIG. 1 is a schematic depicting proximity labeling and target identification applications.

FIG. 2A-FIG. 2E are reaction schemes and Western blots showing ligand-directed labeling. FIG. 2A is a series of reactive species derived from halogenated enamine N-oxides. FIG. 2B is a reaction scheme and Western blot showing protein labeling with latent electrophiles. High concentration of enamine N-oxides 1-3 was added to GFP followed by diboron. Biotin azide was clicked onto the alkynes using copper-catalyzed azide-alkyne cycloaddition (CuAAC). FIG. 2C is a reaction showing the reactivity of Ac-Cys-OH (50 mM), Ac-Lys-OH (50 mM), and water toward bromoiminium ions was evaluated in PBS. FIG. 2D are chemical structures of tethered and untethered labeling agents along with diazirine control. FIG. 2E is a Western blot of HEK293T cell lysate (1 mg/mL) that were treated with probes 11-13 (200 nM) containing sulfonamide, a carbonic anhydrase (CA) inhibitor, then activated by diboron (100 μM, 10 min) or UV irradiation (10 min). A competition experiment was performed with excess sulfonamide 14 (500 μM) where biotin azide was clicked on.

FIG. 3 is a schematic depicting target identification through chemically induced reactive electrophile formation.

FIG. 4A-FIG. 4D show the synthesis of enamine N-oxide-modified drugs and probes for target ID validation studies. Reaction conditions: (a) TsCl, DMAP, NEt₃, CH₂Cl₂, (b) p-NO₂PhOCOCl, ⁱPr₂EtN, (c) PPh₃, I₂, imidazole, CH₂Cl₂, (d) MeNHOH·HCl, NEt₃, DMSO, 70° C., (e) EDC, 16, (f) 20, 20% 2,2,2-trifluoroethanol/CHCl₃, 50° C., (g) K₂CO₃, 17, acetone, (h) 18, ⁱPr₂EtN, CH₂Cl₂. FIG. 4A shows the synthesis of basic building blocks. FIG. 4B depicts rapid modification of commercially available or known intermediates en route to indicated drugs and probes. FIG. 4C are schematics for untethered labeling compounds and proximity labeling. FIG. 4D is a schematic showing ligand-directed labeling with caged electrophile.

FIG. 5 is a schematic showing the probe design with a tethered ligand.

FIG. 6 is a series of Western blots showing the labeling of carbonic anhydrase in vitro with SA-1.

FIG. 7 is a schematic showing the reaction schemes for probe design with a dissociating ligand.

FIG. 8 is a schematic showing the probe design with a dissociating ligand.

FIG. 9 is a series of Western blots showing the labeling of carbonic anhydrase in vitro with SA-2.

FIG. 10A is a series of mass spectra showing the product of dissociating linker. FIG. 10B is the proposed mechanism for the product of the dissociating linker. FIG. 10C shows the products that were observed through the reactivity with amino acids.

FIG. 11 is a series of chemical structures and Western blots showing labeling activity of carbonic anhydrase.

FIG. 12 is a series of chemical structures and a Western blot showing that labeling activity is invariant to α-halogen.

FIG. 13 is a series of chemical structures and a Western blot showing linker length dependence.

FIG. 14A-FIG. 14B show site identification with recombinant carbonic anhydrase using SA-8.

FIG. 15 is a series of chemical structures and mass spectra showing diazirine vs N-oxide extent of carbonic anhydrase labeling.

FIG. 16 is a series of chemical structures and a Western blot showing labeling of endogenous carbonic anhydrase in HEK293T cell lysate.

FIG. 17 is a series of Western blots for labeling conditions with SA-1 and SA-8.

FIG. 18 is a series of plots showing chemoproteomics data with SA-1.

FIG. 19 is a series of plots showing chemoproteomics data with SA-8.

FIG. 20 is a series of chemical structures and Western blots showing validation with dasatinib, a kinase inhibitor.

FIG. 21 is a series of chemical structures and Western blots showing validation with mirdametinib, a kinase inhibitor.

FIG. 22 is a series of chemical structures and Western blots showing validation with lenalidomide and pomalidomide.

FIG. 23A-FIG. 23C are reaction schemes and Western blots showing ligand-directed labeling. FIG. 23A is a reaction scheme and Western blot showing BSA labeling efficiency with excess reactive species. BSA (0.1 mg/mL) was labeled by reductive activation of enamine N-oxides 1-3 (FIG. 2A) with B₂(OH)₄ over 10 min in PBS, conjugated to biotin, and analyzed by streptavidin blot. FIG. 23B shows haloiminium reactions with amino acids. Amino acid addition products. 50 mM N-oxide 7′a was activated with 60 mM B₂(OH)₄ in the presence of 500 mM (a) Ac-Lys-OH, (b) Ac-Cys-OH, and (c) Ac-His-OH. FIG. 23C shows amino acid labeling preferences on pooled proteins. Myoglobin, BSA, CA, and lysozyme were labeled with probe 7′b (R,R′=Me, (CH₂)₃CCH) and diboron, trypsin digested, and analyzed by LC-MS/MS. Number of unique residues labeled by 1,2-, 1,4-, or 1,2/1,4-addition and residue coverage normalized by residue abundance are plotted. BSA, bovine serum albumin; CA, carbonic anhydrase.

FIG. 24A shows the structures of sulfonamide (21′) and corresponding BARS (21′a) and PAL (21b) reagents. FIG. 24B is a Western blot showing ligand-dependent labeling of recombinant protein in vitro. BSA and CA (1:1, 0.1 mg/mL) were treated with N-oxide probe 21′a (200 nM) with or without B₂(OH)₄ (100 μM) and with or without sulfonamide (21′, 50 μM) in PBS, pH 7.4 for 10 min. Tetramethylrhodamine (TAMRA)-azide was conjugated by CuAAC then imaged by in-gel fluorescence. FIG. 24C is a Western blot showing HEK293T cell lysate that was treated as in FIG. 24B. Biotin picolyl azide was conjugated and imaged by streptavidin blot. CA is 29 kDa. FIG. 24D is a Western blot showing HEK293T cell lysate that was treated with BARS (21′a) or PAL (21′b) reagents as in FIG. 24B. Diazirine 21′b was UV irradiated for 10 min. Biotin conjugation, streptavidin pulldown, and immunoblotting against CA2 demonstrates superior labeling by N-oxide 21′a. FIG. 24E shows modified peptides detected in site identification of samples from FIG. 24D. b/y-ions; probe fragment (o); probe neutral loss (Δ); histidine immonium ion (o). Y-axis is cropped. FIG. 24F shows quantification of labeling by intact mass spectrometry of recombinant CA labeled with 21′a (10 M) and B₂(OH)₄ (100 μM) or 21′b (10 μM) and UV irradiation for 10 min. FIG. 24G shows target identification in cell lysate from the indicated cell lines that were treated with either BARS or PAL probe (1 μM), activated with B₂(OH)₄ (100 μM) or UV irradiation for 10 min, biotin conjugated, pulled down with streptavidin, and immunoblotted against validated targets. FIG. 24H shows a diboron concentration screen with HEK293T cell lysate was treated with probe 21′a (200 nM) and activated with varying concentrations of B₂(OH)₄. FIG. 24I depicts a competitor concentration screen with labeling in HEK293T cell lysate by probe 21′a (200 nM) and B₂(OH)₄ (100 μM) was competed away with varying concentrations of sulfonamide 21′. FIG. 24J shows target identification in live HEK293T cells that were treated with probe 21′a or 22′a (5 μM) for 2 h, washed, activated with B₂(OH)₄ (100 μM) for 10 min, washed, then analyzed by blot against CA2 and BRD4. DB, diboron; UV, 365 nm.

FIG. 25A-FIG. 25H show a series of volcano plots of HEK293T cell lysate labeled with probe (FIG. 25A) 21′a with or without B₂(OH)₄, (FIG. 25B) 21′a with or without 21′, (FIG. 25C) 21′b with or without UV irradiation, (FIG. 25D) 21′b with or without 21′, (FIG. 25E) 22′a with or without 22′, (FIG. 25F) 22′b with or without 22′, (FIG. 25G) 22′a with or without 22′, (FIG. 25H) 22′a versus ent-22′a. FIG. 25A-FIG. 25F: Protein identifications were filtered to a minimum of 3 quantified peptides in 3 replicates; FIG. 25G-FIG. 25H: Protein identifications were filtered to a minimum of 1 quantified peptide in 3 replicates. Hit threshold: fold change >2, p-value <0.01. Conditions: Probe (1 μM), B₂(OH)₄ (100 μM), competitor (50 μM).

FIG. 26A shows the structure of compound 1′. FIG. 26B is a Western blot of a 32-base pair sequence of single-stranded DNA (10 μM) that was labeled by reductive activation of enamine NV-oxide 1′ (100 μM) with B₂(OH)₄ (200 μM) over 10 min in TE buffer. FIG. 26C shows full in-gel fluorescence and GelRed stain images for FIG. 26B.

FIG. 27 shows a streptavidin blot and coomassie stain for FIG. 23A. BSA (0.1 mg/mL) was labeled by reductive activation of enamine N-oxides 1-3 with B₂(OH)₄ over 10 min in PBS, conjugated to biotin, and analyzed by streptavidin blot.

FIG. 28A-FIG. 28B are a series of liquid chromatography (LC) traces (220 nm) for the chemical trapping of the bromoiminium reactive intermediate generated from enamine N-oxide 7′a and B₂(OH)₄. LCMS analysis was performed using a C₁₈ reverse phase column (4.6×50 mm, 2.7 μm particle size, eluent: 1 mL/min flow rate, eluent: H₂O+0.1% TFA (1 min), gradient 0→100% MeCN/H₂O+0.1% TFA (5 min). FIG. 28A is a LC trace for 50 mM 7′a with or without tetrahydroxydiboron (60 mM). FIG. 28B is a LC trace for a reaction with Ac-Lys-OH (500 mM). FIG. 28C is a LC trace for a reaction with Ac-Cys-OH (500 mM). FIG. 28D is a LC trace for a reaction with Ac-His-OH (500 mM).

FIG. 29 shows full sequences of recombinant proteins used to determine amino acid labeling preference in FIG. 23C. Residues labeled via 1,2-addition are bold and underlined. Residues labeled via 1,4-addition are bold. Residues labeled via 1,2- and 1,4-addition are bold and italicized.

FIG. 30 is an annotated MS/MS spectrum for lysozyme N37 modified by probe 7′b via 1,4-addition. b/y-ions; probe neutral loss (Δ). Probe neutral loss corresponds to b or y ions with neutral loss of the probe.

FIG. 31 is an annotated MS/MS spectrum for BSA E69 modified by probe 7′b via 1,4-addition. b/y-ions; probe neutral loss (Δ); probe fragment (o, corresponding to the mass ion of the entire probe, m/z=256.1734).

FIG. 32 is an annotated MS/MS spectrum for BSA Y179 modified by probe 7′b via 1,4-addition. b/y-ions; probe neutral loss (Δ); probe fragment (o, corresponding to the mass ion of the entire probe, m/z=256.1657).

FIG. 33 is an annotated MS/MS spectrum for carbonic anhydrase P13 modified by probe 7′b via 1,4-addition. Inset: Representative m/z showing presence of doubly charged ion. b/y-ions; probe neutral loss (Δ); probe fragment (o, corresponding to the mass ion of the entire probe, m/z=256.1680). Note: In this example, the peptide also carries an additional modification on either H17 or K18.

FIG. 34 is an annotated MS/MS spectrum for carbonic anhydrase H15 modified by probe 7′b via 1,4-addition.

FIG. 35 is an annotated MS/MS spectrum for lysozyme C82 modified by probe 7′b via 1,4-addition. b/y-ions; probe neutral loss (Δ); probe fragment (o, corresponding to the mass ion of the entire probe, m/z=256.1698).

FIG. 36 is an annotated MS/MS spectrum for BSA K245 modified by probe 7′b via 1,2-addition. b/y-ions.

FIG. 37 is an annotated MS/MS spectrum for carbonic anhydrase V50 modified by probe 7′b via 1,2-addition. b/y-ions.

FIG. 38 is an annotated MS/MS spectrum for lysozyme G135 modified by probe 7′b via 1,4-addition. b/y-ions; probe neutral loss (Δ).

FIG. 39 is a full in-gel fluorescence and Coomassie stain images for FIG. 24B. Ligand-dependent labeling of recombinant protein in vitro. BSA and CA (1:1, 333 nM) were treated with N-oxide probe 21′a (200 nM) with or without B₂(OH)₄ (100 μM) and with or without sulfonamide (21′, 50 μM) in PBS, pH 7.4 for 10 min. Tetramethylrhodamine (TAMRA)-azide was conjugated by copper-catalyzed azide-alkyne cycloaddition (CuAAC) then imaged by in-gel fluorescence.

FIG. 40A is a full Western blot for FIG. 24D. HEK293T cell lysate (0.1 mg/mL) was treated with N-oxide probe 21′a (200 nM) with or without B₂(OH)₄ (100 μM) and with or without sulfonamide (21′, 50 μM) in PBS, pH 7.4 for 10 min. In parallel, HEK293T cell lysate (0.1 mg/mL) was treated with diazirine probe 21′b (200 nM) with or without UV irradiation (365 nm) and with or without sulfonamide (21′, 50 μM) in PBS, pH 7.4 for 10 min. Biotin picolyl azide conjugation, streptavidin pulldown, and immunoblotting against CA2 followed. FIG. 40B shows the quantification of bands by densitometry analysis. Band intensities are normalized to lane 4.

FIG. 41 is a full MS/MS spectra of carbonic anhydrase N-terminal peptides modified by 21′a in FIG. 24E. b/y-ions; probe neutral loss (Δ) corresponding to b or y ions with neutral loss of the probe or fragments thereof, probe fragment (o), which includes the mass ion of the entire probe (m/z=623.3096) or fragment ions (m/z=184.0063, 241.0640, 277.1909); histidine immonium ion (o, ImmH).

FIG. 42 shows the amino acid residues modified by BARS labeling and identified by site identification in FIG. 24E are highlighted in on the crystal structure (His2 and His3) of bovine carbonic anhydrase 2 (CA2, PDB: 1V9E).

FIG. 43 is a full Western blot for pull-down and input samples using (+)-JQ1 enamine N-oxide 22′a in FIG. 24G. A sample using the (−)-JQ1 analog ent-22′a with diboron activation was also prepared as an additional negative control.

FIG. 44 is a full Western blot for pull-down and input samples using (+)-JQ1 PAL probe 22′b in FIG. 24G.

FIG. 45 is a full Western blot for pull-down and input samples using lenalidomide enamine N-oxide 23′a in FIG. 24G.

FIG. 46 is a full Western blot for pull-down and input samples using lenalidomide PAL probe 23′b in FIG. 24G.

FIG. 47A is a full Western blot for pull-down and input samples using dasatinib enamine N-oxide 24′a and dasatinib PAL probe 24′b in FIG. 24G. FIG. 47B shows the quantification of first six bands (pull-down samples) by densitometry analysis. Band intensities are normalized to lane 2.

FIG. 48 shows a Western blot analysis for additional targets ABL and p38 pulled down using dasatinib enamine N-oxide 24′a. K562 cell lysate was treated with probe 24′a (1 μM), activated with B₂(OH)₄ (100 μM) for 10 min, conjugated with biotin picolyl azide, pulled down with streptavidin, and immunoblotted.

FIG. 49 is a full Western blot for pull-down and input samples using mirdametinib enamine N-oxide 25′a in FIG. 24G.

FIG. 50 is a full Western blot for pull-down and input samples using mirdametinib PAL probe 25′b in FIG. 24G.

FIG. 51A is a full Western blot for pull-down and input samples using staurosporine enamine N-oxide 26′a and staurosporine PAL probe 26′b in FIG. 24G. FIG. 51B shows the quantification of first six bands (pull-down samples) by densitometry analysis. Band intensities are normalized to lane 2.

FIG. 52 shows a Western blot analysis for additional targets PKC and SRC pulled down using staurosporine enamine N-oxide 26′a from FIG. 24G. HepG2 cell lysate was treated with either probe 26′a (1 μM), activated with B₂(OH)₄ (100 μM) for 10 min, biotin conjugated, pulled down with streptavidin, and immunoblotted.

FIG. 53A is a full streptavidin blot for diboron concentration screen in HEK293T cell lysate (FIG. 24H). FIG. 53B shows the quantification of 29 kDa band by densitometry analysis. Band intensities are normalized to lane 9.

FIG. 54A is a full streptavidin blot for competitor (compound 21′) concentration screen in HEK293T cell lysate (FIG. 24I). FIG. 54B shows the quantification of 29 kDa band by densitometry analysis. Band intensities are normalized to lane 2.

FIG. 55 is a full Western blot for pull-down and input samples using sulfonamide BARS probe 21′a treated in live HEK293T cells in FIG. 24J. HEK293T cells (2×10⁷ cells/mL) were treated with probe 21′a (5 μM), activated with B₂(OH)₄ (100 μM) for 10 min, conjugated with biotin picolyl azide, pulled down with streptavidin, and immunoblotted.

FIG. 56 is a full Western blot for pull-down and input samples using (+)-JQ1 enamine N-oxide 22′a treated in live HEK293T cells in FIG. 24J. HEK293T cells (4×10⁷ cells/mL) were treated with probe 22′a (5 μM), activated with B₂(OH)₄ (100 μM) for 10 min, conjugated with biotin picolyl azide, pulled down with streptavidin, and immunoblotted. A sample using the (−)-JQ1 analog ent-22′a with diboron activation was also prepared as an additional negative control.

FIG. 57A is a volcano plot for labeling of HEK293T cell lysate with sulfonamide enamine N-oxide 21′a (1 μM) with or without B₂(OH)₄ (100 μM) as in FIG. 25A, but with all four replicates included. FIG. 57B is a volcano plot for labeling of HEK293T cell lysate with sulfonamide enamine N-oxide 21′a (1 μM) with or without B₂(OH)₄ (100 μM) with more relaxed filters (1 quantified peptide in 3 replicates) than FIG. 25A. FIG. 57C is a volcano plot for labeling of HEK293T cell lysate with sulfonamide enamine N-oxide 21′a (1 μM) with or without competitor 21′ (50 μM) with more relaxed filters (1 quantified peptide in 3 replicates) than FIG. 25B. FIG. 57D is a volcano plot for labeling of HEK293T cell lysate with (+)-JQ1 enamine N-oxide 22′a (1 μM) versus ent-22′a (1 μM). Protein identifications were filtered to a minimum of 3 quantified peptides in 3 replicates. FIG. 57E is a volcano plot for labeling of HEK293T cell lysate with (+)-JQ1 diazirine 22′b (1 μM) with or without competitor 22′ (50 μM) with more relaxed filters (1 quantified peptide in 3 replicates) than FIG. 25F.

FIG. 58A is a schematic showing general affinity labeling strategy for target identification applications. FIG. 58B shows reactive species used in photoaffinity labeling. FIG. 58C is a schematic showing chemically inducible reactive species based on the bioorthogonal reduction of enamine N-oxides with diboron reagents.

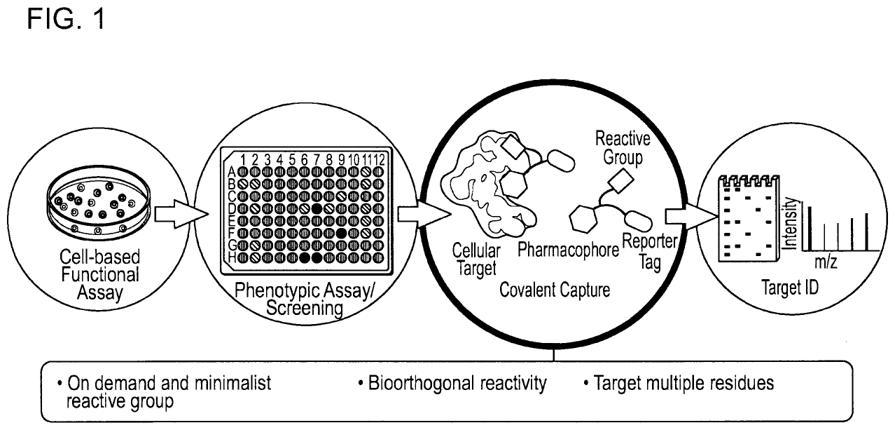

### DETAILED DESCRIPTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” include plural referents unless the context clearly dictates otherwise. Therefore, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2%, or 1%) of the particular value modified by the term “about.”

The transitional term “comprising,” which is synonymous with “including,” “containing,” or “characterized by,” is inclusive or open-ended and does not exclude additional, unrecited elements or method steps. When used in the context of the number of heteroatoms in a heterocyclic structure, it means that the heterocyclic group that that minimum number of heteroatoms. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the disclosure.

The term “bioorthogonal reaction” refers to any chemical reaction that can occur inside of a living system without interfering with native biochemical processes.

With respect to compounds of the present disclosure, and to the extent the following terms are used herein to further describe them, the following definitions apply.

As used herein, the term “alkyl” refers to a saturated linear or branched-chain monovalent hydrocarbon radical. In some embodiments, the alkyl radical is a C₁-C₆ group. In some embodiments, and to the extent not disclosed otherwise for any one or more groups of the compounds of formula (I-VII), the alkyl radical is a C₀-C₆, C₀-C₅, C₀-C₃, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. In some embodiments, an alkyl group is a C₁-C₃ alkyl group. In some embodiments, an alkyl group is a C₁-C₂ alkyl group. In some embodiments, an alkyl group is a methyl group.

As used herein, the term “alkylene” refers to a straight or branched divalent hydrocarbon chain linking the rest of the molecule to a radical group, consisting solely of carbon and hydrogen, containing no unsaturation and having from one to six carbon atoms, for example, methylene, ethylene, propylene, n-butylene, and the like. The alkylene chain may be attached to the rest of the molecule through a single bond and to the radical group through a single bond. In some embodiments, and to the extent not disclosed otherwise for any one or more groups of the compounds of formula (I-VII), an alkylene group contains one to four carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In some embodiments, the alkenyl radical is a C₂-C₁₅ group. In some embodiments, and to the extent not disclosed otherwise for any one or more groups of the compounds of formula (I-VII), the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In some embodiments, the alkynyl radical is a C₂-C₁₅ group. In some embodiments, and to the extent not disclosed otherwise for any one or more groups of the compounds of formula (I-VII), the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. In some embodiments, the alkoxyl group is methoxy, ethoxy, propyloxy, or tert-butoxy. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “halogen” (or “halo” or “halide”) refers to fluorine, chlorine, bromine, or iodine.

As used herein, the term “cyclic group” refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Therefore, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 12 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 10 carbon atoms (C₃-C₁₀). In one embodiment, carbocyclyl includes 3 to 6 carbon atoms (C₃-C₆). In one embodiment, carbocyclyl includes 5 to 6 carbon atoms (C₅-C₆). In some embodiments, carbocyclyl, as a bicycle, includes C₆-C₁₀. In another embodiment, carbocyclyl, as a spiro system, includes C₅-C₁₁. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and phenyl; bicyclic carbocyclyls having 7 to 11 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Therefore, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R′-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-12 carbon atoms. In another embodiment, aryl includes groups having 6-10 carbon atoms. Examples of aryl groups include phenyl, naphthyl, biphenyl, 1,2,3,4-tetrahydronaphthalenyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Therefore, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, 4, or 5) carbon atoms have been replaced with a heteroatom or heteroatom-containing group (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3- to 12-membered heterocyclyl ring system. In some embodiments, a heterocyclyl refers to a saturated ring system, such as a 3- to 12-membered saturated heterocyclyl ring system. In some embodiments, a heterocyclyl refers to a heteroaryl ring system, such as a 5- to 12-membered heteroaryl ring system. The term heterocyclyl also includes C₂-C₈ heterocycloalkyl, which is a saturated or partially unsaturated mono-, bi-, or spiro-ring system containing 2-8 carbons and one or more (e.g., 1, 2, or 3) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from O, N, and S. In some embodiments, heterocyclyl includes 4- to 6-membered monocycles having one or more heteroatoms selected from O, N, and S. In some embodiments, heterocyclyl includes 3-membered monocycles. In some embodiments, heterocyclyl includes 4-membered monocycles. In some embodiments, heterocyclyl includes 5- to 6-membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be substituted (e.g., methyl, isopropyl) and/or quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl (e.g., thiazol-2-yl), thiadiazolyl (e.g., 1,3,4-thiadiazol-5-yl and 1,2,4-thiadiazol-5-yl), oxazolyl (e.g., oxazol-2-yl), and oxadiazolyl (e.g., 1,3,4-oxadiazol-5-yl and 1,2,4-oxadiazol-5-yl). Example of 5-membered heterocyclyls containing 2 to 4 nitrogen atoms include imidazolyl (e.g., imidazol-2-yl), triazolyl (e.g., 1,3,4-triazol-5-yl, 1,2,3-triazol-5-yl, and 1,2,4-triazol-5-yl), and tetrazolyl (e.g., 1H-tetrazol-5-yl). Representative examples of benzo-fused 5-membered heterocyclyls include benzoxazol-2-yl, benzthiazol-2-yl and benzimidazol-2-yl. Example of 6-membered heterocyclyls containing one to three nitrogen atoms and optionally a sulfur or oxygen atom are pyridyl (e.g., pyrid-2-yl, pyrid-3-yl, and pyrid-4-yl), pyrimidyl (e.g., pyrimid-2-yl and pyrimid-4-yl), triazinyl (e.g., 1,3,4-triazin-2-yl and 1,3,5-triazin-4-yl), pyridazinyl (e.g., pyridazin-3-yl), and pyrazinyl. In some embodiments, a heterocyclic group includes a heterocyclic ring fused to one or more (e.g., 1 or 2) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Therefore, the term heterocyclic embraces N-heterocyclyl groups which as used herein refer to a heterocyclyl group containing at least one nitrogen atom and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, 1-pyrazolidinyl, 1-imidazolinyl and 1-imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2- or 3-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”)) refers to a monocyclic, bicyclic or tricyclic ring system having 5 to 12 ring atoms, wherein at least one ring is aromatic and contains at least one heteroatom. In one embodiment, heteroaryl includes 5- to 6-membered monocyclic aromatic groups where one or more ring atoms is O, N, or S. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, and 1,2,3-triazol-5-yl. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1 or 2) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Therefore, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s) in the compounds of formula (I-VII), any of the groups described herein may be substituted or unsubstituted. To the extent not disclosed otherwise for any particular group(s), representative examples of substituents may include alkyl (e.g., C₁-C₆, C₁-C₅, C₁-C₄, C₁-C₃, C₁-C₂, C₁), substituted alkyl (e.g., substituted C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., substituted C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., substituted C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., substituted C₂-C₆, C₂-C₅, C₂-C₄, C₂-C₃, C₂), cyclic (e.g., C₃-C₁₂, C₅-C₆), substituted cyclic (e.g., substituted C₃-C₁₂, C₅-C₆), carbocyclic (e.g., C₃-C₁₂, C₅-C₆), substituted carbocyclic (e.g., substituted C₃-C₁₂, C₅-C₆), heterocyclic (e.g., 3- to 12-membered, 5- to 6-membered), substituted heterocyclic (e.g., substituted 3- to 12-membered, 5- to 6-membered), aryl (e.g., benzyl and phenyl), substituted aryl (e.g., substituted benzyl or substituted phenyl), heteroaryl (e.g., pyridyl or pyrimidyl), substituted heteroaryl (e.g., substituted pyridyl or substituted pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., substituted C₆-C₁₂, C₆), alkylthio (e.g., C₁-C₆), substituted alkylthio (e.g., substituted C₁-C₆), arylthio (e.g., C₆-C₁₂, C₆), substituted arylthio (e.g., substituted C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

As used herein, the term “small molecule” refers to a molecule, whether naturally-occurring or artificially created (e.g., via chemical synthesis) that has a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.).

In one aspect, compounds of the disclosure are represented by any one of formulas I-VI:

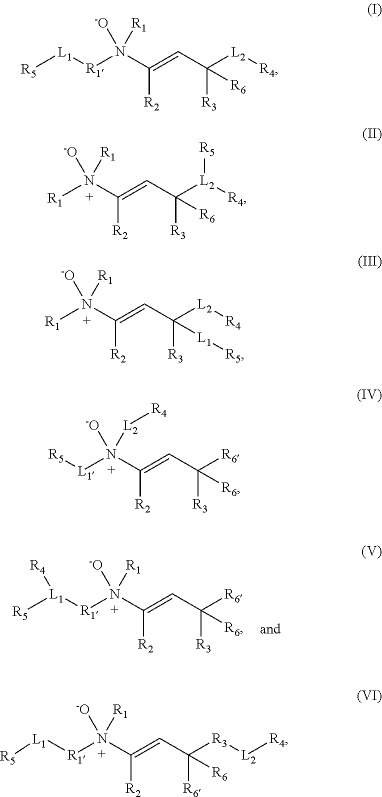

or a pharmaceutically acceptable salt or stereoisomer thereof,
wherein:
each of L₁ and L₂ is independently absent or a linking group, which may be the same or different;
R_{1′} is absent, or
R_{1′} and R₁, together with the nitrogen atom to which they are bound, form an optionally substituted 4- or 10-membered heterocyclyl comprising 1 to 3 heteroatoms selected from O, N, and S;
each R₁ is independently (C₁-C₈) alkyl, (C₃-C₁₀) carbocyclyl, or 4- or 10-membered heterocyclyl comprising 1 to 3 heteroatoms selected from O, N, and S, wherein said alkyl, carbocyclyl or heterocyclyl is further optionally substituted, or
two R₁ groups, together with the nitrogen atom to which they are bound, form an optionally substituted 4- or 10-membered heterocyclyl comprising 1 to 3 heteroatoms selected from O, N, and S;
R₂ is halo or alkoxy;
R₃ is a leaving group;
R₄ is a ligand that binds a cellular protein or a nucleic acid;
R₅ is a bioorthogonal handle; and
R₆ is hydrogen, (C₁-C₆) alkyl, or optionally substituted aryl,
provided that R₃ is not halo for the compound of formula VI.

In some embodiments, L₁ is absent.

In some embodiments, L₂ is absent.

In some embodiments, L₁ is a linking group.

In some embodiments, L₂ is a linking group.

In some embodiments, the linking group is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the alkylene chain is a C₁-C₂₄ alkylene chain. In some embodiments, the alkylene chain is a C₁-C₁₈ alkylene chain. In some embodiments, the alkylene chain is a C₁-C₁₂ alkylene chain. In some embodiments, the alkylene chain is a C₁-C₁₀ alkylene chain. In some embodiments, the alkylene chain is a C₁-C₈ alkylene chain. In some embodiments, the alkylene chain is a C₁-C₆ alkylene chain. In some embodiments, the alkylene chain is a C₁-C₄ alkylene chain. In some embodiments, the alkylene chain is a C₁-C₂ alkylene chain. In some embodiments, the alkylene chain is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, 4- to 6-membered heterocyclyl, or a combination thereof. In some embodiments, the alkylene chain is interrupted by, and/or terminates (at either or both termini) with —N(R′)—. In some embodiments, the alkylene chain is interrupted by, and/or terminates (at either or both termini) with —C(O)—. In some embodiments, the alkylene chain is interrupted by, and/or terminates (at either or both termini) with —C(O)O—. In some embodiments, the alkylene chain is interrupted by, and/or terminates (at either or both termini) with —C(O)N(R′)—. In some embodiments, the alkylene chain is interrupted by, and/or terminates (at either or both termini) with —N(R′)S(O)₂—. In some embodiments, the alkylene chain is interrupted by, and/or terminates (at either or both termini) with a 4- to 6-membered heterocyclyl.

In some embodiments, the linking group is a polyethylene glycol chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the polyethylene glycol chain has 1 to 20 —(CH₂CH₂—O)— units. In some embodiments, the polyethylene glycol chain has 1 to 15 —(CH₂CH₂—O)— units. In some embodiments, the polyethylene glycol chain has 1 to 10 —(CH₂CH₂—O)— units. In some embodiments, the polyethylene glycol chain has 1 to 6 —(CH₂CH₂—O)— units. In some embodiments, the polyethylene glycol chain has 1 to 2 —(CH₂CH₂—O)— units. In some embodiments, the polyethylene glycol is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, 4- to 6-membered heterocyclyl, or a combination thereof. In some embodiments, the polyethylene glycol chain is interrupted by, and/or terminates (at either or both termini) with —N(R′)—. In some embodiments, the polyethylene glycol chain is interrupted by, and/or terminates (at either or both termini) with —C(O)—. In some embodiments, the polyethylene glycol chain is interrupted by, and/or terminates (at either or both termini) with —C(O)O—. In some embodiments, the polyethylene glycol chain is interrupted by, and/or terminates (at either or both termini) with —C(O)N(R′)—. In some embodiments, the polyethylene glycol chain is interrupted by, and/or terminates (at either or both termini) with —N(R′)S(O)₂—. In some embodiments, the polyethylene glycol chain is interrupted by, and/or terminates (at either or both termini) with 4- to 6-membered heterocyclyl.

In some embodiments, R₁ is methyl.

In some embodiments, R_{1′} and R₁, together with the nitrogen atom to which they are bound, form a piperidinyl ring.

In some embodiments, R_{1′} and R₁, together with the nitrogen atom to which they are bound, form a piperazinyl ring.

In some embodiments, two R₁ groups, together with the nitrogen atom to which they are bound, form a piperidinyl ring.

In some embodiments, two R₁ groups, together with the nitrogen atom to which they are bound, form a piperazinyl ring.

In some embodiments, R₂ is bromo.

In some embodiments, R₂ is chloro.

In some embodiments, R₂ is fluoro.

In some embodiments, R₂ is iodo.

In some embodiments, R₂ is alkoxy, e.g., C₁-C₆ alkoxy. In some embodiments, R₂ is C₁-C₃ alkoxy. In some embodiments, R₂ is methoxy.

R₃ is a leaving group, which as used herein, refers to an atom or group of atoms that detaches from the compound during a reaction.

In some embodiments, R₃ is halo. In some embodiments, R₃ is iodo. In some embodiments, R₃ is bromo. In some embodiments, R₃ is chloro. In some embodiments, R₃ is fluoro.

In some embodiments, R₃ is a perfluoroalkylsulfonate (e.g., triflate). In some embodiments, R₃ is a tosylate, mesylate, or a similar sulfonate. In some embodiments, R₃ is a phosphate or another inorganic ester. In some embodiments, R₃ is a carboxylate. In some embodiments, R₃ is a carbamate. In some embodiments, R₃ is phenoxide or a substituted phenoxide.

In some embodiments, R₃ is OC(O), C(O)O, OC(O)O, OC(O)NR′, NR′C(O)O, S(O), S(O)₂, OS(O)₂, S(O)₂O, or OP(OR′)O₂, wherein each R′ is independently hydrogen, (C₁-C₆) alkyl, (C₃-C₁₀) carbocyclyl, 4- or 7-membered heterocyclyl, and wherein said alkyl, carbocyclyl, or heterocyclyl is optionally substituted. In some embodiments, R₃ is OC(O)NMe.

Broadly, R₄ is a ligand that binds a cellular protein or a nucleic acid (e.g., DNA and RNA). Examples of proteins that may be targeted include known (validated) or putative targets for therapeutic intervention. The cellular protein may be enzymatic or non-enzymatic. In some embodiments, R₄ is a ligand that binds a cellular protein other than a cellular enzyme that catalyzes degradation of cellular proteins (such as ubiquitin ligases). Representative examples of cellular proteins that may be targeted by binding ligand of the disclosed compounds that contain a binding ligand include kinases, BET bromodomain-containing protein, cytosolic signaling proteins (e.g., FKBP12), nuclear proteins, histone deacetylases (HDAC), lysine methyltransferase, aryl hydrocarbon receptors (AHR), estrogen receptors, androgen receptors, glucocorticoid receptors, and transcription factors (e.g., SMARCA4, SMARCA2, TRIM24).

In certain embodiments, R₄ is a ligand that binds a tyrosine kinase (e.g., AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES, FGFR1, FGFR2, FGFR3, FGFR4, FGR, FLT1, FLT3, FLT4, FRK, FYN, GSG2, HCK, IGF1R, ILK, INSR, INSRR, IRAK4, ITK, JAK1, JAK2, JAK3, KDR, KIT, KSR1, LCK, LMTK2, LMTK3, LTK, LYN, MATK, MERTK, MET, MLTK, MST1R, MUSK, NPR1, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYK1, SYK, TEC, TEK, TEX14, TIE1, TNK1, TNK2, TNNI3K, TXK, TYK2, TYRO3, YES1, or ZAP70), a serine/threonine kinase (e.g., casein kinase 2, protein kinase A, protein kinase B, protein kinase C, Rafkinases, CaM kinases, AKT1, AKT2, AKT3, ALK1, ALK2, ALK3, ALK4, Aurora A, Aurora B, Aurora C, CHK1, CHK2, CLK1, CLK2, CLK3, DAPK1, DAPK2, DAPK3, DMPK, ERK1, ERK2, ERK5, GCK, GSK3, HIPK, KHS1, LKB1, LOK, MAPKAPK2, MAPKAPK, MNK1, MSSK1, MST1, MST2, MST4, NDR, NEK2, NEK3, NEK6, NEK7, NEK9, NEK11, PAK1, PAK2, PAK3, PAK4, PAK5, PAK6, PIM1, PINM2, PLK1, RIP2, RIP5, RSK1, RSK2, SGK2, SGK3, SIK1, STK33, TAO1, TAO2, TGF-beta, TLK2, TSSK1, TSSK2, ULK1, or ULK2), a cyclin dependent kinase (e.g., Cdk1-Cdk11), or a leucine-rich repeat kinase (e.g., LRRK2).

In certain embodiments, R₄ is a ligand that binds a bromodomain and extraterminal (BET) protein, representative examples of which include ATPase family AAA domain-containing protein 2 (ATAD2), bromodomain adjacent to zinc finger domain protein 1A (BAZ1A), BAZ1B, BAZ2A, BAZ2B, bromodomain containing protein 1 (BRD1), BRD2, BRD3, BRD4, BRD5, BRD6, BRD7, BRD8, BRD9, BRD10, bromodomain testis-specific protein (BRDT), romodomain and PHD finger-containing protein 1 (BRPF1), BRPF3, bromodomain And WD Repeat Domain Containing 3 (BRWD3), cat eye syndrome critical region protein 2 (CECR2), CREB binding protein (CREBBP), E1A binding protein P300 (EP300), general control of amino-acid synthesis 5-like 2 (GCN5L2), histone-lysine N-methyltransferase 2A (KMT2A), P300/CBP-associated factor (PCAF), PH-interacting protein (PHIP), protein kinase C binding protein 1 (PRKCBP1), SWI/SNF Related, Matrix Associated, Actin Dependent Regulator Of Chromatin, Subfamily A, Member 2 (SMARCA2), SMARCA4, Sp100 nuclear body protein (SP100), SP110, SP140, transcription initiation factor TFIID subunit 1 (TAF1), TAF1 μL, TIF1a, tripartite motif-containing 28 (TRIM28), TRIM33, TRIM66, WD repeat protein 9 (WDR9), zinc finger MYND domain-containing protein 11 (ZMYND11), and mixed lineage leukemia-like protein 4 (MLL4). In certain embodiments, the BET bromodomain-containing protein is BRD4.

In certain embodiments, R₄ is a ligand that binds to BRD2, BRD3, BRD4, Antennapedia Homeodomain Protein, BRCA1, BRCA2, a CCAAT-Enhanced-Binding Protein, histone, a Polycomb-group protein, a High Mobility Group Protein, a Telomere Binding Protein, FANCA, FANCD2, FANCE, FANCF, HDAC1, HDAC2, HDAC3, HDAC4, HDAC5, HDAC6, HDAC7, HDAC8, HDAC9, HDAC10, HDAC11, a hepatocyte nuclear factor, Mad2, NF-kappa B, a Nuclear Receptor Coactivator, CREB-binding protein, p55, p107, p130, p53, c-fos, c-jun, c-mdm2, c-myc, or c-rel.

In some embodiments, R₄ is a ligand that binds to BRD. Representative examples of small molecules that bind BRD include:

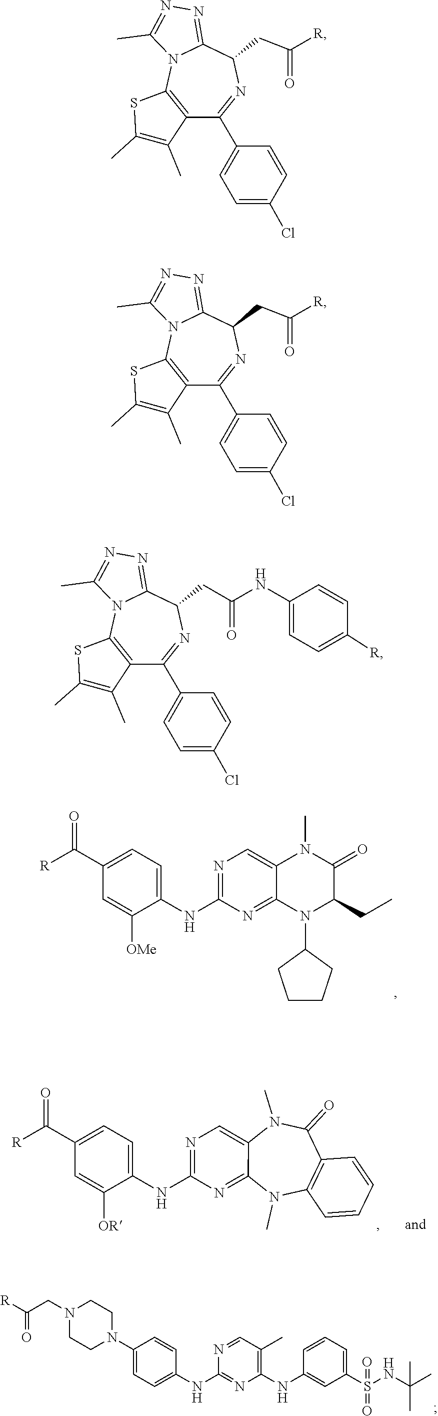

wherein:
R is point of attachment; and
R′ is methyl or ethyl.

In some embodiments, R₄ is a ligand that binds to CREBBP. Representative examples of small molecules that bind CREBBP include:

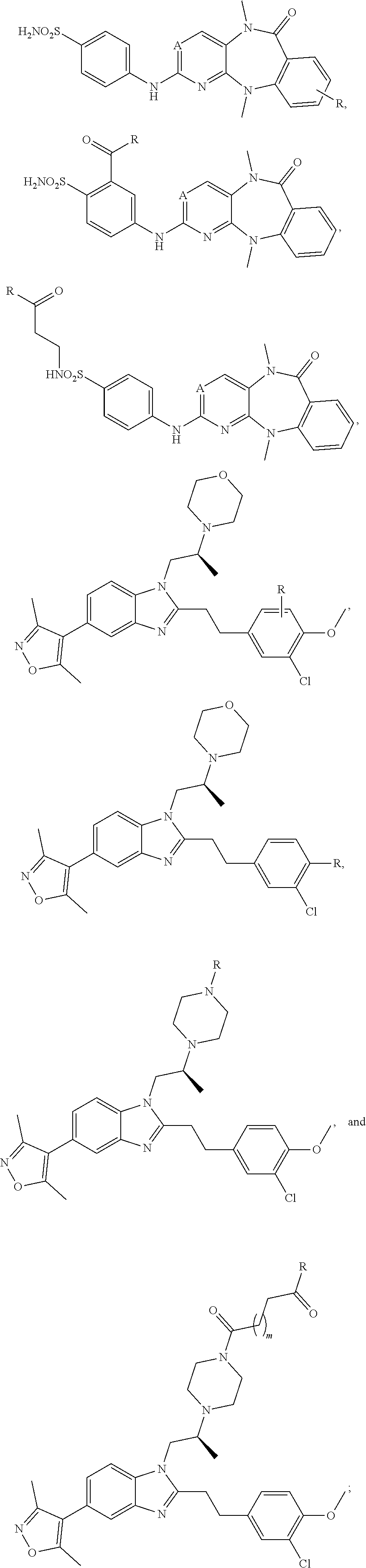

wherein:
R is the point of attachment;
A is N or CH; and
m is 0, 1, 2, 3, 4, 5, 6, 7, or 8.

In some embodiments, R₄ is a ligand that binds to SMARCA4/PB1/SMARCA2.

Representative examples of small molecules that bind SMARCA4/PB1/SMARCA2 include:

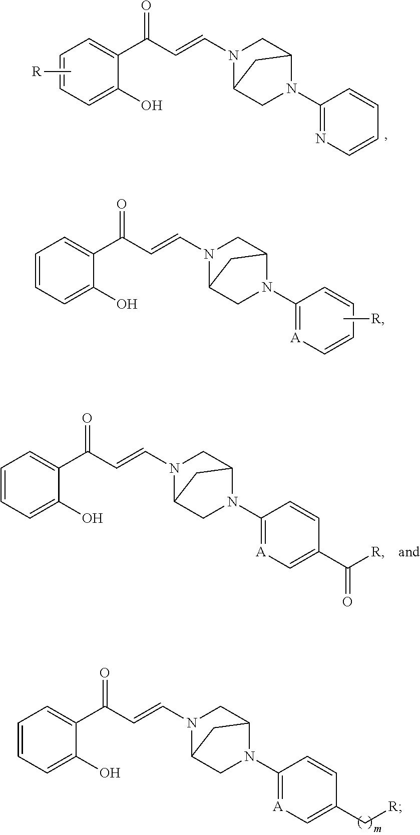

wherein:
R is the point of attachment;
A is N or CH; and
m is 0, 1, 2, 3, 4, 5, 6, 7, or 8.

In some embodiments, R₄ is a ligand that binds to TRIM24/BRPF1. Representative examples of small molecules that bind TRIM24/BRPF1 include:

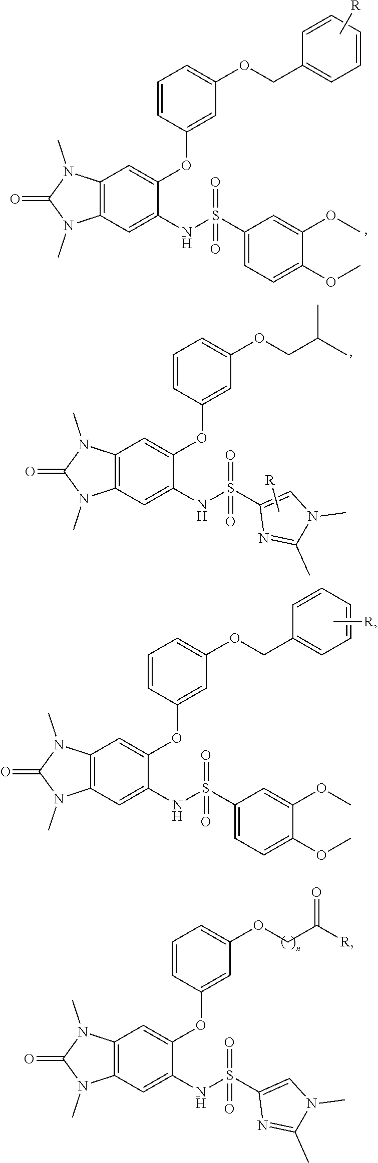

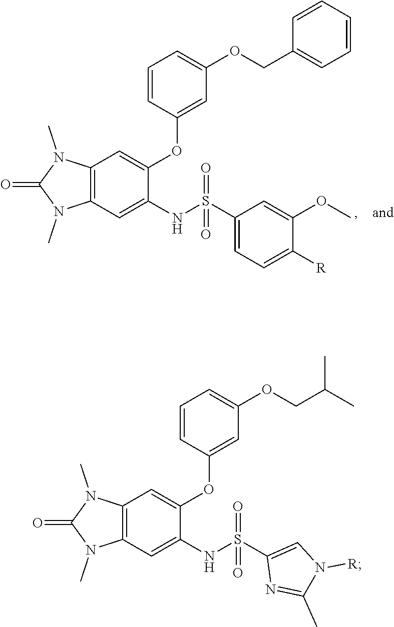

wherein:
R is the point of attachment; and
m is 0, 1, 2, 3, 4, 5, 6, 7, or 8.

In some embodiments, R₄ is a ligand that binds to a glucocorticoid receptor. Representative examples of small molecules that bind a glucocorticoid receptor include:

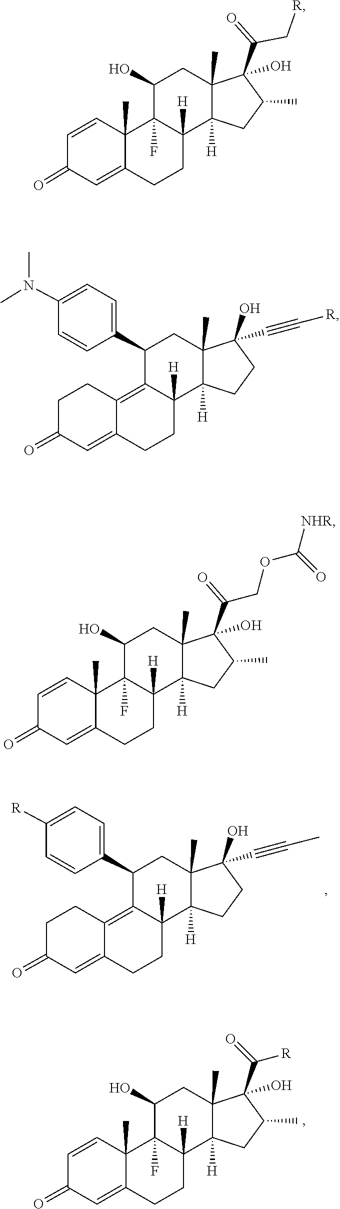

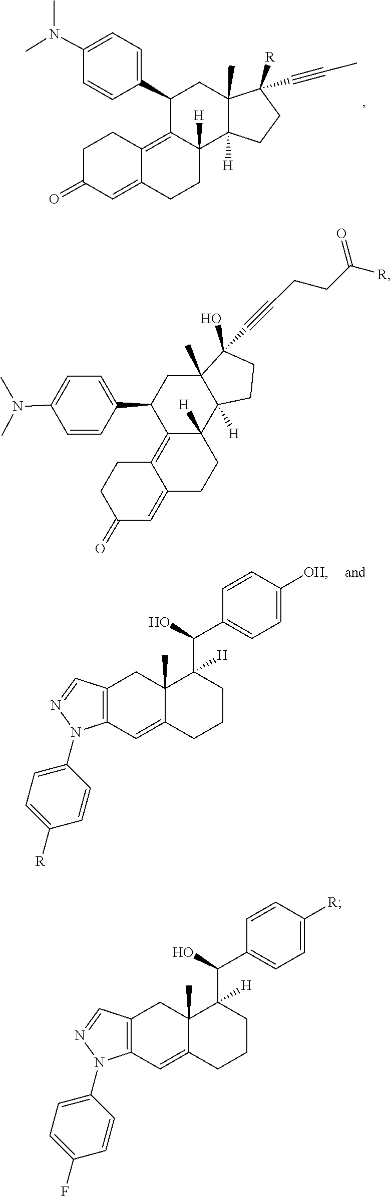

wherein:
R is the point of attachment.

In some embodiments, R₄ is a ligand that binds to an estrogen/androgen receptor. Representative examples of small molecules that bind an estrogen/androgen receptor include:

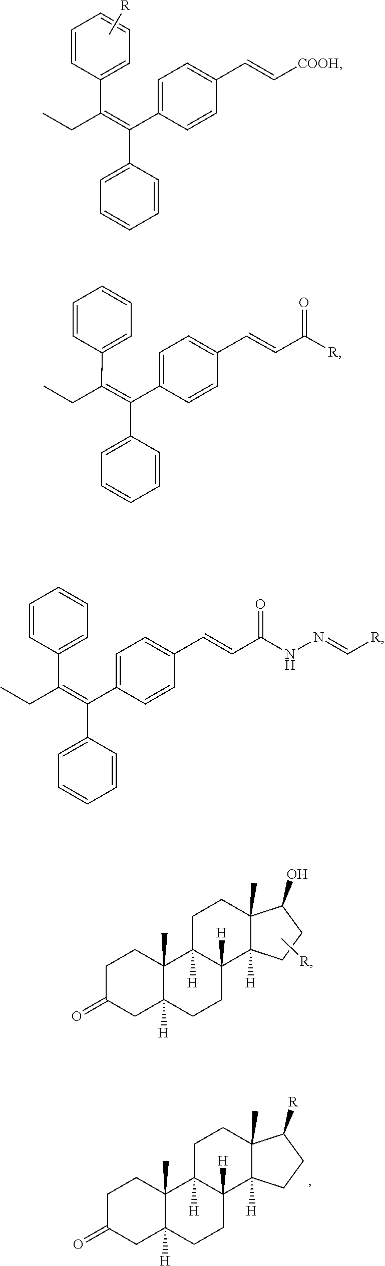

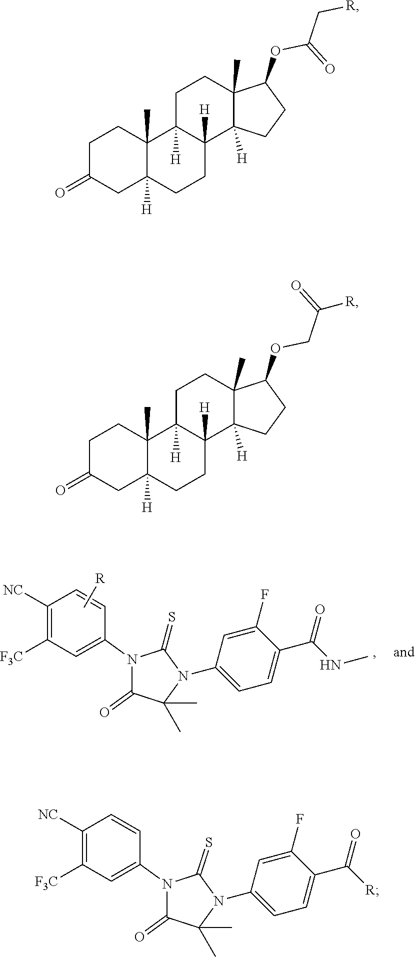

wherein:
R is the point of attachment.

In some embodiments, R₄ is a ligand that binds to DOT1 μL. Representative examples of small molecules that bind DOT1 μL:

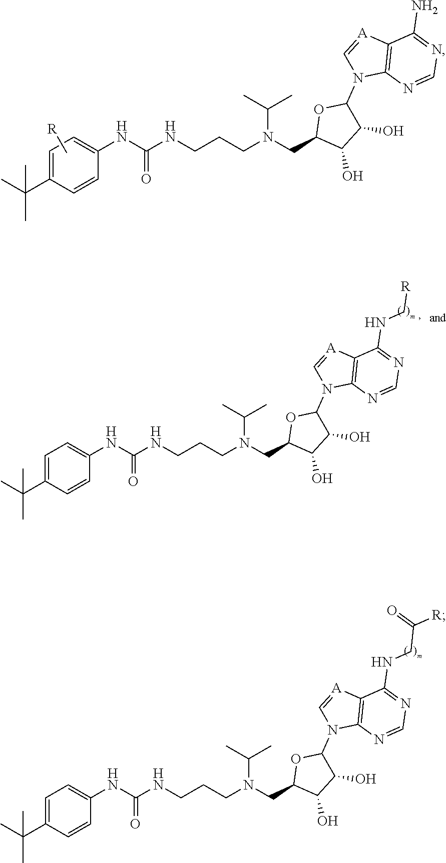

wherein:
R is the point of attachment;
A is N or CH; and
m is 0, 1, 2, 3, 4, 5, 6, 7, or 8.

In some embodiments, R₄ is a ligand that binds to Ras. Representative examples of small molecules that bind Ras:

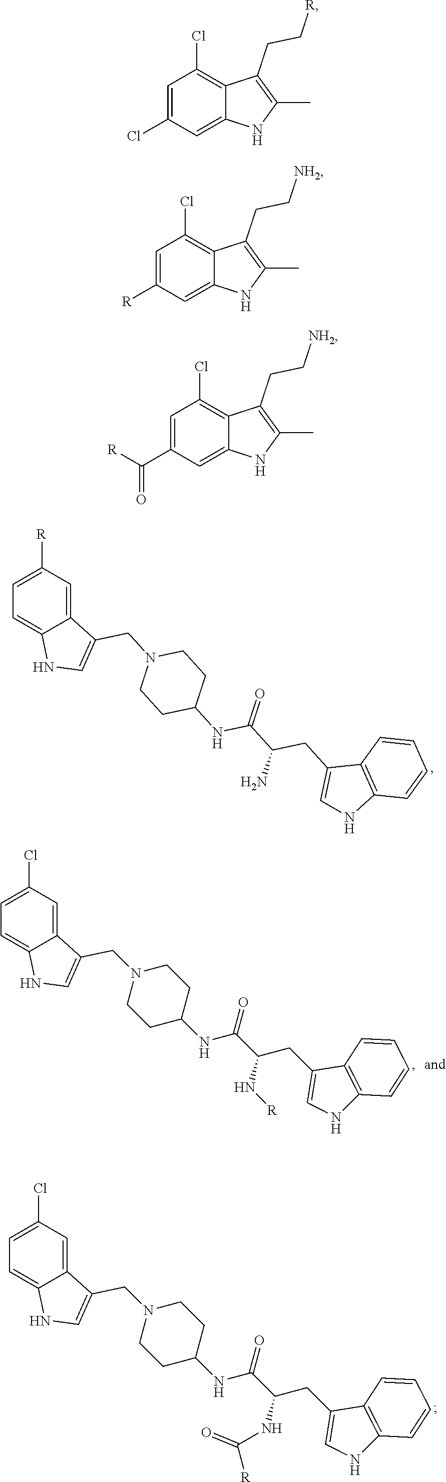

wherein:
R is the point of attachment.

In some embodiments, R₄ is a ligand that binds to RasG12C. Representative examples of small molecules that bind RasG12C:

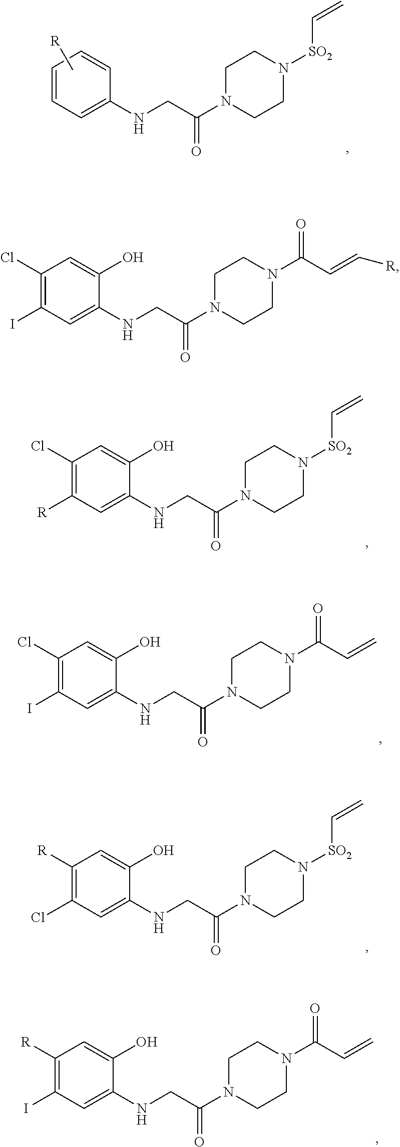

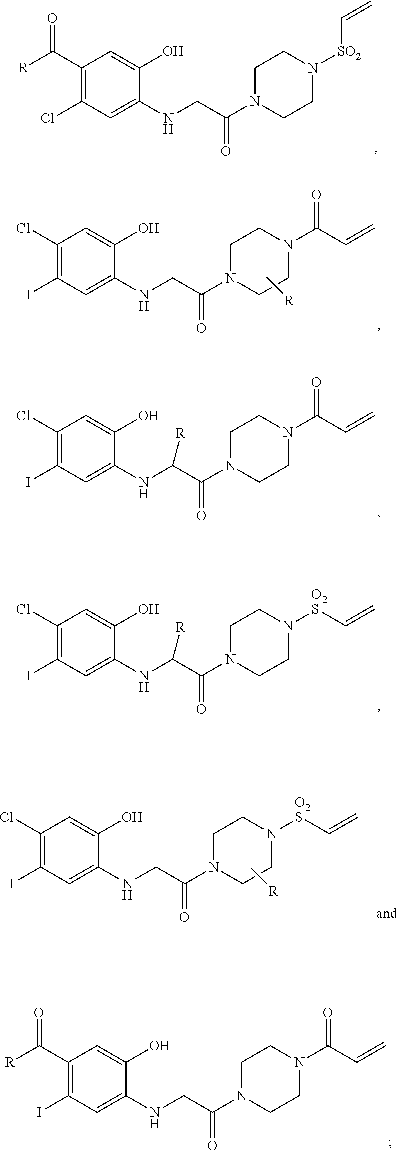

wherein:
R is the point of attachment.

In some embodiments, R₄ is a ligand that binds to Her3. Representative examples of small molecules that bind Her3:

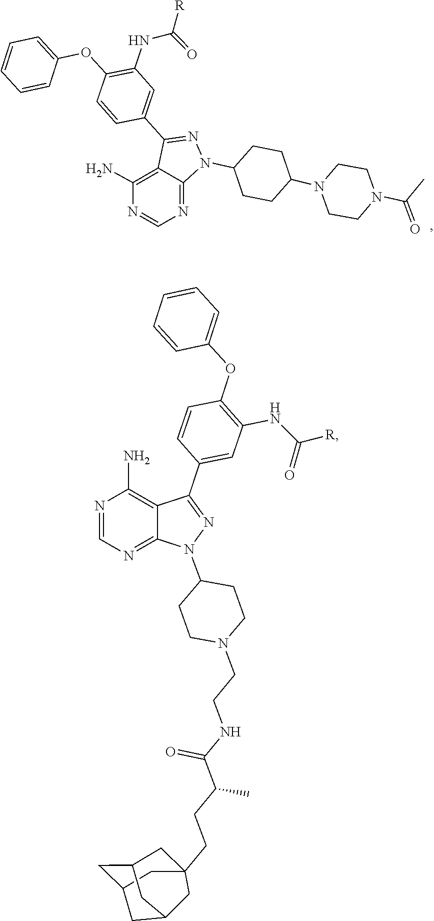

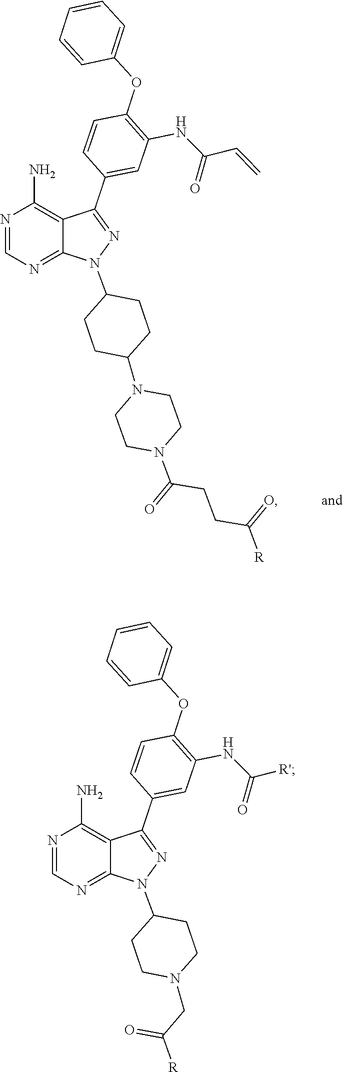

wherein:
R is the point of attachment; and
R′ is

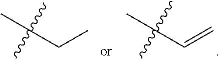

In some embodiments, R₄ is a ligand that binds to Bcl-2/Bcl-XL. Representative examples of small molecules that bind Bcl-2/Bcl-XL:

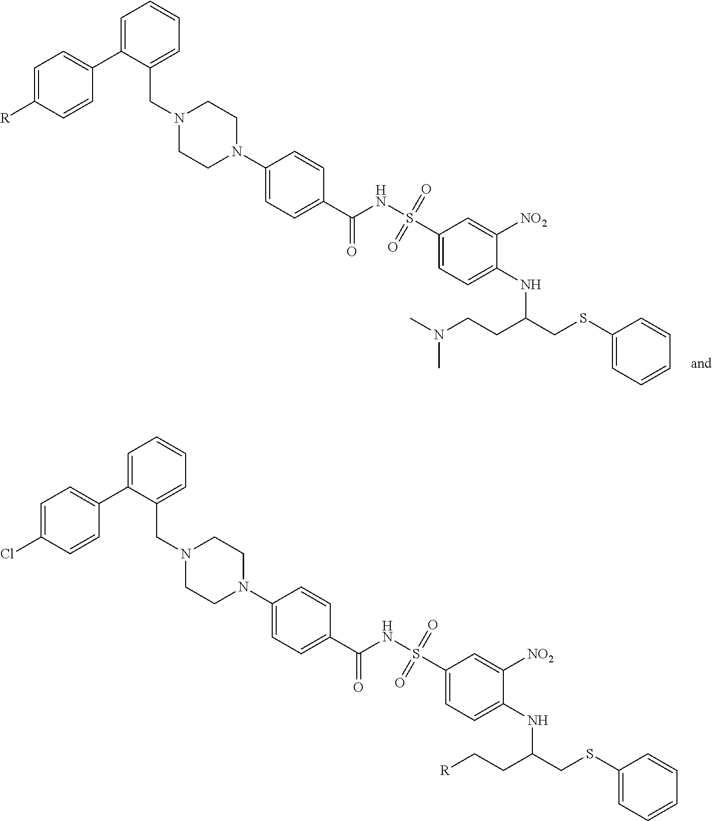

wherein:
R is the point of attachment.

In some embodiments, R₄ is a ligand that binds to HDAC. Representative examples of small molecules that bind HDAC:

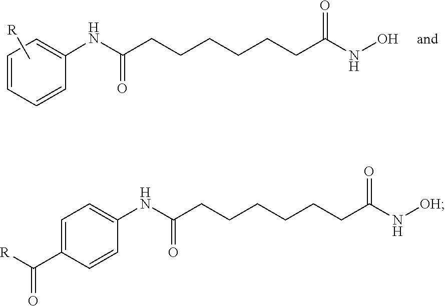

wherein:
R is the point of attachment.

In some embodiments, R₄ is a ligand that binds to PPAR-gamma. Representative examples of small molecules that bind PPAR-gamma:

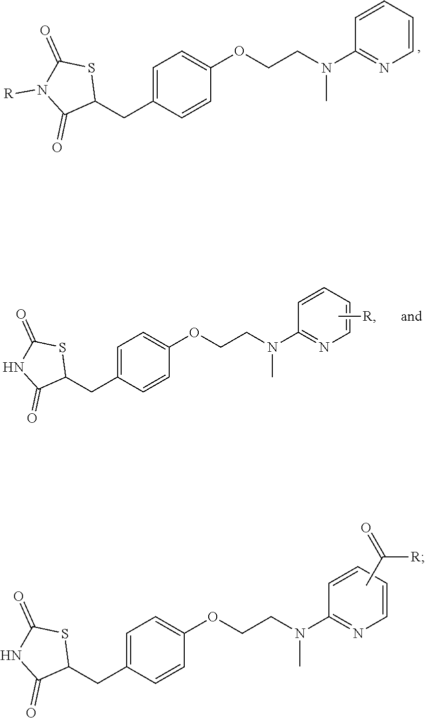

wherein:
R is the point of attachment.

In some embodiments, R₄ is a ligand that binds to RXR. Representative examples of small molecules that bind RXR:

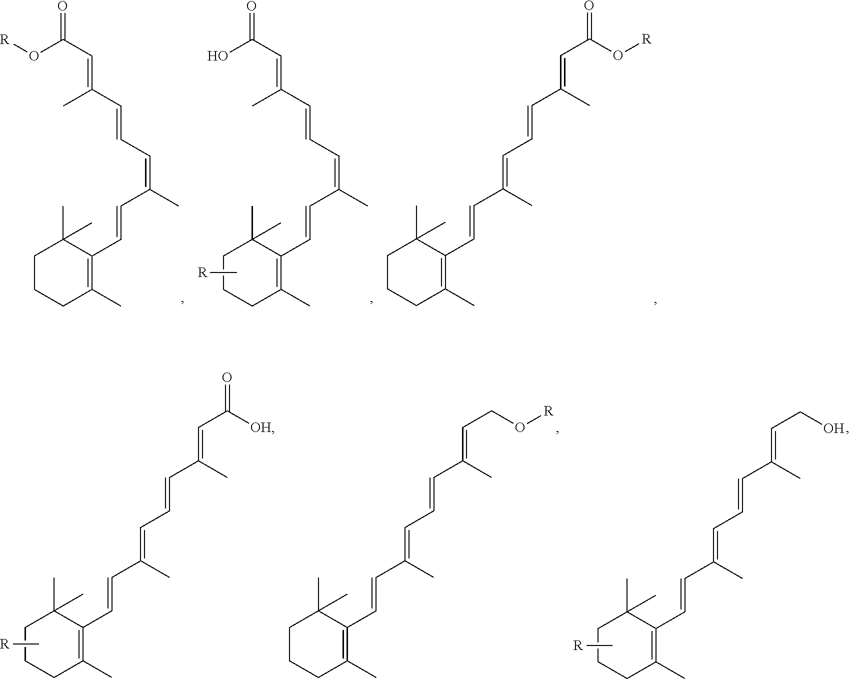

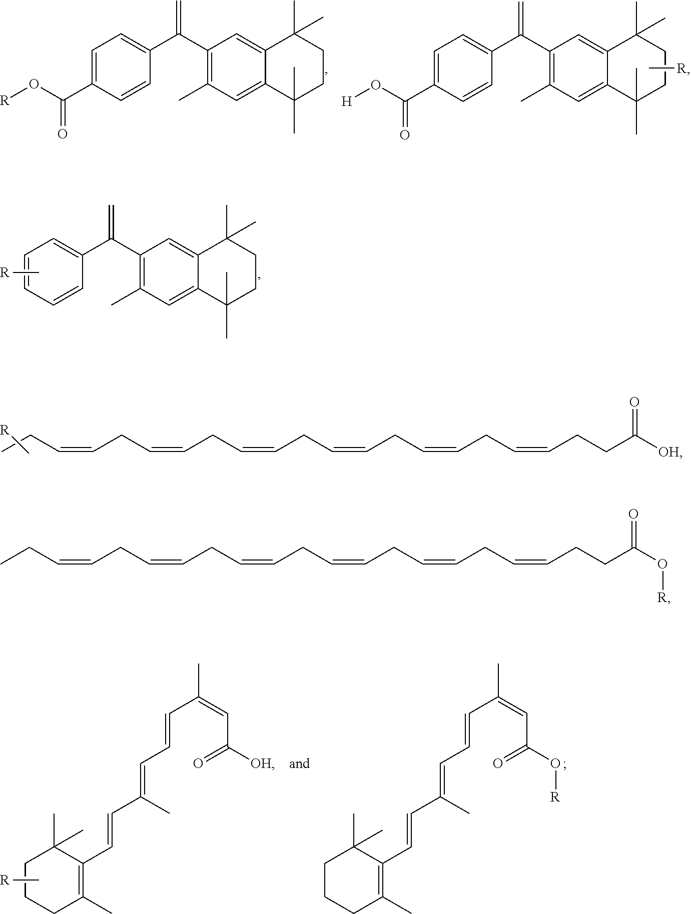

wherein:
R is the point of attachment.

In some embodiments, R₄ is a ligand that binds to DHFR. Representative examples of small molecules that bind DHFR:

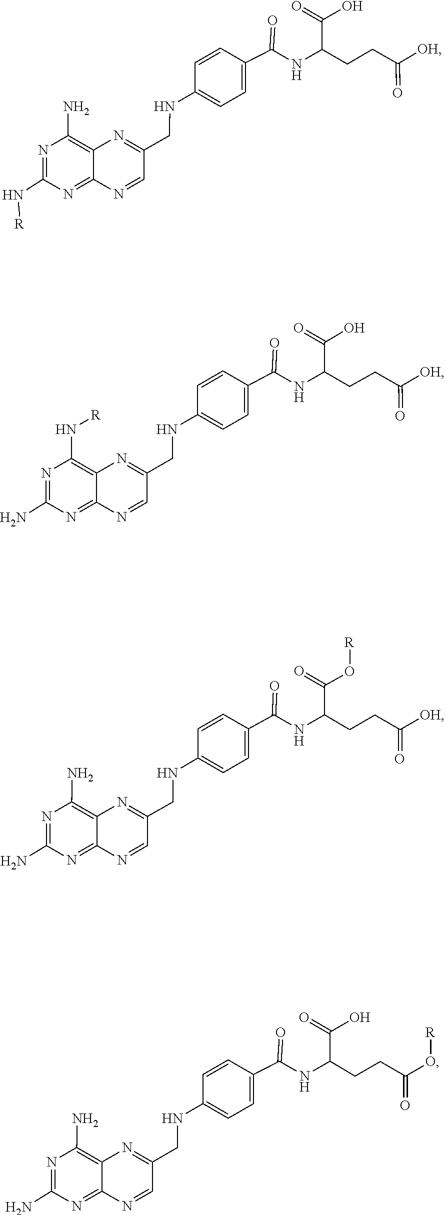

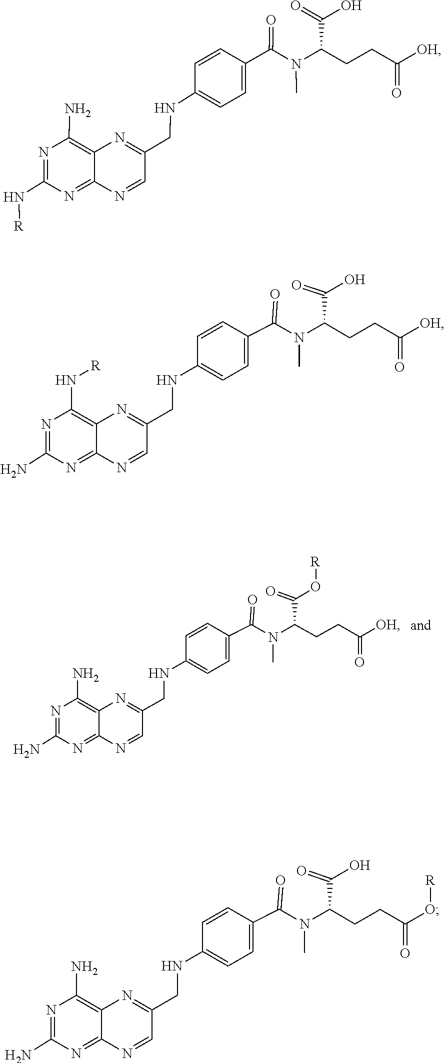

wherein:
R is the point of attachment.

In some embodiments, R₄ is a ligand that binds to BCL2. Representative examples of small molecules that bind BCL2:

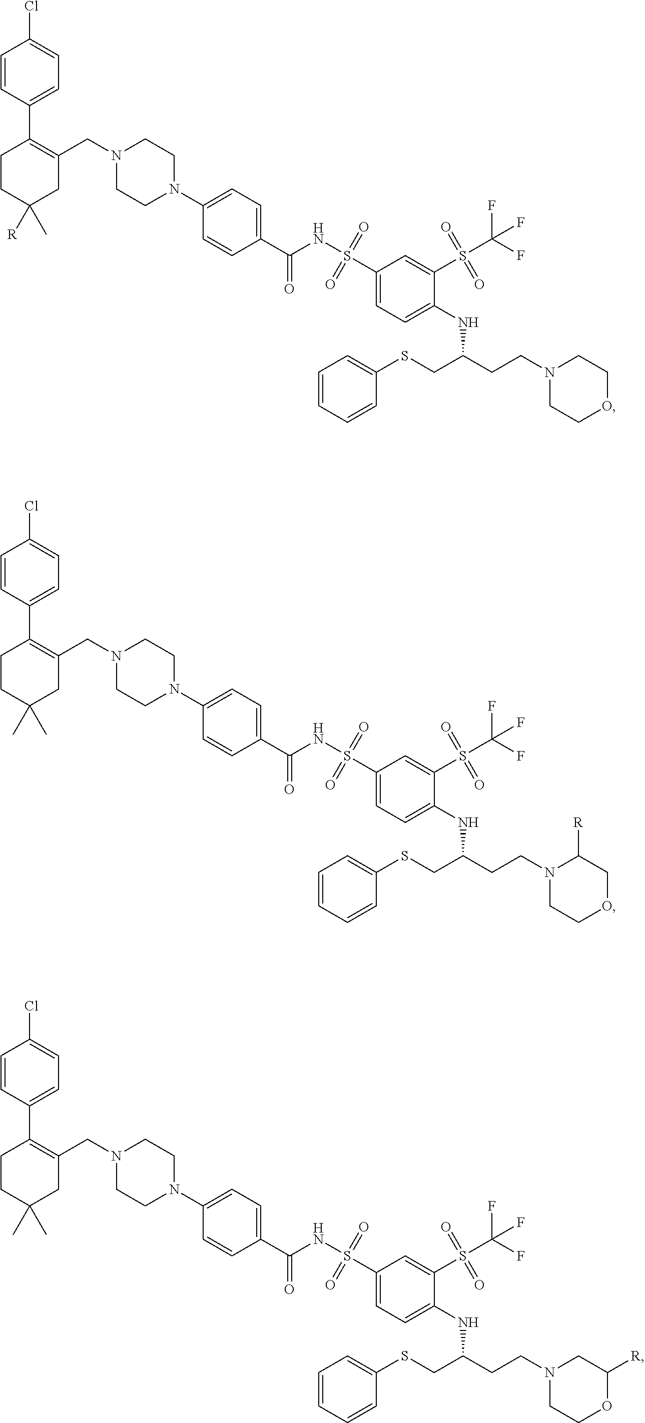

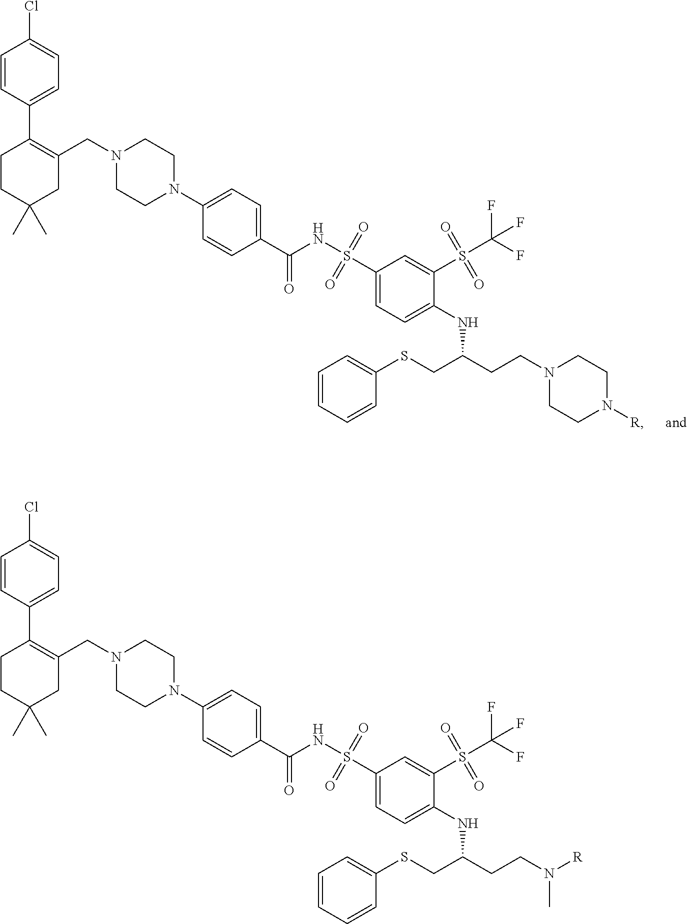

wherein:
R is the point of attachment.

Yet other small molecules that bind cellular proteins and which may be suitable for use as binding ligands in the present disclosure are disclosed in U.S. Patent Application Publication Nos. 2017/0121321 and 2014/0356322.

In some embodiments, R₄ is a ligand that binds cereblon (CRBN). Representative examples of small molecules that bind CRBN are represented by any one of structures (D1-a) to (D1-d):

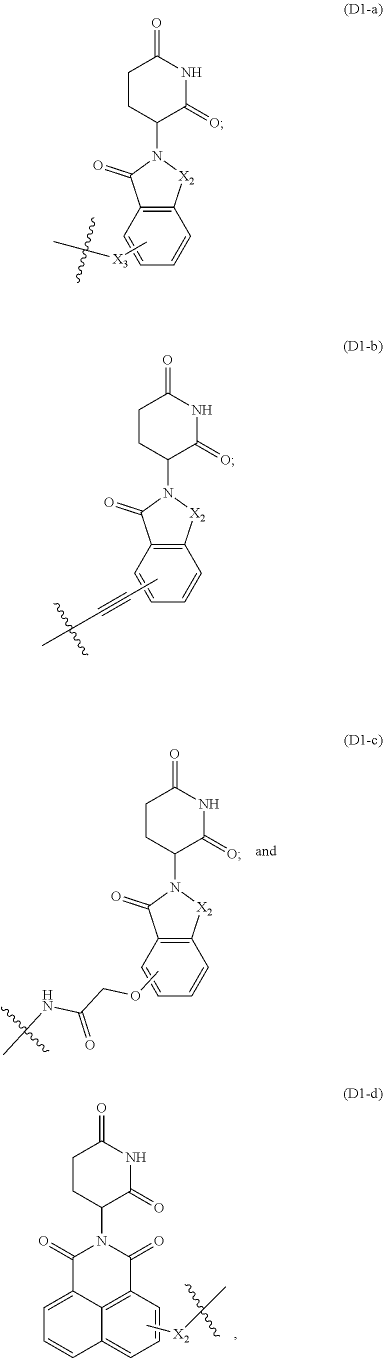

wherein X₂ is CH₂ or C(O) and X₃ is CR″₁R″₂, NR″₁, O, or S, wherein R″₁ and R″₂ are independently hydrogen, halogen, OH, NH₂, C₁-C₃ alkyl, C₁-C₃ alkoxy, or C₁-C₃ alkylamine, or R″₁ and R″₂, together with the atoms to which they are bound, form a C₃-C₇ carbocyclic or C₃-C₇ heterocyclic ring (e.g., azetidine, piperidine, pyrrolidine, cyclobutane, cyclohexane).

Yet other small molecules that bind cereblon and which may be suitable for use as binding ligands in the present disclosure are disclosed in U.S. Pat. No. 9,770,512, and U.S. Patent Application Publication Nos. 2018/0015087, 2018/0009779, 2016/0243247, 2016/0235731, 2016/0235730, and 2016/0176916, and International Patent Publications WO 2017/197055, WO 2017/197051, WO 2017/197036, WO 2017/197056 and WO 2017/197046.

In some embodiments, R₄ is a ligand that binds von Hippel-Lindau (VHL) tumor suppressor. Representative examples of small molecules that bind VHL are represented by any one of structures (D2-a) to (D2-j):

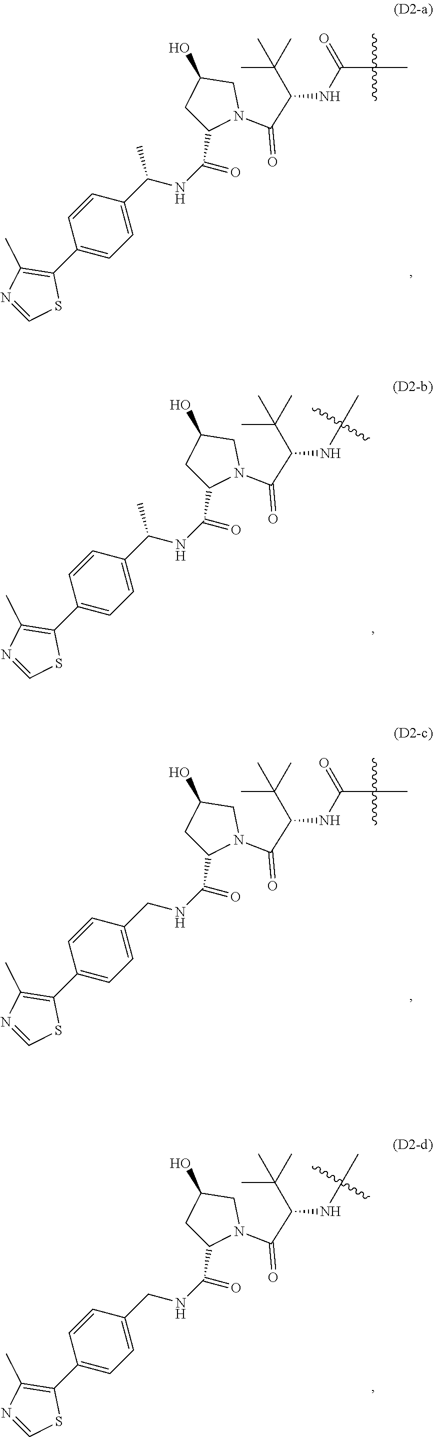

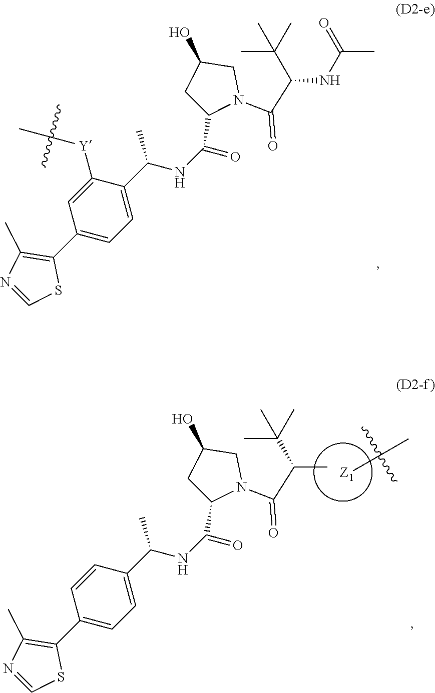

wherein Z₁ is a C₅-C₆ carbocyclic or C₅-C₆ heterocyclic group,

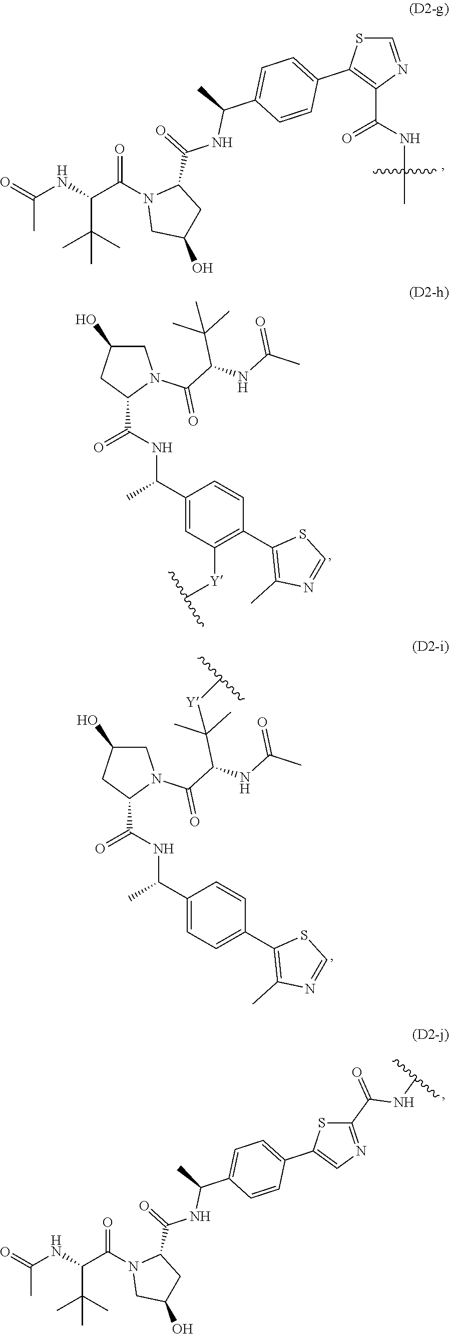

Y′ is a bond, CH₂, NH, NMe, O, or S, or a stereoisomer thereof.

In some embodiments, Zi is phenyl, pyrrolyl, furanyl, thiophenyl, pyrazolyl, imidazolyl, oxazolyl, thiazolyl, pyridinyl, pyridazinyl, or pyrimidinyl. In certain embodiments, Z is

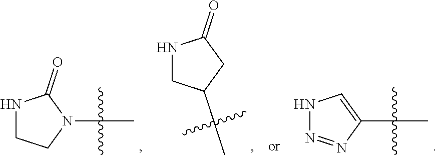 [SMILES: *N1CCNC1=O ; *C1CNC(=O)C1 ; *c1c[nH]nn1]

Yet other small molecules that bind VHL and which may be suitable for use as binding ligands in the present disclosure are disclosed in U.S. Patent Application Publication Nos. 2017/0121321 and 2014/0356322.

In some embodiments, R₄ is a ligand that binds an inhibitor of apoptosis protein (IAP). Representative examples of small molecules that bind IAP are represented by any one of structures (D3-a) to (D3-f):

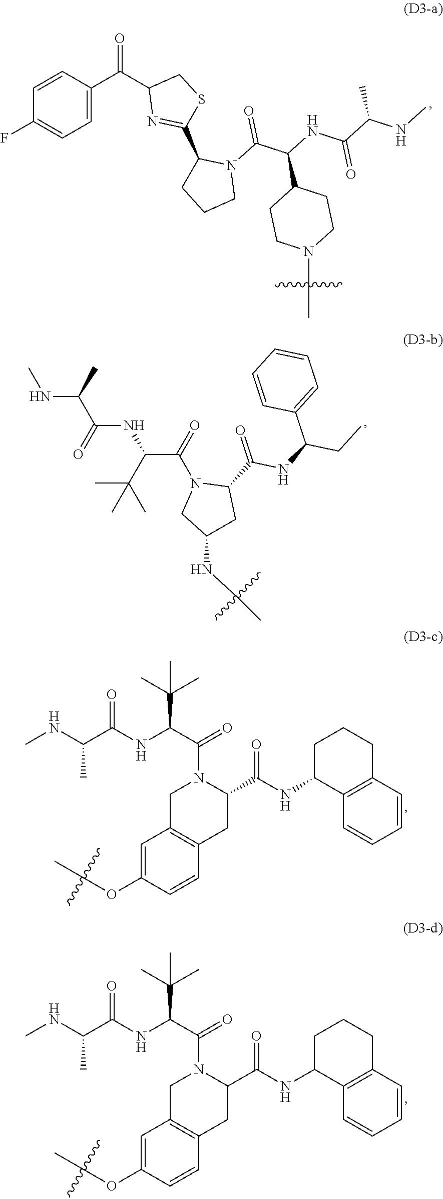

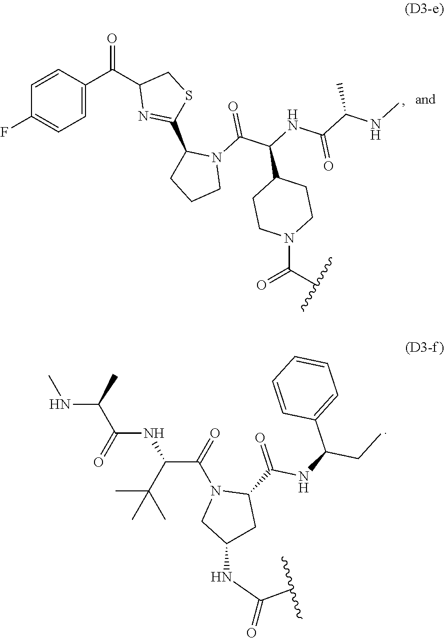

Yet other small molecules that bind IAP and which may be suitable for use as binding ligands in the present disclosure are disclosed in International Patent Application Publication Nos. WO 2008128171, WO 2008/016893, WO 2014/060768, and WO 2014/060767.

In some embodiments, R₄ is a ligand that binds murine double minute 2 (MDM2). Representative examples of small molecules that bind MDM2 are represented by structures (D4-a) and (D4-b):

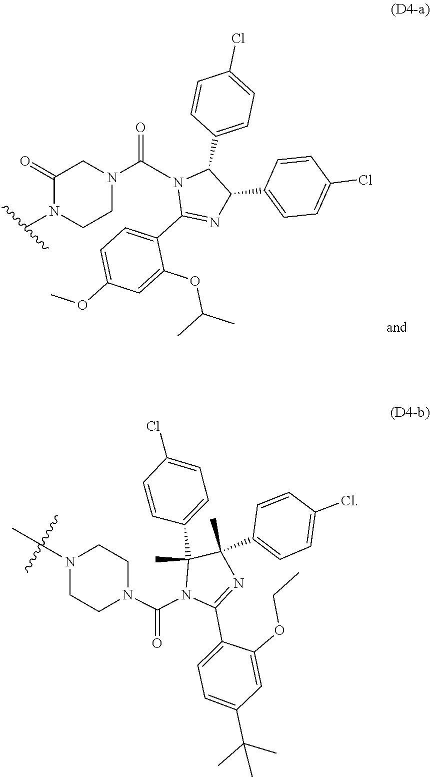

Yet other small molecules that bind MDM2 and which may be suitable for use as binding ligands in the present disclosure are disclosed in U.S. Pat. No. 9,993,472 B2. MDM2 is known in the art to function as an ubiquitin-E3 ligase.

In some embodiments, R₄ is a ligand that binds receptor RPN13. Representative examples of small molecules that bind RPN13 are represented by structures (D5-a), (D5-b), (D5-c), and (D5-d):

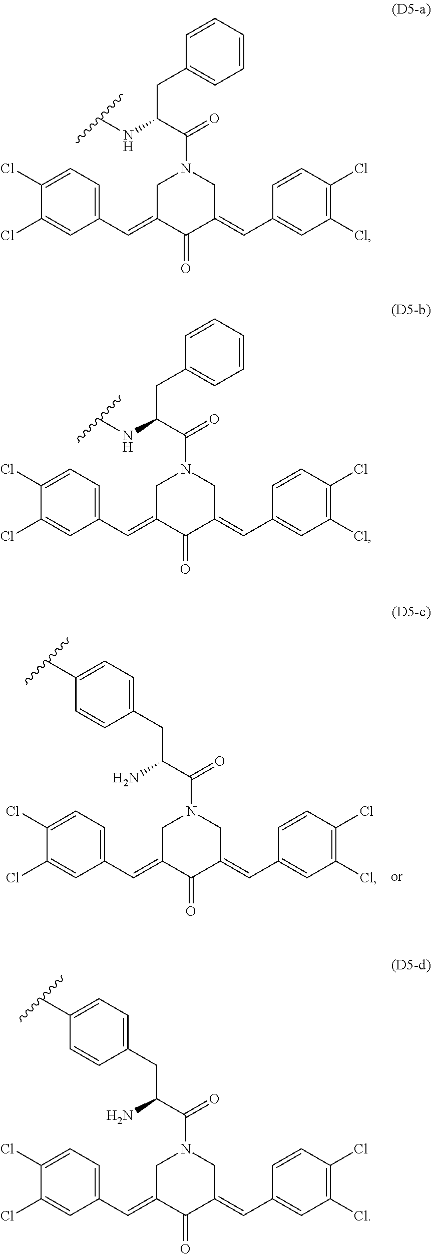 [SMILES: *N[C@H](Cc1ccccc1)C(=O)N1C/C(=C\c2ccc(Cl)c(Cl)c2)C(=O)/C(=C/c2ccc(Cl)c(Cl)c2)C1 ; *N[C@@H](Cc1ccccc1)C(=O)N1C/C(=C\c2ccc(Cl)c(Cl)c2)C(=O)/C(=C/c2ccc(Cl)c(Cl)c2)C1 ; *c1ccc(C[C@@H](N)C(=O)N2C/C(=C\c3ccc(Cl)c(Cl)c3)C(=O)/C(=C/c3ccc(Cl)c(Cl)c3)C2)cc1 ; "or" ; *c1ccc(C[C@H](N)C(=O)N2C/C(=C\c3ccc(Cl)c(Cl)c3)C(=O)/C(=C/c3ccc(Cl)c(Cl)c3)C2)cc1]

Yet other small molecules that bind RPN13 and which may be suitable for use as binding ligands in the present disclosure are disclosed in International Publication No. PCT/US2020/012825. RPN13 is known in the art to function as an ubiquitin receptor.

In some embodiments, R₄ is short peptide sequence (e.g., 2 to 50 amino acids in length, e.g., 4 to 20 amino acids in length, wherein the amino acid residues in the peptide may be the same or different).

In some embodiments, R₄ is a therapeutic moiety. Such moieties include, for example, drugs or active agents that have been clinically validated and drug candidates. The therapeutic moiety may, in some embodiments, be a small molecule. In certain embodiments, the molecular weight of the small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of the small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. In certain embodiments, therapeutic moiety is a therapeutically active agent such as a drug (e.g., a molecule approved by the U.S. Food and Drug Administration as provided in the Code of Federal Regulations (C.F.R.)).

In some embodiments, the therapeutic moiety is an anti-cancer agent. Representative types of anti-cancer agents include anti-angiogenic agents, alkylating agents, antimetabolites, microtubulin polymerization perturbers, platinum coordination complexes, anthracenediones, substituted ureas, methylhydrazine derivatives, adrenocortical suppressants, hormones and antagonists, anti-cancer polysaccharides and anthracycline (e.g., an aclarubicin, daunorubicin, doxorubicin, epirubicin, idarubicin, mitoxantrone, pirarubicin, valrubicine and derivatives and analogs thereof), and kinase inhibitors (e.g., pan-Her inhibitors (e.g., HKI-272, BIBW-2992, PF299, SN29926 and PR-509E)).

Representative examples of anti-cancer agents include alkylating agents (e.g., busulfan, chlorambucil, cyclophosphamide, ifosfamide, mechlorethamine, melphalan, carmustine, streptozocin, dacarbazine, temozolomide, altretamine, and thioTEPA), antimetabolites (e.g., capecitabine, cytarabine, 5′-fluorouracil, gemcitabine, cladribine, fludarabine, 6-mercaptopurine, and pentostatin), folate antagonists (e.g., methotrexate and pemetrexed), mitotic inhibitors (e.g., ocetaxel, paclitaxel, vinblastine, vincristine, vindesine, and vinorelbine), DNA inhibitors (e.g., hydroxyurea, carboplatin, cisplatin, oxaliplatin, mitomycin C, and pyrrolobenzodiazepine), topoisomerase inhibitors (e.g., topotecan, irinotecan, daunorubican, doxorubicin, etoposide, teniposide, and mitoxantrone), inducers of DNA breaks (e.g., bleomycin), ozogamicin, vedotin, emtansine, pasudotox, deruxtecan, govitecan, and mafodotin, or derivatives thereof. Other representative examples of anti-cancer agents include afatinib (EGFR, HER2), axitinib (KIT, PDGFRβ, VEGFR1/2/3), bosutinib (ABL), cabozantinib (FLT3, KIT, MET, RET, VEGFR2), ceritinib (ALK), crizotinib (ALK, MET), dabrafenib (ABL), erlotinib (EGFR), ibrutinib (BTK), idelalisib (PI3Kδ), imatinib (KIT, PDGFR, ABL), lapatinib (HER2, EGFR), lenvatinib (VEGFR2), nilotinib (ABL), olaparib (PARP), palbociclib (CDK4, CDK6), panobinostat (HDAC), pazopanib (VEGFR, PDGFR, KIT), ponatinib (ABL, FGFR1-3, FLT3, VEGFR2), regorafenib (KIT, PDGFRβ, RAF, RET, VEGFR1/2/3), romidepsin (HDAC), ruxolitinib (JAK1/2), sorafenib (VEGFR, PDGFR, KIT, RAT), temsirolimus (mTOR), trametinib (MEK), vandetanib (EGFR, RET, VEGFR2), vemurafenib (BRAF), vismodegib (PTCH), and vorinostat (HDAC).

In some embodiments, the anti-cancer agent is a kinase inhibitor. Representative examples of kinase inhibitors include abemaciclib, acalabrutinib, afatinib, alectinib, avapritinib, axitinib, baricitinib, binimetinib, bosutinib, brigatinib, cabozantinib, ceritinib, capmatinib, cobimetinib, crizotinib, dabrafenib, dacomitinib, dasatinib, encorafenib, entrectinib, erdafitinib, erlotinib, everolimus, fedratinib, fostamatinib, gefitinib, gilteritinib, ibrutinib, icotinib, imatinib, lapatinib, larotrectinib, lenvatinib, lorlatinib, midostaurin, neratinib, netarsudil, nilotinib, nintedanib, osimertinib, palbociclib, pazopanib, pemigatinib, pexidartinib, ponatinib, pralsetinib, regorafenib, ribociclib, ripretinib, ruxolitinib, selpercatinib, selumetinib, sirolimus, sorafenib, sunitinib, temsirolimus, tofacitinib, trametinib, tucatinib, upadacitinib, vandetanib, vemurafenib, and zanubrutinib.

In some embodiments, the therapeutic moiety is an anti-bacterial agent. Representative examples of antibacterial agents include plazomicin, eravacycline, sarecycline, omadacycline, rifamycin, imipenem, cilastatin, relebactam, pretomanid, lefamulin, cefiderocol, sulfaquinoxaline, oxytetracycline, hygromycin B, tylosin, chlortetracycline, virginiamycin, neomycin, luncomycin, pyrantel, melengestrol, lasalocid, fenbendazole, semduramicin, decoquinate, ractopamine, laidlomycin, diclazuril, halifuginone, robenidine, clopidol, zilpaterol, monensin, zoalene, lubabegron, and bacitracin.

In some embodiments, the therapeutic moiety is a non-steroidal anti-inflammatory drug (NSAID). Representative examples of NSAIDs agents include celecoxib, diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, sulindac, and tolmetin.

In some embodiments, the therapeutic moiety is a corticosteroid. Representative examples of corticosteroids agents include deflazacort, dexamethasone, betamethasone, triamcinolone, hydrocortisone, methylprednisolone and prednisone.

In some embodiments, the therapeutic moiety is a disease-modifying antirheumatic drug (DMARD). Representative examples of DMARDs include hydroxychloroquine, leflunomide, methotrexate, sulfasalazine, minocycline, penicillamine, cyclophosphamide, azathiopurine, cyclosporine, apremilast, and mycophenolate mofetil.

In some embodiments, R₅ is a bioorthogonal handle, which as used herein refers to a biologically compatible functional group that enables the attachment of small molecules and biomolecules to other small molecules and biomolecules in biologically relevant contexts. Bioorthogonal handles are known in the art to function as a reactive group for conjugation addition in chemistry. For example, azides and alkynes are chemical handles that permit cycloaddition conjugation. In some embodiments, R₅ is CCH, N₃, tetrazine, trans-cyclooctene, cyclooctyne, norbomene, norbornadiene, quadricyclane, cyclopropene, cyclopropenone, oxanorbornene, sydnone, oxime, hydroxylamine, or nitrile oxide.

Representative examples of bioorthogonal handles which may be suitable for use in the present disclosure are disclosed in U.S. Patent Application Publication No. 2019/0030183; Kang et al., “Synthesis of Push-Pull-Activated Ynol ethers and Their Evaluation in the Bioorthogonal Hydroamination Reaction,” J. Org. Biomol. Chem., 2022; Kang et al., “Bioorthogonal Click and Release: A General, Rapid, Chemically Revertible Bioconjugation Strategy Employing Enamine N-oxides,” J. Chem., 2022, 8:2260-2277; Kang et al., “Bioorthogonal Hydroamination of Push-Pull-Activated Linear Alkynes,” J. Agnew. Chem. Int. Ed., 2021, 60:16947-16952; and Kang et al., “Bioorthogonal Retro-Cope Elimination Reaction of N,N-Dialkylhydroxylamines and Strained Alkynes,” J. Am. Chem. Soc., 2021, 143:5616-5623.

In some embodiments, R₅ is an affinity handle, which as used herein refers to a moiety that that targets the inventive compound to an appropriate site, e.g., a protein or a nucleic acid.

In some embodiments, the affinity handle is biotin or a derivative thereof. Biotin derivatives are known in the art. See, e.g., Molecular Probes Handbook, A Guide to Fluorescent Probes and Labeling Technologies, 11^{th} Ed., Life Technologies Corporation, 2010. Biotin and its derivatives have been widely used as molecular labels in the biotechnology industry for many years. Representative examples of biotin derivatives that may be suitable for use in the present disclosure include biotin (picolyl) azide, desthiobiotin, pyrimethamine biotin, rac selenobiotin, biocytin, 2-iminobiotin, biocytin-L-proline, biotinyl cystamine, and biotinyl tobramycin amide. Other biotin derivatives that may be suitable for use in the present disclosure are described in the art, e.g., U.S. Pat. No. 8,318,696 and U.S. Patent Application Publication No. 2007/0020206, each of which is incorporated by reference.

In some embodiments, the affinity handle is a short peptide sequence (e.g., 2 to 50 amino acids in length, e.g., 4 to 20 amino acids in length, wherein the amino acid residues in the peptide may be the same or different). Representative examples include α-amanitin, antipain, ceruletide, glutathione, leupeptin, netropsin, pepstatin, peptide T, phalloidin, teprotide, tuftsin, ALFA-tag, AviTag, C-tag, calmodulin-tag, polyglutamate tag, poly arginine tag, E-tag, FLAG-tag, HA-tag, His-tag, Myc-tag, NE-tag, Rho1D4-tag, S-tag, SBP-tag, softag 1, softag 3, Spot-tag, Strep-tag, T7-tag, TC tag, Ty tag, V5 tag, VSV-tag, and Xpress tag.

In some embodiments, R₅ is a reporter, which as used herein refers to a moiety that allows the inventive compound to be detected or visualized. The reporter may be directly detectable (i.e., it does not require any further reaction or manipulation to be detectable, e.g., a fluorophore or chromophore is directly detectable).

In some embodiments, the reporter is a fluorophore. Representative examples of fluorophores include green fluorescent protein, phycoerythrin, allophycocyanin, fluorescein and fluorescein dyes (e.g., fluorescein isothiocyanine (FITC), naphthofluorescein, 4′,5′-dichloro-2′,7′-dimethoxy-fluorescein, 6-carboxyfluorescein or FAM), carbocyanine, merocyanine, styryl dyes, oxonol dyes, phycoerythrin, erythrosin, eosin, rhodamine dyes (e.g., 5-carboxytetramethylrhodamine (TAMRA), carboxyrhodamine 6G, carboxy-X-rhodamine (ROX), lissamine rhodamine B, rhodamine 6G, rhodamine Green, rhodamine Red, or tetramethylrhodamine (TMR)), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red™, Spectrum Green™, cyanine dyes (e.g. Cy-3™, Cy-5™, Cy-3.5™, Cy-5.5™) Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R₆G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. For more examples of suitable fluorescent dyes and methods for coupling fluorescent dyes to other chemical entities see, for example, The Handbook of Fluorescent Probes and Research Products, 9th Ed., Molecular Probes, Inc., Eugene, Oregon and Molecular Probes Handbook, A Guide to Fluorescent Probes and Labeling Technologies, 11^{th} Ed., Life Technologies.

In some embodiments, R₆ and R_{6′} are each independently hydrogen, Me, Et, iPr, phenyl, p-OMePh, p-CF₃Ph, or o-MePh.

In some embodiments, each L₁ and L₂ is independently absent or (C₁-C₄) alkylene;
R₁ is Me, Et, iPr, or CH₂tBu;
R₂ is Cl, Br, or OMe;
R₃ is OC(O), C(O)O, OC(O)NH, NHC(O)O, OC(O)NMe, NMeC(O)O, OC(O)O, S(O)₂O, OS(O)₂, or OP(OR′)O₂;
R₅ is CCH, N₃, or biotin; and
R₆ is hydrogen, Me, Et, Pr, phenyl, p-OMePh, p-CF₃Ph, or o-MePh.

In some embodiments, the optional substituent for a compound of formula (I-VI) is independently alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, and azido.

Other inventive compounds of the disclosure are represented by formula VII:

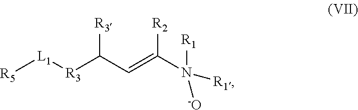

or a pharmaceutically acceptable salt or stereoisomer thereof,
wherein:
L is a linking group;
R₁ and R_{1′} are independently (C₁-C₈) alkyl, (C₃-C₁₀) carbocyclyl, or 4- or 10-membered heterocyclyl comprising 1 to 3 heteroatoms selected from O, N, and S, wherein said alkyl, carbocyclyl or heterocyclyl is further optionally substituted, or
R₁ and R_{1′} together with the nitrogen atom to which they are attached, form a 4- to 7-membered heterocyclyl comprising 1 to 3 heteroatoms selected from O, N, and S;
R₂ is halo or alkoxy;
R₃ is absent, OC(O), C(O)O, OC(O)O, OC(O)NR′, NR′C(O)O, S(O), S(O)₂, OS(O)₂, S(O)₂O, OP(OR′)O₂, or a leaving group, wherein each R′ is independently hydrogen, (C₁-C₆) alkyl, (C₃-C₁₀) carbocyclyl, 4- or 7-membered heterocyclyl, and wherein said alkyl, carbocyclyl, or heterocyclyl is optionally substituted;
R_{3′} is hydrogen, (C₁-C₆) alkyl, (C₃-C₁₀) carbocyclyl, or 4- or 10-membered heterocyclyl comprising 1 to 3 heteroatoms selected from O, N, and S, or a leaving group, wherein said alkyl, carbocyclyl or heterocyclyl is further optionally substituted; and
R₅ is a biorthogonal handle, an affinity handle, a reporter, or a ligand that binds a cellular protein or a nucleic acid, provided that one of R₃ or R_{3′} is a leaving group.

In some embodiments, R₁ is methyl or ethyl.

In some embodiments, R_{1′} is methyl or ethyl.

In some embodiments, R_{1′} and R₁, together with the nitrogen atom to which they are bound, form a piperidinyl ring.

In some embodiments, R_{1′} and R₁, together with the nitrogen atom to which they are bound, form a piperazinyl ring.

In some embodiments, R₂ is bromo.

In some embodiments, R₂ is chloro.

In some embodiments, R₂ is fluoro.

In some embodiments, R₂ is iodo.

In some embodiments, R₂ is alkoxy, e.g., C₁-C₆ alkoxy. In some embodiments, R₂ is C₁-C₃ alkoxy. In some embodiments, R₂ is methoxy.

In some embodiments, R_{3′} is a leaving group. In some embodiments, leaving group is halo.

In some embodiments, R₃ is OC(O)NMe.

In some embodiments, L is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the alkylene chain is a C₁-C₆ alkylene chain.

In some embodiments, L is a polyethylene glycol chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

In some embodiments, the polyethylene glycol chain has 1 to 6 —(CH₂CH₂—O)— units.

In some embodiments, R₅ is a biorthogonal handle as described above in formulas I-VI.

In some embodiments, R₅ is an affinity handle as described above in formulas I-VI.

In some embodiments, R₅ is a reporter as described above in formulas I-VI.

In some embodiments, R₅ is a ligand that binds a cellular protein as described above in formulas I-VI.

Compounds of the present disclosure may be in the form of a free acid or free base, or a pharmaceutically acceptable salt. A pharmaceutically acceptable salt of the compounds of this disclosure can be formed, for example, by reaction of an appropriate free base of a compound of the disclosure and an appropriate pharmaceutically acceptable acid in a suitable solvent under standard conditions well known in the art. See, for example, Gould, P. L., “Salt selection for basic drugs,” International Journal of Pharmaceutics, 33:201-217 (1986); Bastin, R. J., et al., “Salt Selection and Optimization Procedures for Pharmaceutical New Chemical Entities,” Organic Process Research and Development, 4:427-435 (2000); and Berge, S. M., et al., “Pharmaceutical Salts,” Journal of Pharmaceutical Sciences, 66:1-19 (1977).

Compounds of the present disclosure may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present disclosure may be made and used in the form of individual isomers and substantially free of other isomers, or in the form of a mixture of various isomers, e.g., racemic mixtures of stereoisomers.

In some embodiments, the compound of formula (I-VII) is an isotopic derivative in that it has at least one desired isotopic substitution of an atom, at an amount above the natural abundance of the isotope, i.e., enriched. In one embodiment, the compound includes deuterium or multiple deuterium atoms. As used herein, the term “compound” embraces isotopic derivatives.

Compounds of formula (I-VII) may also be in the form of N-oxides, crystalline forms (also known as polymorphs), co-crystals, active metabolites of the compounds having the same type of activity, prodrugs, tautomers, and unsolvated as well as solvated (e.g., hydrated) forms with pharmaceutically acceptable solvents such as water, ethanol, and the like, of the compounds. As used herein, the term “compound” embraces all these forms.

The compounds of formula (I-VII) may be prepared by crystallization under different conditions and may exist as one or a combination of polymorphs of the compound. For example, different polymorphs may be identified and/or prepared using different solvents, or different mixtures of solvents for recrystallization, by performing crystallizations at different temperatures, or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other known techniques.

In some embodiments, the pharmaceutical composition comprises a co-crystal of a compound of formula (I-VII). The term “co-crystal”, as used herein, refers to a stoichiometric multi-component system comprising a compound of formula (I-VII) and a co-crystal former wherein the compound of formula (I-VII) and the co-crystal former are connected by non-covalent interactions. The term “co-crystal former”, as used herein, refers to compounds which can form intermolecular interactions with a compound of formula (I-VII) and co-crystallize with it. Representative examples of co-crystal formers include benzoic acid, succinic acid, fumaric acid, glutaric acid, trans-cinnamic acid, 2,5-dihydroxybenzoic acid, glycolic acid, trans-2-hexanoic acid, 2-hydroxycaproic acid, lactic acid, sorbic acid, tartaric acid, ferulic acid, suberic acid, picolinic acid, salicylic acid, maleic acid, saccharin, 4,4′-bipyridine p-aminosalicylic acid, nicotinamide, urea, isonicotinamide, methyl-4-hydroxybenzoate, adipic acid, terephthalic acid, resorcinol, pyrogallol, phloroglucinol, hydroxyquinol, isoniazid, theophylline, adenine, theobromine, phenacetin, phenazone, etofylline, and phenobarbital.

### Methods of Synthesis

In another aspect, the present disclosure is directed to a method for making an inventive compound, or a pharmaceutically acceptable salt or stereoisomer thereof. Broadly, the inventive compounds and their pharmaceutically acceptable salts and stereoisomers may be prepared by any process known to be applicable to the preparation of chemically related compounds. The compounds of the present disclosure will be better understood in connection with the synthetic schemes that are described in various working examples and which illustrate non-limiting methods by which the compounds may be prepared, e.g. compounds of Formulas I-VI.

In one of these aspects, the present disclosure is directed to methods for preparing compounds of formula I:

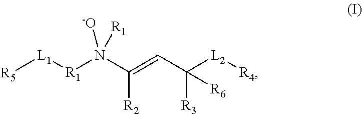

comprising reacting a compound of formula VIII:

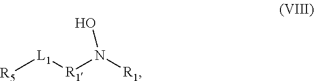

with a compound of formula IX:

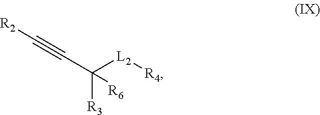

or
compounds of formula II:

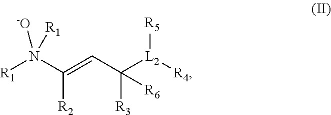

comprising reacting a compound of formula X:

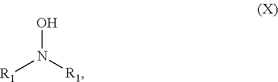

with a compound of formula XI:

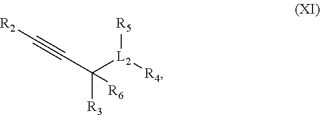

or
compounds of formula III:

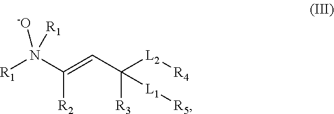

comprising reacting a compound of formula X:

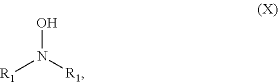

with a compound of formula XII:

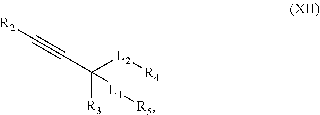

or
compounds of formula IV:

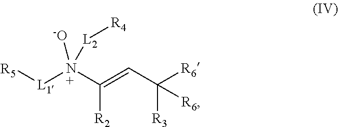

comprising reacting a compound of formula XIII:

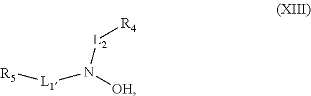

with a compound of formula XIV:

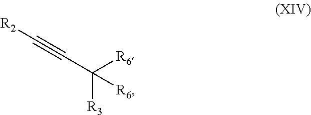

or
compounds of formula V:

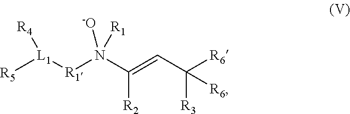

comprising reacting a compound of formula XV:

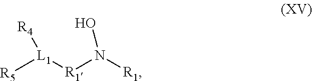

with a compound of formula XIV:

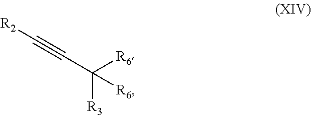

or
compounds of formula VI:

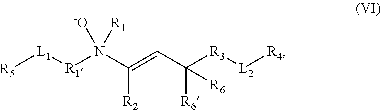

comprising reacting a compound of formula VIII:

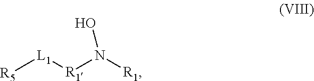

with a compound of formula XV:

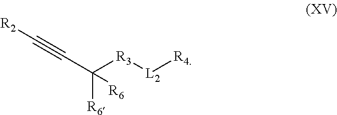

In some embodiments, the reacting is carried out in the presence of a solvent.

In some embodiments, the solvent is an aprotic solvent. In some embodiments, the aprotic solvent is DCM, CHCl₃, CCl₄, DCE, toluene, MeCN, or THF.

In some embodiments, the solvent is a protic solvent. In some embodiments, the protic solvent is MeOH, EtOH, iPrOH, nBuOH, TFE, or HFIP.

In some embodiments, the solvent is a solvent mixture. In some embodiments, the solvent mixture is a mixture of an aprotic solvent and a protic solvent. In some embodiments, the solvent mixture is 0-100% protic to aprotic. In some embodiments, the solvent mixture is 0-100% TFE in CHCl₃. In some embodiments, the solvent mixture is about 20% TFE in CHCl₃.

In some embodiments, the reaction is carried out at a temperature from about 0° C. to 100° C. In some embodiments, the reacting is carried out at a temperature between about 50° C.-60° C. In some embodiments, the reaction is carried out at a temperature of about 50° C. In some embodiments, the reacting is carried out at a temperature is about 60° C.

In some embodiments, the reaction is carried out over a week. In some embodiments, the reaction is carried out over five days. In some embodiments, the reaction is carried out over three days. In some embodiments, the reaction is carried out over a period of 24 hours. In some embodiments, the reaction is carried out over a period of 18 hours. In some embodiments, the reaction is carried out over a period of 12 hours. In some embodiments, the reaction is carried out over a period of 6 hours. In some embodiments, the reaction is carried out over a period of 3 hours. In some embodiments, the reaction is carried out over a period of 2 hours. In some embodiments, the reaction is carried out over a period of 1 hour. In some embodiments, the reaction is carried out over a period of 45 minutes. In some embodiments, the reaction is carried out over a period of 30 minutes. In some embodiments, the reaction is carried out over a period of 15 minutes. In some embodiments, the reaction is carried out over a period of 5 minutes. In some embodiments, the reaction is carried out over a period of 1 minute.

### Diagnostic Compositions

Another aspect of the present disclosure is directed to a diagnostic composition that includes a diagnostically effective amount of an inventive compound or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present disclosure to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, compounds of the disclosure and their pharmaceutically acceptable salts, or stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). In some embodiments, the inventive compounds are administered parenterally (e.g., intravenously).

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

### Dosage Amounts

As used herein, the term, “diagnostically effective amount” refers to an amount of a disclosed compound, or a pharmaceutically acceptable salt or stereoisomer thereof that is effective in producing the desired detectable response in vitro or in a patient. In some embodiments, the diagnostically effective amount is greater than 0 up to about 200 mg/kg. In some embodiments for in vitro use, the diagnostically effective amount ranges from about 1 μM to about 10 mM.

### Methods of Use

In some aspects, the present disclosure is directed to methods of protein and nucleic acid labeling, comprising contacting a cell or lysate thereof with the diagnostically effective amount of a compound of formula I-VII or a pharmaceutically acceptable salt or stereoisomer thereof and a diboron reagent.

In some embodiments, the present method comprises:
a) contacting a cell or lysate thereof with the diagnostically effective amount of a compound of formula I-VII or a pharmaceutically acceptable salt or stereoisomer thereof;
b) incubating the cells or lysate thereof, and the compound for a suitable period of time;
c) contacting the incubated cells or lysate thereof and the compound with a diboron reagent;
d) another period of incubation; and
e) analyzing the sample to detect binding of the binding ligand and the protein or nucleic acid.

The incubations may be conducted over the course of a suitable period of time, which in general, ranges from less than a minute to about 24 hours. In some embodiments, the period of incubation is 24 hours. In some embodiments, the period of incubation is 18 hours. In some embodiments, the period of incubation is 12 hours. In some embodiments, the period of incubation is 6 hours. In some embodiments, the period of incubation is 3 hours. In some embodiments, the period of incubation is 2 hours. In some embodiments, the period of incubation is 1 hour. In some embodiments, the period of incubation is 45 minutes. In some embodiments, the period of incubation is 30 minutes. In some embodiments, the period of incubation is 15 minutes. In some embodiments, the period of incubation is 5 minutes. In some embodiments, the period of incubation is 1 minute. In some embodiments, the period of incubation is less than 1 minute.

In some embodiments, the diboron reagent is a symmetrical diboron reagent. In some embodiments, the diboron reagent is an unsymmetrical diboron reagent. In some embodiments, the diboron reagent is B₂(OH)₄, B₂pin₂,

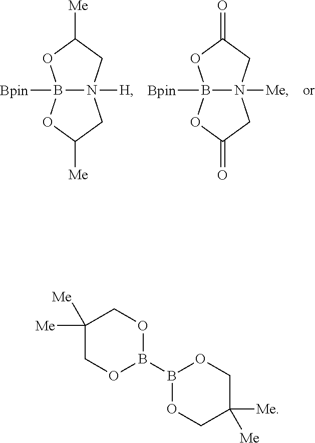

Other representative examples of diboron reagents include bis(catecholato)diboron, bis(hexylene glycolato)diboron, bis[(−)pinanediolato]diboron, bis(diisopropyl-1-tartrate glycolato)diboron, bis(N,N,N′,N′-tetramethyl-d-tartaramide glycolato)diboron, and 2,2′-bi-1,3,2-dioxaborinane. Yet other diboron reagents which may be suitable for use in the present disclosure are disclosed in Ali et al., Studies in Inorganic Chemistry, “Chapter 1—Chemistry of the diboron compounds” 22:1-57 (2005); Neeve et al., Chem. Rev. 116(16):9091-9161 (2016); Ding et al., Molecules 24(7):1325 (2019).

In some embodiments, the diboron reagent is used at a concentration of about 1 pM to about 1 M. In some embodiments, the diboron reagent is used at a concentration of about 1 pM to about 100 mM. In some embodiments, the diboron reagent is used at a concentration of about 1 pM to about 10 mM. In some embodiments, the diboron reagent is used at a concentration of about 1 pM to about 1 mM. In some embodiments, the diboron reagent is used at a concentration of about 1 pM to about 100 μM. In some embodiments, the diboron reagent is used at a concentration of about 1 pM to about 10 μM. In some embodiments, the diboron reagent is used at a concentration of about 1 pM to about 1 μM. In some embodiments, the diboron reagent is used at a concentration of about 1 pM to about 100 nM. In some embodiments, the diboron reagent is used at a concentration of about 1 pM to about 10 nM. In some embodiments, the diboron reagent is used at a concentration of about 1 pM to about 1 nM. In some embodiments, the diboron reagent is used at a concentration of about 1 pM to about 100 pM. In some embodiments, the diboron reagent is formulated in DMSO.

Analysis of the cells or lysate thereof to detect binding may be conducted in accordance with methods and instrumentation known in the art. In some embodiments, the analysis is conducted by in-gel fluorescence or Western blot.

In some embodiments, the analysis is conducted by affinity purification. Affinity purification involves the separation of molecules in solution (mobile phase) based on differences in binding interaction with a ligand that is immobilized to a stationary material (solid phase). Affinity purification techniques which may be suitable for use in the present disclosure are disclosed in Michael et al., Affinity Chromatography: Methods and Protocols, 2^{nd} ed., Totowa, N.J.: Taylor & Francis Group; Uhlen M., BioTechniques 44(5):649-654 (2008).

In some embodiments, the analysis is conducted by mass spectrometry. In some embodiments, mass spectrometry is LC-MS/MS, CE-MS/MS, MALID-MS/MS, or direct sample infusion-MS/MS. Other suitable methods and materials known in the art can also be used in the present disclosure are described in International Patent Application No. WO/2018098473.

These and other aspects of the present disclosure will be further appreciated upon consideration of the following Examples, which are intended to illustrate certain particular embodiments of the disclosure but are not intended to limit its scope, as defined by the claims.

### EXAMPLES

### Example 1: General Information, Materials, and Instrumentations

### General Information

All reactions were conducted in flame-dried round-bottom flasks under a positive pressure of nitrogen unless otherwise stated. Gas-tight syringes with stainless steel needles or cannulae were used to transfer air- and moisture-sensitive liquids. Flash column chromatography was performed using granular silica gel (60-Å pore size, 40-63 μm, Silicycle). Analytical thin layer chromatography (TLC) was performed using glass plates pre-coated with 0.25 mm silica gel impregnated with a fluorescent indicator (254 nm, Silicycle). TLC plates were visualized by exposure to short wave ultraviolet light (254 nm) and/or an aqueous solution of potassium permanganate (KMnO₄). Organic solutions were concentrated at 20° C. on rotary evaporators capable of achieving a minimum pressure of ˜2 torr unless otherwise stated. Room temperature (“rt”) is defined as 22.5±2.5° C. Reaction heating was performed using a UCON™ fluid heating bath.

### General Chemical Materials

All solvents were purchased from Fisher Scientific or Sigma-Aldrich. Unless otherwise stated chemical reagents were purchased from Fisher Scientific, Sigma-Aldrich, Alfa Aesar, Oakwood Chemical, Acros Organics, Combi-Blocks, TCI America, Chem-Impex, Click Chemistry Tools, Broadpharm, or MedChemExpress. CMA refers to a solution of 80:18:2 v/v/v chloroform:methanol (MeOH):ammonium hydroxide (28-30% ammonia solution). Chloroform used in CMA solutions and as co-eluents in silica gel column chromatography were stabilized with 0.75% v/v ethanol. Chloroform used in all hydroamination reactions were stabilized with pentene.

### General Chemical Instrumentation

Proton nuclear magnetic resonance (¹H NMR) spectra, recorded with a 500 MHz Avance III Spectrometer with multi-nuclear Smart probe, are reported in parts per million on the δ scale, and are referenced from the residual protium in the NMR solvent (CDCl₃: δ 7.24, CD₃OD: δ 3.31 (CHD₂OD), CD₃CN: δ 1.94). Data are reported as follows: chemical shift [multiplicity (s=singlet, d=doublet, t=triplet, dd=doublet of doublets, dt=doublet of triplets, dq=doublet of quartets, ddd=doublet of doublets of doublets, tt=triplet of triplets, td=triplet of doublets, tq=triplet of quatets, m=multiplet), coupling constant(s) in Hertz, integration, assignment]. Carbon-13 nuclear magnetic resonance (¹³C NMR) spectra are referenced from the carbon resonances of the solvent (CDCl₃: δ 77.23, CD₃OD: δ 49.15, CD₃CN: δ 1.37). Fluorine-19 nuclear magnetic resonance (¹⁹F NMR) is calibrated from the fluorine resonances of benzotrifluoride (CDCl₃: δ −62.76, CD₃OD: δ −64.24, CD₃CN: δ −63.22). Data are reported as follows: chemical shift (assignment). Infrared data (IR) were obtained with a Cary 630 Fourier transform infrared spectrometer equipped with a diamond ATR objective and are reported as follows: frequency of absorption (cm⁻¹), intensity of absorption (s=strong, m=medium, w=weak, br=broad). High resolution mass spectra (HRMS) were recorded on a Q Exactive™ Plus Hybrid Quadrupole-Orbitrap™ Mass Spectrometer using an electrospray ionization (ESI), atmospheric pressure ionization (API), or electron ionization (EI) source. Automated C₁₈ reverse phase chromatography was performed using an Isolera™ One (Biotage®) purification system. High performance liquid chromatography (HPLC) purification was performed using an Agilent 1260 Infinity system.

### General Biological Instrumentation

In-gel fluorescence imaging was performed on a GE Healthcare Life Sciences Typhoon™ FLA 9500. Images were processed with Fiji ImageJ software. Western blots imaging was performed using a chemiluminescence imager (Amersham Image 600, GE). Images were processed with Image Studio software. UV crosslinking was performed using a Spectrolinker XL-1000 UV Crosslinker with 5×8W 365 nm BL tubes (Spectroline #BLE-8T365).

### Example 2: Development, Validation and Employment of a Chemoproteomics Tool for Protein Labeling and Target Identification Using Chemically Activated Reactive Species

Haloiminium ions are used for the covalent labeling of proteins in a ligand-directed manner for target ID applications. These reactive species are derived from halogenated enamines, which are in turn accessed from caged enamine N-oxides. Given the reactivity of Ghosez's reagent (Ghosez et al., 1-chloro-N,N,2-trimethylpropenylamine, doi.org/10.1002/047084289X.rc155m), haloiminium ions react with the nucleophilic protein side chains and quenched rapidly by water, exhibiting a short half-life with a narrow labeling radius under aqueous conditions.

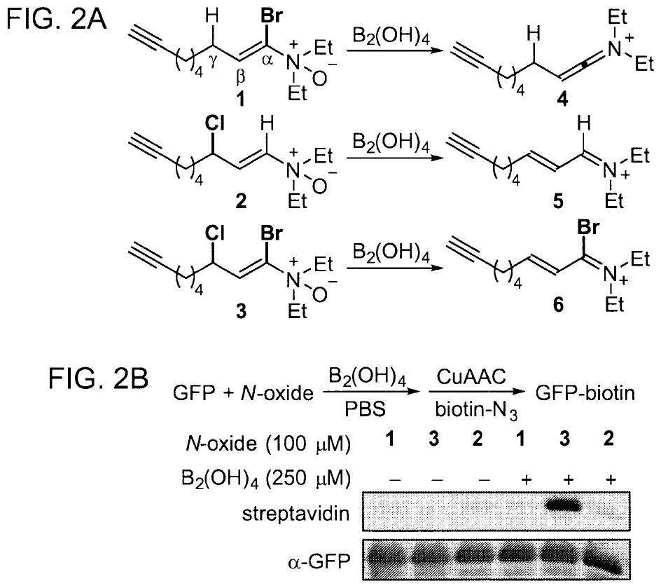

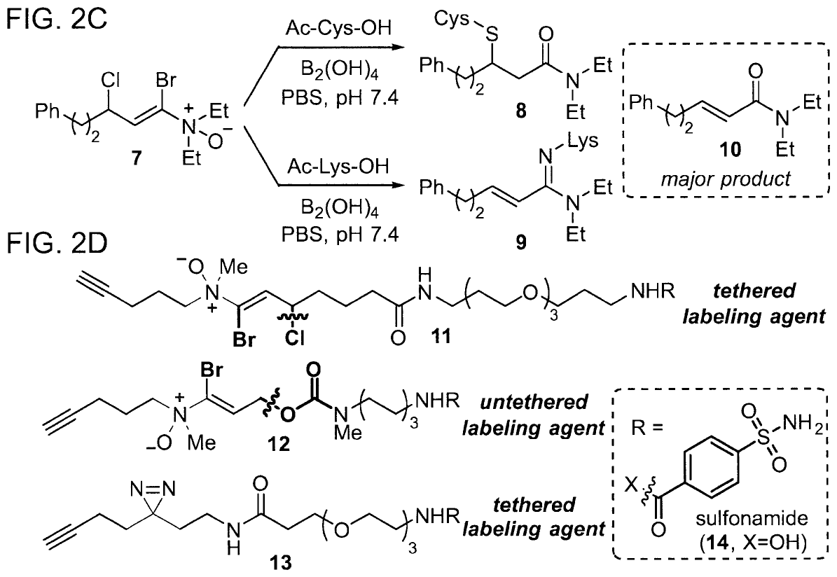

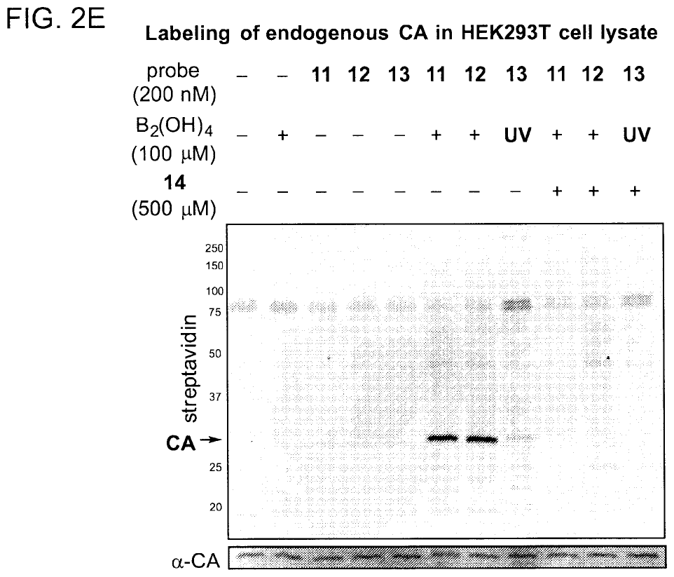

Enamine N-oxides are synthesized through the retro-Cope elimination reaction between terminal alkynes and N,N-dialkylhydroxylamines. 1-Halogenation of alkynes resulted in rapid formation of anti-Markovnikov hydroamination products such as α-halogenated enamine N-oxide 1. Diboron-mediated reduction of high concentrations of α and/or γ-halogenated enamine N-oxides 1-3 (100 μM) in PBS was explored (FIG. 2A). Ligation of a fluorophore azide onto the alkyne handles after the labeling event and in-gel fluorescence analysis indicated that the doubly halogenated enamine N-oxide 3 labeled proteins best (FIG. 2B). To elucidate the mode of reactivity, N-oxide 7 was activated in the presence of N-acetyl cysteine and lysine (FIG. 2C). These nucleophiles both formed covalent adducts with the reactive iminium chloride intermediate, adding in a 1,4- and 1,2-manner, respectively. Formation of amide 10 indicated that the reactive iminium chloride intermediate can be quenched by water, consistent with the mechanism of restricting labeling radius. Cysteine did not add directly to amide 10 itself, which was the major product of each reaction. Next, the enamine N-oxide was attached to sulfonamide (14), a carbonic anhydrase (CA) inhibitor (FIG. 2D), and demonstrated in vitro that recombinant CA is selectively labeled by the probe over bovine serum albumin (BSA), which was also present in equimolar ratio. Labeling was diboron-dependent and could be competed away with excess sulfonamide. Furthermore, intact mass spectrometry analysis of CA treated with either N-oxide probe 11 plus diboron or diazirine probe 13 plus UV irradiation revealed that 60% of the protein was labeled by the former and <1% by the latter.

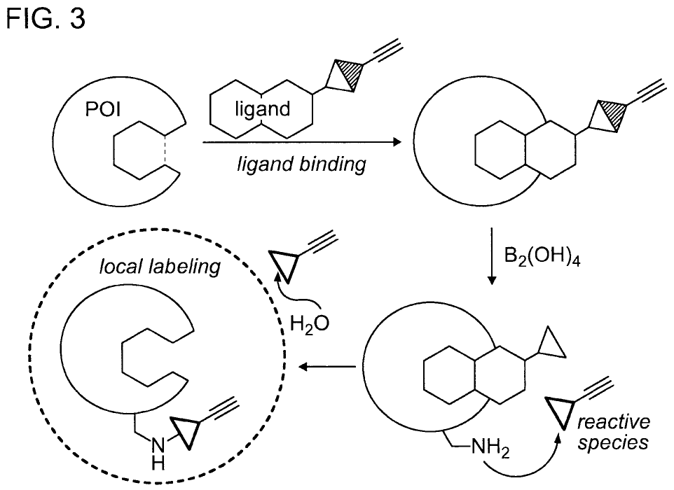

Probe 12 was synthesized with a γ-carbamate to explore untethered reactive species and eliminate linker dependence (FIG. 3). Intact mass spectrometry indicated that carbonic anhydrase labeling was achieved. Probe 12 labeled recombinant CA selectively over BSA in vitro in a diboron and sulfonamide competitor-dependent manner (data not shown). The probe was activated directly in HEK293T cell lysates to label endogenous levels of CA (FIG. 2E). N-oxide probe (200 nM) was treated with diboron (100 μM) in cell lysate without washout of the excess probe. Labeling by untethered probe 12 mirrors that of tethered probe 11, and labeling occurs in a diboron and sulfonamide competitor-dependent manner. Labeling by both probes is markedly superior to labeling by diazirine probe 13, which is barely perceptible. Probes 11 and 12 exhibited minimal background labeling despite being present in lysate in large excess relative to endogenous CA when activated.

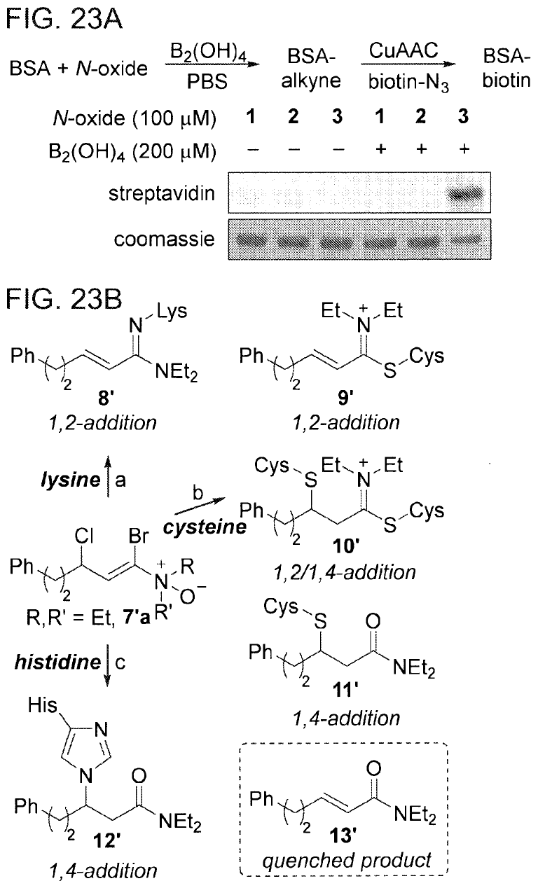

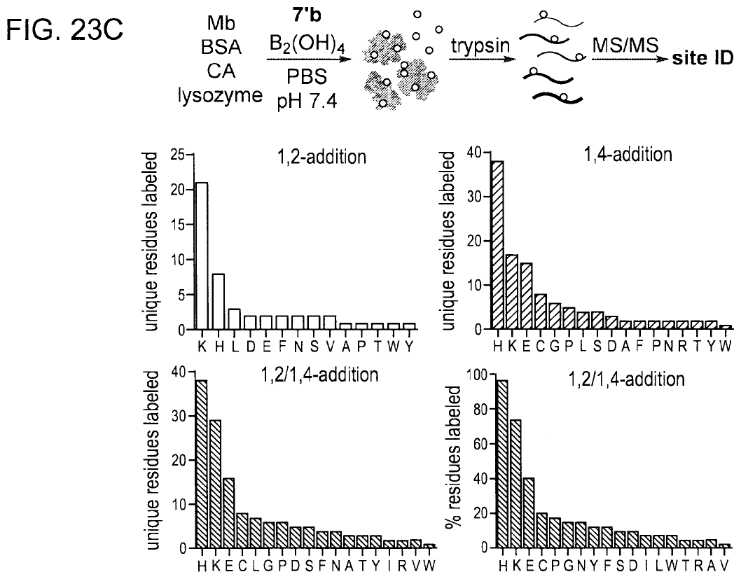

To select the reactive species most appropriate for protein labeling, a high concentration of enamine N-oxides 1-3 (100 μM) was combined with bovine serum albumin (BSA, 0.1 mg/mL) in PBS, pH 7.4, then treated with 200 μM B₂(OH)₄ (FIG. 23A). Alkyne-labeled BSA was further functionalized with biotin by copper-catalyzed azide-alkyne cycloaddition (CuAAC) and analyzed by streptavidin blot. Bromoiminium ion 6 labeled protein best, and its precursor N-oxide 3 was advanced.

Bromoiminium ions react with a variety of nucleophiles in either 1,2- or 1,4-fashion (FIG. 23B). When enamine N-oxide 7′a (50 mM) was reduced with B₂(OH)₄ (60 mM) in the presence of Ac-Lys-OH (500 mM), 1,2-addition product amidine 8′ was obtained. In contrast, when Ac-His-OH was employed, only the 1,4-adduct was observed. A combination of 1,2- and 1,4-adducts 9′-11′ was observed when Ac-Cys-OH was employed.

A significant byproduct of each amino acid reaction was amide 13′. Haloiminium ions react rapidly with water, providing an innate mechanism for reactive species quenching and ensuring a means of inhibiting the indiscriminate labeling of protein. Relative rates of quenching, on-target protein reaction, and diffusion dictates the balance between false positive and negative rates in target identification.

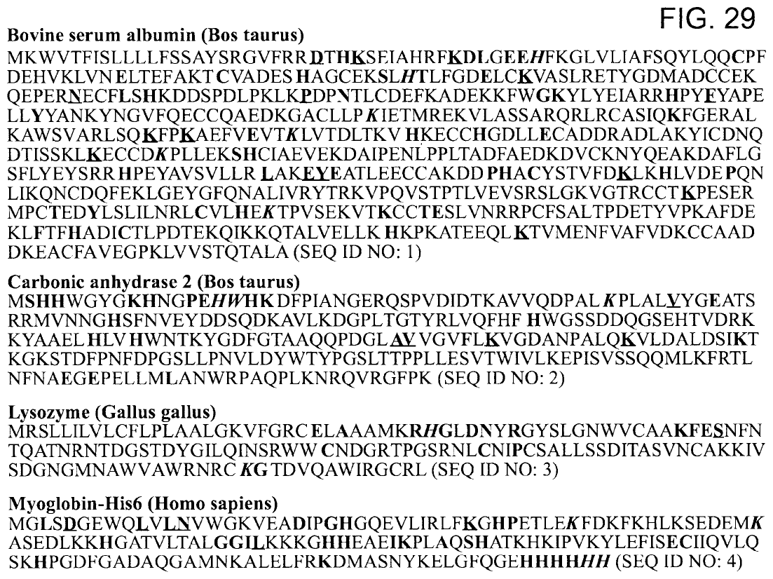

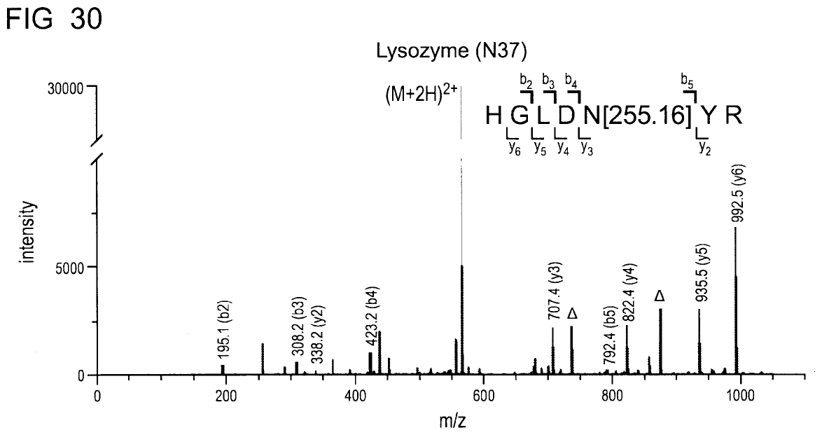

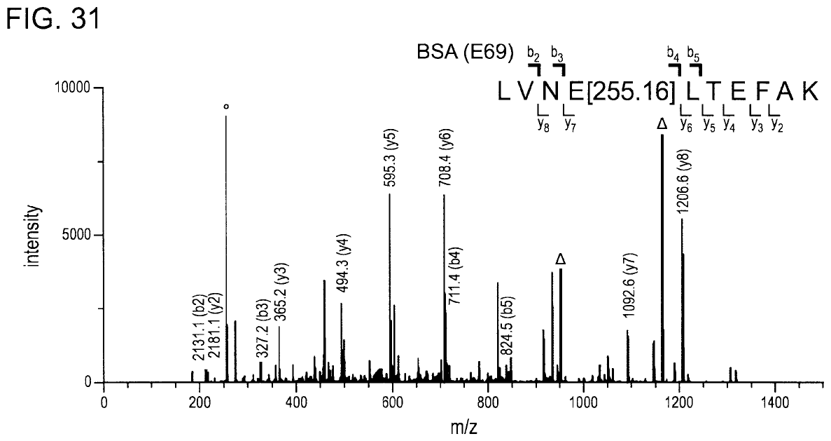

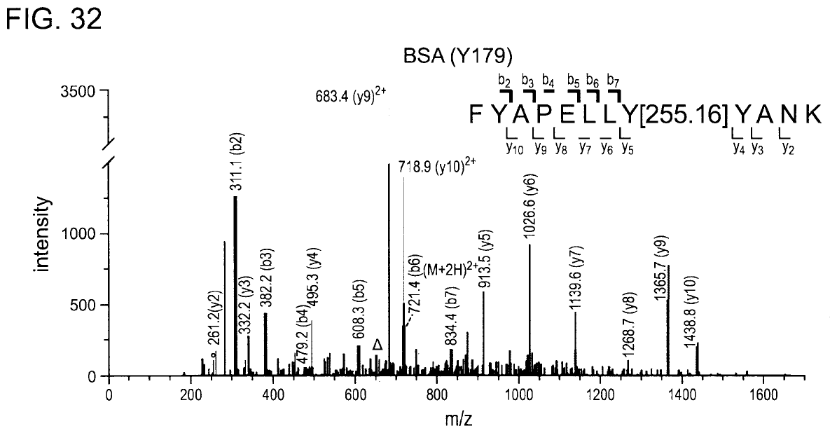

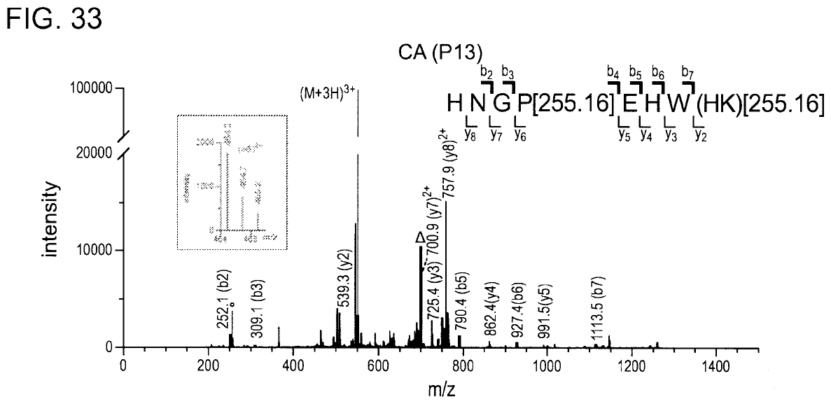

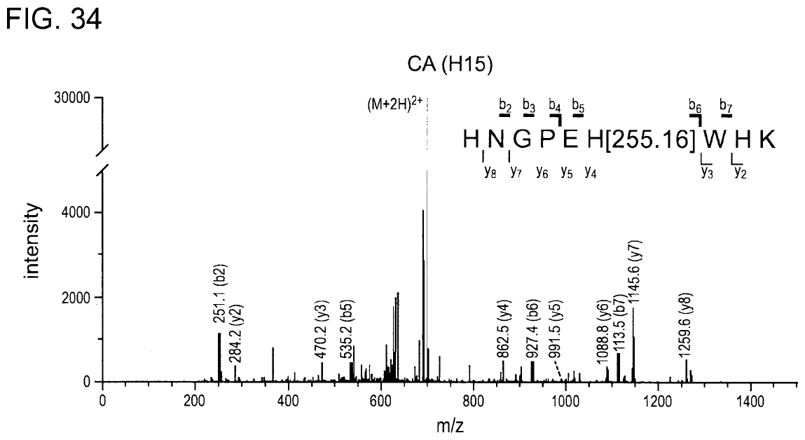

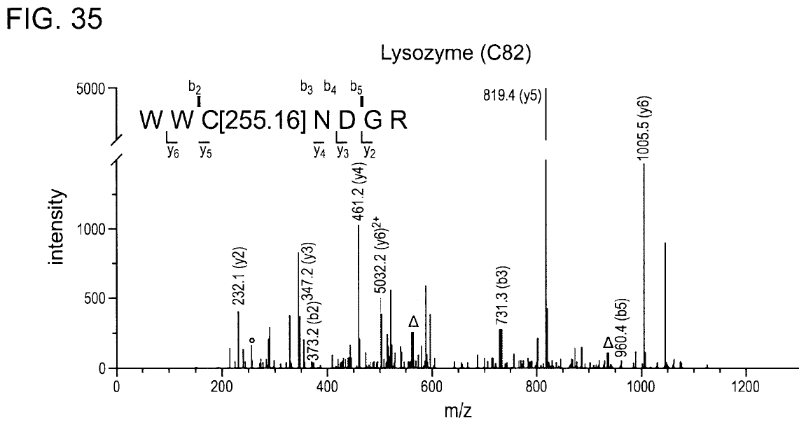

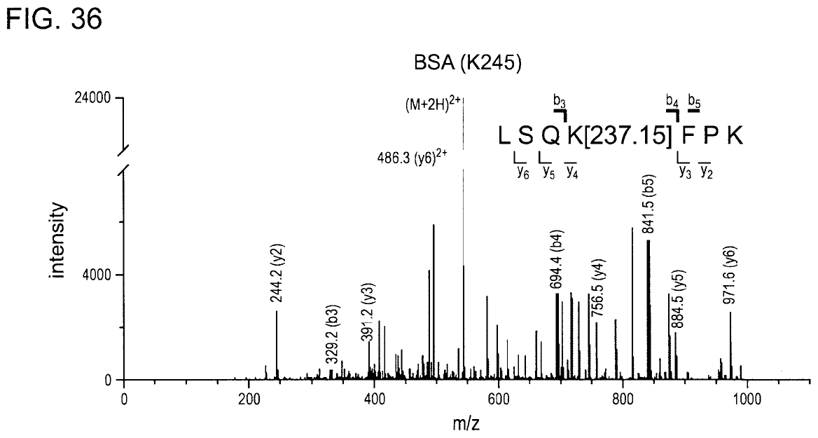

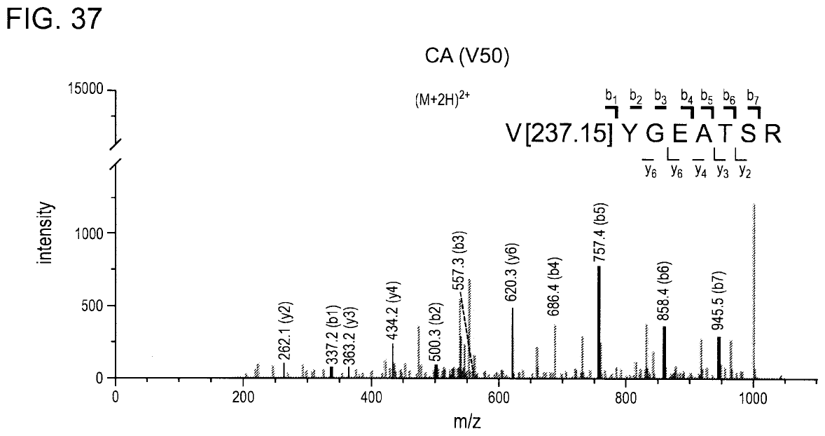

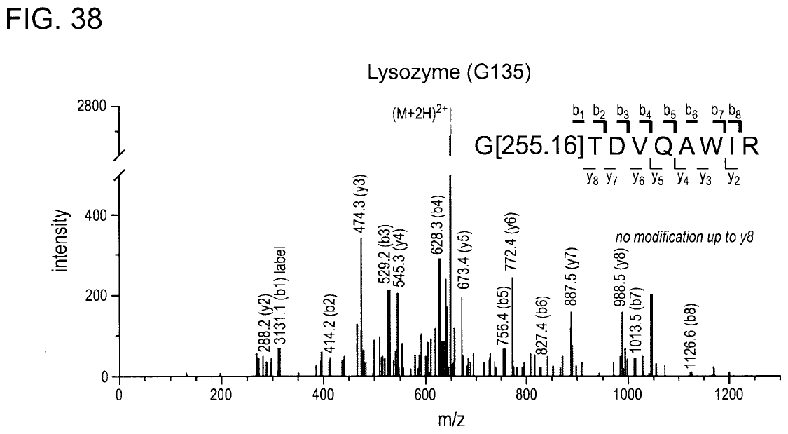

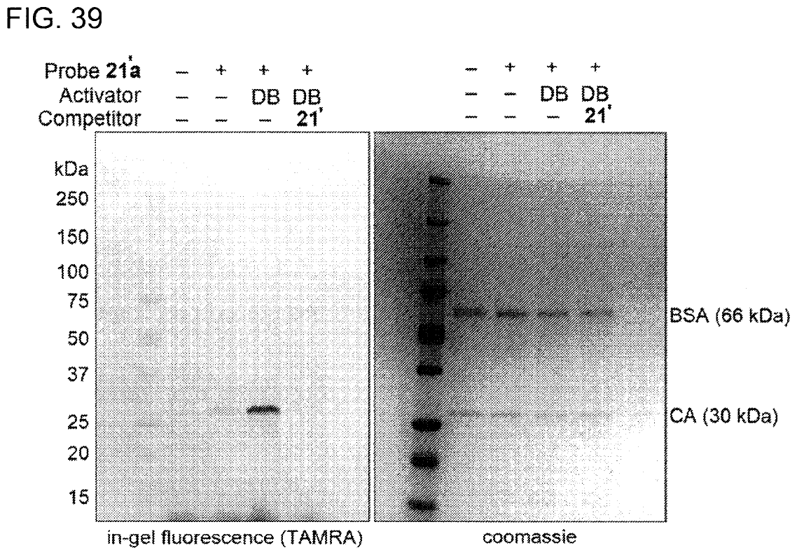

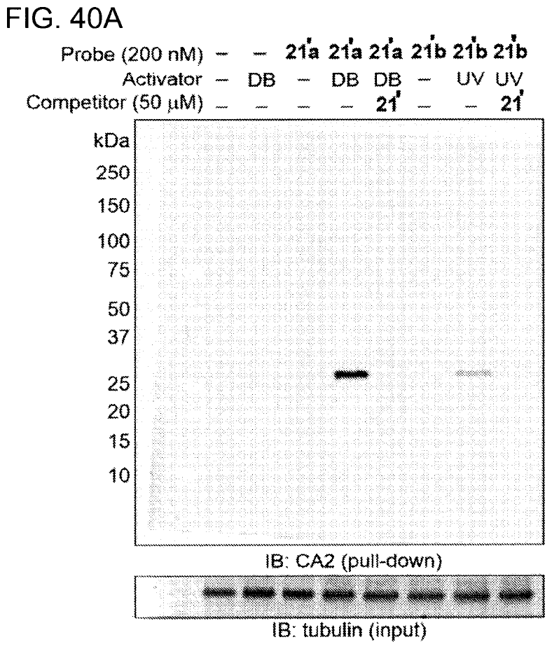

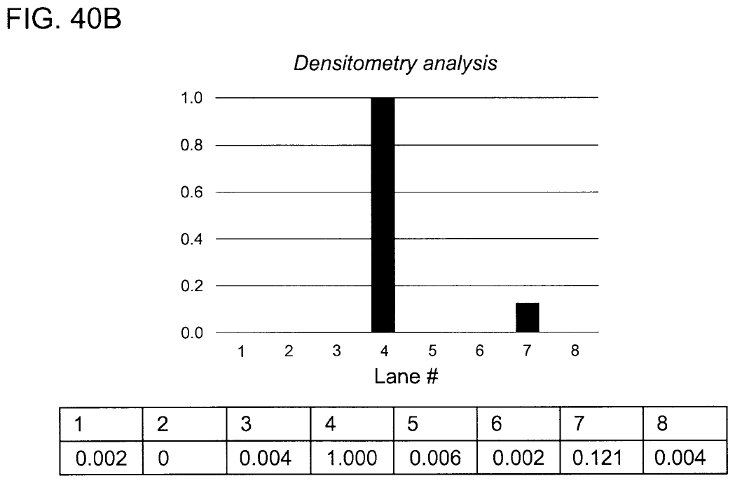

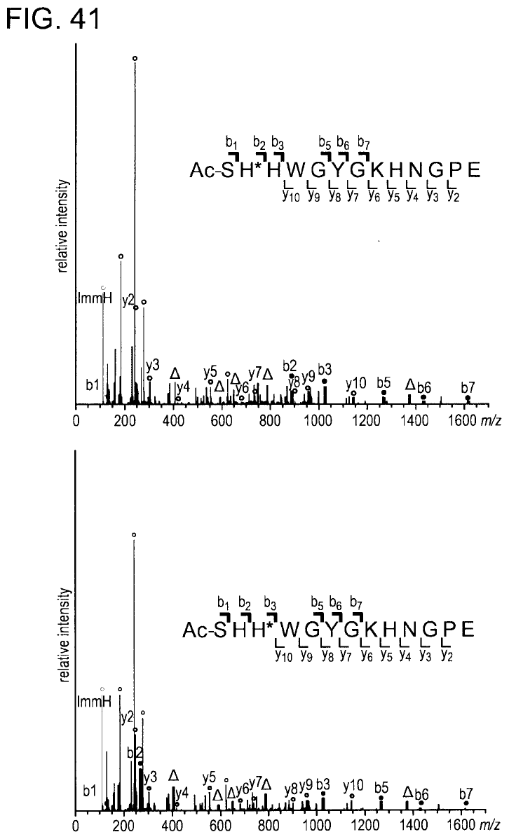

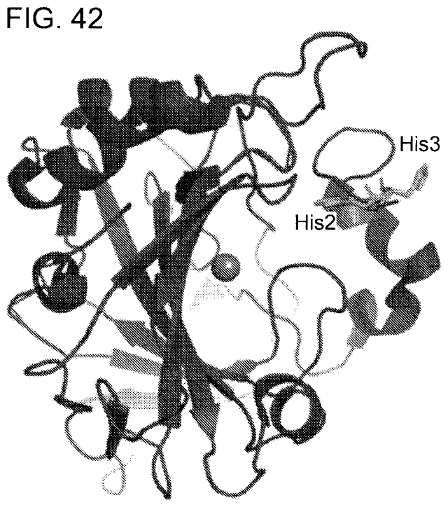

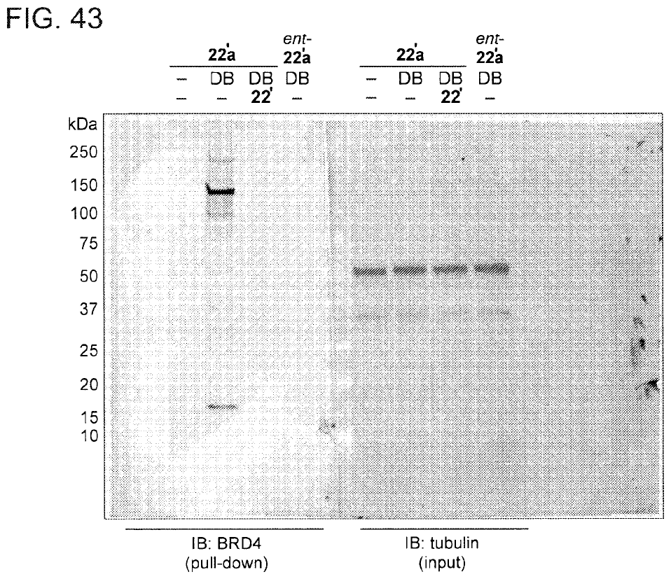

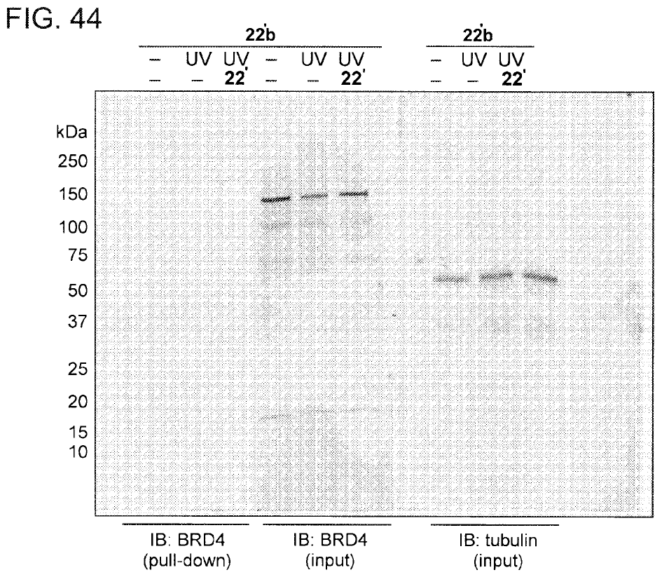

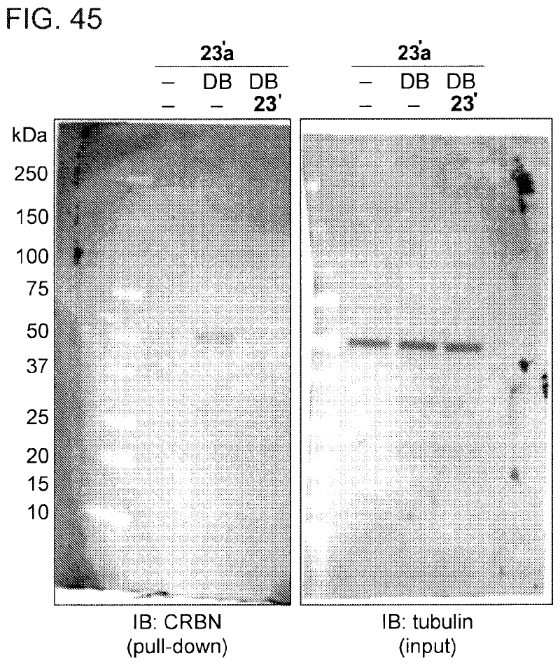

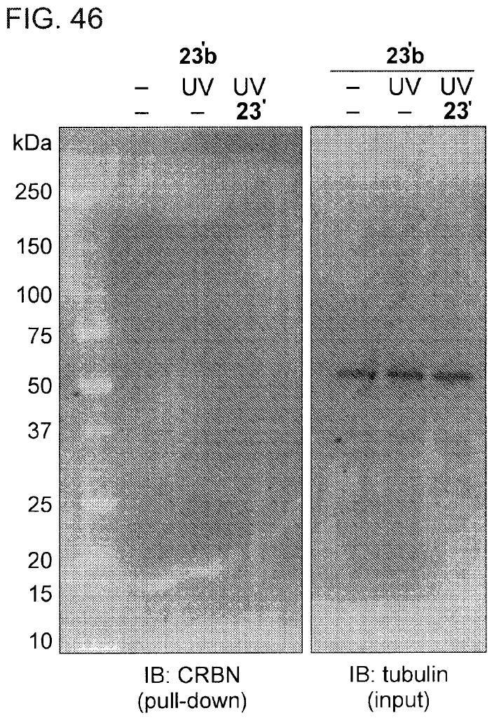

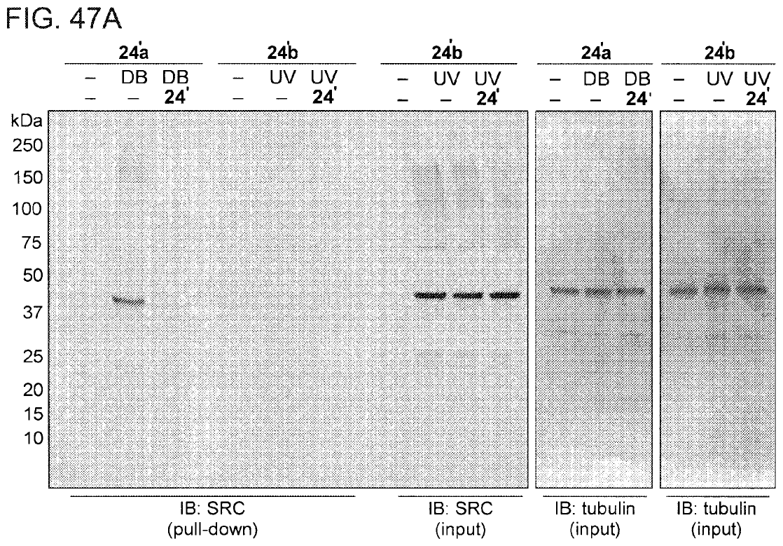

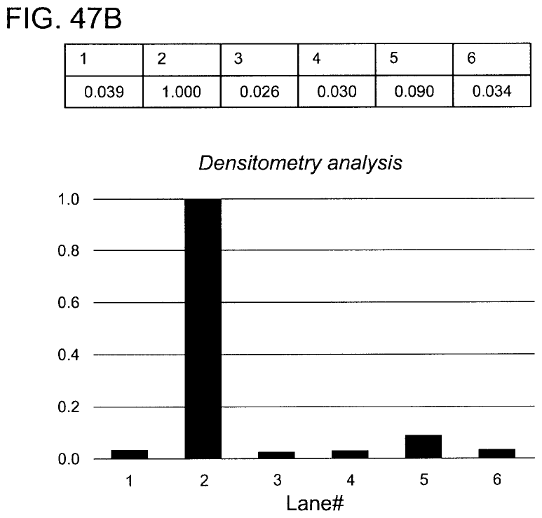

To probe how the amino acid residue labeling preference on proteins would compare to that of isolated amino acids, site identification studies were performed on a protein cocktail consisting of myoglobin (Mb), BSA, carbonic anhydrase (CA), and lysozyme (FIG. 23C, FIG. 29). Following reductive activation of N-oxide 7′b (100 μM) and labeling, proteins were trypsin digested, and the labeling sites were determined by LC-MS/MS. Residues labeled by 1,2- or 1,4-addition were identified by +237 or +255 Da modifications, respectively. 1,2-addition was primarily isolated to lysine and histidine residues while 1,4-addition was more accommodating of diverse amino acids such as cysteine, tyrosine, glutamic acid, and serine. Across both modifications, histidine and lysine exhibited the greatest number of unique residues modified, with histidine outpacing lysine when normalized by residue abundance. The low but non-negligible incidence of labeling of non-nucleophilic residues such as phenylalanine, proline, alanine, leucine, and valine is consistent with peptide backbone modification by the potent electrophiles (FIG. 30-FIG. 38).

### Example 3: Validation of Enamine N-oxide Based Target Identification with Selective and Promiscuous Drugs

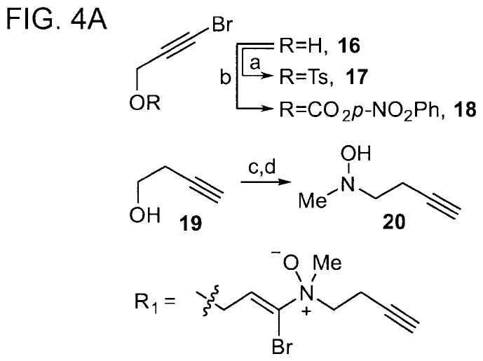

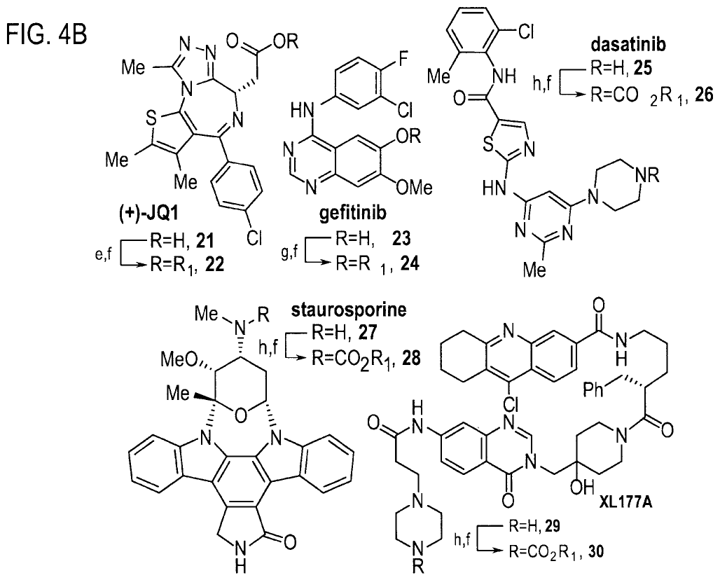

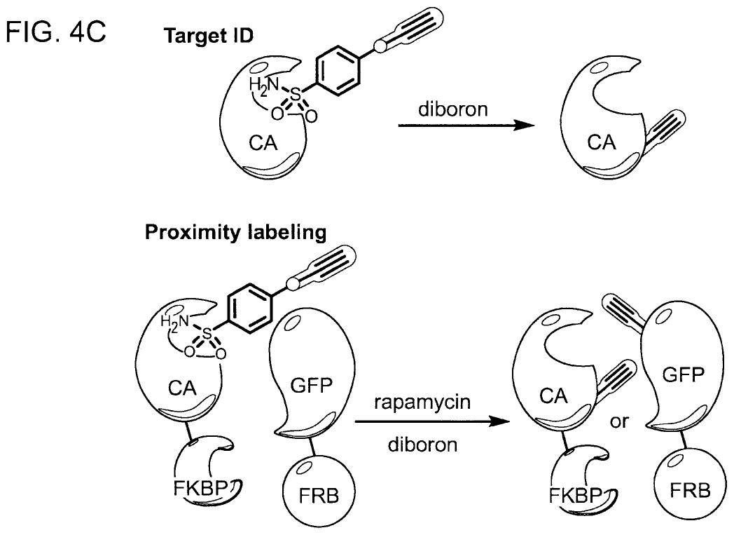

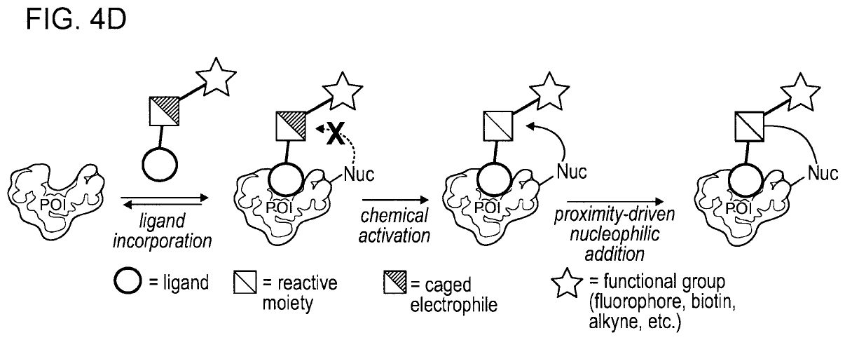

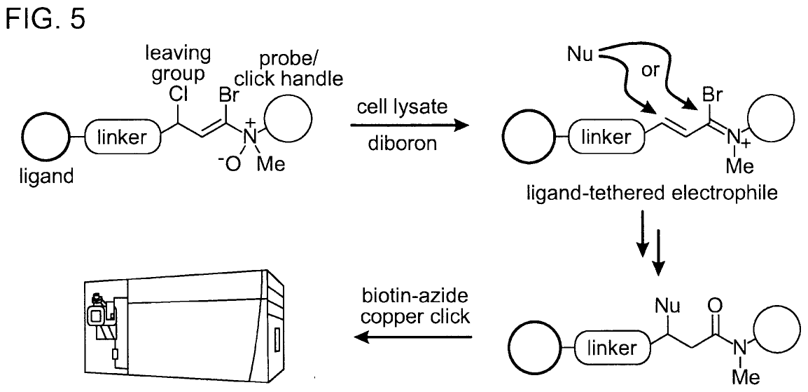

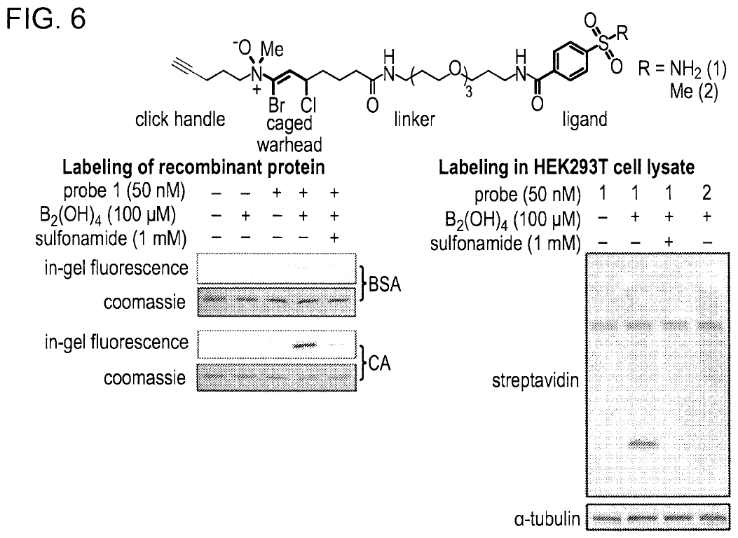

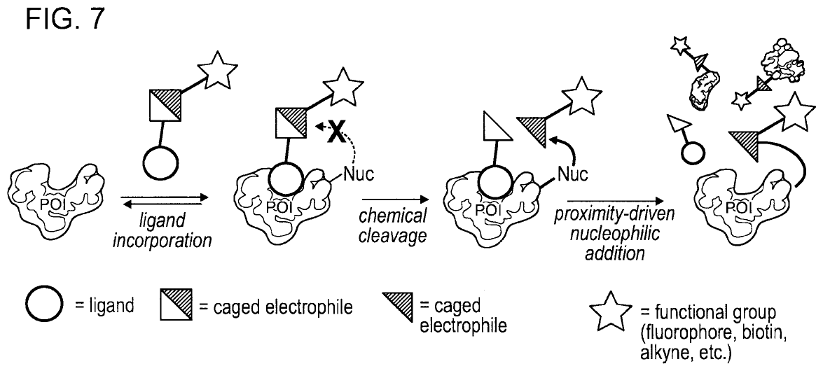

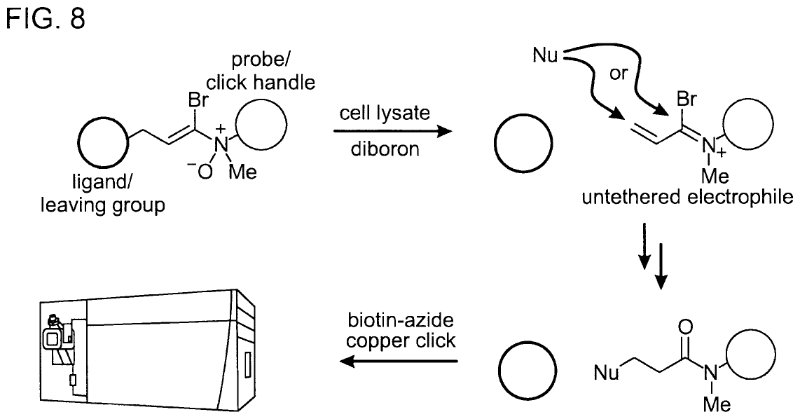

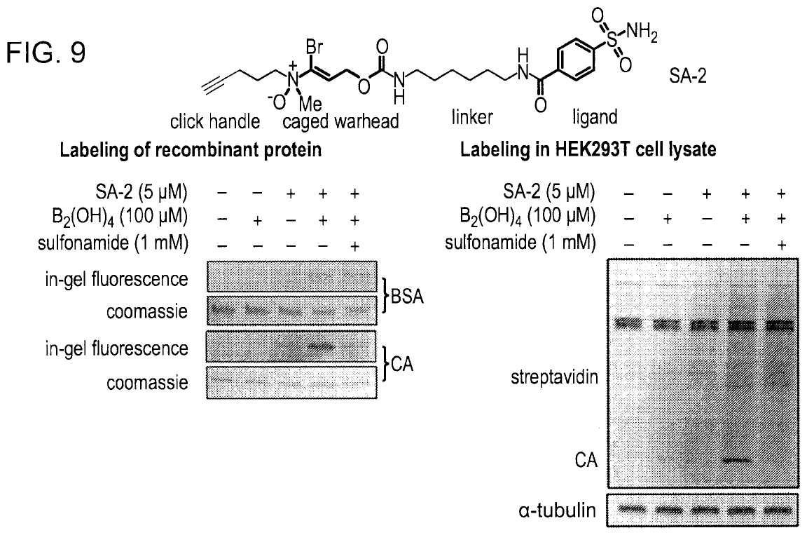

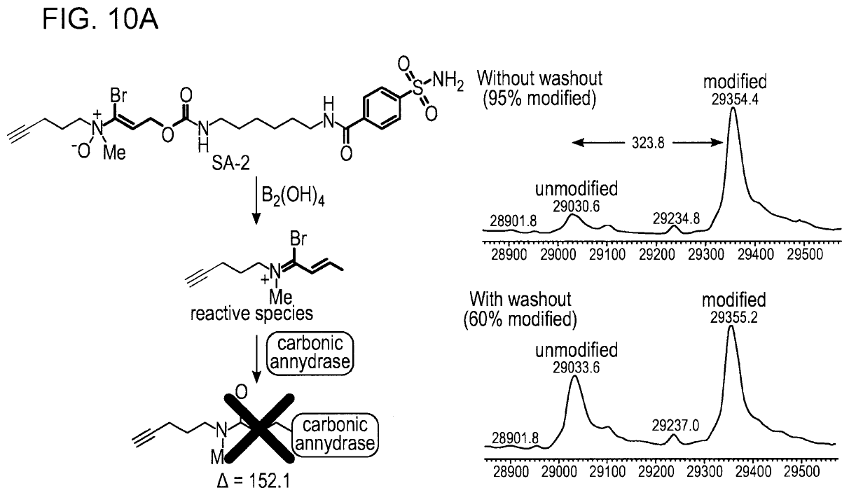

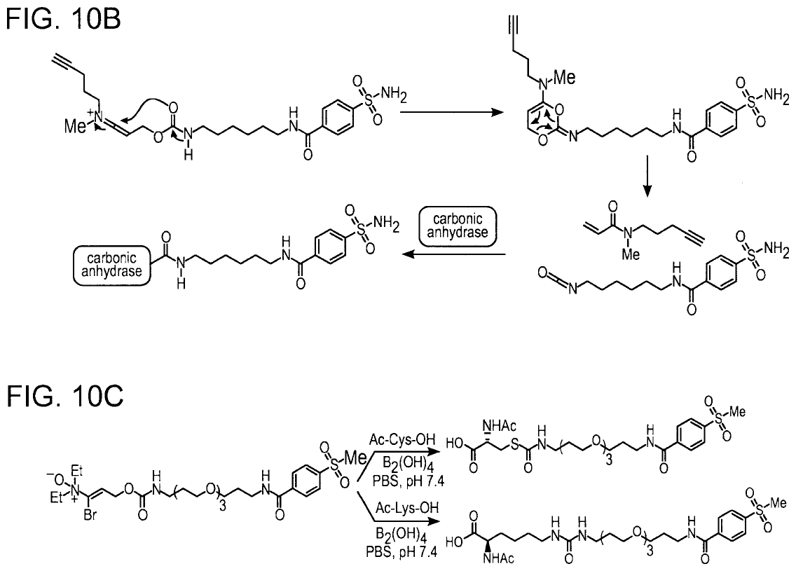

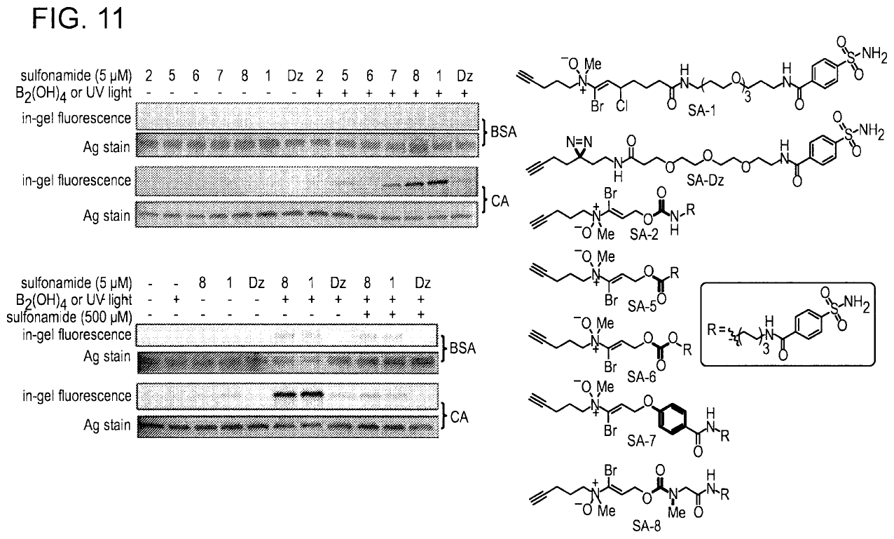

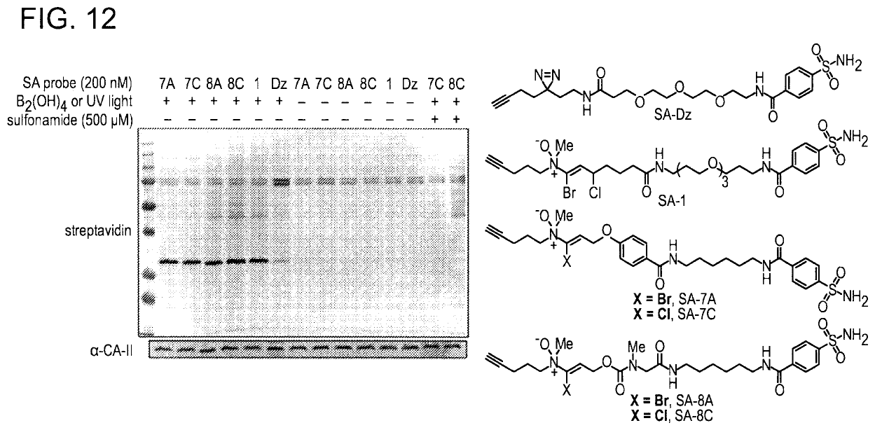

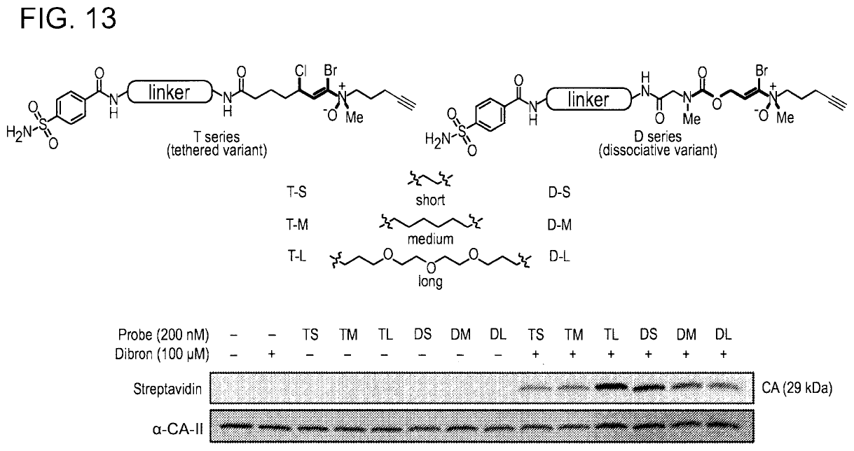

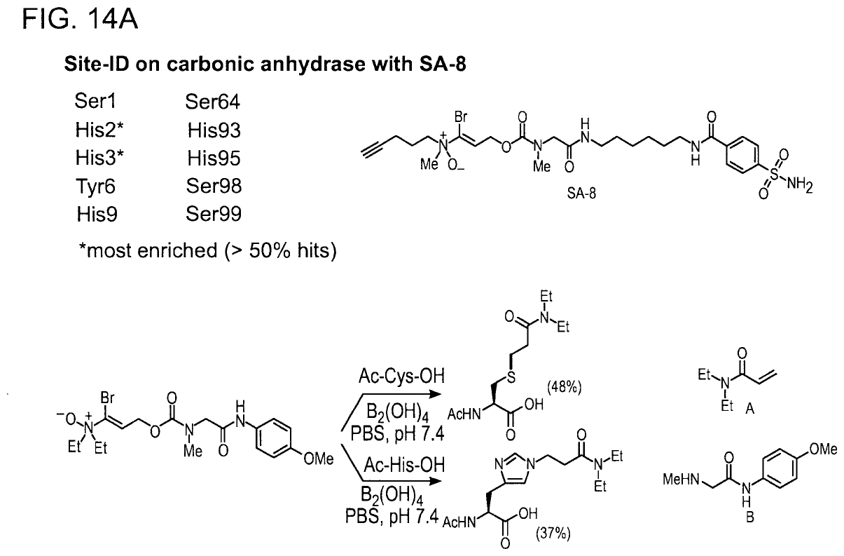

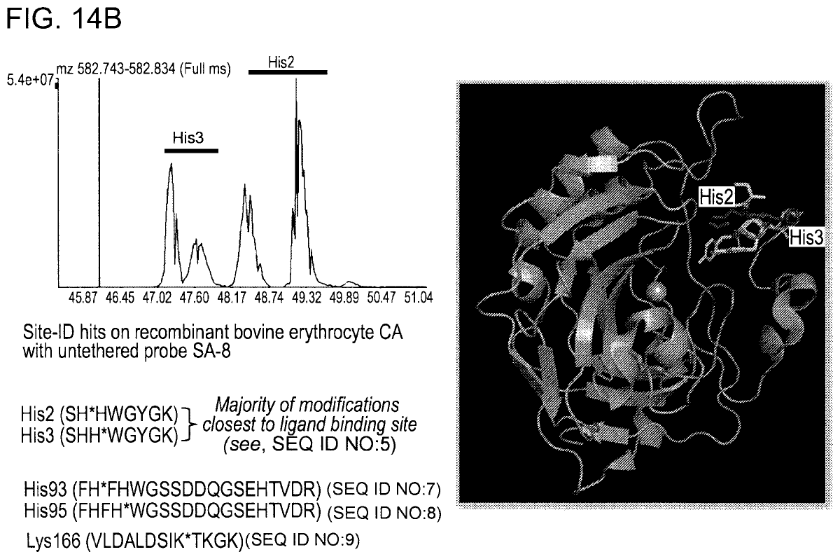

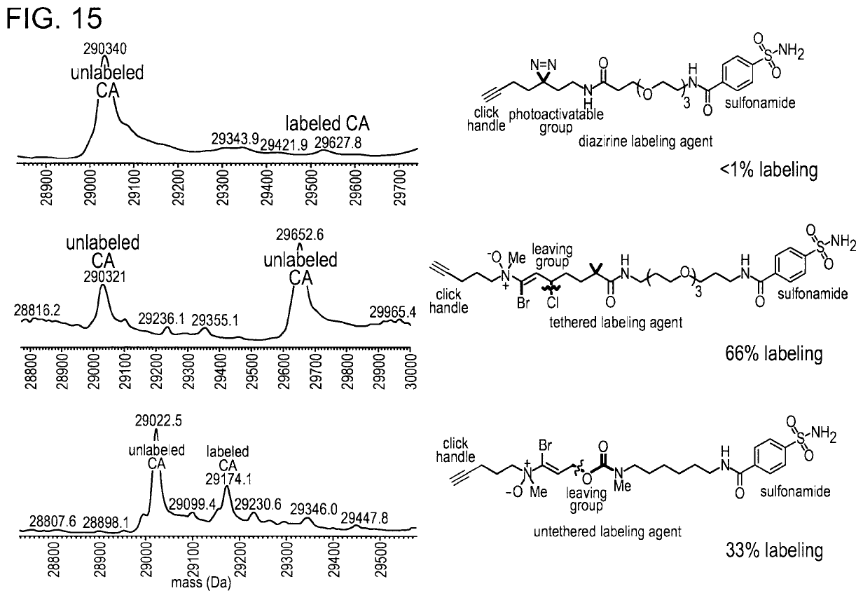

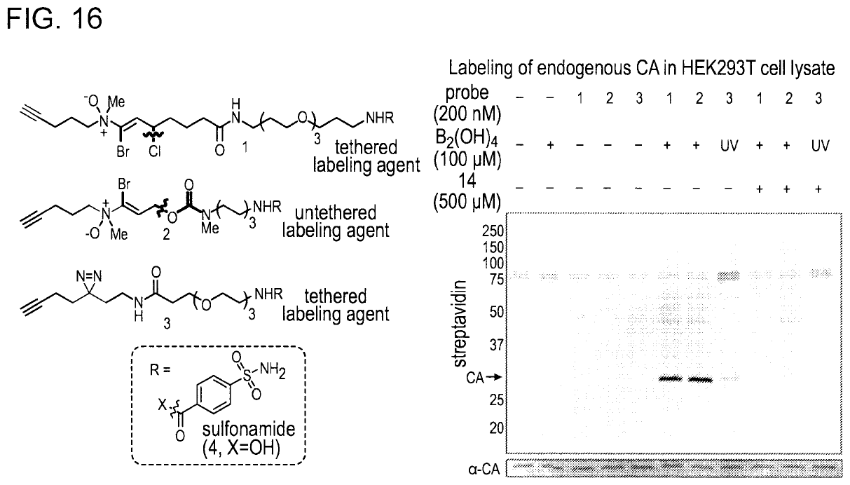

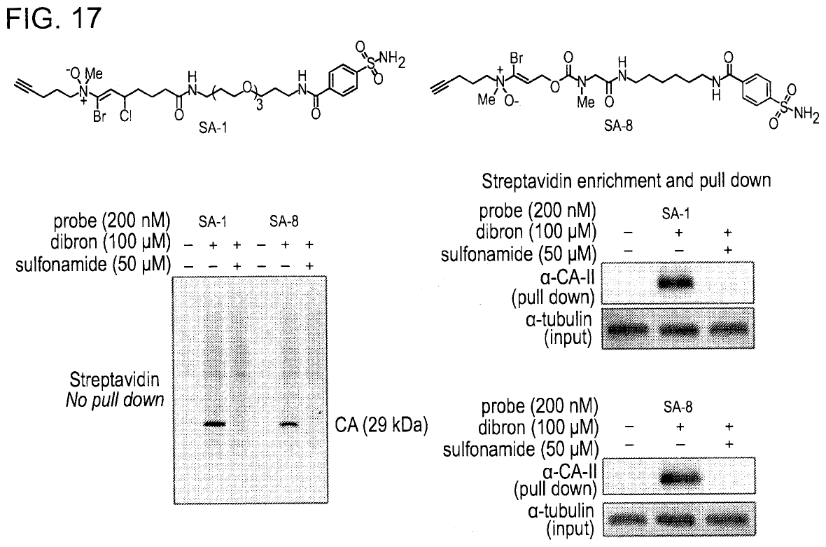

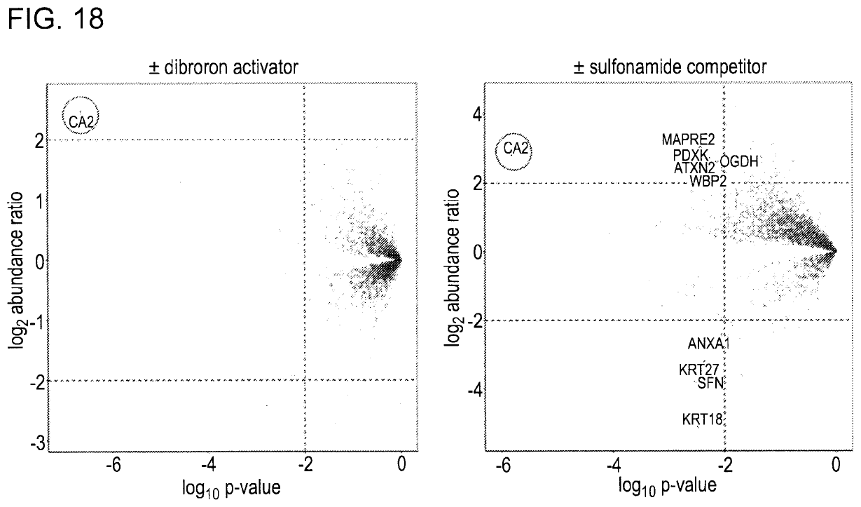

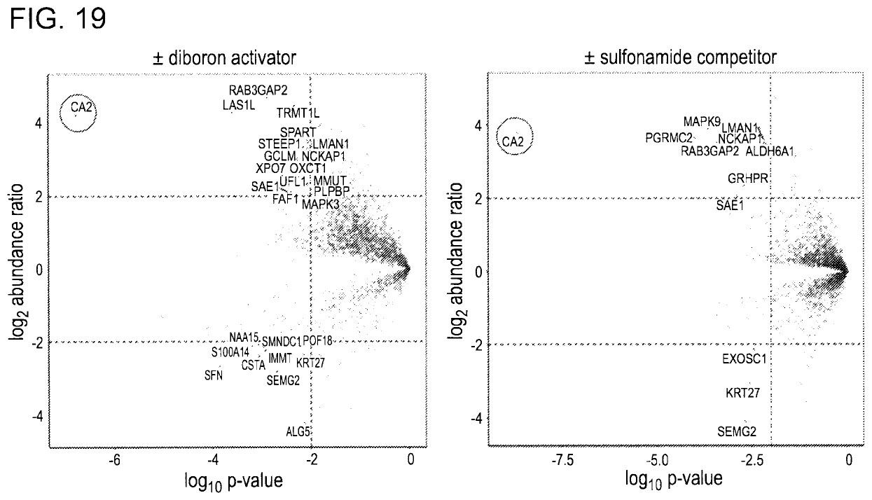

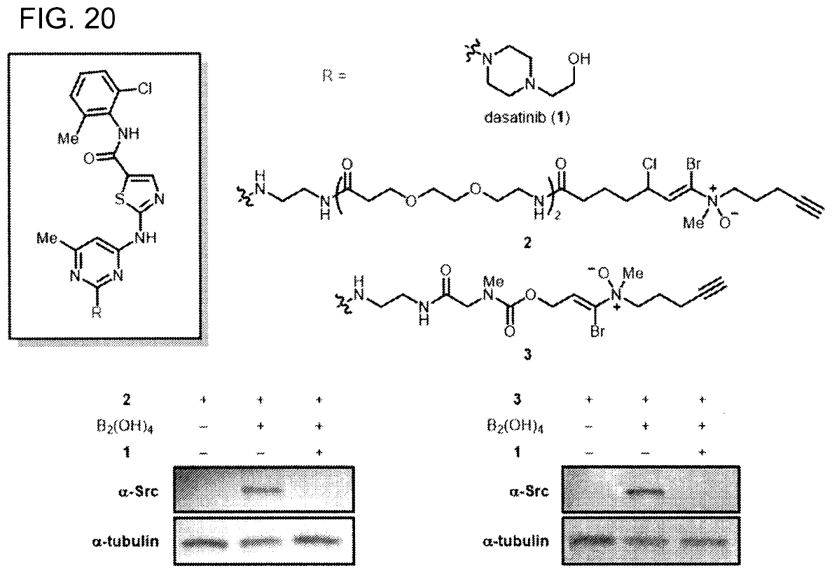

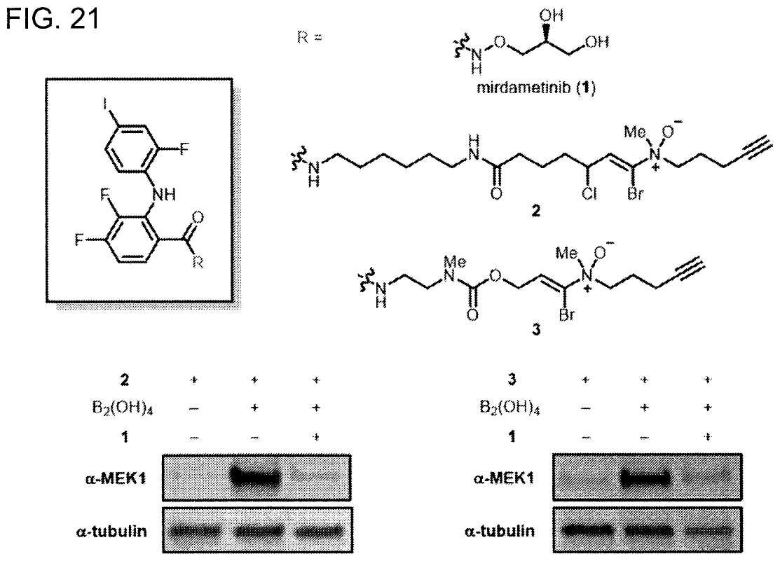

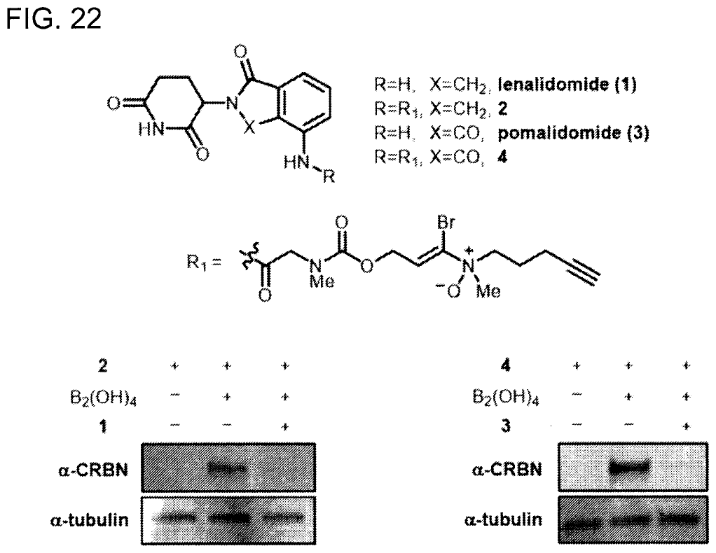

Proteomics experiments were performed with our protein labeling system. Representative set of molecules include: (+)-JQ1, gefitinib, dasatinib, staurosporine, and XL177A (FIG. 4B). Each molecule was synthesized from commercially available intermediates and consistent with known SAR (FIG. 4A and FIG. 4B).

Two sets of proteomics studies were performed with each of the probes—one in cell lysate, the other in live cells. As with all tools requiring the modification of small molecules, the impact of the enamine N-oxide motif on the compound's cell permeability is hard to predict a priori. Cell lysate experiments were done to identify on/off-targets at least under one set of biologically relevant conditions. The proteomics experiments for each probe were performed using label-free techniques, and two sets of controls were employed. The first is a ±diboron control. The second is a ±competition control where the original drug is used to off-compete the N-oxide probe. Based on our in-gel fluorescence studies, the competition-based control experiment provides a higher degree of differentiation between background and on/off-target labeling. All experiments were performed in parallel with a diazirine-based PAL reagent with the appropriate ±UV irradiation and ±competition controls so that the methods were fairly benchmarked with the existing state-of-the-art method. In the cellular experiments, cells are treated first with the N-oxide probe for 3 h, washed, then incubated with 100 μM diboron for 10 min. The wash step, which is not feasible in cell lysate, serves to remove excess unbound probe and further reduce background labeling.

An important component of validating the method is understanding the labeling radius of the probes. FKBP-carbonic anhydrase and GFP-FRB fusion proteins are co-expressed in HEK293T cells then direct enamine N-oxide probe 12 to the protein via its sulfonamide ligand (FIG. 4C). GFP is the acceptor protein in the scheme, and rapamycin-dependent labeling of GFP is observed upon diboron-mediated probe activation when GFP is within the labeling radius of the reactive species. When GFP is labeled in a rapamycin-dependent manner, fusing FRB to large protein complexes such as the ATP-dependent chromatin remodeling BAF complex to see which proteins in the complex are be labeled using the method. The radial distance of these labeling sites, identified through site-ID mass spectrometry, relative to carbonic anhydrase characterizes the labeling radius.

### Example 4: Synthetic Procedures

### Hydroamination of α,γ-Substituted Alkynes (General Procedure A)

A 2 mL LCMS glass vial was charged with N-methyl-N-(pent-4-yn-1-yl)hydroxylamine (3 equiv) at rt. A solution of bromoalkene (1 equiv) in 20% v/v TFE in chloroform (25-50 mM with respect to alkyne) was then added via syringe. The vial was flushed with nitrogen, sealed with a septum cap and Parafilm™, and heated to 50° C. in an oil bath. After 3 h, the reaction was removed from heat, allowed to cool to rt, and concentrated under reduced pressure. The concentrate was then purified by flash column chromatography using chloromethamphetamine (CMA) in chloroform (10-70% CMA in chloroform) as the eluent system. Fractions containing the desired compound were combined, and the solvent was removed under reduced pressure to provide product enamine N-oxide.

### N-Methyl-N-(pent-4-yn-1-yl)hydroxylamine

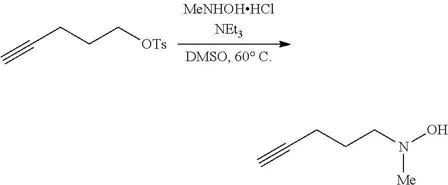

N-methylhydroxylamine hydrochloride (334 mg, 4 mmol, 2 equiv) and triethylamine (1.16 mL, 8 mmol, 4 equiv) were added sequentially to a solution of alkyl tosylate (476 mg, 2 mmol, 1 equiv) in dimethylsulfoxide (4 mL). The reaction mixture was stirred at 60° C. for 2 h. The resulting mixture was then diluted with diethyl ether and extracted with diethyl ether (3×40 mL). The combined organic layers were washed with brine×2, dried over anhydrous sodium sulfate, filtered, and concentrated under reduced pressure. The resulting crude residue was purified by flash column chromatography on silica gel (eluent: 50% ether in pentanes) to give the title compound as a colorless oil. Rf=0.16 in 50% ether/pentanes (KMnO₄).

### Methyl 7-bromo-5-hydroxyhept-6-ynoate

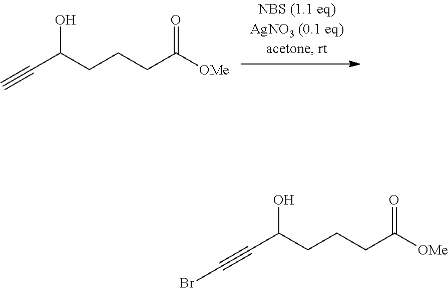

Acetone (16 mL) was added to a flask charged with methyl 5-hydroxyhept-6-ynoate (500 mg, 3.2 mmol, 1 equiv). Silver nitrate (54 mg, 0.32 mmol, 10 mol %) and N-bromosuccinimide (626 mg, 3.52 mmol, 1.1 equiv) were sequentially added. The reaction mixture was stirred at rt for 1 h. The resulting mixture was then filtered through a pad of Celite® and the filtrate was concentrated under reduced pressure. The resulting concentrate was purified by flash column chromatography on silica gel (eluent: 20% ethyl acetate/hexanes) to give the title compound as a light yellow oil (90%). ¹H NMR (500 MHz, CDCl₃) δ 4.34 (t, J=6.1 Hz, 1H), 3.61 (s, 3H), 3.24 (s, 1H), 2.31 (t, J=7.2 Hz, 2H), 1.79-1.61 (m, 4H). ¹³C NMR (126 MHz, CDCl₃) δ 174.1, 80.9, 62.6, 51.7, 44.9, 36.7, 33.5, 20.4. FTIR (thin film) cm⁻¹: 3422 (br), 2952 (w), 2210 (w), 1718 (s), 1438 (m), 1200 (s), 1156 (s), 1074 (m), 1014 (m). HRMS (ESI) (m/z): calc'd for C₈H₁₁BrO₃ [M+H]⁺: 234.9970, found: 234.9965. TLC (25% ethyl acetate in hexanes), Rf: 0.26 (KMnO₄).

### Methyl 7-bromo-5-chlorohept-6-ynoate

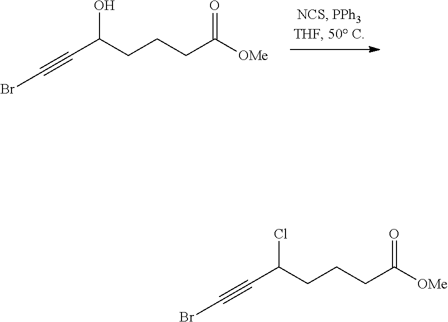 [SMILES: COC(=O)CCCC(O)C#CBr ; COC(=O)CCCC(Cl)C#CBr]

Tetrahydrofuran (12.5 mL) was added to a flask charged with methyl 7-bromo-5-hydroxyhept-6-ynoate (587 mg, 2.5 mmol, 1 equiv), N-chlorosuccinimide (734 mg, 5.5 mmol, 2.2 equiv) and triphenylphosphine (1.31 g, 5 mmol, 2 equiv). The reaction mixture was stirred at 50° C. for 1.5 h. The resulting mixture was then quenched with two drops of water and then concentrated under reduced pressure. The resulting concentrate was purified by flash column chromatography on silica gel (eluent: 5% ethyl acetate/hexanes) to give the title compound as a light yellow oil (55%). ¹H NMR (500 MHz, CDCl₃) δ 4.53 (t, J=6.5 Hz, 1H), 3.66 (s, 3H), 2.35 (t, J=7.3 Hz, 2H), 2.01-1.93 (m, 2H), 1.89-1.80 (m, 2H). ¹³C NMR (126 MHz, CDCl₃) δ 173.4, 77.7, 51.7, 48.6, 47.7, 38.2, 33.1, 21.5. FTIR (thin film) cm⁻¹: 2952 (w), 2222 (w), 1730 (s), 1435 (m), 1197 (m), 1152 (m), 995 (w), 731 (w). HRMS (APCI) (m/z): calc'd for C₈H₁₀BrClO₂ [M+H]⁺: 252.9625, found: 252.9622. TLC (10% ethyl acetate in hexanes), Rf: 0.50 (KMnO₄).

### 7-Bromo-5-chlorohept-6-ynoic acid

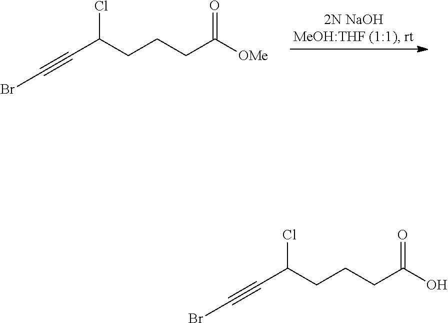 [SMILES: COC(=O)CCCC(Cl)C#CBr ; O=C(O)CCCC(Cl)C#CBr]

A 1:1 mixture of methanol (5 mL) and tetrahydrofuran (5 mL) was added to a flask charged with methyl 7-bromo-5-chlorohept-6-ynoate (507 mg, 2 mmol, 1 equiv). An aqueous solution of 2 N NaOH (5 mL) was subsequently added to the reaction mixture. The resulting solution was stirred at rt for 1 h. The reaction mixture was then acidified with 1 N HCl (15 mL), diluted in dichloromethane (50 mL) and extracted with dichloromethane (3×50 mL). The organic layers were combined and washed with brine×2, dried over anhydrous sodium sulfate, filtered, and concentrated under reduced pressure. The resulting concentrate was purified by flash column chromatography on silica gel (eluent: 30% ethyl acetate in hexanes) to the title compound as a colorless oil (92%). ¹H NMR (500 MHz, CDCl₃) δ 4.56 (t, J=6.5 Hz, 1H), 2.43 (t, J=7.3 Hz, 2H), 2.08-1.95 (m, 2H), 1.93-1.83 (m, 2H). ¹³C NMR (126 MHz, CDCl₃) δ 179.6, 77.7, 48.6, 47.9, 38.0, 33.2, 21.2. FTIR (thin film) cm⁻¹: 2937 (br), 2217 (w), 1700 (s), 1412 (w), 1223 (w), 928 (w), 775 (w), 731 (w). HRMS (ESI) (m/z): calc'd for C₇H₈BrClO₂ [M−H]⁻: 236.9323, found: 236.9323. TLC (30% ethyl acetate in hexanes), Rf: 0.24 (KMnO₄).

### N-(21-Bromo-19-chloro-15-oxo-4,7,10-trioxa-14-azahenicos-20-yn-1-yl)-4-sulfamoylbenzamide

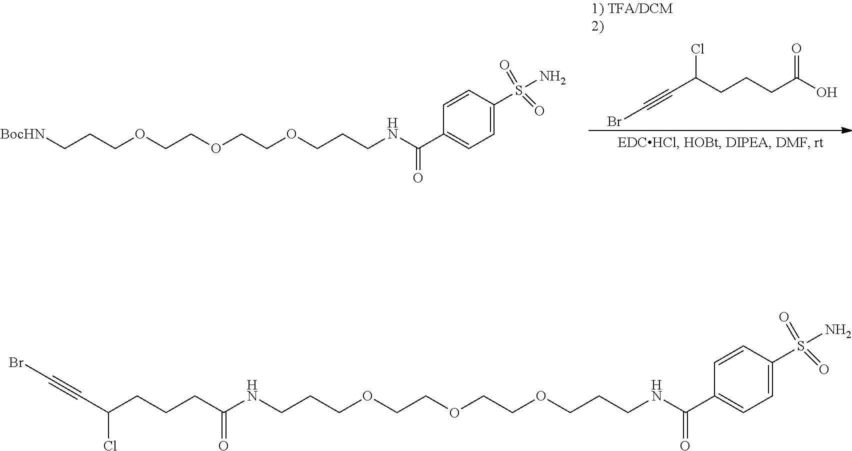 [SMILES: CC(C)(C)OC(=O)NCCCOCCOCCOCCCNC(=O)c1ccc(S(N)(=O)=O)cc1 ; O=C(O)CCCC(Cl)C#CBr ; NS(=O)(=O)c1ccc(C(=O)NCCCOCCOCCOCCCNC(=O)CCCC(Cl)C#CBr)cc1]

An 8 mL glass vial was charged with tert-butyl (1-oxo-1-(4-sulfamoylphenyl)-6,9,12-trioxa-2-azapentadecan-15-yl)carbamate (O'Herin et al., J. Med. Chem. 66:2789-2803 (2023)) (34.2 mg, 0.068 mmol, 1 equiv) at rt. A solution of trifluoroacetic acid in dichloromethane (20% v/v, 500 μL) was introduced via syringe. After 1 h, the reaction mixture was concentrated under reduced pressure and the crude residue was azeotroped with toluene (3×1 mL). The crude residue was then dissolved in anhydrous dimethylformamide (340 μL) and subsequently added to a solution of 7-bromo-5-chlorohept-6-ynoic acid (16.2 mg, 0.068 mmol, 1 equiv), N-ethyl-N-(3-dimethylaminopropyl)carbodiimide (EDC) hydrochloride (19.6 mg, 0.102 mmol, 1.5 equiv), and N,N-diisopropylethylamine (47 μL, 0.272 mmol, 4 equiv) in anhydrous dimethylformamide (340 μL). After 18 h, the reaction was purified by automated C₁₈ reverse phase column chromatography (30 g C₁₈ silica gel, 25 μm spherical particles, eluent: H₂O+0.1% TFA (6 CV), gradient 0→100% MeCN/H₂O+0.1% TFA (10 CV), and further purified on silica gel (eluent: 30%→50% CMA in chloroform) to give the title compound as a colorless oil (58%). ¹H NMR (500 MHz, CD₃OD) δ 8.03-7.94 (m, 4H), 4.75 (t, J=6.5 Hz, 1H), 3.69-3.60 (m, 8H), 3.59-3.55 (m, 2H), 3.52 (q, J=6.5 Hz, 4H), 3.26 (t, J=6.8 Hz, 2H), 2.24 (t, J=7.4 Hz, 2H), 1.97-1.89 (m, 4H), 1.89-1.81 (m, 2H), 1.76 (p, J=6.5 Hz, 2H). ¹³C NMR (126 MHz, CD₃OD) δ 175.3, 168.6, 147.6, 139.1, 129.0, 127.3, 79.0, 71.5, 71.5, 71.3, 71.2, 70.2, 69.9, 49.7, 39.5, 39.4, 38.9, 37.8, 36.0, 30.4, 30.3, 23.6. FTIR (thin film) cm⁻¹: 3287 (br), 2930 (w), 2218 (w), 1640 (s), 1543 (s), 1331 (m), 1163 (s), 1096 (s), 857 (m), 731 (m). HRMS (ESI) (m/z): calc'd for C₂₄H₃₅BrClN₃O₇S [M+H]⁺: 624.1146, found: 624.1145. TLC (40% CMA in chloroform), Rf: 0.26 (UV).

### 23-Bromo-21-chloro-N-methyl-1,17-dioxo-N-(pent-4-yn-1-yl)-1-(4-sulfamoylphenyl)-6,9,12-trioxa-2,16-diazatricos-22-en-23-amine oxide (SA-1, 21′a)

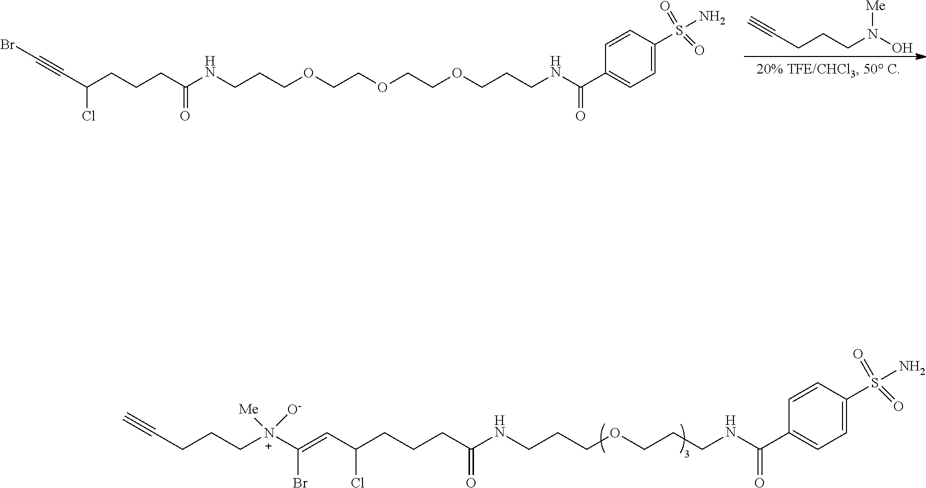

The title compound was synthesized using hydroamination of α,γ-substituted alkynes as described in general procedure A. ¹H NMR (500 MHz, CD₃OD) δ 8.02-7.92 (m, 4H), 7.45 (d, J=9.9 Hz, 1H), 4.75-4.66 (m, 1H), 3.92 (dtd, J=16.8, 11.8, 4.9 Hz, 1H), 3.67-3.58 (m, 8H), 3.55 (dd, J=6.1, 3.3 Hz, 2H), 3.52-3.46 (m, 5H), 3.43 (s, 3H), 3.24 (td, J=6.9, 1.1 Hz, 2H), 2.37-2.19 (m, 5H), 2.09-1.98 (m, 1H), 1.98-1.85 (m, 4H), 1.85-1.77 (m, 1H), 1.77-1.58 (m, 4H). ¹³C NMR (126 MHz, CD₃OD) δ 173.7, 167.2, 146.3, 137.8, 132.0, 130.8, 127.6, 126.0, 81.7, 70.2, 69.9, 69.7, 68.9, 68.5, 68.3, 59.4, 59.0, 57.7, 37.5, 36.8, 36.4, 34.7, 34.7, 29.1, 28.9, 22.3, 21.9, 14.7. FTIR (thin film) cm⁻¹: 3265 (br), 2926 (w), 2870 (w), 2117 (w), 1640 (s), 1543 (s), 1450 (m), 1331 (m), 1163 (s), 1096 (s), 857 (w), 764 (w), 731 (w). HRMS (ESI) (m/z): calc'd for C₃₀H₄₆BrClN₄O₈S [M+H]⁺: 737.1986, found: 737.1986. TLC (50% CMA in chloroform), Rf: 0.12 (UV).

### 3-Bromoprop-2-yn-1-yl (4-nitrophenyl) carbonate

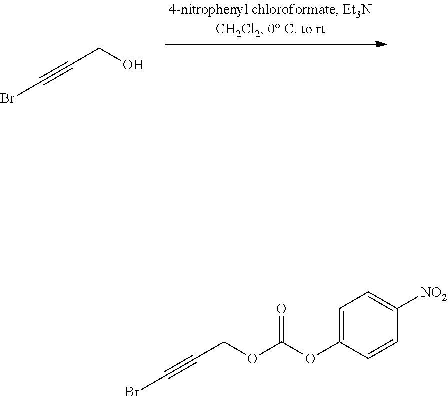 [SMILES: OCC#CBr ; O=C(OCC#CBr)Oc1ccc([N+](=O)[O-])cc1]

Dichloromethane (46 mL) was added to a flask charged with 3-bromoprop-2-yn-1-ol (1.24 g, 9.18 mmol, 1 equiv) and cooled to 0° C. 4-nitrochloroformate (2.03 g, 10.1 mmol, 1.1 equiv) and triethylamine (1.8 mL, 12.85 mmol, 1.4 equiv) were subsequently added at 0° C. The reaction mixture was then stirred at rt for 3 h. The reaction mixture was then quenched with saturated aqueous ammonium chloride and then extracted with dichlormethane (3×50 mL). The organic layers were combined and washed with brine×2, dried over anhydrous sodium sulfate, filtered, and concentrated under reduced pressure. The concentrate with purified by flash column chromatography on silica gel (eluent: 10% EtOAc/hexane) to give the title compound as a white solid.

### Methyl N-(((3-bromoprop-2-yn-1-yl)oxy)carbonyl)-N-methylglycinate

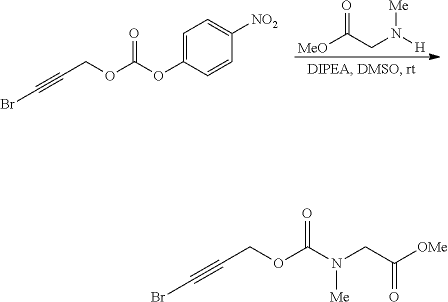 [SMILES: O=C(OCC#CBr)Oc1ccc([N+](=O)[O-])cc1 ; CNCC(=O)OC ; COC(=O)CN(C)C(=O)OCC#CBr]

Dimethylsulfoxide (4.6 mL) was added to a flask charged with methyl methylglycinate (193 mg, 1.38 mmol, 1.5 equiv) and 3-bromoprop-2-yn-1-yl (4-nitrophenyl) carbonate (277 mg, 0.923 mmol, 1 equiv). N,N-diisopropylethylamine (482 μL, 2.77 mmol, 3 equiv) was subsequently added via syringe. The resulting solution was stirred at rt for 2 h. The reaction mixture was then quenched with 5% citric acid (15 mL), diluted in ethyl acetate and extracted with ethyl acetate (3×40 mL). The organic layers were combined and washed with brine×2, dried over anhydrous sodium sulfate, filtered, and concentrated under reduced pressure. The concentrate was purified by flash column chromatography on silica gel (eluent: 25% ethyl acetate/hexanes) to give the title compound as a white solid.

### N-(((3-bromoprop-2-yn-1-yl)oxy)carbonyl)-N-methylglycine

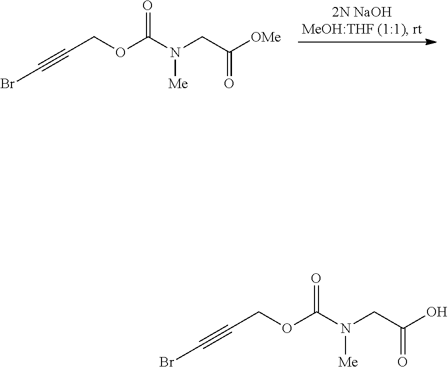 [SMILES: COC(=O)CN(C)C(=O)OCC#CBr ; CN(CC(=O)O)C(=O)OCC#CBr]

A 1:1 mixture of methanol (2.2 mL) and tetrahydrofuran (2.2 mL) were added to a flask charged with methyl N-(((3-bromoprop-2-yn-1-yl)oxy)carbonyl)-N-methylglycinate (240 mg, 0.908 mmol, 1 equiv). An aqueous solution of 2 N NaOH (2.2 mL) was subsequently added to the reaction mixture. The resulting solution was stirred at rt for 1 h. The reaction mixture was then acidified with 1 N HCl (20 mL), diluted in ethyl acetate (40 mL) and extracted with ethyl acetate (3×40 mL). The organic layers were combined and washed with brine×2, dried over anhydrous sodium sulfate, filtered, and concentrated under reduced pressure. The crude carboxylic acid was taken to the next step without further purification.

### 3-Bromoprop-2-yn-1-yl methyl(2-oxo-2-((6-(4-sulfamoylbenzamido)hexyl)amino)ethyl)carbamate

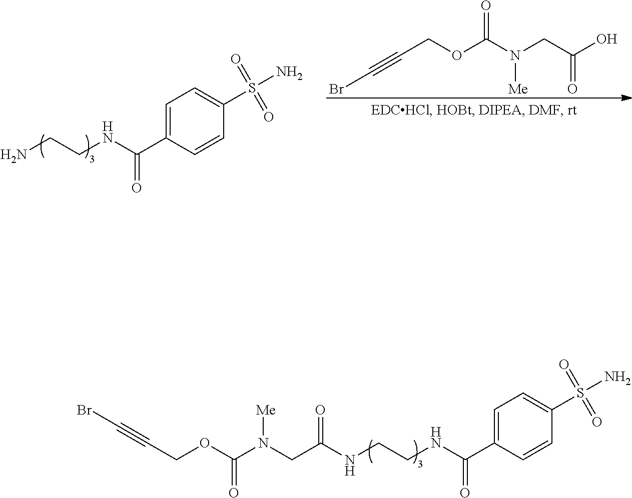

To a solution of N-(((3-bromoprop-2-yn-1-yl)oxy)carbonyl)-N-methylglycine (47 mg, 0.187 mmol, 1 equiv), N-(3-dimethylaminopropyl)-N′-ethylcarbodiimide hydrochloride (54 mg, 0.281 mmol, 1.5 equiv), 1-hydroxybenzotriazole (wetted with not less than 20 wt % water) (47 mg, 0.281 mmol, 1.5 equiv), and N,N-diisopropylethylamine (98 μL, 0.562 mmol, 3 equiv) in dimethylformamide (900 μL) was added a solution of N-(6-aminohexyl)-4-sulfamoylbenzamide (56 mg, 0.187 mmol, 1 equiv) in dimethylformamide (900 μL). The reaction mixture was stirred at rt for 3 h and purified by automated C18 reverse phase column chromatography (30 g C18 silica gel, 25 m spherical particles, eluent: H₂O+0.1% TFA (6 CV), gradient 0→100% MeCN/H₂O+0.1% TFA (9 CV) followed by flash column chromatography on silica gel (eluent: 66% CMA in chloroform) to give the title compound as a white solid.

### 17-Bromo-N,12-dimethyl-1,10,13-trioxo-N-(pent-4-yn-1-yl)-1-(4-sulfamoylphenyl)-14-oxa-2,9,12-triazaheptadec-16-en-17-amine oxide (SA-8)

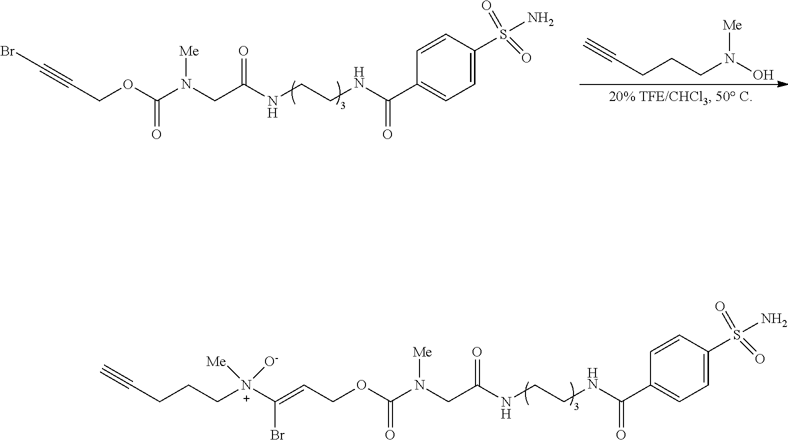

The title compound was synthesized using hydroamination of α,γ-substituted alkynes as described in general procedure A.

### tert-Butyl 1-oxo-1-(4-sulfamoylphenyl)-5,8,11-trioxa-2-azatetradecan-14-oate

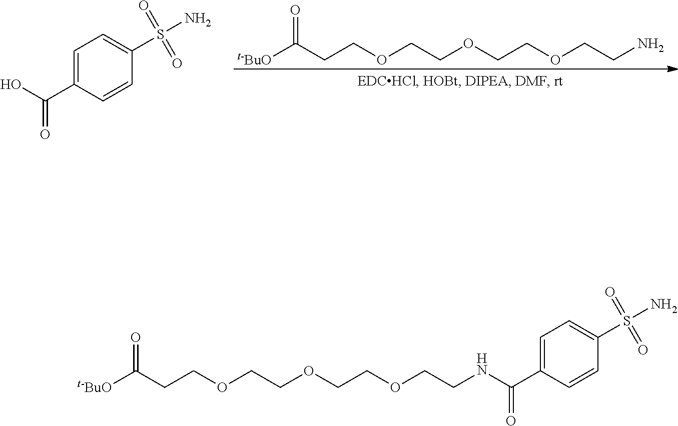 [SMILES: NS(=O)(=O)c1ccc(C(=O)O)cc1 ; CC(C)(C)OC(=O)CCOCCOCCOCCN ; CC(C)(C)OC(=O)CCOCCOCCOCCNC(=O)c1ccc(S(N)(=O)=O)cc1]

A round-bottom flask was charged with 4-sulfamoylbenzoic acid (131 mg, 0.648 mmol, 1 equiv), N-ethyl-N-(3-dimethylaminopropyl)carbodiimide (EDC) hydrochloride (186 mg, 0.972 mmol, 1.50 equiv), and 1-hydroxybenzotriazole hydrate (wetted with no less than 20% wt in water, 164 mg, 0.972 mmol, 1.50 equiv). Anhydrous dimethylformamide (4 mL) and N,N-diisopropylethylamine (339 μL, 1.94 mmol, 3 equiv) were then sequentially added via syringe. After stirring for 5 min, a solution of tert-butyl 3-(2-(2-(2-aminoethoxy)ethoxy)ethoxy)propanoate (131 mg, 0.648 mmol, 1 equiv) dissolved in 2 mL anhydrous dimethylformamide was added. After stirring for 4 h at rt, the reaction was then purified by automated C₁₈ reverse phase column chromatography (30 g C₁₈ silica gel, 25 m spherical particles, eluent: H₂O+0.1% TFA (6 CV), gradient 0→100% MeCN/H₂O+0.1% TFA (10 CV) and further purified on silica gel (eluent: 30% CMA in chloroform) to give the title compound as a colorless oil (33%). ¹H NMR (500 MHz, CD₃OD) δ 8.03-7.93 (m, 4H), 3.68-3.63 (m, 8H), 3.63-3.54 (m, 6H), 2.45 (t, J=6.2 Hz, 2H), 1.44 (s, 9H). ¹³C NMR (126 MHz, CD₃OD) δ 172.8, 168.8, 147.7, 139.0, 129.0, 127.3, 81.8, 71.5, 71.5, 71.3, 71.3, 70.4, 67.9, 41.1, 37.2, 28.4. FTIR (thin film) cm⁻¹: 3257 (br), 2874 (w), 1722 (m), 1648 (m), 1543 (m), 1334 (s), 1156 (s), 1096 (s), 850 (m), 731 (m). HRMS (ESI) (m/z): calc'd for C₂₀H₃₂N₂O₈S [M+H]⁺: 461.1958, found: 461.1952. TLC (30% CMA in chloroform), Rf: 0.26 (UV).

### N-(15-(3-(but-3-yn-1-yl)-3H-diazirin-3-yl)-12-oxo-3,6,9-trioxa-13-azapentadecyl)-4-sulfamoylbenzamide (SA-Dz, 21′b)

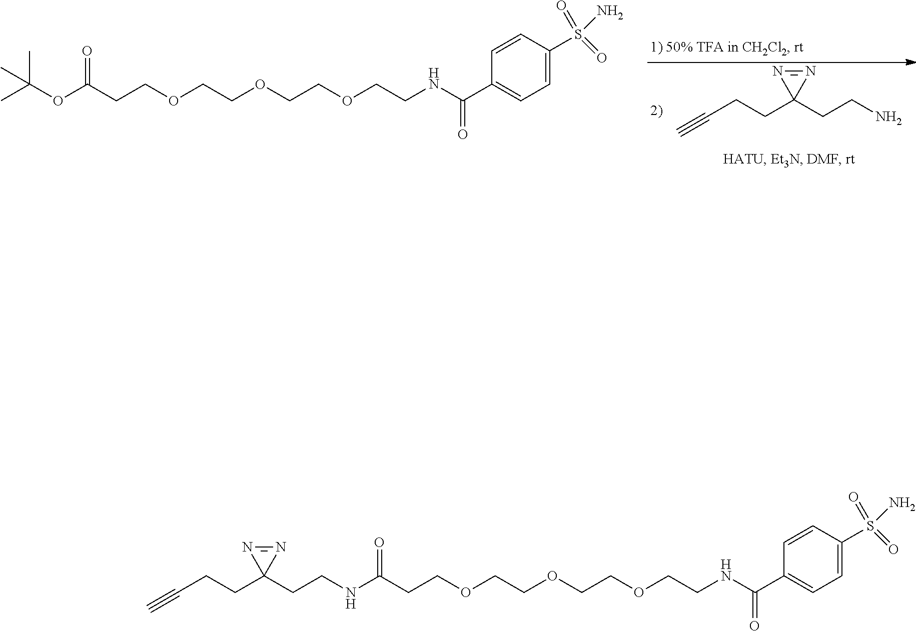

An 8 mL glass vial was charged with tert-butyl 1-oxo-1-(4-sulfamoylphenyl)-5,8,11-trioxa-2-azatetradecan-14-oate (18 mg, 0.039 mmol, 1 equiv) at rt. A solution of trifluoroacetic acid in dichloromethane (50% v/v, 500 μL) was introduced via syringe. After 1 h, the reaction mixture was concentrated under reduced pressure and the crude residue was azeotroped with toluene (3×1 mL). The crude residue was then dissolved in anhydrous dimethylformamide (400 μL), and N-ethyl-N-(3-dimethylaminopropyl)carbodiimide (EDC) hydrochloride (11.2 mg, 0.059 mmol 1.50 equiv) and N,N-diisopropylethylamine (27 μL, 0.156 mmol, 4.00 equiv) were subsequently added at rt. After stirring for 5 min, a solution of 2-(3-(but-3-yn-1-yl)-3H-diazirin-3-yl)ethan-1-amine (5.3 mg, 0.039 mmol, 1 equiv) in anhydrous dimethylformamide (400 μL) was then added at rt. After 3 h stirring in the dark, the reaction was purified by automated C₁₈ reverse phase column chromatography (30 g C₁₈ silica gel, 25 m spherical particles, eluent: H₂O+0.1% TFA (6 CV), gradient 0→100% MeCN/H₂O+0.1% TFA (10 CV) and further purified on silica gel (eluent: 5% methanol in dichloromethane) to give the title compound as a colorless oil (50%). ¹H NMR (500 MHz, CD₃OD) δ 8.01-7.95 (m, 4H), 3.71-3.66 (m, 4H), 3.65 (s, 4H), 3.64-3.56 (m, 6H), 3.06 (t, J=7.1 Hz, 2H), 2.40 (t, J=6.2 Hz, 2H), 2.28 (t, J=2.7 Hz, 1H), 2.01 (td, J=7.4, 2.7 Hz, 2H), 1.61 (td, J=7.3, 5.4 Hz, 4H). ¹³C NMR (126 MHz, CD₃OD) δ 173.9, 168.9, 147.7, 139.0, 129.1, 127.3, 83.6, 71.6, 71.5, 71.3, 71.3, 70.4, 70.4, 68.2, 41.1, 37.6, 35.3, 33.4, 33.3, 27.9, 13.8. FTIR (thin film) cm⁻¹: 3284 (br), 3086 (w), 2922 (w), 1644 (s), 1543 (s), 1439 (w), 1334 (m), 1167 (s), 1096 (s), 857 (m), 764 (m), 731 (m). HRMS (ESI) (m/z): calc'd for C₂₃H₃₃N₅O₇S [M+H]⁺: 524.2179, found: 524.2175. TLC (5% methanol in dichloromethane), Rf: 0.11 (UV).

### tert-Butyl (6-(7-bromo-5-chlorohept-6-ynamido)hexyl)carbamate

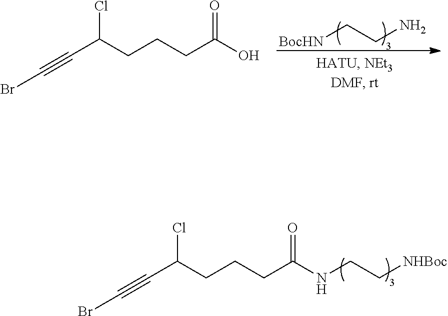

An 8 mL glass vial was charged with 7-bromo-5-chlorohept-6-ynoic acid (55 mg, 0.230 mmol, 1 equiv), 1-[bis(dimethylamino)methylene]-1H-1,2,3-triazolo[4,5-b]pyridinium 3-oxide hexafluorophosphate (HATU, 131 mg, 0.345 mmol, 1.50 equiv), and dissolved in anhydrous dimethylformamide (575 μL) at rt. Triethylamine (64 μL, 0.460 mmol, 2.00 equiv) was then added via syringe. After stirring for 5 min, a solution of tert-butyl (6-aminohexyl)carbamate (49.5 mg, 0.230 mmol, 1 equiv) dissolved in anhydrous dimethylformamide (575 μL) was added via syringe. After 3 h, the reaction was extracted with ethyl acetate (3×10 mL) and the combined organic layers were washed with brine (2×20 mL), dried over sodium sulfate and concentrated under reduced pressure. The crude residue was purified on silica gel (eluent: 50% ethyl acetate in hexanes) to give the title compound as a colorless oil (79%). ¹H NMR (500 MHz, CDCl₃) δ 5.85 (s, 1H), 4.58 (s, 1H), 4.54 (t, J=6.5 Hz, 1H), 3.22 (td, J=7.0, 5.8 Hz, 2H), 3.09 (q, J=6.8 Hz, 2H), 2.21 (t, J=7.3 Hz, 2H), 2.00-1.93 (m, 2H), 1.90-1.81 (m, 2H), 1.47 (dt, J=14.0, 7.0 Hz, 4H), 1.42 (s, 9H), 1.34-1.30 (m, 4H). ¹³C NMR (126 MHz, CDCl₃) δ 172.1, 156.3, 79.2, 77.9, 48.8, 47.6, 40.2, 39.2, 38.4, 35.6, 30.1, 29.5, 28.5, 26.2, 26.0, 22.4. FTIR (thin film) cm⁻¹: 3314 (br), 2930 (m), 2859 (w), 2218 (w), 1689 (s), 1640 (s), 1525 (s), 1454 (m), 1390 (m), 1342 (m), 1249 (s), 1167 (s), 731 (m). HRMS (ESI) (m/z): calc'd for C₁₈H₃₀BrClN₂O₃ [M+H]⁺: 437.1207, found: 437.1203. TLC (50% ethyl acetate in hexanes), Rf: 0.28 (KMnO₄).

### 3-Bromoprop-2-yn-1-yl (6-(4-sulfamoylbenzamido)hexyl)carbamate

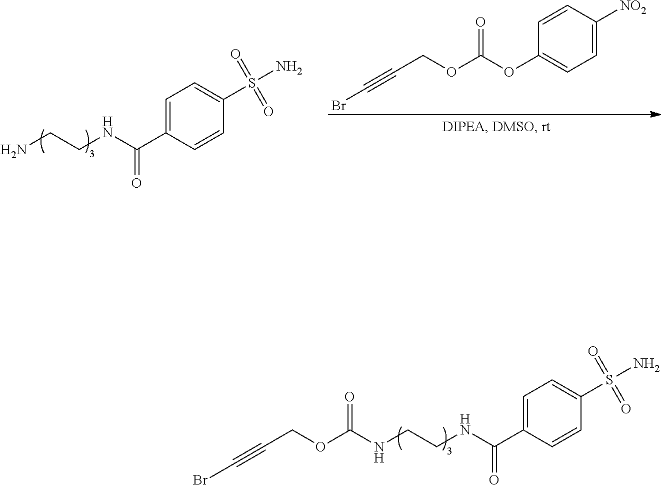

To a solution of a solution of 3-bromoprop-2-yn-1-yl (4-nitrophenyl) carbonate (40 mg, 0.132 mmol, 1.2 equiv) in dimethylsulfoxide (1.3 mL) was added N-(6-aminohexyl)-4-sulfamoylbenzamide (33 mg, 0.11 mmol, 1 equiv) and N,N-diisopropylethylamine (60 μL, 0.33 mmol, 3 equiv). The reaction mixture was stirred at rt for 3 h and purified by automated C18 reverse phase column chromatography (30 g C18 silica gel, 25 m spherical particles, eluent: H₂O+0.1% TFA (6 CV), gradient 0→100% MeCN/H₂O+0.1% TFA (9 CV) followed by flash column chromatography on silica gel (eluent: 66% CMA in chloroform) to give the title compound as a white solid.

### N-(1-Bromo-3-(((6-(4-sulfamoylbenzamido)hexyl)carbamoyl)oxy)prop-1-en-1-yl)-N-methylpent-4-yn-1-amine oxide (SA-2)

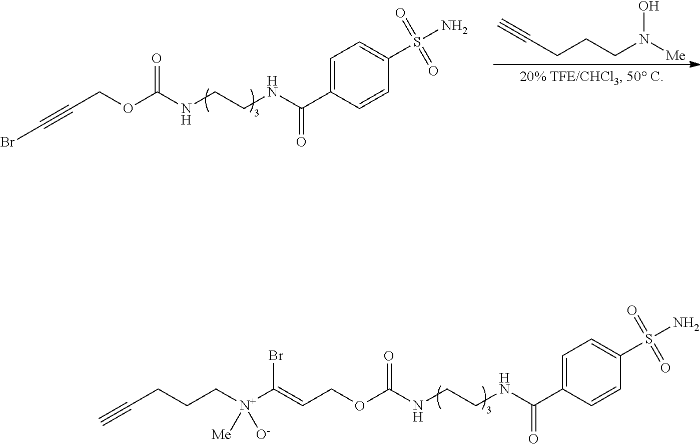

The title compound was synthesized using hydroamination of α,γ-substituted alkynes as described in general procedure A.

### Methyl 8-(4-sulfamoylbenzamido)octanoate

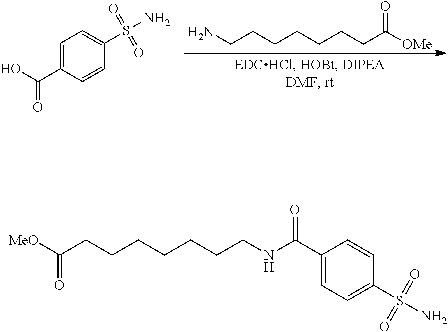 [SMILES: NS(=O)(=O)c1ccc(C(=O)O)cc1 ; COC(=O)CCCCCCCN ; COC(=O)CCCCCCCNC(=O)c1ccc(S(N)(=O)=O)cc1]

Dimethylformamide (10 mL) was added to a flask charged with 4-sulfamoylbenzoic acid (581 mg, 2.89 mmol, 1 equiv), N-(3-dimethylaminopropyl)-N′-ethylcarbodiimide hydrochloride (828 mg, 4.32 mmol, 1.5 equiv), 1-hydroxybenzotriazole (wetted with not less than 20 wt % water) (731 mg, 4.32 mmol, 1.5 equiv) at rt. N,N-diisopropylethylamine (1.51 mL, 8.67 mmol, 3 equiv) was then added via syringe. After 5 min, a solution of methyl 8-aminooctanoate (500 mg, 2.89 mmol, 1 equiv) in dimethylformamide (5 mL) was added via syringe. The reaction mixture was stirred at rt for 12 h. The resulting mixture was then diluted with water and purified by automated C18 reverse phase column chromatography (30 g C18 silica gel, 25 m spherical particles, eluent: H₂O+0.1% TFA (6 CV), gradient 0→100% MeCN/H₂O+0.1% TFA (9 CV). Fractions containing the desired product were collected and concentrated under reduced pressure to give the title compound as a white solid.

### 8-(4-Sulfamoylbenzamido)octanoic acid

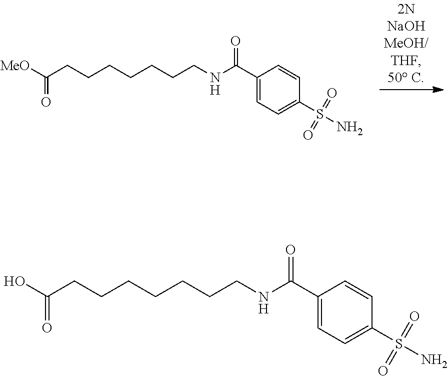 [SMILES: COC(=O)CCCCCCCNC(=O)c1ccc(S(N)(=O)=O)cc1 ; NS(=O)(=O)c1ccc(C(=O)NCCCCCCCC(=O)O)cc1]

A 1:1 mixture of methanol (1.5 mL) and tetrahydrofuran (1.5 mL) were added to a flask charged with methyl 8-(4-sulfamoylbenzamido)octanoate (197 mg, 0.553 mmol, 1 equiv). An aqueous solution of 2 N NaOH (1.5 mL) was subsequently added to the reaction mixture. The resulting solution was stirred at 50° C. for 1 h. The reaction mixture was then acidified with 1 N HCl (5 mL), diluted in ethyl acetate (20 mL) and extracted with ethyl acetate (3×20 mL). The organic layers were combined and washed with brine×2, dried over anhydrous sodium sulfate, filtered, and concentrated under reduced pressure. The crude carboxylic acid was taken to the next step without further purification.

### 3-Bromoprop-2-yn-1-yl 8-(4-sulfamoylbenzamido)octanoate

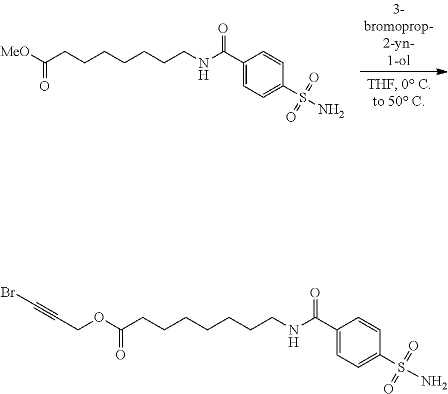 [SMILES: COC(=O)CCCCCCCNC(=O)c1ccc(S(N)(=O)=O)cc1 ; NS(=O)(=O)c1ccc(C(=O)NCCCCCCCC(=O)OCC#CBr)cc1]

Tetrahydrofuran (400 μL) was added to a vial charged with triphenylphosphine (20 mg, 0.074 mmol, 1.2 equiv) at 0° C. after which diisopropyl azodicarboxylate (15 μL, 0.074 mmol, 1.2 equiv) was subsequently added via syringe. After 5 min, the reaction mixture was subsequently transferred to a solution of 8-(4-sulfamoylbenzamido)octanoic acid (21 mg, 0.061 mmol, 1 equiv) dissolved in tetrahydrofuran (400 μL) at 0° C. Then a solution of 3-bromoprop-2-yn-1-ol (10 mg, 0.074 mmol, 1.2 equiv) in tetrahydrofuran (400 μL) was subsequently added via syringe. The reaction mixture was stirred at 50° C. for 8 h. The resulting mixture was then concentrated under reduced pressure. The concentrate with purified by flash column chromatography on silica gel (eluent: 1→4% methanol/dichloromethane) to give the title compound as a white solid.

### N-(1-Bromo-3-((8-(4-sulfamoylbenzamido)octanoyl)oxy)prop-1-en-1-yl)-N-methylpent-4-yn-1-amine oxide (SA-5)

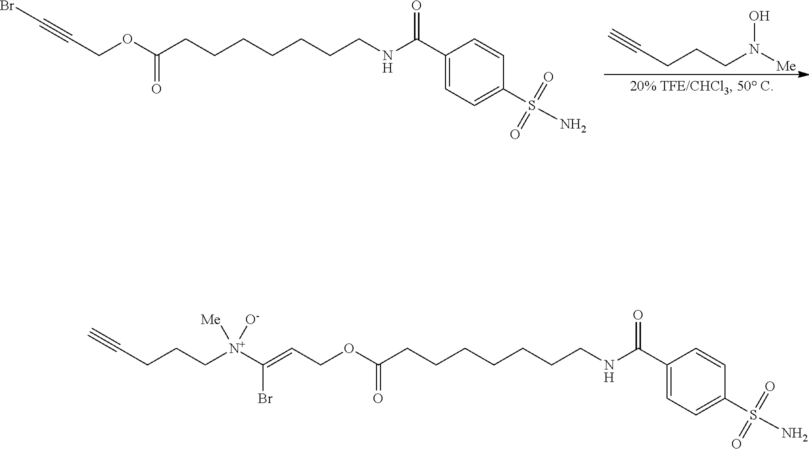

The title compound was synthesized using hydroamination of α,γ-substituted alkynes as described in general procedure A.

### Prop-2-yn-1-yl (6-(4-sulfamoylbenzamido)hexyl) carbonate

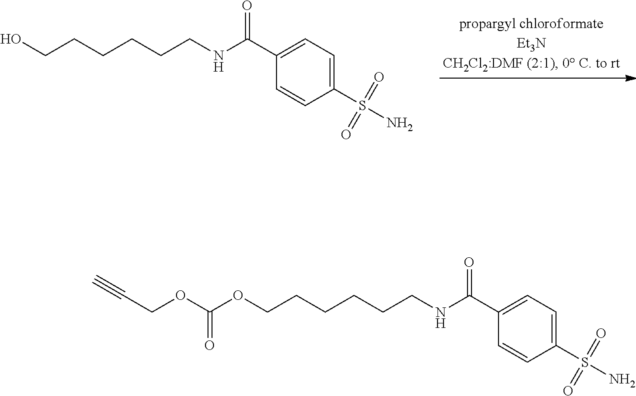 [SMILES: NS(=O)(=O)c1ccc(C(=O)NCCCCCCO)cc1 ; C#CCOC(=O)OCCCCCCNC(=O)c1ccc(S(N)(=O)=O)cc1]

A 2:1 cosolvent of dichloromethane (1 mL) and dimethylformamide (500 μL) was added to a vial charged with N-(6-hydroxyhexyl)-4-sulfamoylbenzamide (25 mg, 0.083 mmol, 1 equiv) at 0° C. Propargyl chloroformate (12.2 μL, 0.124 mmol, 1.5 equiv) and triethylamine (23 μL, 0.166 mmol, 2 equiv) were subsequently added at 0° C. The reaction mixture was stirred at rt for 15 h. The resulting mixture was then diluted with water and purified by automated C18 reverse phase column chromatography (30 g C18 silica gel, 25 m spherical particles, eluent: H₂O+0.1% TFA (6 CV), gradient 0→100% MeCN/H₂O+0.1% TFA (9 CV). Fractions containing the desired product were collected and concentrated under reduced pressure to give the title compound as a white solid.

### 3-Bromoprop-2-yn-1-yl (6-(4-sulfamoylbenzamido)hexyl) carbonate

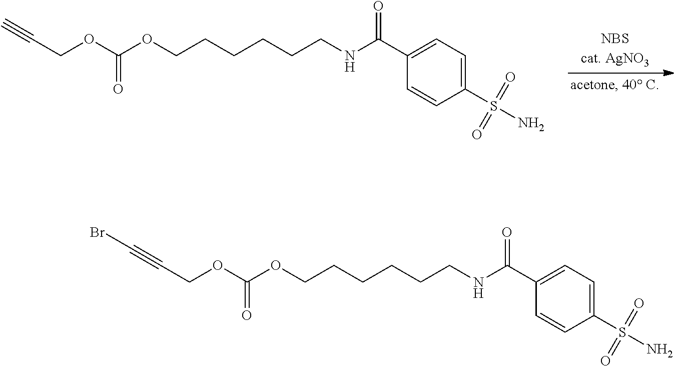 [SMILES: C#CCOC(=O)OCCCCCCNC(=O)c1ccc(S(N)(=O)=O)cc1 ; NS(=O)(=O)c1ccc(C(=O)NCCCCCCOC(=O)OCC#CBr)cc1]

Acetone (520 μL) was added to a vial charged with prop-2-yn-1-yl (6-(4-sulfamoylbenzamido)hexyl) carbonate (10 mg, 0.026 mmol, 1 equiv). Silver nitrate (1 mg, 5.9 mol, 0.22 equiv) and N-bromosuccinimide (5.1 mg, 0.029 mmol, 1.1 equiv) were subsequently added. The reaction mixture was stirred at 40° C. for 1.5 h. The resulting mixture was then concentrated and purified by flash column chromatography on silica gel (eluent: 5→10% methanol/dichloromethane) to give the title compound as a white solid.

### N-(1-Bromo-3-((((6-(4-sulfamoylbenzamido)hexyl)oxy)carbonyl)oxy)prop-1-en-1-yl)-N-methylpent-4-yn-1-amine oxide (SA-6)

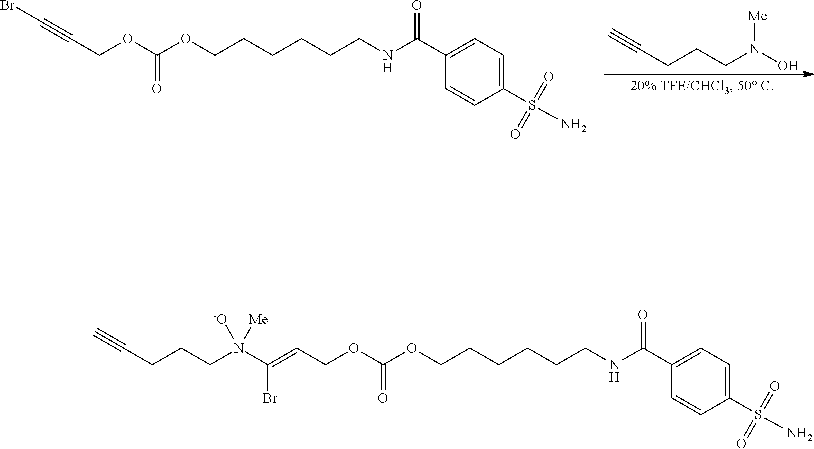

The title compound was synthesized using hydroamination of α,γ-substituted alkynes as described in general procedure A. General Procedure A is applied for the reaction setup. For purification, upon completion of the reaction, the reaction mixture was concentrated and purified by automated C18 reverse phase column chromatography (30 g C18 silica gel, 25 μm spherical particles, eluent: H₂O+0.1% TFA (6 CV), gradient 0→100% MeCN/H₂O+0.1% TFA (9 CV). Fractions containing the desired product were collected and concentrated under reduced pressure to give the title compound as a white solid.

### 4-((3-Bromoprop-2-yn-1-yl)oxy)-N-(6-(4-sulfamoylbenzamido)hexyl)benzamide

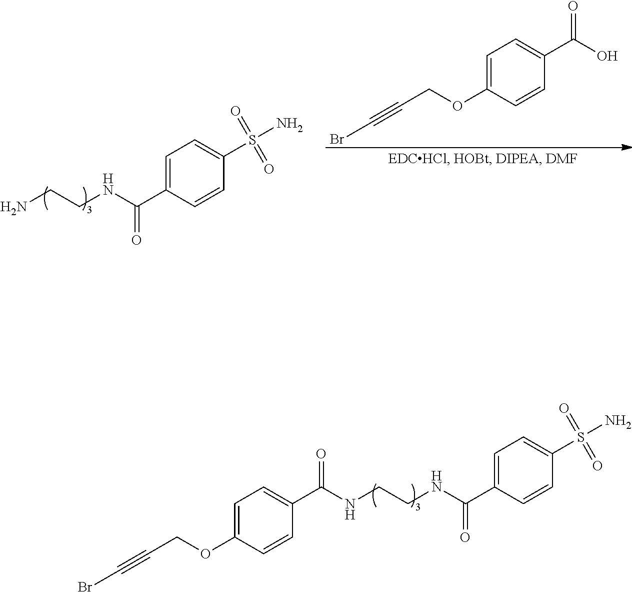

Dimethylformamide (1 mL) was added to a flask charged with 4-((3-bromoprop-2-yn-1-yl)oxy)benzoic acid (50 mg, 0.196 mmol, 1 equiv), N-(3-dimethylaminopropyl)-N′-ethylcarbodiimide hydrochloride (56 mg, 0.294 mmol, 1.5 equiv), 1-hydroxybenzotriazole (wetted with not less than 20 wt % water) (50 mg, 0.294 mmol, 1.5 equiv) at rt. N,N-diisopropylethylamine (102 μL, 0.588 mmol, 3 equiv) was then added via syringe. After 5 min, a solution of N-(6-aminohexyl)-4-sulfamoylbenzamide (59 mg, 0.196 mmol, 1 equiv) in dimethylformamide (1 mL) was added via syringe. The reaction mixture was stirred at rt for 12 h. The resulting mixture was then diluted with water and purified by automated C18 reverse phase column chromatography (30 g C18 silica gel, 25 m spherical particles, eluent: H₂O+0.1% TFA (6 CV), gradient 0→100% MeCN/H₂O+0.1% TFA (9 CV). Fractions containing the desired product were collected and concentrated under reduced pressure to give the title compound as a white solid.

### (N-(1-Bromo-3-(((6-(4-sulfamoylbenzamido)hexyl)carbamoyl)oxy)prop-1-en-1-yl)-N-methylpent-4-yn-1-amine oxide (SA-7)

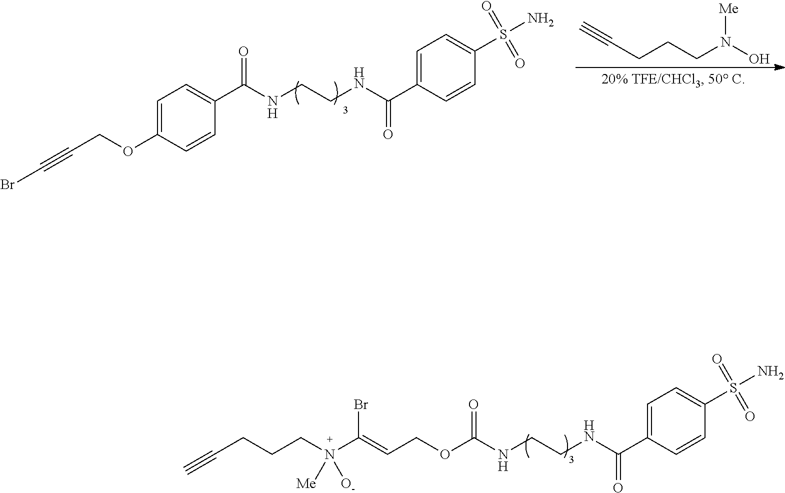

The title compound was synthesized using hydroamination of α,γ-substituted alkynes as described in general procedure A.

### N-(6-(7-Bromo-5-chlorohept-6-ynamido)hexyl)-3,4-difluoro-2-((2-fluoro-4-iodophenyl)amino)benzamide

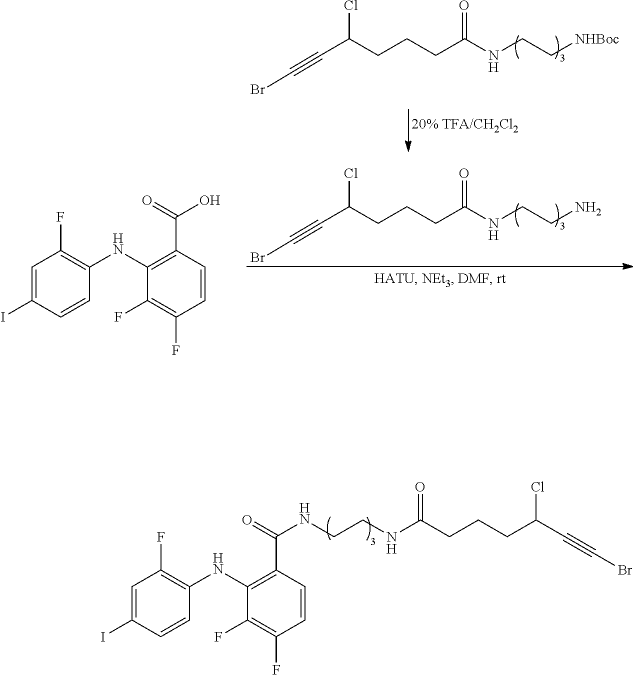

Trifluoracetic acid (80 μL) was added to a solution of tert-butyl (6-(7-bromo-5-chlorohept-6-ynamido)hexyl)carbamate (16 mg, 0.035 mmol, 1 equiv) in dichloromethane (320 μL). The resulting solution was stirred at rt for 1 h then concentrated under reduced pressure. The resulting residue was subsequently dissolved in dimethylformamide (200 μL), and added to a solution of 3,4-difluoro-2-((2-fluoro-4-iodophenyl)amino)benzoic acid (14 mg, 0.035 mmol, 1 equiv), 1-[bis(dimethylamino)methylene]-1H-1,2,3-triazolo[4,5-b]pyridinium 3-oxide hexafluorophosphate (HATU, 20 mg, 0.053 mmol, 1.5 equiv), and triethylamine (25 μL, 0.176 mmol, 5 equiv) in dimethylformamide (200 μL). The reaction mixture was stirred at rt for 3 h. The reaction mixture was then quenched with 5% citric acid (15 mL), diluted in ethyl acetate and extracted with ethyl acetate (3×30 mL). The organic layers were combined and washed with brine×2, dried over anhydrous sodium sulfate, filtered, and concentrated under reduced pressure. The concentrate was purified by flash column chromatography on silica gel (eluent: 2:1 ethyl acetate/hexanes) to give the title compound as a beige solid.

### 1-Bromo-3-chloro-7-((6-(3,4-difluoro-2-((2-fluoro-4-iodophenyl)amino)benzamido)hexyl)amino)-N-methyl-7-oxo-N-(pent-4-yn-1-yl)hept-1-en-1-amine oxide

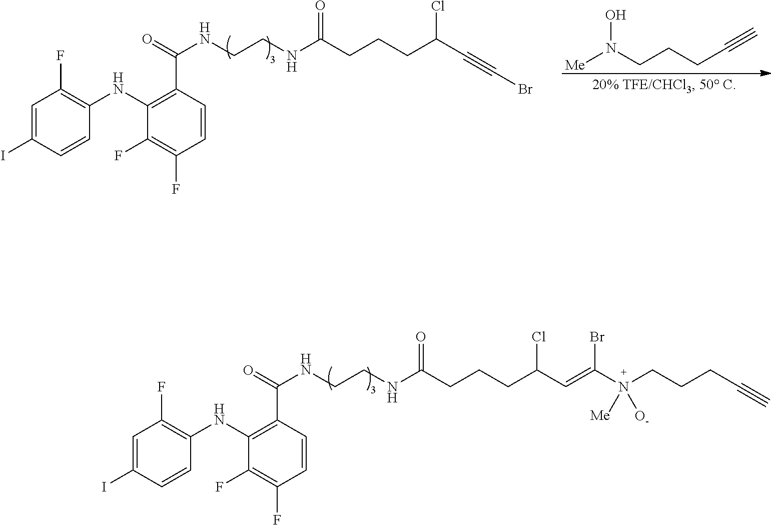

The title compound was synthesized using hydroamination of α,γ-substituted alkynes as described in general procedure A.

### 3-Bromoprop-2-yn-1-yl (2-((tert-butoxycarbonyl)amino)ethyl)(methyl)carbamate

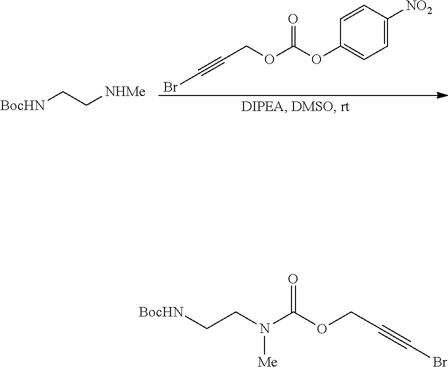 [SMILES: O=C(OCC#CBr)Oc1ccc([N+](=O)[O-])cc1 ; CNCCNC(=O)OC(C)(C)C ; CN(CCNC(=O)OC(C)(C)C)C(=O)OCC#CBr]

Dimethylsulfoxide (2.4 mL) was added to a flask charged with tert-butyl (2-(methylamino)ethyl)carbamate (84 mg, 0.482 mmol, 1.05 equiv) and 3-bromoprop-2-yn-1-yl (4-nitrophenyl) carbonate (138 mg, 0.459 mmol, 1 equiv). N,N-diisopropylethylamine (160 μL, 0.918 mmol, 2 equiv) was subsequently added via syringe. The resulting solution was stirred at rt for 3 h. The reaction mixture was then quenched with 5% citric acid (15 mL), diluted in ethyl acetate and extracted with ethyl acetate (3×30 mL). The organic layers were combined and washed with brine×2, dried over anhydrous sodium sulfate, filtered, and concentrated under reduced pressure. The concentrate was purified by flash column chromatography on silica gel (eluent: 30% ethyl acetate/hexanes) to give the title compound as a white solid. Rf=0.32 in 30% ethyl acetate/hexanes (KMnO₄).

### 3-Bromoprop-2-yn-1-yl (2-(3,4-difluoro-2-((2-fluoro-4-iodophenyl)amino)benzamido)ethyl)(methyl)carbamate

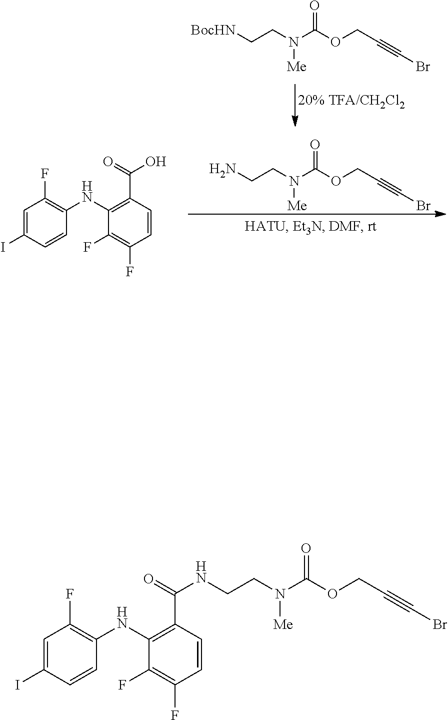 [SMILES: CN(CCNC(=O)OC(C)(C)C)C(=O)OCC#CBr ; O=C(O)c1ccc(F)c(F)c1Nc1ccc(I)cc1F ; CN(CCN)C(=O)OCC#CBr ; CN(CCNC(=O)c1ccc(F)c(F)c1Nc1ccc(I)cc1F)C(=O)OCC#CBr]

Trifluoracetic acid (80 μL) was added to a solution of carbamate (25 mg, 0.076 mmol, 1 equiv) in dichloromethane (320 μL). The resulting solution was stirred at rt for 1 h then concentrated under reduced pressure. The resulting residue was subsequently dissolved in dimethylformamide, and added to a solution of 3,4-difluoro-2-((2-fluoro-4-iodophenyl)amino)benzoic acid (30 mg, 0.076 mmol, 1 equiv), 1-[bis(dimethylamino)methylene]-1H-1,2,3-triazolo[4,5-b]pyridinium 3-oxide hexafluorophosphate (HATU, 43 mg, 0.114 mmol, 1.5 equiv), and triethylamine (43 μL, 0.304 mmol, 5 equiv) in dimethylformamide (800 μL). The reaction mixture was stirred at rt for 3 h. The resulting mixture was then diluted with ethyl acetate and extracted with ethyl acetate (3×10 mL). The organic layers were combined and washed with brine, then 5% citric acid, then saturated aqueous sodium bicarbonate, dried over anhydrous sodium sulfate, filtered, and concentrated under reduced pressure. The concentrate was purified by flash column chromatography on silica gel (eluent: 40% ethyl acetate/hexanes) to give the title compound as a light brown oil.

### N-(1-Bromo-3-(((2-(3,4-difluoro-2-((2-fluoro-4-iodophenyl)amino)benzamido)ethyl)(methyl)carbamoyl)oxy)prop-1-en-1-yl)-N-methylpent-4-yn-1-amine oxide

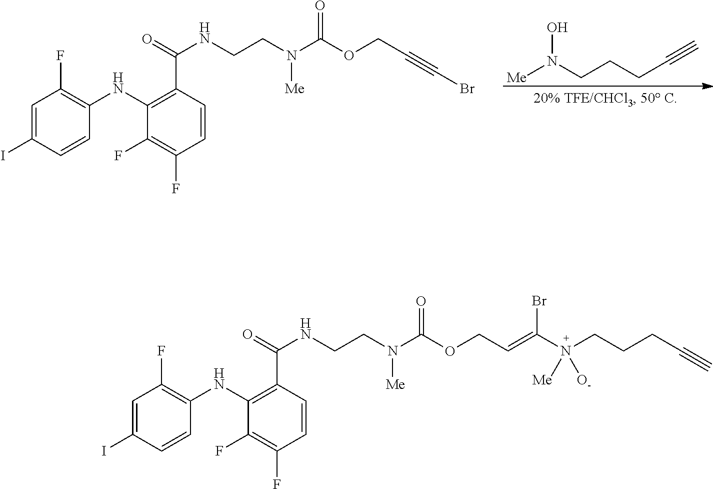

The title compound was synthesized using hydroamination of α,γ-substituted alkynes as described in general procedure A.

### tert-Butyl 3-(2-(2-(7-bromo-5-chlorohept-6-ynamido)ethoxy)ethoxy)propanoate

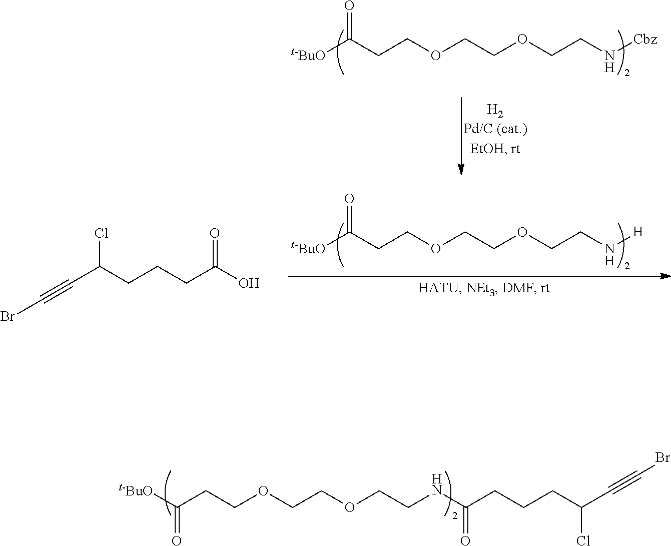

A solution of tert-butyl 3-oxo-1-phenyl-2,7,10-trioxa-4-azatridecan-13-oate (100 mg, 0.1875 mmol, 1 equiv) in ethanol (4 mL) was added to a flask charged with palladium (10%) on carbon (type 487, 10 mg, 9.4 μmol, 5 mol %). Upon evacuation of the flask with vacuum, hydrogen gas was introduced via a hydrogen-filled balloon. The reaction was allowed to stir at rt for 2 h. After completion of the reaction as determined by TLC, the reaction mixture was filtered over a pad of Celite® and the filtrate was concentrated under reduced pressure. The crude concentrate was subsequently dissolved in dimethylformamide (950 μL) and added to a solution of 7-bromo-5-chlorohept-6-ynoic acid (45 mg, 0.187 mmol, 1 equiv), 1-[bis(dimethylamino)methylene]-1H-1,2,3-triazolo[4,5-b]pyridinium 3-oxide hexafluorophosphate (HATU, 107 mg, 0.280 mmol, 1.5 equiv) and triethylamine (78 μL, 0.560 mmol, 3 equiv) in dimethylformamide (950 μL). The reaction mixture was stirred at rt for 15 h and purified by automated C18 reverse phase column chromatography (30 g C18 silica gel, 25 m spherical particles, eluent: H₂O+0.1% TFA (6 CV), gradient 0→100% MeCN/H₂O+0.1% TFA (9 CV) to give the title compound as a clear oil.

### 2-((6-((20-Bromo-18-chloro-4,14-dioxo-7,10-dioxa-3,13-diazaicos-19-yn-1-yl)amino)-2-methylpyrimidin-4-yl)amino)-N-(2-chloro-6-methylphenyl)thiazole-5-carboxamide

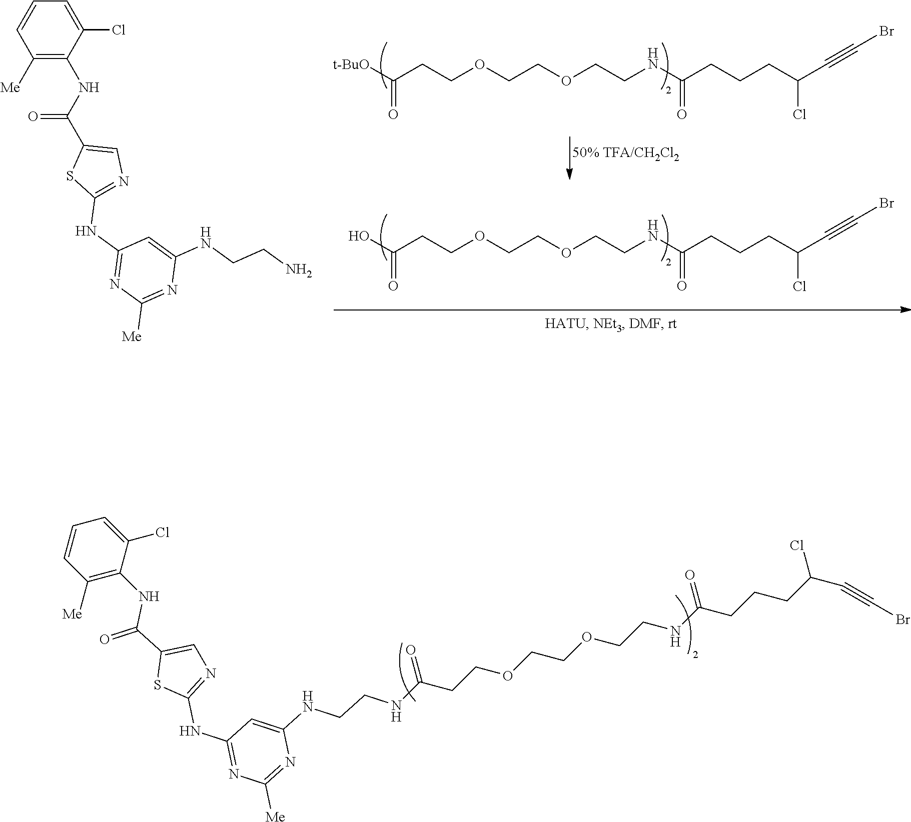

Trifluoracetic acid (350 μL) was added to a solution of tert-butyl 3-(2-(2-(7-bromo-5-chlorohept-6-ynamido)ethoxy)ethoxy)propanoate (34 mg, 0.074 mmol, 1 equiv) in dichloromethane (350 μL). The resulting solution was stirred at rt for 1 h then concentrated under reduced pressure. The resulting residue was subsequently dissolved in dimethylformamide (350 μL), after which 1-[bis(dimethylamino)methylene]-1H-1,2,3-triazolo[4,5-b]pyridinium 3-oxide hexafluorophosphate (HATU, 42 mg, 0.111 mmol, 1.5 equiv) and triethylamine (52 μL, 0.370 mmol, 5 equiv) were added. After stirring for 5 min, a solution of dasatinib-C₂-amine (31 mg, 0.074 mmol, 1 equiv) in dimethylformamide (350 μL) was added. The reaction mixture was stirred at rt for 12 h and purified by automated C18 reverse phase column chromatography (30 g C18 silica gel, 25 μm spherical particles, eluent: H₂O+0.1% TFA (6 CV), gradient 0→100% MeCN/H₂O+0.1% TFA (9 CV) followed by flash column chromatography on silica gel (eluent: 40% CMA in chloroform) to give the title compound as a clear oil.

### 20-Bromo-18-chloro-1-((6-((5-((2-chloro-6-methylphenyl)carbamoyl)thiazol-2-yl)amino)-2-methylpyrimidin-4-yl)amino)-N-methyl-4,14-dioxo-N-(pent-4-yn-1-yl)-7,10-dioxa-3,13-diazaicos-19-en-20-amine oxide

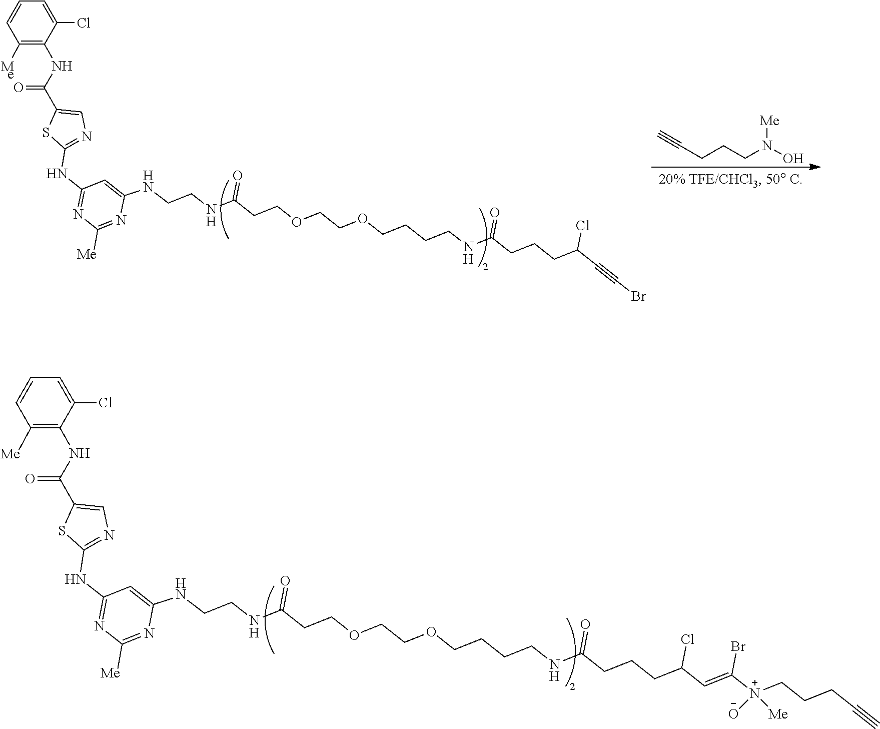

The title compound was synthesized using hydroamination of α,γ-substituted alkynes as described in general procedure A.

### 3-Bromoprop-2-yn-1-yl (2-((2-((6-((5-((2-chloro-6-methylphenyl)carbamoyl)thiazol-2-yl)amino)-2-methylpyrimidin-4-yl)amino)ethyl)amino)-2-oxoethyl)(methyl)carbamate

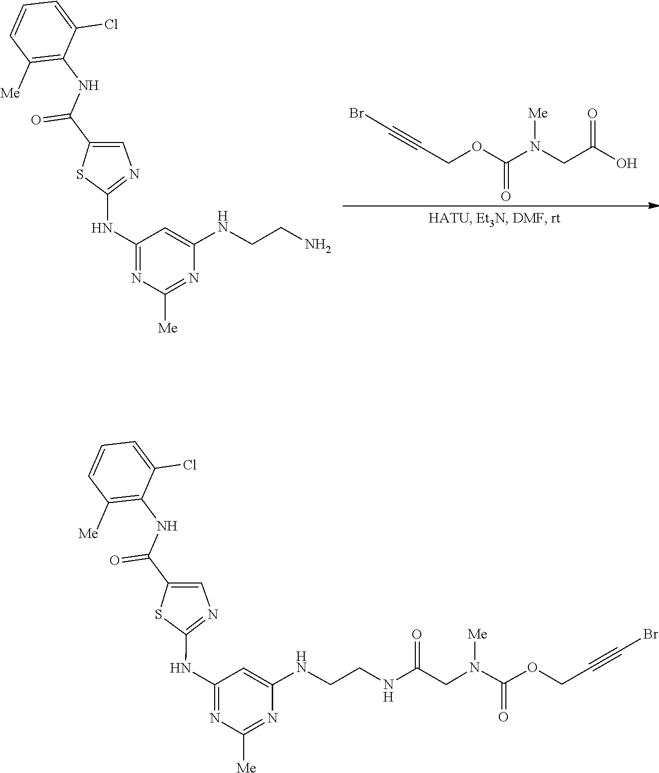 [SMILES: Cc1nc(NCCN)cc(Nc2ncc(C(=O)Nc3c(C)cccc3Cl)s2)n1 ; CN(CC(=O)O)C(=O)OCC#CBr ; Cc1nc(NCCNC(=O)CN(C)C(=O)OCC#CBr)cc(Nc2ncc(C(=O)Nc3c(C)cccc3Cl)s2)n1]

Dimethylformamide (1 mL) was added to a vial charged with N-(((3-bromoprop-2-yn-1-yl)oxy)carbonyl)-N-methylglycine (27 mg, 0.108 mmol, 1 equiv) and 1-[bis(dimethylamino)methylene]-1H-1,2,3-triazolo[4,5-b]pyridinium 3-oxide hexafluorophosphate (HATU, 62 mg, 0.162 mmol, 1.5 equiv) at rt. Triethylamine (45 μL, 0.324 mmol, 3 equiv) was then added via syringe. After 5 min, a solution of 2-((6-((2-aminoethyl)amino)-2-methylpyrimidin-4-yl)amino)-N-(2-chloro-6-methylphenyl)thiazole-5-carboxamide (45 mg, 0.108 mmol, 1 equiv) in dimethylformamide was added via syringe. The reaction mixture was stirred at rt for 4 h and purified by automated C18 reverse phase column chromatography (30 g C18 silica gel, 25 μm spherical particles, eluent: H₂O+0.1% TFA (6 CV), gradient 0→100% MeCN/H₂O+0.1% TFA (9 CV) followed by flash column chromatography on silica gel (eluent: 30% CMA in chloroform) to give the title compound as a white solid.

### N-(1-Bromo-3-(((2-((2-((6-((5-((2-chloro-6-methylphenyl)carbamoyl)thiazol-2-yl)amino)-2-methylpyrimidin-4-yl)amino)ethyl)amino)-2-oxoethyl)(methyl)carbamoyl)oxy)prop-1-en-1-yl)-N-methylpent-4-yn-1-amine oxide

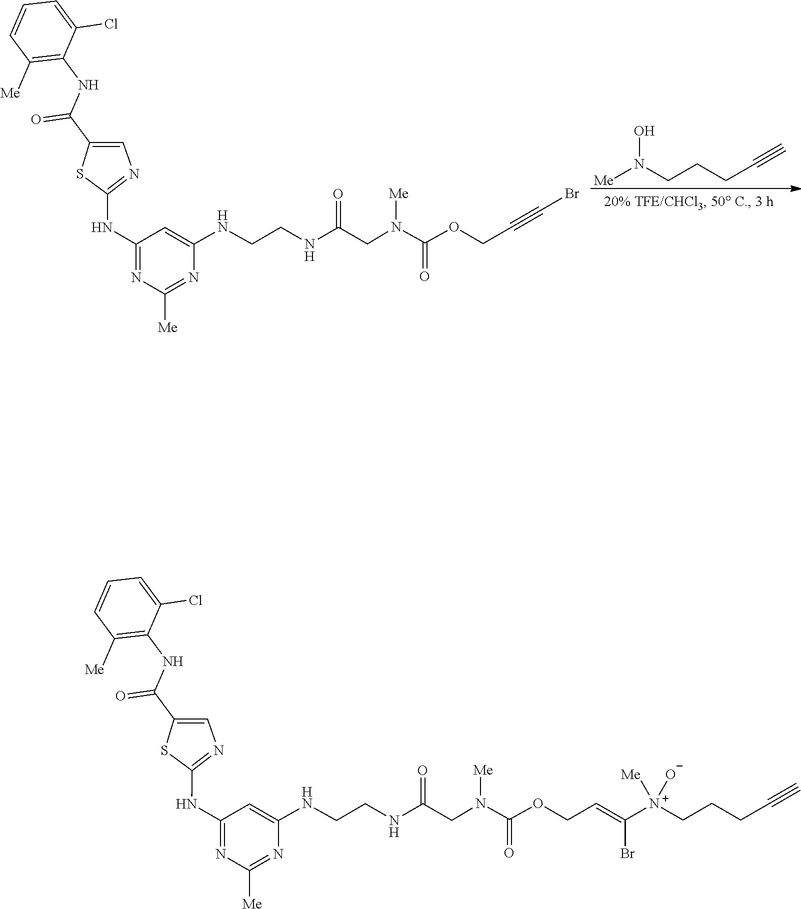

The title compound was synthesized using hydroamination of α,γ-substituted alkynes as described in general procedure A.

### (Z)—N-(1-bromo-3-(((2-((2-(2,6-dioxopiperidin-3-yl)-1-oxoisoindolin-4-yl)amino)-2-oxoethyl)(methyl)carbamoyl)oxy)prop-1-en-1-yl)-N-methylpent-4-yn-1-amine oxide

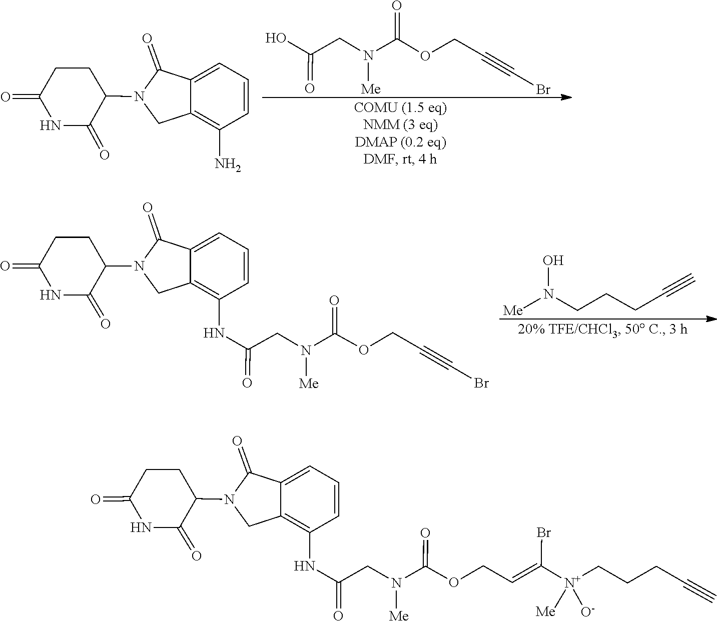

### (Z)—N-(1-bromo-3-(((2-((2-(2,6-dioxopiperidin-3-yl)-1,3-dioxoisoindolin-4-yl)amino)-2-oxoethyl)(methyl)carbamoyl)oxy)prop-1-en-1-yl)-N-methylpent-4-yn-1-amine oxide

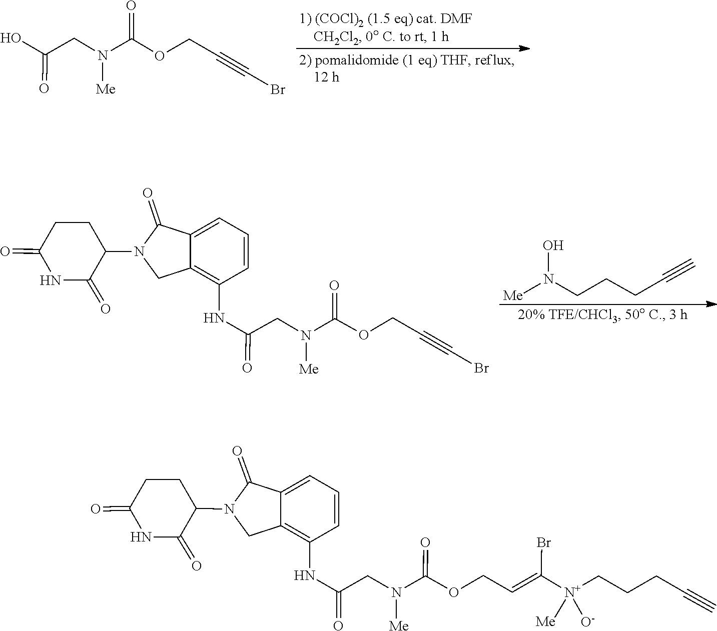

### General Procedure A: Hydroamination (of α,γ-Substituted Alkynes)

A 2 mL LCMS glass vial was charged with N-methyl-N-(pent-4-yn-1-yl)hydroxylamine (3.00 equiv) at rt. A solution of 1-bromoalkene in 20% v/v 2,2,2-trifluoroethanol (TFE) in chloroform (1 equiv, 25-50 mM) was then added via syringe. The vial was flushed with nitrogen, sealed with a septum cap and Parafilm, and heated to 50° C. in an oil bath. After 3 h, the reaction was removed from heat, allowed to cool to rt, and concentrated. The crude residue was then purified by flash column chromatography typically using CMA (10-70% CMA in chloroform) as the eluent system. Fractions containing the desired compound were combined, and the solvent was removed under reduced pressure to provide enamine N-oxide product.

### 1-Bromonona-1,8-diyne

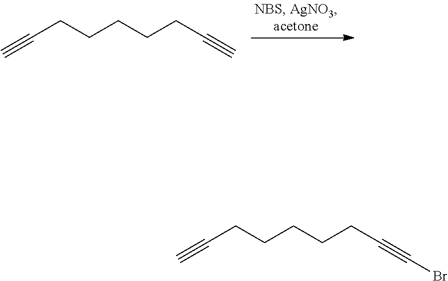

A round-bottom flask was charged with nona-1,8-diyne (300 mg, 2.50 mmol, 1 equiv) and dissolved in acetone (25 mL) at rt. Silver nitrate (42.5 mg, 0.250 mmol, 0.100 equiv) and N-bromosuccinimide (444 mg, 2.50 mmol, 1 equiv) were then added to the reaction mixture and stirred at rt. After 2 h, the reaction was filtered over Celite® and concentrated under reduced pressure. The crude residue was purified on silica gel (eluent: hexanes) to give the title compound as a colorless oil (44%). ¹H NMR (500 MHz, CDCl₃) δ 2.24-2.15 (m, 4H), 1.94 (t, J=2.7 Hz, 1H), 1.61-1.42 (m, 6H). ¹³C NMR (126 MHz, CDCl₃) δ 84.5, 80.2, 68.5, 37.9, 28.1, 28.0, 27.9, 19.7, 18.4. FTIR (thin film) cm⁻¹: 3302 (w), 2937 (m), 2863 (w), 2117 (w), 1461 (w), 1331 (w). TLC (hexanes), Rf: 0.52 (KMnO₄). HRMS (APCI) (m/z): calc'd for C₉H₁₁Br [M+H]⁺: 199.0117, found: 199.0115.

### 1-Bromo-N,N-dimethylnon-1-en-8-yn-1-amine oxide

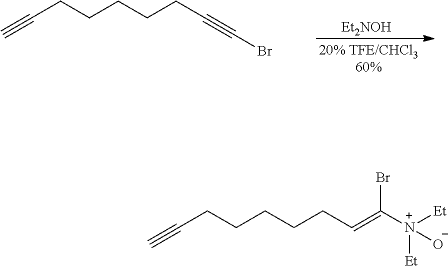

General Procedure A was followed for converting 1-bromononan-1,8-diyne (110 mg, 0.552 mmol) to the title compound as a pale yellow oil (54%) with the following changes: The reaction was conducted in an 8 mL vial sealed with a Teflon cap, with stirring. 5.5 mL of 20% 2,2,2-trifluoroethanol/chloroform solvent were used for the reaction (0.100 M bromoalkene). Additionally, the reaction was heated to 60° C. and the reaction time was extended to 16 h. ¹H NMR (500 MHz, CD₃OD) δ 7.21 (t, J=7.5 Hz, 1H), 3.82 (dq, J=12.4, 7.0 Hz, 2H), 3.35 (dq, J=12.8, 7.3 Hz, 2H), 2.30 (q, J=7.1 Hz, 2H), 2.23-2.15 (m, 3H), 1.59-1.42 (m, 6H), 1.21 (t, J=7.1 Hz, 6H). ¹³C NMR (126 MHz, CD₃OD) δ 132.6, 125.3, 83.4, 68.2, 63.6, 29.4, 28.0, 27.9, 27.2, 17.5, 6.8. FTIR (thin film) cm⁻¹: 3232 (m), 2937 (s), 2113 (w), 1640 (s), 1543 (m), 1450 (s), 1375 (m), 1133 (m), 790 (s). HRMS (ESI) (m/z): calc'd for C₁₃H₂₂BrNO [M+H]⁺: 288.0963, found: 288.0958. TLC (20% CMA in chloroform), Rf: 0.22 (KMnO₄).

### 9-(Trimethylsilyl)nona-1,8-diyn-3-ol

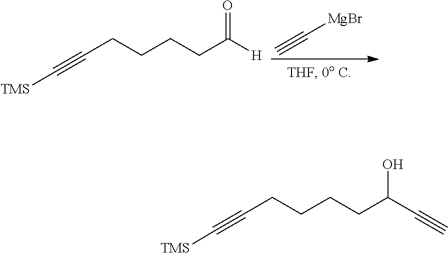

A round-bottom flask was charged with 7-(trimethylsilyl)hept-6-ynal (Goh et al., Org. Lett., 14:6278-6281 (2012)) (510 mg, 2.82 mmol, 1 equiv) and dissolved in anhydrous tetrahydrofuran (13 mL), and then cooled to 0° C. using an ice-water bath. A solution of ethynylmagnesium bromide (0.5 M in tetrahydrofuran, 6.20 mL, 3.10 mmol, 1.10 equiv) was then added dropwise via syringe. After stirring at 0° C. for 15 min, the reaction was quenched with saturated aqueous ammonium chloride solution (10 mL) and extracted with diethyl ether (3×30 mL) and washed with brine (2×50 mL). The organic layer was dried over sodium sulfate, filtered, and concentrated under reduced pressure. The resulting residue was purified by flash column chromatography on silica gel (eluent: 20% ethyl acetate in hexanes) to give the title compound as a colorless oil (72%). ¹H NMR (500 MHz, CDCl₃) δ 4.37 (td, J=6.6, 2.1 Hz, 1H), 2.46 (d, J=2.1 Hz, 1H), 2.37-2.04 (m, 2H), 1.93 (s, 1H), 1.80-1.67 (m, 2H), 1.62-1.44 (m, 4H), 0.13 (s, 9H). ¹³C NMR (126 MHz, CDCl₃) δ 107.3, 84.9, 84.8, 73.1, 62.3, 37.2, 28.3, 24.4, 19.9, 0.3. FTIR (thin film) cm⁻¹: 3295 (br), 2945 (w), 2173 (w), 1249 (m), 1025 (w), 835 (s). HRMS (ESI) (m/z): calc'd for C₁₂H₂₀OSi [M+H]⁻: 209.1362, found: 209.1357. TLC (20% ethyl acetate in hexanes), Rf: 0.38 (KMnO₄).

### 1-Bromonona-1,8-diyn-3-ol

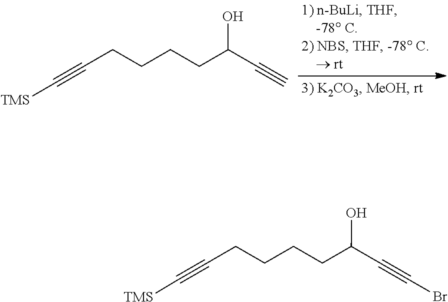

A round-bottom flask was charged with 9-(trimethylsilyl)nona-1,8-diyn-3-ol (424 mg, 2.01 mmol, 1 equiv), dissolved in anhydrous tetrahydrofuran (6 mL) and cooled to −78° C. using an acetone-dry ice bath. A solution of n-butyllithium (2.5 M in hexanes, 1.77 mL, 4.42 mmol, 2.20 equiv) was then added dropwise via syringe and the reaction mixture was stirred at −78° C. for 15 min. Then, N-bromosuccinimide (715 mg, 4.02 mmol, 2.00 equiv) dissolved in anhydrous tetrahydrofuran (4 mL) was added to the reaction mixture dropwise via syringe at −78° C. After the addition was complete, the dry ice bath was removed, and the reaction was allowed to warm to rt. After 2 h, the reaction was quenched with saturated aqueous ammonium chloride solution (10 mL) and extracted with ethyl acetate (3×30 mL) and washed with brine (2×50 mL). The organic layer was dried over sodium sulfate, filtered, and concentrated under reduced pressure. The resulting residue was then dissolved in 10 mL methanol and solid potassium carbonate (555 mg, 4.02 mmol, 2.00 equiv) was added. After stirring at rt for 1 h, the suspension was filtered over Celite and concentrated under reduced pressure. The crude residue was purified on silica gel (eluent: 15% ethyl acetate in hexanes) to give the title compound as a pale yellow oil (36%). ¹H NMR (500 MHz, CDCl₃) δ 4.41 (td, J=6.5, 1.8 Hz, 1H), 2.25-2.17 (m, 2H), 1.95 (t, J=2.7 Hz, 1H), 1.79 (s, 1H), 1.75-1.70 (m, 2H), 1.64-1.51 (m, 4H). ¹³C NMR (126 MHz, CDCl₃) δ 84.4, 81.1, 68.6, 63.2, 45.2, 37.1, 28.1, 24.3, 18.4. FTIR (thin film) cm⁻¹: 3299 (br), 2940 (w), 2863 (m), 2210 (w), 1461 (w), 1331 (w), 1081 (m), 1018 (s). HRMS (ESI) (in z): calc'd for C₉H₁₂BrO [M+H]⁺: 215.0072, found: 215.0069. TLC (15% ethyl acetate in hexanes), Rf: 0.32 (KMnO₄).

### 1-Bromo-3-chloronona-1,8-diyne

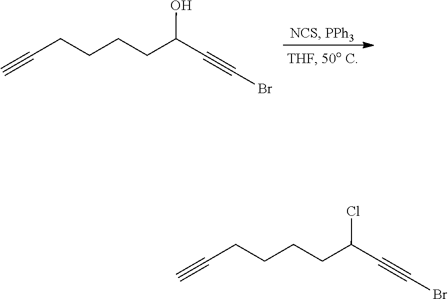

A round-bottom flask was charged with 1-bromonona-1,8-diyn-3-ol (150 mg, 0.697 mmol, 1 equiv) and dissolved in anhydrous tetrahydrofuran (7 mL) at rt. Solid triphenylphosphine (366 mg, 1.40 mmol, 2.00 equiv) and N-chlorosuccinimide (205 mg, 1.534 mmol, 2.20 equiv) were then added to the reaction mixture. After stirring at 50° C. for 1.5 h, two drops of water were added to the reaction mixture and then concentrated under reduced pressure. The crude residue was purified on silica gel (eluent: 10% dichloromethane in hexanes) to give the title compound as a pale yellow oil (72%). ¹H NMR (500 MHz, CDCl₃) δ 4.53 (t, J=6.7 Hz, 1H), 2.22 (td, J=6.9, 2.7 Hz, 2H), 2.02-1.91 (m, 3H), 1.68-1.61 (m, 2H), 1.61-1.52 (m, 2H). ¹³C NMR (126 MHz, CDCl₃) δ 84.0, 78.0, 68.8, 48.9, 47.4, 38.5, 27.7, 25.3, 18.4. FTIR (thin film) cm⁻¹: 3299 (w), 2937 (w), 2218 (w), 1703 (m), 1562 (w), 1223 (m), 846 (m). HRMS (APCI) (m/z): calc'd for C₉H₁₀BrCl [M+H]⁺: 232.9727, found: 232.9729. TLC (10% dichloromethane in hexanes), Rf: 0.37 (KMnO₄).

### 1-Bromo-3-chloro-N,N-dimethylnon-1-en-8-yn-1-amine oxide

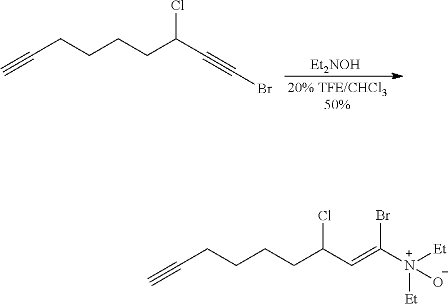 [SMILES: "Et2NOH" ; C#CCCCCC(Cl)C#CBr ; C#CCCCCC(Cl)/C=C(\Br)[N+]([O-])(CC)CC]

General Procedure A was followed for converting 1-bromo-3-chloronona-1,8-diyne (52.3 mg, 0.224 mmol) to the title compound as a colorless oil (71%). ¹H NMR (500 MHz, CD₃OD) δ 7.38 (d, J=10.1 Hz, 1H), 4.73 (dt, J=10.1, 7.0 Hz, 1H), 3.91-3.79 (m, 2H), 3.43-3.32 (m, 2H), 2.24-2.17 (m, 3H), 2.04-1.86 (m, 2H), 1.61-1.51 (m, 4H), 1.22 (td, J=7.1, 4.9 Hz, 6H). ¹³C NMR (126 MHz, CD₃OD) δ 134.5, 130.2, 84.5, 69.9, 65.4, 65.3, 59.2, 38.6, 28.9, 26.5, 18.9, 8.3, 8.0. FTIR (thin film) cm⁻¹: 3299 (m), 2937 (s), 2117 (w), 1647 (s), 1595 (m), 1454 (s), 1375 (s), 1200 (s), 962 (s). HRMS (ESI) (m/z): calc'd for C₁₃H₂₁BrClNO [M+H]⁺: 322.0573, found: 322.0567. TLC (20% CMA in chloroform), Rf: 0.16 (KMnO₄).

### 1-Bromo-5-phenylpent-1-yn-3-ol

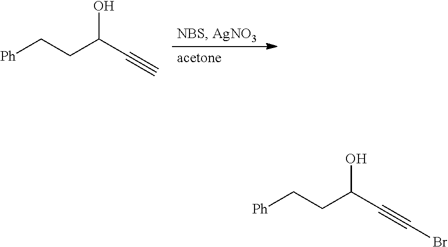

A round-bottom flask was charged with 5-phenylpent-1-yn-3-ol (Cheng et al., Angew. Chem. Int. Ed., 54:13734-13738 (2015)) (450 mg, 2.81 mmol, 1 equiv) and dissolved in acetone (14 mL) at rt. Silver nitrate (48 mg, 0.28 mmol, 0.100 equiv) and N-bromosuccinimide (550 mg, 3.08 mmol, 1.10 equiv) were sequentially added. The flask was covered in aluminum foil and the reaction mixture was stirred at rt in the dark for 18 h. The resulting mixture was then filtered over Celite® and the filtrate was concentrated under reduced pressure. The resulting residue was purified by flash column chromatography on silica gel (eluent: 10% ethyl acetate in hexanes) to give the title compound as a pale yellow oil (60%). ¹H NMR (500 MHz, CDCl₃) δ 7.36-7.28 (m, 2H), 7.23 (d, J=7.4 Hz, 3H), 4.40 (t, J=6.6 Hz, 2H), 2.81 (t, J=7.8 Hz, 2H), 2.23 (s, 1H), 2.12-1.98 (m, 2H). ¹³C NMR (126 MHz, CDCl₃) δ 141.1, 128.6, 126.2, 81.0, 62.7, 45.6, 39.1, 31.4. FTIR (thin film) cm⁻¹: 3325 (br), 2926 (w), 2210 (w), 1495 (w), 1454 (w), 1047 (m), 1010 (m), 697 (s). HRMS (ESI) (m/z): calc'd for C₁₁H₁₁BrO [M+Na]⁺: 260.9885, found: 260.9886. TLC (10% ethyl acetate in hexanes), Rf: 0.26 (UV, KMnO₄).

### (5-Bromo-3-chloropent-4-yn-1-yl)benzene

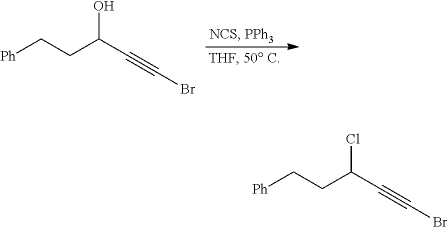 [SMILES: OC(C#CBr)CCc1ccccc1 ; ClC(C#CBr)CCc1ccccc1]

A round-bottom flask was charged with 1-bromo-5-phenylpent-1-yn-3-ol (547 mg, 2.28 mmol, 1 equiv) and dissolved in anhydrous tetrahydrofuran (12 mL) at rt. Solid triphenylphosphine (1.20 g, 4.58 mmol, 2.00 equiv) and N-chlorosuccinimide (672 mg, 5.03 mmol, 2.20 equiv) were then added to the reaction mixture and stirred at 50° C. After 1.5 h, two drops of water were added to the reaction mixture and then concentrated under reduced pressure. The crude residue was purified on silica gel (eluent: 10% dichloromethane in hexanes) to give the title compound as a pale yellow oil (75%). ¹H NMR (500 MHz, CDCl₃) δ 7.34 (dd, J=8.1, 6.8 Hz, 2H), 7.28-7.21 (m, 3H), 4.51 (t, J=6.8 Hz, 1H), 2.89 (dd, J=8.5, 6.7 Hz, 2H), 2.34-2.26 (m, 2H). ¹³C NMR (126 MHz, CDCl₃) δ 140.0, 128.7, 128.6, 78.0, 48.4, 47.7, 40.5, 32.3. FTIR (thin film) cm⁻¹: 3027 (w), 2218 (w), 1603 (w), 1495 (w), 1454 (w), 842 (w), 746 (m), 697 (s). HRMS (APCI) (m/z): calc'd for C₁₁H₁₀BrCl [M]⁺: 255.9649, found: 255.9651. TLC (10% dichloromethane in hexanes), Rf: 0.50 (KMnO₄).

### 1-Bromo-3-chloro-N,N-diethyl-5-phenylpent-1-en-1-amine oxide (7′a)

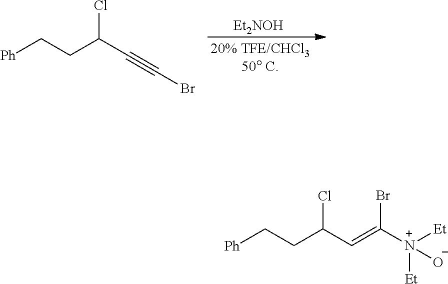 [SMILES: "Et2NOH" ; ClC(C#CBr)CCc1ccccc1 ; CC[N+]([O-])(CC)/C(Br)=C/C(Cl)CCc1ccccc1]

General Procedure A was followed for converting 5-bromo-3-chloropent-4-yn-1-yl)benzene (400 mg, 1.55 mmol) to the title compound as a colorless oil (80%) with the following changes: The reaction was conducted in a 50 mL round-bottom flask with stirring. Additionally, 15.5 mL of 20% 2,2,2-trifluoroethanol/chloroform solvent was used for the reaction (0.100 M bromoalkene). ¹H NMR (500 MHz, CD₃OD) δ 7.43 (d, J=9.9 Hz, 1H), 7.29 (td, J=7.4, 1.4 Hz, 2H), 7.23-7.16 (m, 3H), 4.65 (ddd, J=10.0, 7.5, 6.4 Hz, 1H), 3.89-3.78 (m, 2H), 3.46-3.33 (m, 2H), 2.83-2.70 (m, 2H), 2.30-2.11 (m, 2H), 1.23 (td, J=7.1, 4.7 Hz, 6H). ¹³C NMR (126 MHz, CD₃OD) δ 141.4, 134.4, 130.0, 129.6, 129.5, 127.4, 65.3, 58.7, 40.6, 33.2, 8.2, 8.0. FTIR (thin film) cm⁻¹: 3302 (w), 1778 (m), 1640 (m), 1454 (m), 1137 (s), 798 (m), 701 (s). HRMS (ESI) (m/z): calc'd for C₁₅H₂₁BrClNO [M+H]⁺: 346.0573, found: 346.0568. TLC (15% CMA in chloroform), Rf: 0.14 (KMnO₄).

### 1-Bromo-3-chloro-N-methyl-N-(pent-4-yn-1-yl)-5-phenylpent-1-en-1-amine oxide (7′b)

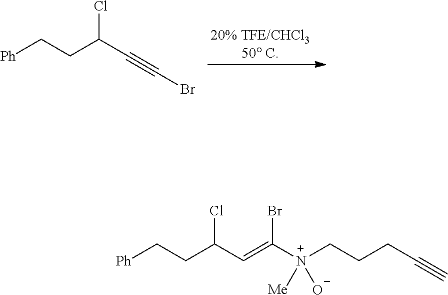 [SMILES: ClC(C#CBr)CCc1ccccc1 ; C#CCCC[N+](C)([O-])/C(Br)=C/C(Cl)CCc1ccccc1]

General Procedure A was followed for converting 5-bromo-3-chloropent-4-yn-1-yl)benzene (18.0 mg, 0.070 mmol) to the title compound as a colorless oil (72%). ¹H NMR (500 MHz, CD₃OD) δ 7.48 (dd, J=9.9, 4.1 Hz, 1H), 7.29 (td, J=7.6, 2.0 Hz, 2H), 7.25-7.17 (m, 3H), 4.67-4.57 (m, 1H), 3.99-3.85 (m, 1H), 3.51 (qd, J=12.1, 4.8 Hz, 1H), 3.45 (d, J=3.2 Hz, 3H), 2.85-2.67 (m, 2H), 2.40-2.13 (m, 5H), 2.10-1.97 (m, 1H), 1.73-1.61 (m, 1H). ¹³C NMR (126 MHz, CD₃OD) δ 141.4, 133.2, 132.3, 129.7, 129.6, 127.4, 83.0, 71.1, 69.7, 60.6, 60.2, 58.7, 40.4, 33.1, 23.4, 23.1, 16.1. FTIR (thin film) cm⁻¹: 3373 (br), 2117 (w), 1648 (w), 1453 (m), 1375 (w), 1200 (w), 1133 (w), 749 (m), 701 (s). HRMS (ESI) (m/z): calc'd for C₁₇H₂₁BrClNO [M+H]⁺: 370.0573, found: 370.0569. TLC (10% CMA in chloroform), Rf: 0.12 (KMnO₄).

### 7-Bromo-5-chloro-N-(6-(2-((S)-4-(4-chlorophenyl)-2,3,9-trimethyl-6H-thieno[3,2-f][1,2,4]triazolo[4,3-a][1,4]diazepin-6-yl)acetamido)hexyl)hept-6-ynamide

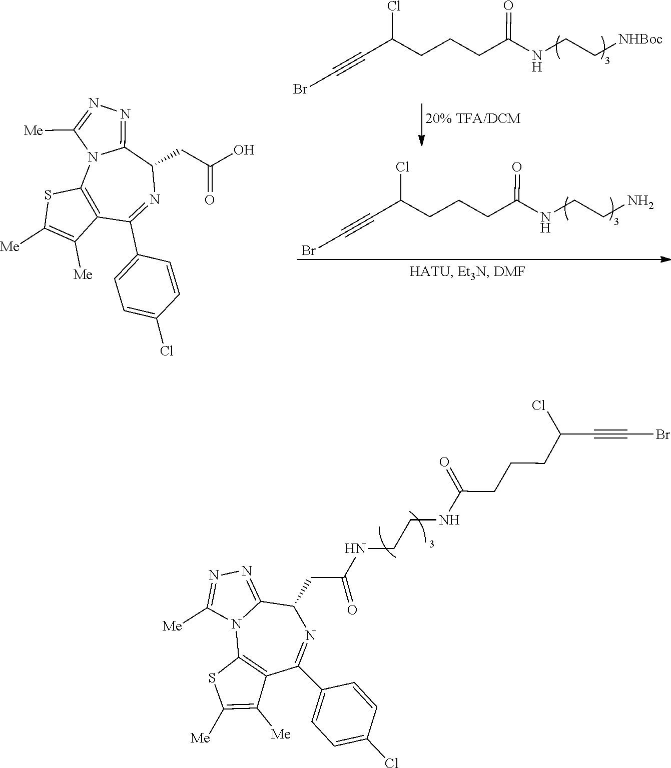

An 8 mL glass vial was charged with tert-butyl (6-(7-bromo-5-chlorohept-6-ynamido)hexyl)carbamate (10.5 mg, 0.024 mmol, 1.10 equiv) at rt. A solution of trifluoroacetic acid in dichloromethane (20% v/v, 500 μL) was introduced via syringe. After 1 h, the reaction mixture was concentrated under reduced pressure and the crude residue was coevaporated with toluene three times (3×1 mL). The crude residue was then dissolved in anhydrous dimethylformamide (150 μL) and subsequently added to a solution of (+)-JQ1 carboxylic acid (9.0 mg, 0.022 mmol, 1 equiv), HATU (12.5 mg, 0.033 mmol, 1.50 equiv), and triethylamine (14 μL, 0.100 mmol, 4.50 equiv) in anhydrous dimethylformamide (150 μL). After 3 h, the reaction was diluted with ethyl acetate (10 mL) and washed with brine (10 mL), aqueous citric acid (5% w/v, 10 mL), and saturated aqueous sodium bicarbonate (10 mL). The combined organic layers were dried over sodium sulfate and concentrated under reduced pressure. The crude residue was purified on silica gel (eluent: 5% methanol in dichloromethane) to give the title compound as a pale yellow oil (73%). ¹H NMR (500 MHz, CDCl₃) δ 7.40 (d, J=8.5 Hz, 1H), 7.32 (d, J=8.8 Hz, 1H), 6.65 (s, 1H), 6.08 (s, 1H), 4.61 (dd, J=8.2, 5.8 Hz, 1H), 4.54 (td, J=6.6, 5.1 Hz, 1H), 3.57 (dd, J=8.3, 0.9 Hz, 1H), 3.39-3.02 (m, 5H), 2.66 (s, 3H), 2.40 (s, 3H), 2.20 (t, J=7.3 Hz, 2H), 2.01-1.92 (m, 2H), 1.90-1.75 (m, 2H), 1.67 (s, 3H), 1.60-1.43 (m, 4H), 1.44-1.26 (m, 4H). ¹³C NMR (126 MHz, CDCl₃) δ 172.3, 170.7, 164.1, 155.8, 150.1, 137.0, 136.7, 132.2, 131.2, 131.1, 130.6, 130.0, 128.9, 77.9, 54.7, 48.8, 47.6, 39.6, 39.3, 39.1, 38.4, 35.6, 29.8, 29.4, 29.3, 26.1, 22.4, 14.5, 13.3, 11.9. FTIR (thin film) cm⁻¹: 3288 (br), 2930 (m), 2855 (m), 2214 (w), 1644 (s), 1532 (s), 1420 (s), 1279 (w), 1178 (w), 1088 (m), 1029 (s), 842 (s). HRMS (ESI) (m/z): calc'd for C₃₂H₃₇BrCl₂N₆O₂S [M+H]⁺: 719.1337, found: 719.1337. TLC (5% methanol in dichloromethane), Rf: 0.10 (UV).

### (Z)-1-Bromo-3-chloro-7-((6-(2-((S)-4-(4-chlorophenyl)-2,3,9-trimethyl-6H-thieno[3,2-f][1,2,4]triazolo[4,3-al][1,4]diazepin-6-yl)acetamido)hexyl)amino)-N-methyl-7-oxo-N-(pent-4-yn-1-yl)hept-1-en-1-amine oxide (22′a)

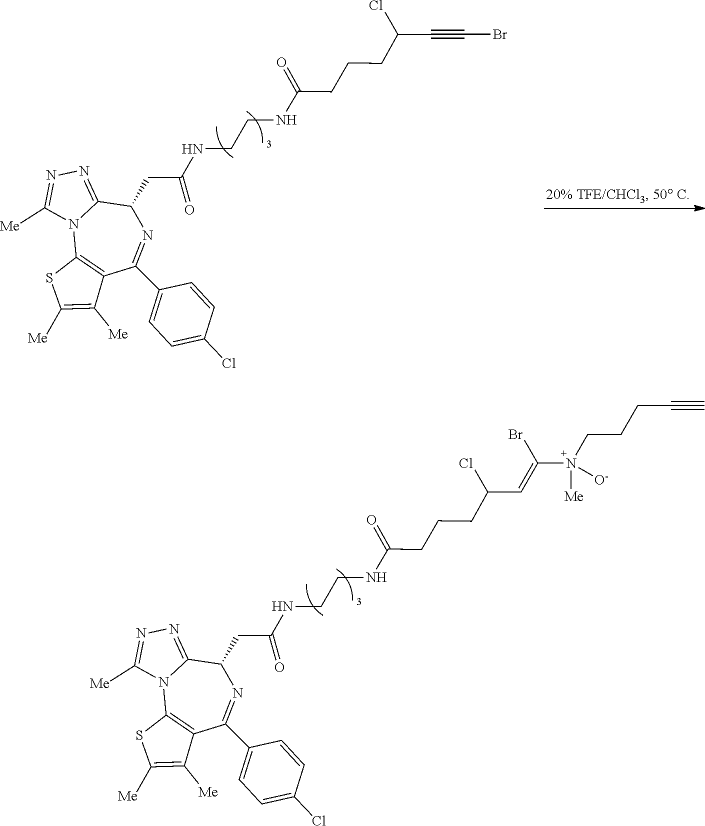

General Procedure A was followed for converting 7-bromo-5-chloro-N-(6-(2-((S)-4-(4-chlorophenyl)-2,3,9-trimethyl-6H-thieno[3,2-f][1,2,4]triazolo[4,3-a][1,4]diazepin-6-yl)acetamido)hexyl)hept-6-ynamide (4.9 mg, 6.80 mol) to give the title compound as a colorless oil (64%). ¹H NMR (500 MHz, CD₃OD) δ 8.34 (s, 1H), 7.97 (s, 1H), 7.48-7.38 (m, 5H), 4.73-4.67 (m, 1H), 4.63 (dd, J=8.9, 5.4 Hz, 1H), 4.00-3.86 (m, 1H), 3.47 (dt, J=11.8, 5.9 Hz, 1H), 3.43 (s, 3H), 3.42-3.38 (m, 1H), 3.29-3.23 (m, 2H), 3.17 (t, J=6.6 Hz, 2H), 2.70 (s, 3H), 2.45 (s, 3H), 2.36-2.26 (m, 3H), 2.23 (td, J=7.3, 1.8 Hz, 2H), 2.09-1.99 (m, 1H), 1.99-1.87 (m, 2H), 1.87-1.77 (m, 1H), 1.76-1.63 (m, 5H), 1.58 (p, J=7.0 Hz, 2H), 1.52 (p, J=7.0 Hz, 2H), 1.40 (dt, J=8.6, 5.1 Hz, 5H). ¹³C NMR (126 MHz, CD₃OD) δ 175.1, 172.7, 166.2, 157.0, 152.2, 138.1, 138.0, 133.6, 133.5, 133.3, 132.1, 132.0, 132.0, 131.3, 129.8, 83.0, 71.1, 71.0, 69.8, 60.8, 60.4, 59.2, 55.3, 40.3, 40.2, 38.8, 38.2, 36.1, 30.4, 27.6, 23.7, 23.7, 23.3, 23.1, 16.1, 14.4, 12.9, 11.6. FTIR (thin film) cm⁻¹; 3299 (br), 2930 (m), 2400 (w), 1636 (s), 1595 (w), 1461 (s), 1420 (s), 1364 (w), 1178 (w), 1088 (m), 1036 (w), 832 (w), 731 (w). HRMS (ESI) (m/z): calc'd for C₃₈H₄₈BrCl₂N₇O₃S [M+H]⁺: 832.2173, found: 832.2179. TLC (50% CMA in chloroform), Rf 0.16 (UV).

### 7-Bromo-5-chloro-N-(6-(2-((R)-4-(4-chlorophenyl)-2,3,9-trimethyl-6H-thieno[3,2-f]/1,2,4triazolo[4,3-a][1,4]diazepin-6-yl)acetamido)hexyl)hept-6-ynamide

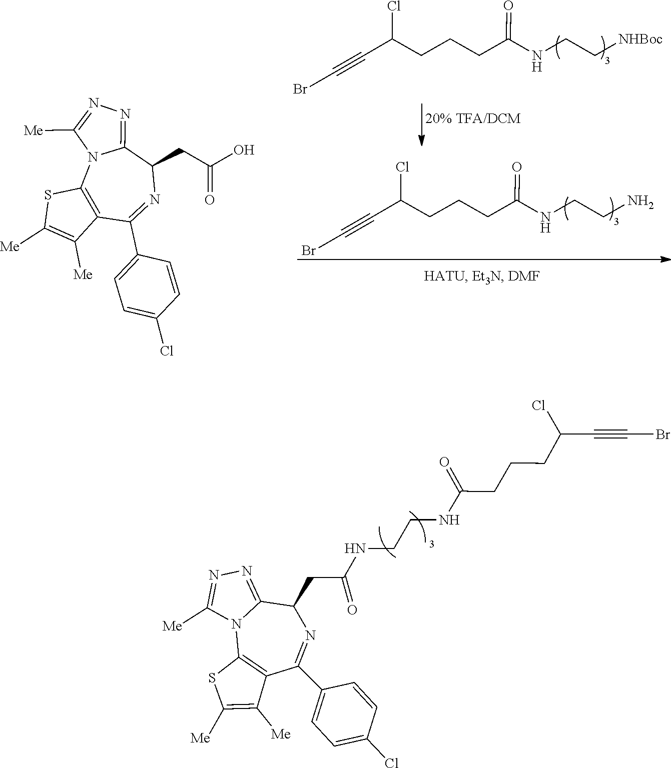

An 8 mL glass vial was charged with tert-butyl (6-(7-bromo-5-chlorohept-6-ynamido)hexyl)carbamate (10.5 mg, 0.024 mmol, 1.10 equiv) at rt. A solution of trifluoroacetic acid in dichloromethane (20% v/v, 500 μL) was introduced via syringe. After 1 h, the reaction mixture was concentrated under reduced pressure, and the crude residue was azeotroped with toluene (3×1 mL). The crude residue was then dissolved in anhydrous dimethylformamide (150 μL) and subsequently added to a solution of (−)-JQ1 carboxylic acid (9.0 mg, 0.022 mmol, 1 equiv), HATU (12.5 mg, 0.033 mmol, 1.50 equiv), and triethylamine (14 μL, 0.100 mmol, 4.50 equiv) in anhydrous dimethylformamide (150 μL). After 3 h, the reaction was diluted with ethyl acetate (10 mL) and washed with brine (10 mL), aqueous citric acid (5% w/v, 10 mL), and saturated aqueous sodium bicarbonate (10 mL). The combined organic layers were dried over sodium sulfate and concentrated under reduced pressure. The crude residue was purified on silica gel (eluent: 5% methanol in dichloromethane) to give the title compound as a pale yellow oil (66%). ¹H NMR (500 MHz, CDCl₃) δ 7.40 (d, J=8.5 Hz, 1H), 7.33 (d, J=8.8 Hz, 1H), 6.66 (s, 1H), 6.06 (s, 1H), 4.61 (dd, J=8.2, 5.6 Hz, 1H), 4.54 (td, J=6.5, 5.4 Hz, 1H), 3.57 (dd, J=14.3, 8.3 Hz, 1H), 3.35-3.15 (m, 5H), 2.67 (s, 3H), 2.41 (s, 3H), 2.21 (t, J=7.3 Hz, 2H), 1.99-1.93 (m, 2H), 1.90-1.80 (m, 2H), 1.67 (s, 3H), 1.56-1.42 (m, 4H), 1.36-1.29 (m, 4H). ¹³C NMR (126 MHz, CDCl₃) δ 172.2, 170.7, 164.1, 155.8, 150.0, 137.0, 136.7, 132.2, 131.2, 131.1, 130.6, 130.0, 128.9, 77.9, 54.7, 48.9, 47.6, 39.7, 39.3, 39.1, 38.4, 35.6, 29.8, 29.4, 29.3, 26.1, 22.4, 14.5, 13.3, 12.0. FTIR (thin film) cm⁻¹: 3288 (br), 2926 (m), 2855 (m), 2214 (w), 1644 (s), 1532 (s), 1416 (s), 1267 (w), 1178 (w), 1088 (m), 1029 (s), 842 (s). HRMS (ESI) (m/z): calc'd for C₃₂H₃₇BrCl₂N₆O₂S [M+H]⁺: 719.1337, found: 719.1337. TLC (5% methanol in dichloromethane), Rf: 0.10 (UV).

### (Z)-1-Bromo-3-chloro-7-((6-(2-((R)-4-(4-chlorophenyl)-2,3,9-trimethyl-6H-thieno[3,2-f][1,2,4]triazolo[4,3-a][1,4]diazepin-6-yl)acetamido)hexyl)amino)-N-methyl-7-oxo-N-(pent-4-yn-1-yl)hept-1-en-1-amine oxide (ent-22′a)

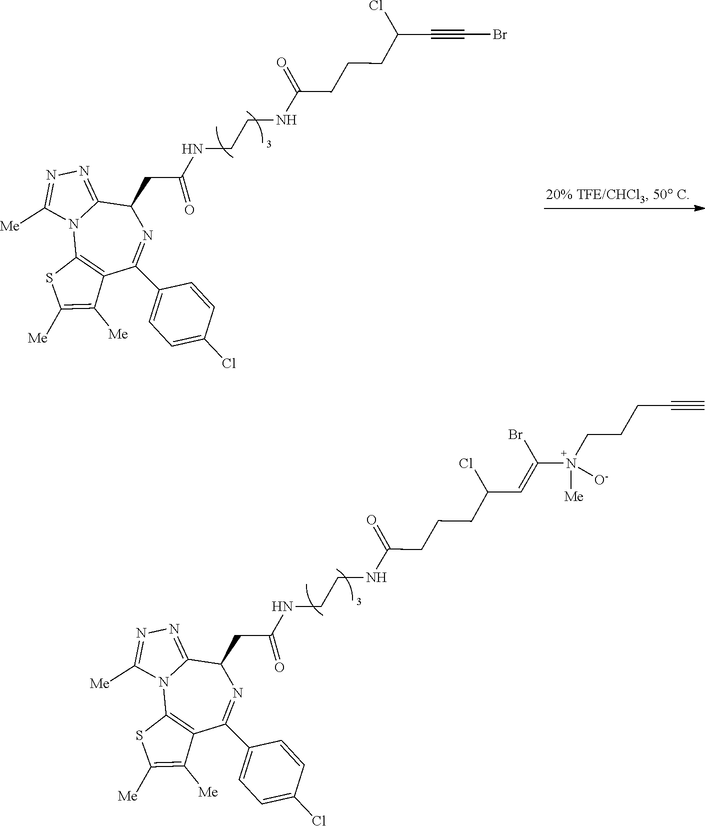

General Procedure A was followed for converting 7-bromo-5-chloro-N-(6-(2-((R)-4-(4-chlorophenyl)-2,3,9-trimethyl-6H-thieno[3,2-f][1,2,4]triazolo[4,3-a][1,4]diazepin-6-yl)acetamido)hexyl)hept-6-ynamide (5.3 mg, 7.35 mol) to give the title compound as a colorless oil (68%). ¹H NMR (500 MHz, CD₃OD) δ 8.34 (s, 1H), 7.97 (s, 1H), 7.48-7.38 (m, 5H), 4.74-4.67 (m, 1H), 4.63 (dd, J=8.9, 5.4 Hz, 1H), 4.00-3.86 (m, 1H), 3.48 (dt, J=12.0, 4.7 Hz, 1H), 3.43 (s, 3H), 3.42-3.37 (m, 1H), 3.29-3.23 (m, 2H), 3.17 (t, J=6.5 Hz, 2H), 2.70 (s, 3H), 2.45 (s, 3H), 2.38-2.27 (m, 3H), 2.23 (td, J=6.5, 1.7 Hz, 2H), 2.10-1.99 (m, 1H), 1.98-1.86 (m, 2H), 1.86-1.76 (m, 1H), 1.74-1.63 (m, 5H), 1.58 (p, J=6.7 Hz, 2H), 1.51 (p, J=7.0 Hz, 2H), 1.40 (dt, J=8.6, 5.1 Hz, 5H). ¹³C NMR (126 MHz, CD₃OD) δ 175.1, 172.7, 166.2, 157.0, 152.2, 138.1, 138.0, 133.6, 133.5, 133.3, 132.1, 132.0, 132.0, 131.3, 129.8, 83.0, 71.1, 71.0, 69.8, 60.8, 60.4, 59.2, 55.3, 40.3, 40.2, 38.8, 38.2, 36.1, 30.4, 27.6, 23.7, 23.6, 23.3, 23.1, 16.1, 14.4, 12.9, 11.6. FTIR (thin film) cm⁻¹: 3287 (br), 2926 (m), 2400 (w), 1636 (s), 1592 (w), 1457 (s), 1420 (s), 1364 (w), 1178 (w), 1088 (m), 1036 (w), 842 (w), 730 (w). HRMS (ESI) (m/z): calc'd for C₃₈H₄₈BrCl₂N₇O₃S [M+H]⁺: 832.2173, found: 832.2178. TLC (50% CMA in chloroform), Rf 0.28 (UV).

### 7-Bromo-5-chloro-N-(2-(2,6-dioxopiperidin-3-yl)-1-oxoisoindolin-5-yl)hept-6-ynamide

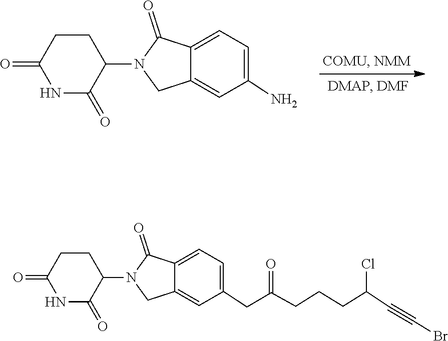 [SMILES: Nc1ccc2c(c1)CN(C1CCC(=O)NC1=O)C2=O ; O=C(CCCC(Cl)C#CBr)Cc1ccc2c(c1)CN(C1CCC(=O)NC1=O)C2=O]

An 8 mL glass vial was charged with 7-bromo-5-chlorohept-6-ynoic acid (24 mg, 0.100 mmol, 1 equiv), (1-cyano-2-ethoxy-2-oxoethylidenaminooxy)dimethylamino-morpholino-carbenium hexafluorophosphate (COMU, 64 mg, 0.15 mmol, 1.50 equiv), 4-dimethylaminopyridine (DMAP, 2.44 mg, 0.020 mmol, 0.200 equiv), and then dissolved in anhydrous dimethylformamide (1 mL). N-Methylmorpholine (NMM, 33 μL, 0.300 mmol, 3.00 equiv) was then added at rt. After stirring for 5 min, solid C5-lenalidomide (25.9 mg, 0.100 mmol, 1 equiv) was added at rt. After 21 h, the reaction was purified by automated C₁₈ reverse phase column chromatography (30 g C₁₈ silica gel, 25 μm spherical particles, eluent: H₂O+0.1% TFA (6 CV), gradient 0→100% MeCN/H₂O+0.1% TFA (10 CV) and further purified on silica gel (eluent: 30% CMA in chloroform) to give the title compound as a white solid (54%). ¹H NMR (500 MHz, CD₃OD) δ 8.04 (d, J=1.7 Hz, 1H), 7.73 (d, J=8.3 Hz, 1H), 7.54 (dd, J=8.3, 1.8 Hz, 1H), 5.13 (dd, J=13.4, 5.1 Hz, 1H), 4.77 (t, J=6.4 Hz, 1H), 4.52-4.40 (m, 2H), 2.90 (ddd, J=17.6, 13.6, 5.4 Hz, 1H), 2.78 (ddd, J=17.6, 4.6, 2.4 Hz, 1H), 2.54-2.42 (m, 3H), 2.16 (dtd, J=12.8, 5.3, 2.4 Hz, 1H), 2.06-1.88 (m, 4H). ¹³C NMR (126 MHz, CD₃OD) δ 174.7, 174.0, 172.2, 171.2, 145.0, 143.9, 127.8, 125.0, 120.7, 115.0, 78.9, 53.6, 49.7, 49.0, 48.7, 39.5, 36.9, 32.4, 24.1, 23.2. FTIR (thin film) cm⁻¹: 3283 (br), 2926 (w), 2214 (w), 1670 (s), 1621 (w), 1547 (m), 1491 (w), 1454 (m), 1368 (m), 1234 (s), 1197 (m), 842 (w), 772 (w). HRMS (ESI) (m/z): calc'd for C₂₀H₁₉BrClN₃O₄ [M+H]⁺: 480.0326, found: 480.0323. TLC (30% CMA in chloroform), Rf: 0.17 (UV).

### (Z)-1-Bromo-3-chloro-7-((2-(2,6-dioxopiperidin-3-yl)-1-oxoisoindolin-5-yl)amino)-N-methyl-7-oxo-N-(pent-4-yn-1-yl)hept-1-en-1-amine oxide (23′a)

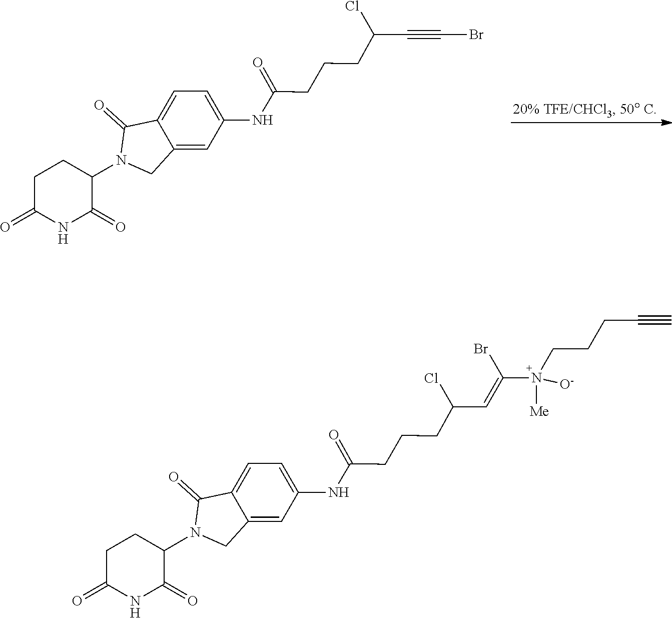

General Procedure A was followed for converting 7-bromo-5-chloro-N-(2-(2,6-dioxopiperidin-3-yl)-1-oxoisoindolin-5-yl)hept-6-ynamide (13.5 mg, 28 mol) to title compound as a white solid (71%). ¹H NMR (500 MHz, CD₃OD) δ 8.04 (s, 1H), 7.73 (d, J=8.3 Hz, 1H), 7.55 (dd, J=8.4, 1.8 Hz, 1H), 7.48 (d, J=9.9 Hz, 1H), 5.13 (dd, J=13.3, 5.1 Hz, 1H), 4.82-4.72 (m, 1H), 4.61-4.21 (m, 2H), 3.99-3.86 (m, 1H), 3.52-3.45 (m, 1H), 3.44 (s, 3H), 2.90 (ddd, J=17.6, 13.5, 5.4 Hz, 1H), 2.78 (ddd, J=17.7, 4.7, 2.4 Hz, 1H), 2.55-2.40 (m, 3H), 2.39-2.22 (m, 3H), 2.22-2.12 (m, 1H), 2.10-1.87 (m, 4H), 1.86-1.76 (m, 1H), 1.72-1.59 (m, 1H). ¹³C NMR (126 MHz, CD₃OD) δ 174.7, 173.8, 172.2, 171.2, 145.1, 144.0, 133.6, 132.2, 127.8, 125.0, 120.6, 115.0, 83.0, 71.0, 69.8, 60.8, 60.4, 59.2, 53.6, 38.2, 37.0, 32.4, 24.1, 23.3, 23.2, 16.1. FTIR (thin film) cm⁻¹: 3261 (br), 2955 (w), 2117 (w), 1670 (s), 1551 (m), 1491 (m), 1368 (w), 1260 (m), 1234 (m), 1200 (m), 842 (w), 772 (w). HRMS (ESI) (m/z): calc'd for C₂₆H₃₀BrClN₄O₅ [M+H]⁺: 593.1166, found: 593.1165. TLC (66% CMA in chloroform), Rf: 0.27 (UV).

### 2-((6-((6-Aminohexyl)amino)-2-methylpyrimidin-4-yl)amino)-N-(2-chloro-6-methylphenyl)thiazole-5-carboxamide

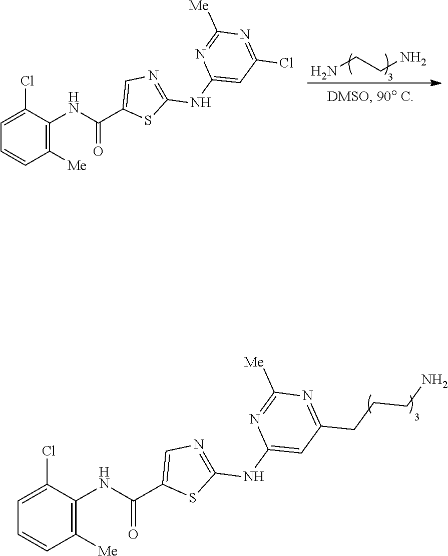

An 8 mL glass vial was charged with 2-((6-chloro-2-methylpyrimidin-4-yl)amino)-N-(2-chloro-6-methylphenyl)thiazole-5-carboxamide (200 mg, 0.507 mmol, 1 equiv) and dissolved in dimethylsulfoxide (2 mL). Hexane-1,6-diamine (295 mg, 2.54 mmol, 5.00 equiv) was added to the suspension at rt. The reaction was then sealed and stirred at 90° C. for 6 h. After cooling to rt, the reaction was purified by automated C₁₈ reverse phase column chromatography (30 g C₁₈ silica gel, 25 m spherical particles, eluent: H₂O+0.1% TFA (6 CV), gradient 0→100% MeCN/H₂O+0.1% TFA (10 CV) to give the title compound as a white solid as the TFA salt (82%). ¹H NMR (500 MHz, DMSO-d₆) δ 9.95 (s, 1H), 8.25 (s, 1H), 7.76 (s, 3H), 7.40 (dd, J=7.6, 1.8 Hz, 1H), 7.32-7.06 (m, 2H), 6.06 (s, 1H), 3.23 (s, 1H), 2.94-2.64 (m, 2H), 2.45 (s, 3H), 2.24 (s, 3H), 1.53 (q, J=7.2 Hz, 4H), 1.38-1.31 (m, 4H). ¹³C NMR (126 MHz, DMSO-d₆) δ 159.7, 159.0, 158.7, 158.5, 158.2, 140.8, 138.8, 133.4, 132.4, 129.1, 128.3, 127.0, 117.6, 115.3, 38.8, 26.9, 25.8, 25.5, 18.3. ¹⁹F NMR (471 MHz, DMSO-d₆) δ −74.2. FTIR (thin film) cm⁻¹: 3243 (w), 2945 (w), 1677 (s), 1625 (s), 1635 (m), 1472 (m), 1398 (s), 1297 (w), 1200 (s), 1133 (m), 835 (w). HRMS (ESI) (m/z): calc'd for C₂₂H₂₈ClN₇OS [M+H]⁺: 474.1837, found: 474.1838.

### 2-((6-((6-(7-Bromo-5-chlorohept-6-ynamido)hexyl)amino)-2-methylpyrimidin-4-yl)amino)-N-(2-chloro-6-methylphenyl)thiazole-5-carboxamide

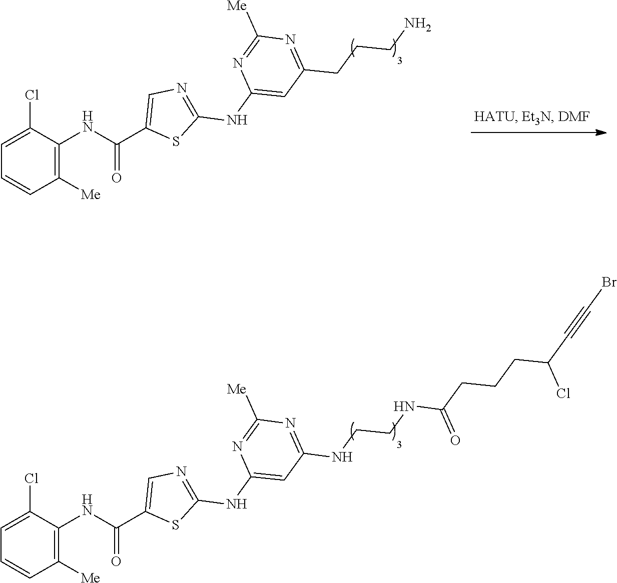

An 8 mL glass vial was charged with 7-bromo-5-chlorohept-6-ynoic acid (24.8 mg, 0.104 mmol, 1 equiv) and HATU (59.3 mg, 0.156 mmol, 1.50 equiv) then dissolved in anhydrous dimethylformamide (500 μL). Triethylamine (58 μL, 0.416 mmol, 4.00 equiv) was added to the solution via syringe. After stirring for 5 min, solid 2-((6-((2-aminoethyl)amino)-2-methylpyrimidin-4-yl)amino)-N-(2-chloro-6-methylphenyl)thiazole-5-carboxamide ammonium salt (3.4 mg, 0.104 mmol, 1 equiv) was added at rt. After 17 h, the reaction was purified by automated C₁₈ reverse phase column chromatography (30 g C₁₈ silica gel, 25 m spherical particles, eluent: H₂O+0.1% TFA (6 CV), gradient 0→100% MeCN/H₂O+0.1% TFA (10 CV) and further purified on silica gel (eluent: 25% CMA in chloroform) to give the title compound as a white solid (52%). ¹H NMR (500 MHz, DMSO-d₆) δ 11.34 (s, 1H), 9.89 (s, 1H), 8.17 (s, 1H), 7.84 (t, J=5.6 Hz, 1H), 7.37 (dd, J=7.6, 1.9 Hz, 1H), 7.30-7.20 (m, 2H), 7.06 (s, 1H), 5.82 (s, 1H), 4.93 (t, J=6.5 Hz, 1H), 3.16 (d, J=5.2 Hz, 2H), 3.01 (q, J=6.4 Hz, 2H), 2.34 (s, 3H), 2.21 (s, 3H), 2.08 (t, J=7.3 Hz, 2H), 1.86-1.77 (m, 2H), 1.64 (p, J=7.0 Hz, 2H), 1.47 (q, J=7.1 Hz, 2H), 1.37 (p, J=7.0 Hz, 2H), 1.26 (dd, J=8.2, 4.5 Hz, 4H). ¹³C NMR (126 MHz, DMSO-d₆) δ 172.2, 172.1, 165.7, 163.4, 163.1, 160.5, 141.2, 139.2, 133.7, 132.7, 129.4, 128.6, 127.3, 125.7, 78.5, 49.9, 49.6, 39.8, 38.8, 38.6, 38.1, 34.6, 34.6, 29.3, 26.4, 26.4, 25.6, 22.3, 18.6. FTIR (thin film) cm⁻¹: 3228 (br), 2930 (w), 1588 (s), 1491 (m), 1413 (s), 1290 (m), 1197 (s), 1025 (w), 984 (w), 772 (w). HRMS (ESI) (m/z): calc'd for C₂₉H₃₄BrCl₂N₇O₂S [M+H]⁺: 694.1133, found: 694.1134. TLC (25% CMA in chloroform), Rf: 0.23 (UV).

### (Z)-1-Bromo-3-chloro-7-((6-((6-((5-((2-chloro-6-methylphenyl)carbamoyl)thiazol-2-yl)amino)-2-methylpyrimidin-4-yl)amino)hexyl)amino)-N-methyl-7-oxo-N-(pent-4-yn-1-yl)hept-1-en-1-amine oxide (24′a)

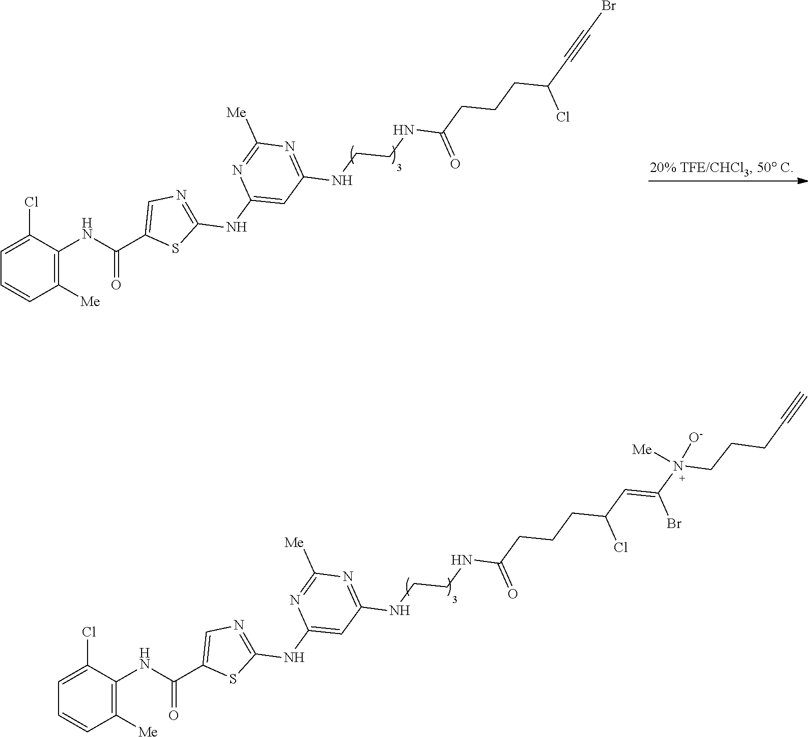

General Procedure A was followed for converting 2-((6-((6-(7-bromo-5-chlorohept-6-ynamido)hexyl)amino)-2-methylpyrimidin-4-yl)amino)-N-(2-chloro-6-methylphenyl)thiazole-5-carboxamide (17 mg, 24.4 μmol) to give the title compound as a white solid (66%). ¹H NMR (500 MHz, CD₃OD) δ 8.15 (s, 1H), 7.45 (dd, J=9.9, 1.3 Hz, 1H), 7.35 (dd, J=7.6, 2.0 Hz, 1H), 7.28-7.19 (m, 2H), 5.84 (s, 1H), 4.73-4.65 (m, 1H), 4.00-3.84 (m, 1H), 3.53-3.46 (m, 1H), 3.43 (d, J=5.1 Hz, 3H), 3.26 (s, 2H), 3.18 (t, J=7.0 Hz, 2H), 2.45 (s, 3H), 2.32 (s, 4H), 2.31-2.26 (m, 2H), 2.23 (tt, J=7.1, 1.6 Hz, 2H), 2.09-1.97 (m, 1H), 1.96-1.86 (m, 2H), 1.84-1.76 (m, 1H), 1.74-1.58 (m, 4H), 1.53 (p, J=7.1 Hz, 2H), 1.48-1.34 (m, 4H). ¹³C NMR (126 MHz, CD₃OD) δ 175.2, 175.1, 167.5, 165.4, 164.9, 163.3, 142.2, 140.4, 134.4, 133.6, 133.3, 132.2, 130.1, 129.5, 128.3, 126.7, 83.0, 71.1, 69.7, 60.8, 60.4, 59.1, 42.2, 40.3, 38.3, 36.1, 30.4, 30.1, 27.6, 27.6, 25.3, 23.8, 23.3, 18.7, 16.1. FTIR (thin film) cm⁻¹: 3250 (br), 2930 (w), 2856 (w), 1595 (s), 1495 (m), 1461 (m), 1416 (s), 1293 (m), 1200 (m), 865 (w), 816 (w), 775 (w). HRMS (ESI) (m/z): calc'd for C₃₅H₄₅BrCl₂N₈O₃S [M+H]⁺: 807.1968, found: 807.1972. TLC (50% CMA in chloroform), Rf 0.26 (UV).

### N-(6-(7-Bromo-5-chlorohept-6-ynamido)hexyl)-3,4-difluoro-2-((2-fluoro-4-iodophenyl)amino)benzamide

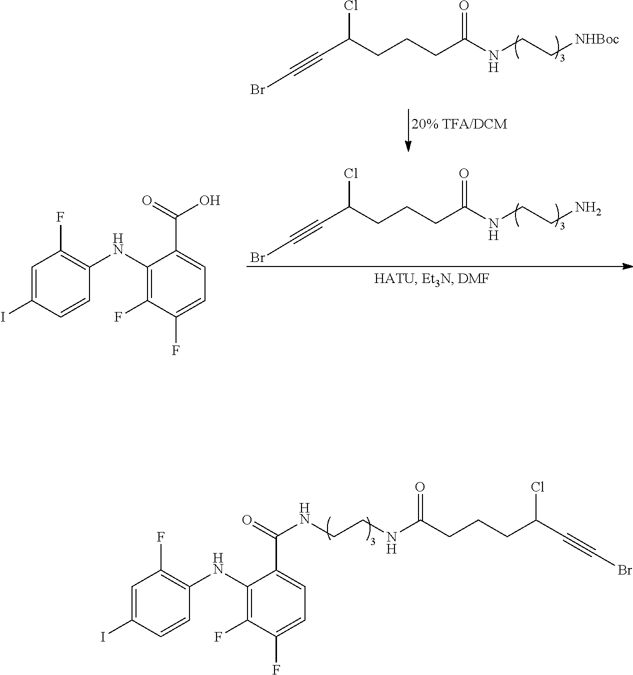

An 8 mL glass vial was charged with tert-butyl (6-(7-bromo-5-chlorohept-6-ynamido)hexyl)carbamate (15.3 mg, 0.035 mmol, 1.10 equiv) at rt. A solution of trifluoroacetic acid in dichloromethane (20% v/v, 500 μL) was introduced via syringe. After 1 h, the reaction mixture was concentrated under reduced pressure and the crude residue was azeotroped with toluene (3×1 mL). The crude residue was then dissolved in anhydrous dimethylformamide (160 μL) and subsequently added to a solution of 3,4-difluoro-2-((2-fluoro-4-iodophenyl)amino)benzoic acid (12.6 mg, 0.032 mmol, 1 equiv), HATU (18.2 mg, 0.048 mmol, 1.50 equiv), and triethylamine (18 μL, 0.130 mmol, 4.00 equiv) in anhydrous dimethylformamide (160 μL). After 3 h, the reaction was diluted with ethyl acetate (10 mL) and washed with brine (10 mL), aqueous citric acid (5% w/v, 10 mL), and saturated aqueous sodium bicarbonate (10 mL), respectively. The combined organic layers were dried over sodium sulfate and concentrated under reduced pressure. The crude residue was purified on silica gel (eluent: 60% ethyl acetate in hexanes) to give the title compound as a white solid (74%). ¹H NMR (500 MHz, DMSO-d₆) δ 9.22 (s, 1H), 8.67 (t, J=5.6 Hz, 1H), 7.76 (t, J=5.6 Hz, 1H), 7.58 (dd, J=10.8, 1.9 Hz, 1H), 7.55-7.50 (m, 1H), 7.40-7.35 (m, 1H), 7.24-7.15 (m, 1H), 6.67 (td, J=8.8, 5.0 Hz, 1H), 4.98 (t, J=6.5 Hz, 1H), 3.22-3.15 (m, 2H), 3.00 (q, J=6.5 Hz, 2H), 2.08 (t, J=7.4 Hz, 2H), 1.89-1.79 (m, 2H), 1.69-1.58 (m, 2H), 1.45 (t, J=7.0 Hz, 2H), 1.34 (t, J=6.8 Hz, 2H), 1.24 (p, J=2.9 Hz, 4H). ¹³C NMR (126 MHz, DMSO-d₆) δ 171.3, 166.6, 153.6, 151.6, 150.7, 133.2, 131.6, 130.9, 124.7, 123.7, 122.6, 119.9, 109.9, 82.1, 78.1, 49.8, 49.3, 39.1, 38.3, 37.8, 34.2, 29.1, 28.7, 26.1, 26.1, 22.0. ¹⁹F NMR (471 MHz, DMSO-d₆) δ −128.0, −133.3, −143.6. FTIR (thin film) cm⁻¹: 3294 (w) 2933 (w), 2851 (w), 2422 (w), 2214 (w), 1629 (s), 1602 (s), 1558 (m), 1498 (s), 1469 (s), 1323 (m), 1286 (m), 1126 (m), 1018 (m), 861 (m), 801 (m), 701 (m). HRMS (ESI) (m/z): calc'd for C₂₆H₂₇BrClF₃IN₃O₂ [M+H]⁺: 712.0050, found: 712.0048. TLC (60% ethyl acetate in hexanes), Rf: 0.29 (UV).

### (Z)-1-Bromo-3-chloro-7-((6-(3,4-difluoro-2-((2-fluoro-4-iodophenyl)amino)benzamido)hexyl)amino)-N-methyl-7-oxo-N-(pent-4-yn-1-yl)hept-1-en-1-amine oxide (25′a)

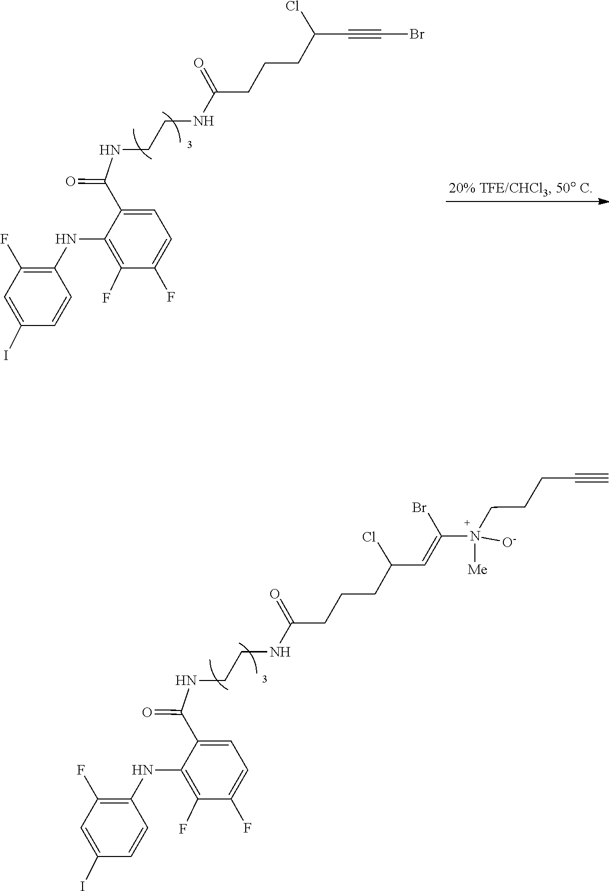

General Procedure A was followed for converting N-(6-(7-bromo-5-chlorohept-6-ynamido)hexyl)-3,4-difluoro-2-((2-fluoro-4-iodophenyl)amino)benzamide (8.5 mg, 11.9 μmol) to give the title compound as a colorless oil (71%). ¹H NMR (500 MHz, CD₃OD) δ 7.94 (s, 1H), 7.49-7.41 (m, 3H), 7.36-7.30 (m, 1H), 7.04 (td, J=9.3, 7.1 Hz, 1H), 6.56 (td, J=8.7, 4.4 Hz, 1H), 4.75-4.66 (m, 1H), 4.00-3.86 (m, 1H), 3.52-3.45 (m, 1H), 3.44 (d, J=1.2 Hz, 3H), 3.27 (t, J=7.0 Hz, 2H), 3.15 (td, J=7.0, 5.0 Hz, 2H), 2.36-2.26 (m, 3H), 2.23 (tt, J=7.2, 1.7 Hz, 2H), 2.10-1.99 (m, 1H), 1.99 (s, 2H), 1.98-1.86 (m, 2H), 1.85-1.75 (m, 1H), 1.75-1.60 (m, 2H), 1.48 (dq, J=14.1, 4.9 Hz, 4H), 1.37-1.27 (m, 4H). ¹³C NMR (126 MHz, CD₃OD) δ 175.1, 169.2, 155.3, 155.0, 153.3, 153.0, 146.2, 144.2, 134.5, 133.5, 132.2, 125.5, 125.3, 120.3, 111.6, 83.0, 81.8, 71.1, 69.7, 60.8, 60.4, 59.1, 40.7, 40.2, 38.2, 36.1, 30.3, 30.2, 27.6, 23.7, 23.3, 16.1. ¹⁹F NMR (471 MHz, CD₃OD) δ −130.1, −134.9, −145.5. FTIR (thin film) cm⁻¹: 3299 (br), 2933 (m), 2856 (w), 2407 (br), 1640 (s), 1602 (m), 1498 (s), 1461 (s), 1286 (m), 1193 (m), 1148 (w), 1044 (w), 947 (w), 876 (w), 775 (w). HRMS (ESI) (m/z): calc'd for C₃₂H₃₈BrClF₃IN₄O₃ [M+H]⁺: 825.0891, found: 825.0891. TLC (40% CMA in chloroform), Rf: 0.17 (UV).

### 7-Bromo-5-chloro-N-((5S,6R,7R,9R)-6-methoxy-5-methyl-14-oxo-6,7,8,9,15,16-hexahydro-5H,14H-17-oxa-4b,9a,15-triaza-5,9-methanodibenzo[b,h]cyclonona[jkl]cyclopenta[e]-as-indacen-7-yl)-N-methylhept-6-ynamide

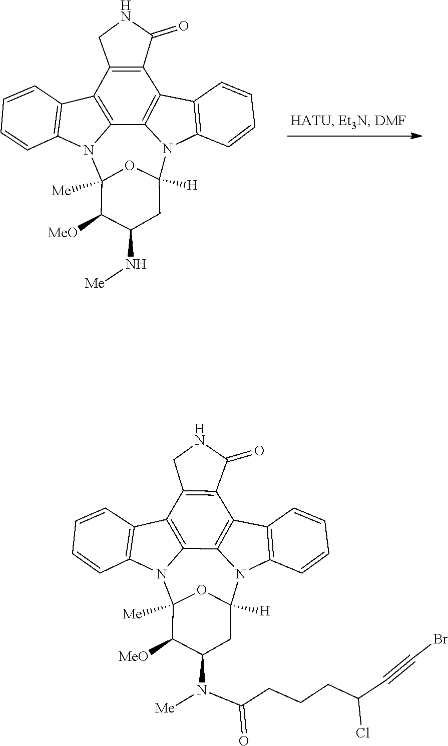 [SMILES: CN[C@@H]1C[C@H]2O[C@@](C)([C@@H]1OC)n1c3ccccc3c3c4c(c5c6ccccc6n2c5c31)C(=O)NC4 ; CO[C@@H]1[C@H](N(C)C(=O)CCCC(Cl)C#CBr)C[C@H]2O[C@]1(C)n1c3ccccc3c3c4c(c5c6ccccc6n2c5c31)C(=O)NC4]

An 8 mL glass vial was charged with 7-bromo-5-chlorohept-6-ynoic acid (24 mg, 0.100 mmol, 1 equiv) and HATU (57 mg, 0.150 mmol, 1.50 equiv) then dissolved in anhydrous dimethylformamide (1 mL). Triethylamine (42 μL, 0.300 mmol, 3.00 equiv) was then added via syringe at rt. After stirring for 5 min, solid staurosporine (46.6 mg, 0.100 mmol, 1 equiv) was added, the reaction vial was covered in aluminum foil, and the solution was stirred at rt in the dark. After 16 h, the reaction was purified by automated C₁₈ reverse phase column chromatography (30 g C₁₈ silica gel, 25 μm spherical particles, eluent: H₂O+0.1% TFA (6 CV), gradient 0→100% MeCN/H₂O+0.1% TFA (10 CV) and further purified on silica gel (eluent: 50%→80% ethyl acetate in hexanes) to give the title compound as a white solid (77%). ¹H NMR (500 MHz, DMSO-d₆) δ 9.29 (d, J=7.8 Hz, 1H), 8.60 (s, 1H), 8.05 (d, J=7.9 Hz, 1H), 7.99 (d, J=8.4 Hz, 1H), 7.65 (d, J=8.2 Hz, 1H), 7.55-7.45 (m, 2H), 7.39-7.33 (m, 1H), 7.33-7.27 (m, 1H), 7.02 (dd, J=8.5, 6.6 Hz, 1H), 5.05 (td, J=6.5, 1.9 Hz, 1H), 5.00 (d, J=4.0 Hz, 2H), 4.24 (dd, J=2.7, 1.5 Hz, 1H), 2.78 (s, 3H), 2.74 (s, 3H), 2.67-2.58 (m, 2H), 2.42 (dt, J=10.9, 3.9 Hz, 2H), 2.35 (s, 3H), 2.22 (td, J=13.0, 6.5 Hz, 1H), 2.10-1.91 (m, 2H), 1.85-1.67 (m, 2H). ¹³C NMR (126 MHz, DMSO-d₆) δ 172.4, 171.9, 138.9, 136.3, 132.7, 129.2, 125.7, 125.4, 125.1, 125.0, 123.8, 122.7, 121.5, 120.3, 119.5, 119.4, 115.2, 114.1, 113.6, 109.0, 94.7, 83.3, 82.3, 78.3, 60.5, 49.8, 49.5, 48.0, 45.5, 37.9, 31.9, 30.8, 29.4, 26.8, 21.2. FTIR (thin film) cm⁻¹: 2922 (m), 2851 (w), 1674 (s), 1629 (s), 1454 (s), 1387 (m), 1346 (s), 1286 (m), 1122 (m), 835 (s). HRMS (ESI) (m/z): calc'd for C₃₅H₃₂BrClN₄O₄ [M+H]⁺: 687.1374, found: 687.1366. TLC (80% ethyl acetate in hexanes), Rf 0.27 (UV).

### (Z)-1-Bromo-3-chloro-7-(((5S,6R,7R,9R)-6-methoxy-5-methyl-14-oxo-6,7,8,9,15,16-hexahydro-5H,14H-17-oxa-4b,9a,15-triaza-5,9-methanodibenzo[b,h]cyclonona[jkl]cyclopenta[e]-as-indacen-7-yl)(methyl)amino)-N-methyl-7-oxo-N-(pent-4-yn-1-yl)hept-1-en-1-amine oxide (26′a)

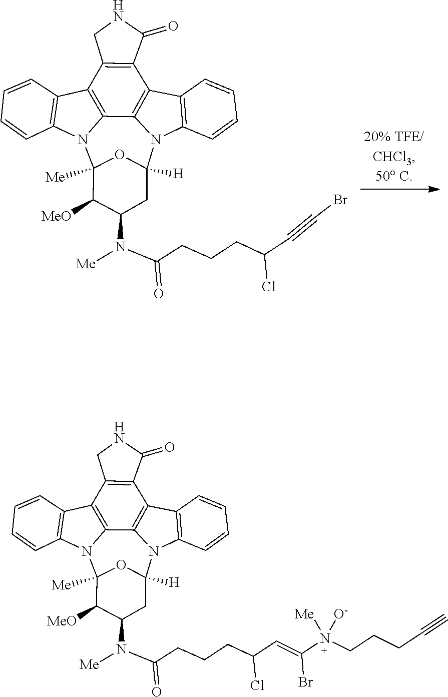

General Procedure A was followed for converting 7-bromo-5-chloro-N-((5S,6R,7R,9R)-6-methoxy-5-methyl-14-oxo-6,7,8,9,15,16-hexahydro-5H,14H-17-oxa-4b,9a,15-triaza-5,9-methanodibenzo[b,h]cyclonona[jkl]cyclopenta[e]-as-indacen-7-yl)-N-methylhept-6-ynamide (16 mg, 23.2 μmol) to give the title compound as a white solid (32%). ¹H NMR (500 MHz, CD₃OD) δ 9.26 (d, J=8.0 Hz, 1H), 7.99 (dd, J=7.9, 1.3 Hz, 1H), 7.91-7.89 (m, 1H), 7.52-7.44 (m, 3H), 7.42-7.33 (m, 2H), 7.28 (ddd, J=8.0, 6.9, 1.1 Hz, 1H), 6.79 (dd, J=9.0, 5.1 Hz, 1H), 5.14 (ddd, J=13.4, 5.2, 2.3 Hz, 1H), 4.97 (d, J=5.8 Hz, 2H), 4.80-4.70 (m, 1H), 4.10 (d, J=1.7 Hz, 1H), 4.03-3.85 (m, 1H), 3.57-3.48 (m, 1H), 3.45 (s, 3H), 2.85 (s, 3H), 2.70-2.61 (m, 1H), 2.51 (s, 3H), 2.50-2.39 (m, 6H), 2.38-2.27 (m, 3H), 2.13-1.93 (m, 3H), 1.91-1.57 (m, 3H). ¹³C NMR (126 MHz, CD₃OD) δ 175.4, 175.3, 140.3, 138.1, 134.2, 133.6, 132.3, 131.9, 127.4, 127.1, 126.6, 126.2, 125.8, 124.5, 122.5, 121.7, 120.7, 120.0, 117.1, 115.8, 114.1, 109.4, 96.1, 85.7, 84.0, 83.1, 71.1, 71.0, 69.8, 60.9, 60.8, 60.5, 59.4, 38.3, 34.0, 31.8, 29.6, 28.7, 23.3, 22.8, 16.2. FTIR (thin film) cm⁻¹: 3283 (br), 2930 (m), 2117 (w), 1677 (s), 1629 (s), 1454 (s), 1398 (m), 1346 (s), 1312 (s), 1249 (w), 1152 (m), 1118 (m), 1074 (m), 746 (s). HRMS (ESI) (m/z): calc'd for C₄₁H₄₃BrClN₅O₅ [M+H]⁺: 800.2214, found: 800.2214. TLC (30% CMA in chloroform), Rf: 0.13 (UV).

### Example 5: General Building Blocks

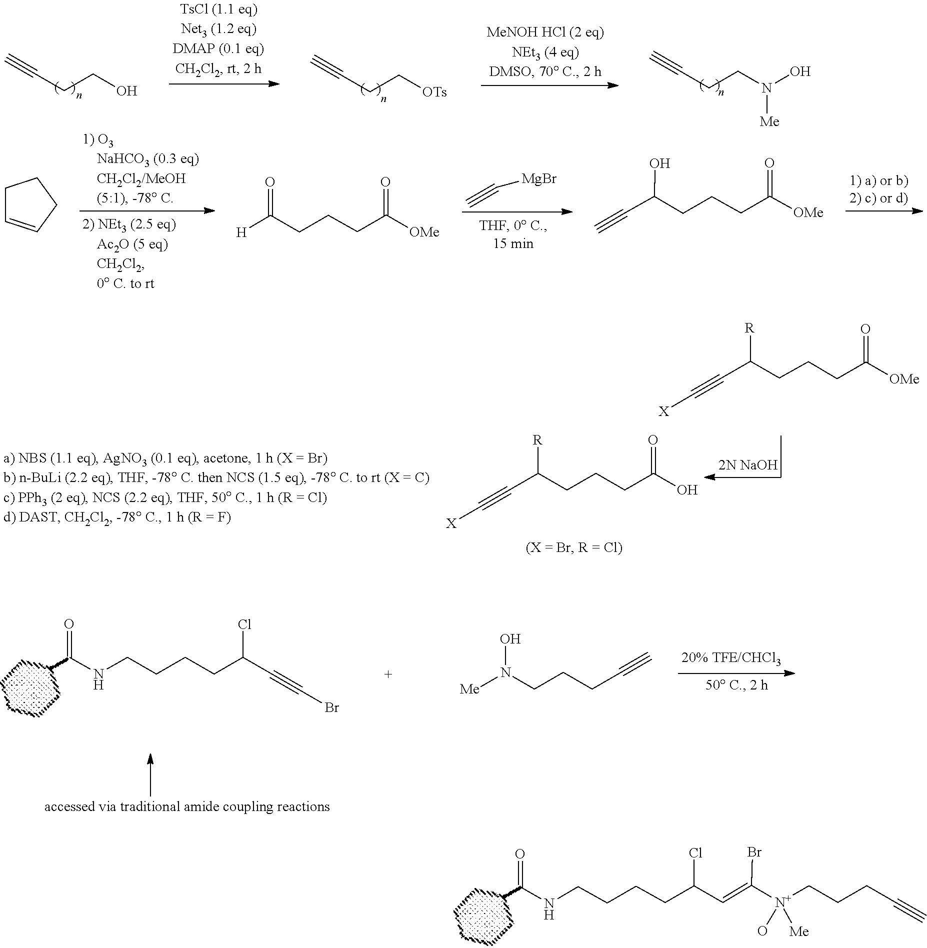

### Example 6: Characterization of Trapped Adducts

### General Procedure: Chemical Trapping of Bromoiminium Ion Intermediate with Enamine N-Oxide 7′a and N-Acetylated Amino Acids in Aqueous Buffer

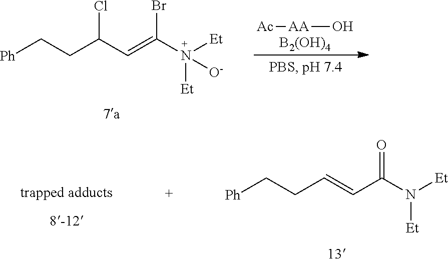

Enamine N-oxide 7′a (50.0 mg, 0.144 mmol, 50 mM final concentration) and solid tetrahydroxydiboron (15.5 mg, 0.172 mmol, 60 mM final concentration) were added sequentially to a solution of N-acetylated amino acid (500 mM, 1.44 mmol, Combi-Blocks) in PBS, pH 7.4 (2.88 mL). The solution was stirred at rt for 10 min after which it was immediately purified by preparatory high-performance liquid chromatography (HPLC) using a C₁₈ reverse phase column (250×21.2 mm, 5 μm particle size, 20 mL/min flow rate, eluent: H₂O+0.1% TFA (2 min), gradient 0→100% MeCN/H₂O+0.1% TFA (24 min), t_{R}=6.0-22.0 min). Fractions containing relevant products were collected and further characterized by NMR, FTIR, and HRMS.

### Lysine 1,2-adduct (8′)

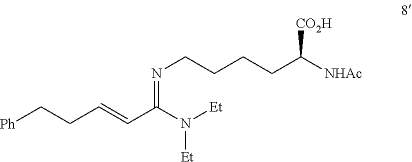 [SMILES: CCN(CC)C(/C=C/CCc1ccccc1)=N\CCCC[C@H](NC(C)=O)C(=O)O]

Yield: 8.2% (colorless oil). ¹H NMR (500 MHz, CD₃OD) δ 7.42-7.14 (m, 5H), 6.26 (dt, J=16.5, 6.8 Hz, 1H), 6.02 (dt, J=16.5, 1.5 Hz, 1H), 4.46-4.31 (m, 1H), 4.08-3.97 (m, 1H), 3.61-3.34 (m, 4H), 3.23-3.05 (m, 2H), 2.94-2.86 (m, 1H), 2.82-2.68 (m, 2H), 2.22-2.08 (m, 1H), 1.99 (s, 3H), 1.91-1.78 (m, 1H), 1.75-1.59 (m, 1H), 1.56-1.44 (m, J=6.9 Hz, 1H), 1.40 (td, J=7.1, 3.4 Hz, 1H), 1.36-1.26 (m, 3H), 1.24-1.06 (m, 4H). ¹³C NMR (126 MHz, CD₃OD) S 175.2, 163.4, 146.7, 141.8, 141.5, 129.8, 129.7, 129.6, 129.4, 127.6, 127.4, 118.3, 92.1, 63.6, 62.8, 56.4, 53.1, 46.4, 43.3, 41.6, 41.4, 36.2, 34.8, 34.7, 34.3, 33.4, 32.9, 32.7, 32.3, 32.0, 31.5, 30.2, 23.7, 22.4, 14.3, 13.1, 11.7. FTIR (thin film) cm⁻¹: 3265 (br), 2937 (w), 1670 (s), 1618 (s), 1454 (m), 1200 (s), 1133 (s), 831 (w), 798 (w), 719 (m). HRMS (ESI) (m/z): calc'd for C₂₃H₃₅N₃O₃ [M+H]⁺: 402.2751, found: 402.2751.

### Cysteine 1,2-adduct (9)

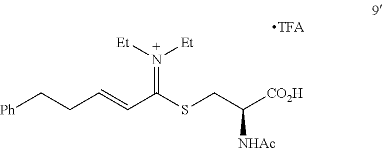

Yield: 28% (colorless oil). ¹H NMR (500 MHz, CD₃OD) δ 7.38-7.24 (m, 4H), 7.23-7.17 (m, 1H), 6.63 (dt, J=16.1, 6.8 Hz, 1H), 6.39 (dt, J=16.1, 1.5 Hz, 1H), 4.68 (dd, J=8.1, 4.9 Hz, 1H), 3.96 (q, J=7.3 Hz, 2H), 3.84-3.56 (m, 3H), 3.38-3.32 (m, 1H), 2.94 (td, J=7.1, 3.4 Hz, 2H), 2.86-2.79 (m, 2H), 2.02 (s, 3H), 1.36 (t, J=7.3 Hz, 3H), 1.22 (t, J=7.2 Hz, 3H). ¹³C NMR (126 MHz, CD₃OD) δ 188.3, 173.4, 171.7, 152.4, 141.7, 129.8, 129.6, 127.5, 121.4, 53.1, 52.7, 51.7, 38.9, 35.4, 34.6, 22.4, 12.8, 11.7. ¹⁹F NMR (471 MHz, CD₃OD) δ −77.3. FTIR (thin film) cm⁻¹: 3288 (br), 2933 (w), 1666 (s), 1543 (m), 1428 (w), 1379 (w), 1185 (s), 1133 (s), 842 (w), 801 (w). HR-MS (ESI) (m/z): calc'd for C₂₀H₂₉N₂O₃S [M+H]⁺: 377.1899, found: 377.1892.

### Cysteine 1,211,4-adduct (10′)

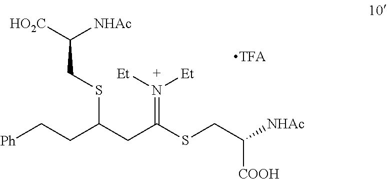

Yield: 10% (colorless oil). ¹H NMR (500 MHz, CD₃OD, mixture of diastereomers) δ 7.34-7.26 (m, 4H), 7.23-7.18 (m, 1H), 4.77 (ddd, J=8.0, 5.4, 2.0 Hz, 1H), 4.64-4.50 (m, 1H), 4.26-4.07 (m, 2H), 4.04-3.83 (m, 2H), 3.80-3.75 (m, 1H), 3.58 (ddd, J=13.6, 7.5, 4.6 Hz, 1H), 3.44-3.34 (m, 1H), 3.21-3.01 (m, 2H), 2.98-2.79 (m, 4H), 2.25-2.07 (m, 2H), 2.06-1.92 (m, 6H), 1.48-1.25 (m, 6H). ¹³C NMR (126 MHz, CD₃OD, mixture of diastereomers) δ 192.0, 191.8, 173.7, 173.6, 173.3, 173.2, 173.0, 173.0, 171.6, 171.5, 142.0, 142.0, 129.8, 129.8, 129.6, 129.6, 127.4, 56.0, 54.0, 53.7, 53.6, 53.6, 52.9, 52.9, 52.8, 52.7, 46.8, 46.4, 46.3, 38.5, 37.0, 34.7, 33.5, 33.4, 26.7, 22.5, 22.5, 22.4, 13.3, 13.2, 13.0, 12.4, 11.5, 11.4. ¹⁹F NMR (471 MHz, CD₃OD, mixture of diastereomers) 6-77.1. FTIR (thin film) cm⁻¹: 3280 (br), 1655 (m), 1580 (m), 1543 (m), 1375 (w), 1305 (w), 1170 (s), 1133 (s), 798 (m). HRMS (ESI) (m/z): calc'd for C₂₅H₃₈N₃O₆S₂ [M+H]⁺: 541.2275, found: 541.2231.

### Cysteine 1,2/1,4-adduct (11′)

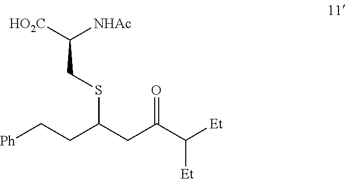 [SMILES: CCC(CC)C(=O)CC(CCc1ccccc1)SC[C@H](NC(C)=O)C(=O)O]

Yield: 48% (colorless oil). ¹H NMR (500 MHz, CD₃OD, mixture of diastereomers) δ 7.31-7.22 (m, 2H), 7.23-7.15 (m, 2H), 7.15 (td, J=7.1, 1.7 Hz, 1H), 4.62 (ddd, J=29.7, 7.5, 4.7 Hz, 1H), 3.44-3.33 (m, 4H), 3.26-3.14 (m, 1H), 3.14-2.99 (m, 1H), 2.99-2.79 (m, 2H), 2.79-2.65 (m, 2H), 2.65-2.54 (m, 1H), 2.00 (d, J=1.9 Hz, 3H), 1.98-1.77 (m, 2H), 1.23-1.01 (m, 6H). ¹³C NMR (126 MHz, CD₃OD, mixture of diastereomers) δ 173.7, 173.5, 173.3, 172.5, 172.4, 143.0, 143.0, 129.5, 129.5, 129.5, 127.0, 126.9, 54.4, 54.0, 44.5, 43.8, 43.7, 41.9, 41.8, 40.7, 40.3, 38.8, 38.1, 34.1, 34.1, 34.0, 33.4, 22.4, 22.4, 14.6, 14.6, 13.3, 13.3. FTIR (thin film) cm⁻¹: 3265 (br), 2974 (w), 2933 (w), 1733 (m), 1618 (s), 1454 (m), 1211 (m), 1182 (m), 750 (w). HRMS (ESI) (m/z): calc'd for C₂₀H₃₀N₂O₄S [M+H]⁺: 395.2005, found: 395.1999.

### Histidine 1,4-adduct (12′)

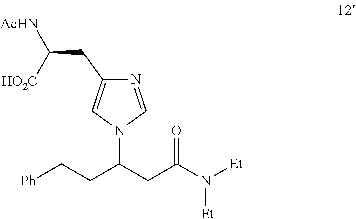 [SMILES: CCN(CC)C(=O)CC(CCc1ccccc1)n1cnc(C[C@H](NC(C)=O)C(=O)O)c1]

Yield: 37% (colorless oil). ¹H NMR (500 MHz, CD₃OD, mixture of diastereomers) δ 8.86 (dd, J=5.9, 1.6 Hz, 1H), 7.55 (dd, J=7.3, 1.6 Hz, 1H), 7.30-7.22 (m, 2H), 7.22-7.09 (m, 3H), 4.84-4.79 (m, 1H), 4.75 (td, J=9.0, 5.4 Hz, 1H), 3.34 (d, J=7.7 Hz, 3H), 3.29-3.22 (m, 2H), 3.11-2.94 (m, 3H), 2.65-2.54 (m, 1H), 2.49 (dt, J=14.2, 7.3 Hz, 1H), 2.28 (dt, J=11.2, 7.9 Hz, 2H), 1.94 (d, J=10.6 Hz, 3H), 1.17-1.12 (m, 3H), 1.08-0.99 (m, 3H). ¹³C NMR (126 MHz, CD₃OD, mixture of diastereomers) δ 173.2, 173.0, 169.8, 141.3, 136.5, 136.3, 132.3, 132.2, 129.6, 129.4, 127.4, 119.1, 118.9, 59.4, 59.3, 52.7, 52.6, 43.1, 41.6, 41.5, 39.0, 39.0, 36.8, 36.7, 33.0, 32.9, 28.5, 28.4, 22.5, 14.3, 13.1. FTIR (thin film) cm⁻¹: 3280 (br), 2937 (w), 1662 (m), 1621 (s), 1439 (w), 1379 (w), 1178 (s), 1133 (s), 831 (w), 798 (w), 749 (w). HRMS (ESI) (m/z): calc'd for C₂₃H₃₂N₄O₄ [M+H]⁺: 429.2502, found: 429.2498.

### N,N-diethyl-5-phenylpent-2-enamide (13)

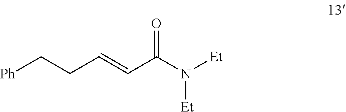 [SMILES: CCN(CC)C(=O)/C=C/CCc1ccccc1]

¹H NMR (500 MHz, CDCl₃) δ 7.41-7.31 (m, 2H), 7.27-7.21 (m, 3H), 6.99 (dt, J=15.2, 7.0 Hz, 1H), 6.20 (d, J=15.1 Hz, 2H), 3.50 (q, J=7.2 Hz, 2H), 3.37 (q, J=7.2 Hz, 2H), 2.85 (t, J=7.6 Hz, 2H), 2.72-2.54 (m, 2H), 1.22 (td, J=7.0, 3.5 Hz, 6H). ¹³C NMR (126 MHz, CDCl₃) δ 167.2, 146.4, 141.0, 128.6, 128.5, 126.2, 120.7, 42.7, 41.5, 34.6, 34.4, 14.6, 13.1. FTIR (thin film) cm⁻¹: 2978 (w), 2937 (w), 1777 (w), 1654 (s), 1603 (m), 1454 (m), 1383 (w), 1170 (s), 1085 (w), 969 (w). HRMS (ESI) (m/z): calc'd for C₁₅H₂₁NO [M+H]⁺: 232.1701, found: 232.1696.

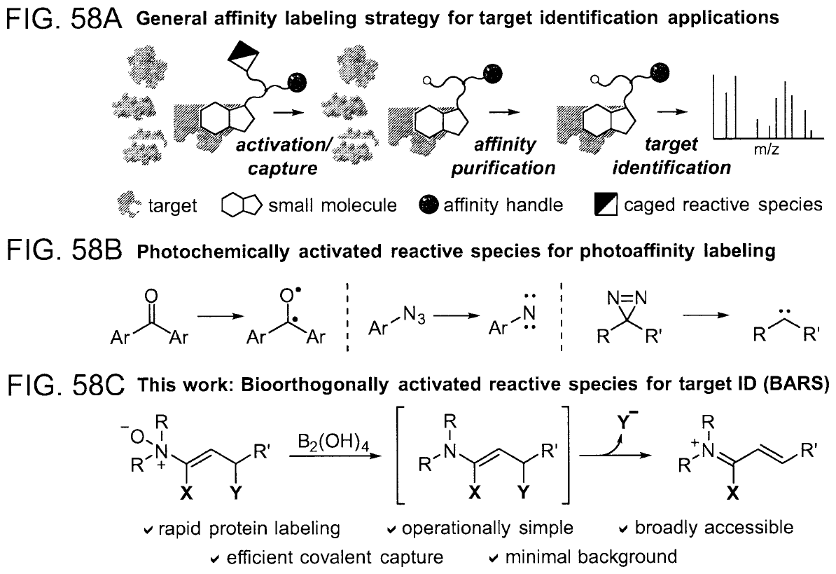

The following examples illustrate the utility of the BARS platform for biomacromolecule target identification (FIG. 58C). First, it was demonstrated that enamine N-oxides were activated through bioorthogonal chemical reduction by diboron reagents and that the activated reactive species covalently modify proteins and nucleic acids. Covalent modification with a reagent containing an affinity or click handle enabled visualization and/or purification of these biomacromolecules. For protein labeling, the amino acid labeling preference for the bioorthogonally activated reactive species were characterized, demonstrating that both nucleophilic residues and the peptide backbone undergo covalent modification. When the enamine N-oxide reagents were conjugated to a small molecule ligand, the BARS platform enabled selective modification and identification of the validated targets of the small molecule ligand in the presence of other proteins with which the ligand does not interact. This target identification platform was demonstrated to function on purified recombinant proteins, with endogenous levels of proteins in cell lysate, and with endogenous level of proteins in intact live cells. The platform is used in unbiased chemoproteomics experiment to engage in target identification with lower false positive and negative rates when compared to dialkyldiazirine reagents.

### Example 7: Ligand-Directed Labeling and Target Identification

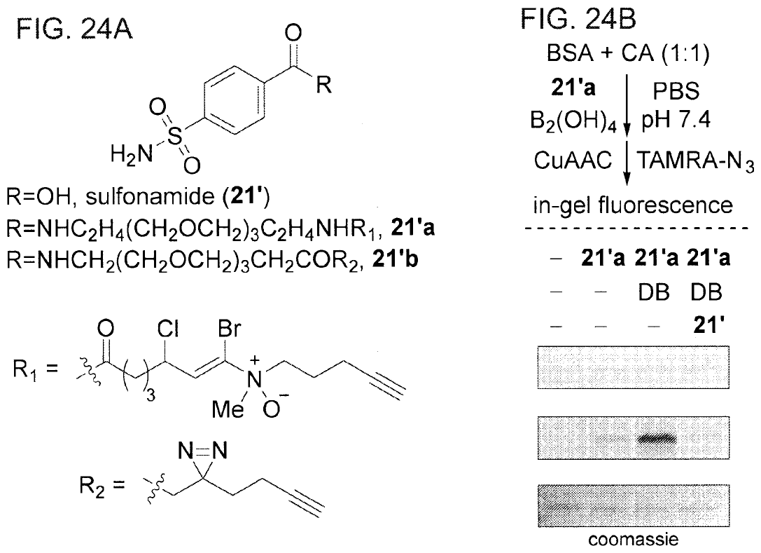

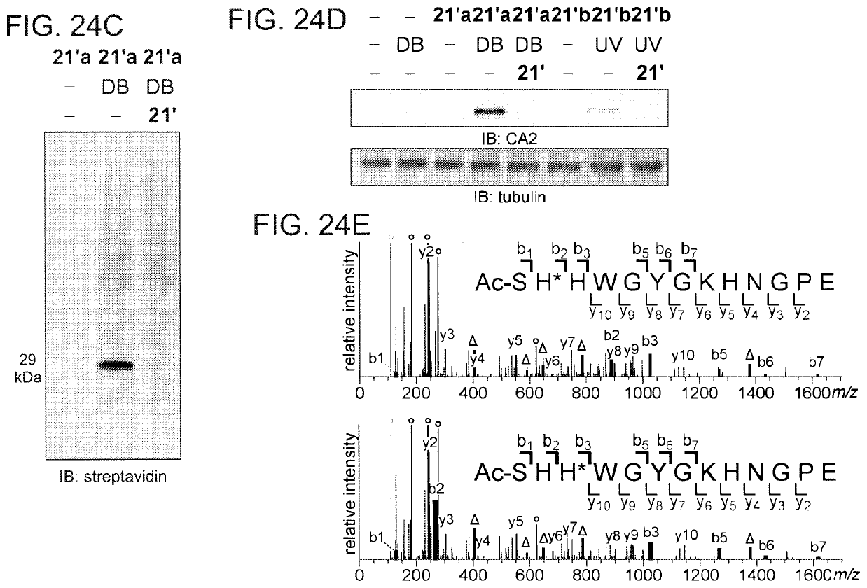

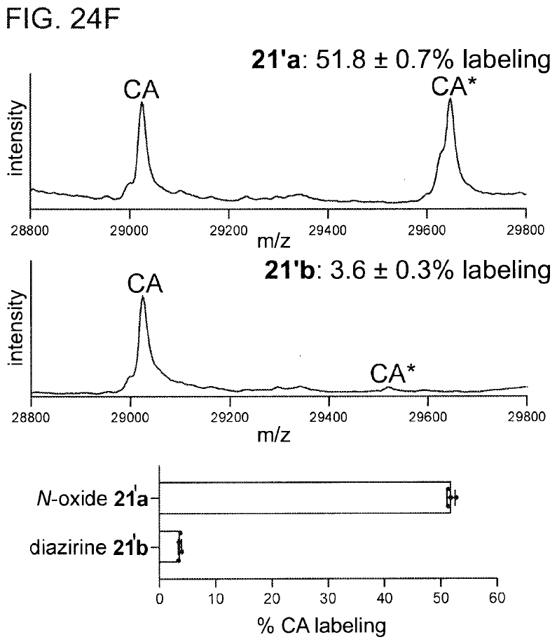

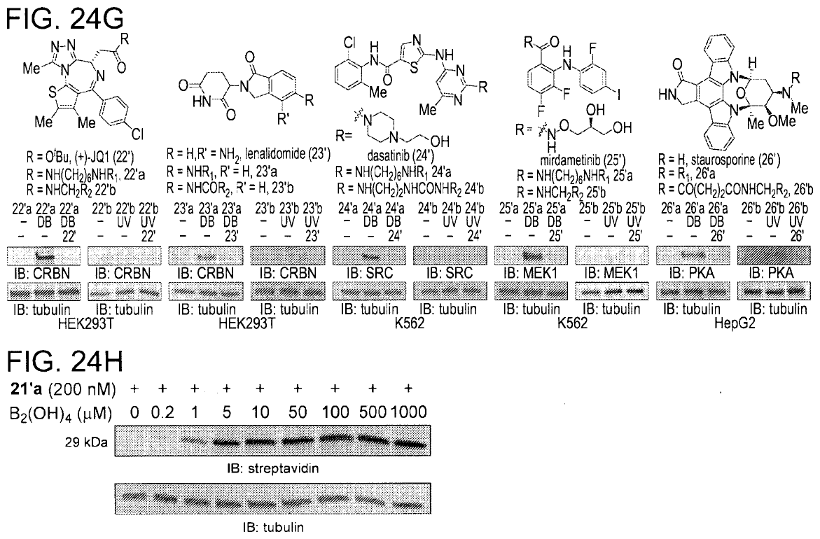

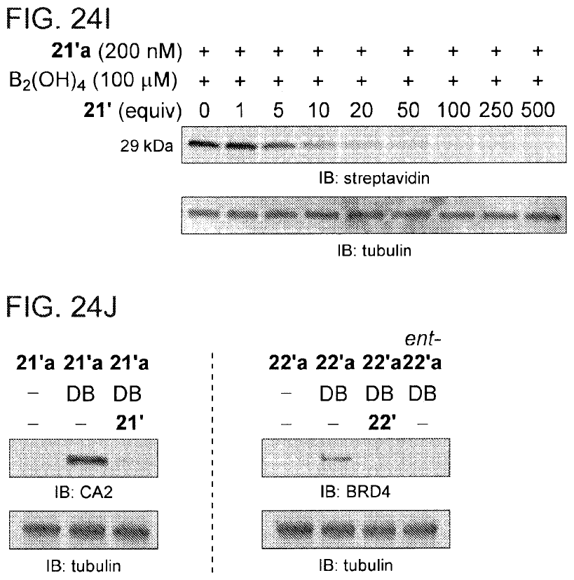

Ligand-directed labeling was performed in vitro with recombinant protein (FIG. 24B). An equimolar mixture of BSA and CA was treated with probe 21′a (200 nM) in PBS, pH 7.4 and activated with B₂(OH)₄ (100 μM) for 10 min. Protein labeling was analyzed by click conjugation of TAMRA-azide and in-gel fluorescence imaging. CA was preferentially labeled over BSA, and critically, labeling of CA but not BSA could be competed away with excess sulfonamide (21′, 50 μM). Endogenous levels of CA in HEK293T cell lysate could also be labeled using N-oxide 21′a (200 nM) and B₂(OH)₄ (100 μM). Here, biotin was conjugated to the alkyne-modified protein and analyzed by streptavidin blot (FIG. 24C). Labeling was again dependent on diboron and could be competed away with excess ligand.

Ligand-directed labeling of CA in HEK293T cell lysate was performed analogously with diazirine 21′b. Only, the treatment with diboron was substituted with UV irradiation (365 nm) for 10 min. Biotin modification and protein pulldown followed by immunoblotting against CA revealed the superior labeling efficiency of BARS over PAL, the current gold standard, for this substrate (FIG. 24D).

Intact mass spectrometry of recombinant CA treated with either probe 21′a (10 μM) and B₂(OH)₄ (100 μM) or 21′b and UV irradiation revealed 51.8% labeling of CA by the enamine N-oxide and 3.6% by the diazirine, a 14.4-fold improvement (FIG. 24F). CA modified in like manner and purified from excess reagent was then subjected to site identification studies. Two peptides labeled at either His2 or His3 of the N-terminal SHHWGYGKHNGPE (SEQ ID NO: 5) sequence were identified (FIG. 24E) (West et al., J. Am. Chem. Soc., 144:21174-21183 (2022); Trowbridge et al., Proc. Natl. Acad. Sci. USA, 119:e2208077119 (2022); Ficarro et al., Anal. Chem., 88:12248-12254 (2016); Conway et al., Chem. Sci., 12:7839-7847 (2021)). Both histidines are located at the lip of the CA ligand binding pocket and within reaction distance of the tethered reactive species (FIG. 42).

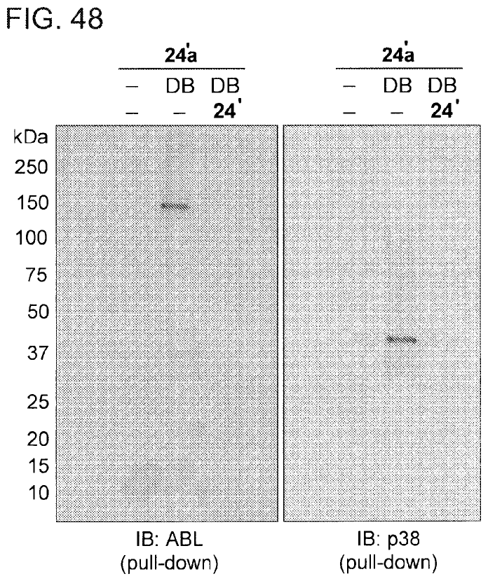

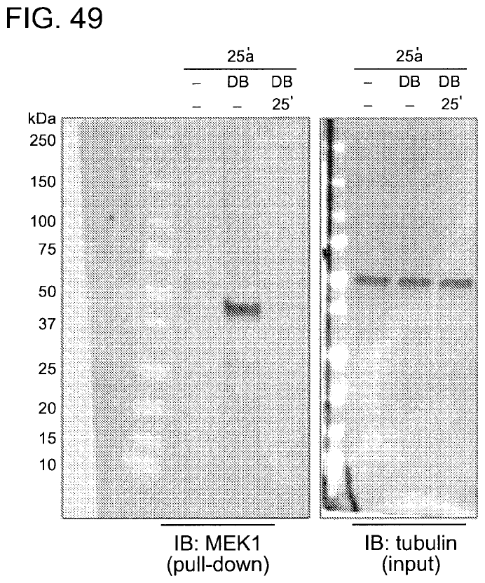

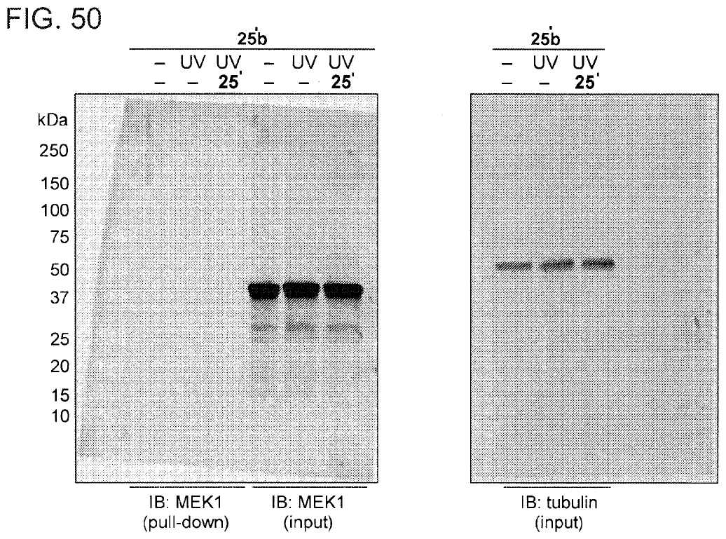

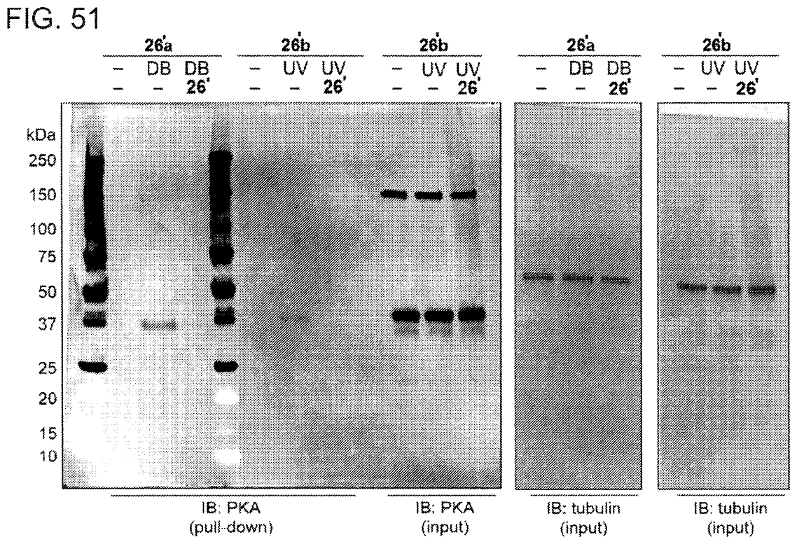

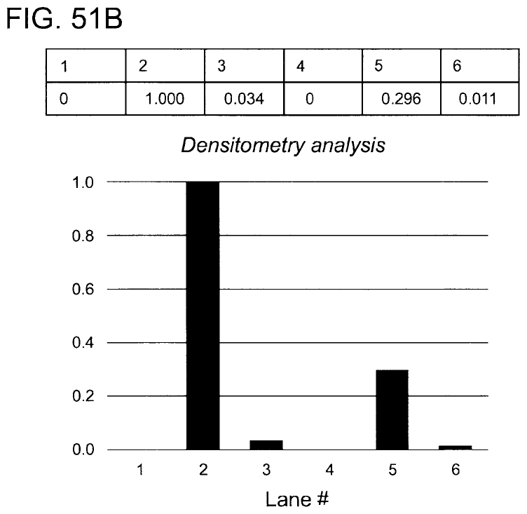

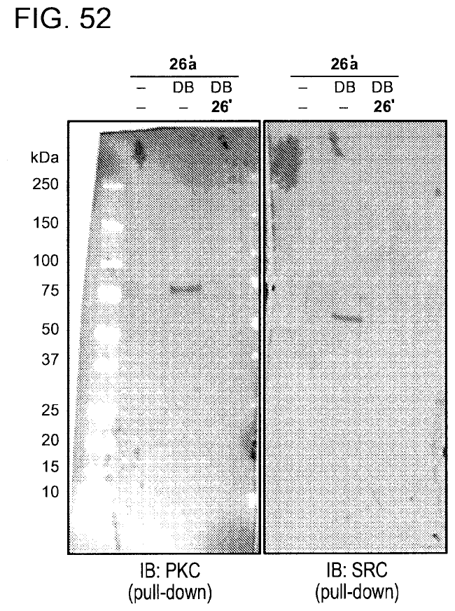

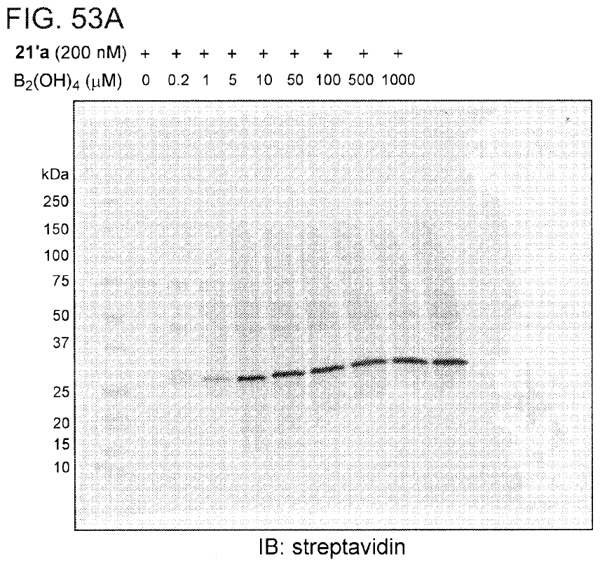

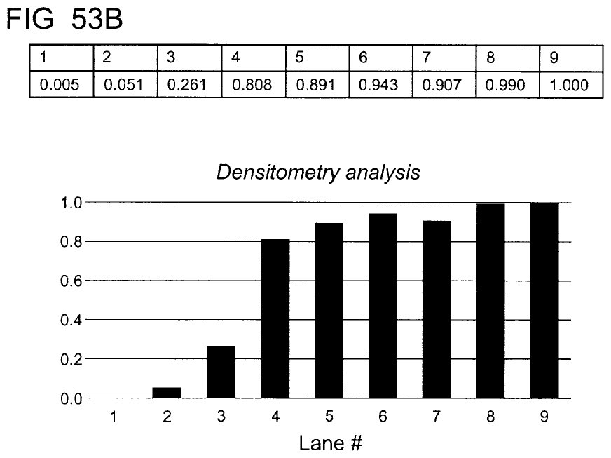

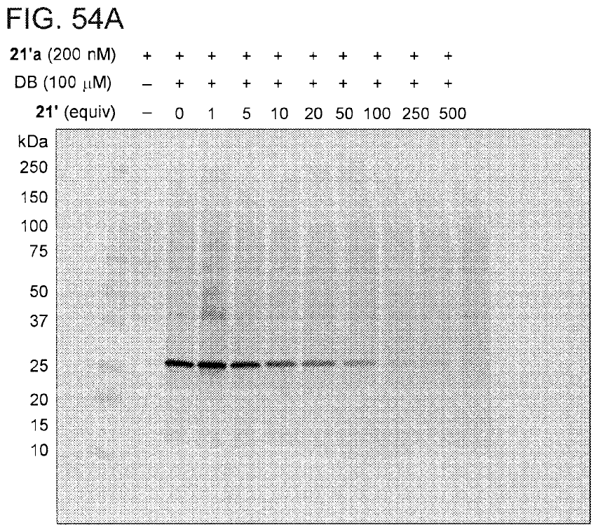

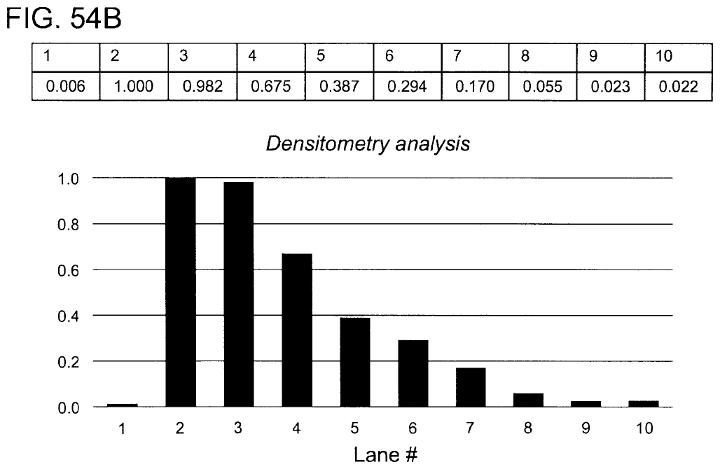

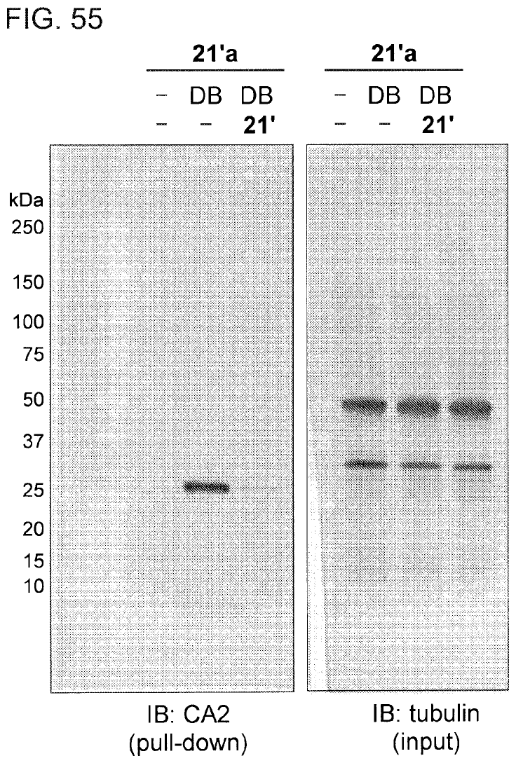

A collection of molecules [BET bromodomain inhibitor (+)-JQ1 (22′), cereblon inhibitor lenalidomide (23′), promiscuous Src kinase/BCR-ABL1 inhibitor dasatinib (24′), MEK1 kinase inhibitor mirdametinib (25′), and pro-apoptotic pan-kinase inhibitor staurosporine (26′)] were assembled to illustrate the generality of the BARS platform. Each was modified with an N-oxide or diazirine (West et al., J. Am. Chem. Soc., 144:21174-21183 (2022); Trowbridge et al., Proc. Natl. Acad. Sci. USA, 119:e2208077119 (2022); Conway et al., Chem. Sci., 12:7839-7847 (2021); Lin et al., J. Am. Chem. Soc. 144:606-614 (2022)) and evaluated for their capacity to identify targets in an activation- and ligand-dependent manner (FIG. 24G). For promiscuous probes with additional validated targets (FIG. 48, FIG. 52). Each BARS probe positively identified validated targets without fail. The low incidence of false negatives is notable.

Standard conditions for BARS labeling involve treatment of cell lysate with 1 μM probe and 100 μM B₂(OH)₄ for 10 min, and competition is performed using 50-fold excess of the parent ligand; however, B₂(OH)₄ concentrations as low as 5 μM can be used to identical effect (FIG. 24H), and significant competition can be observed with as low as 10-fold excess ligand (FIG. 24I). Given the remarkable biorthogonality of B₂(OH)₄, minimal strategic value was found in using the reagents at their lower limits and the built-in tolerances afford a more robust method.

The use of this platform was evaluated in live cells (FIG. 24J). HEK293T cells were treated with probe 21′a (5 μM) for 2 h, washed, treated with B₂(OH)₄ (100 μM) for 10 min, washed, pelleted, lysed, and immunoblotted against CA2, the target was positively identified. Labeling could be ablated by withdrawing B₂(OH)₄ or by competition with 10-fold excess sulfonamide 21′ (50 μM). Target identification (ID) was also successfully executed in live HEK293T cells using JQ1-derived enamine N-oxide probe 22′a.

Successful target identification in cells with probes 21′a and 22′a using the BARS platform indicates that the enamine N-oxide motif alone is not prohibitive for cell permeability, and that diboron penetrates the cell membrane, activates the enamine N-oxide, and successfully initiates selective protein target labeling inside cells; however, the cell permeability of the ligand-enamine N-oxide composite should be evaluated on a ligand-by-ligand basis. That the physicochemical properties of a ligand are altered by chemical modification is a fact to which neither BARS nor any other label transfer method is immune (Conway et al., Chem. Sci., 12:7839-7847 (2021)).

Positive identification of validated targets through Western blot analysis provides an indication of the low false negative rate of the method described; however, target ID experiments are more commonly performed in a prospective manner without prior knowledge of the true target or targets. In this instance, a method with a low false positive rate is desirable. To gain a better understanding of how the method performs in an unbiased target ID study, pulldown-MS chemoproteomics experiments were carried out.

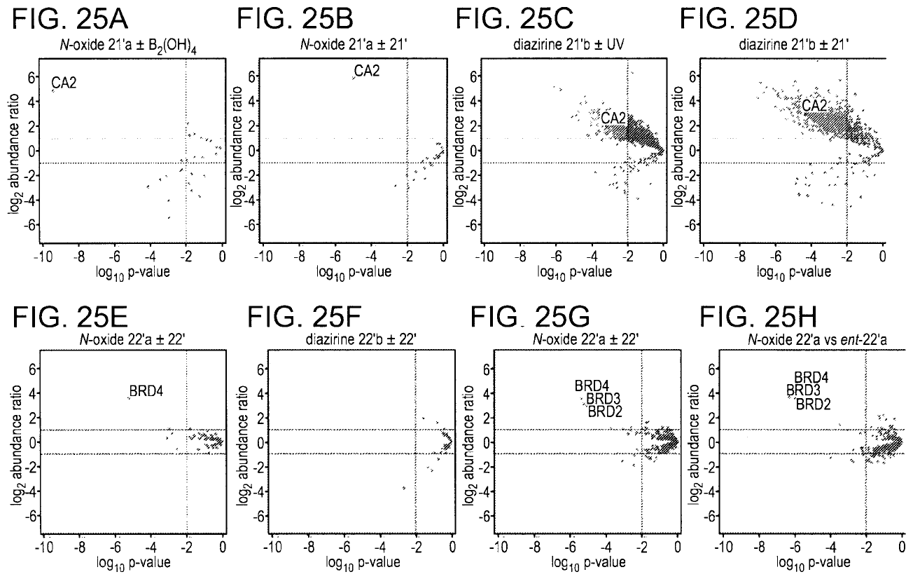

With sulfonamide (21′) as the first subject, samples were prepared identically to prior Western blot experiments through the streptavidin pulldown step. Captured proteins were then eluted from the solid support and subjected to trypsin digestion, LC-MS/MS, and label-free quantification. Experiments were performed in quadruplicate, and a hit was defined to be proteins with an abundance ratio >2 and a p-value <0.01. Data are displayed in volcano plots featuring pairwise comparisons of probe 21′a against unactivated probe or in competition with excess sulfonamide (21′) (FIG. 25A and FIG. 25B). In each case, CA2 was identified as the sole hit. The data are notable not just for the exclusivity of the true positive but also for the exceptional fold change in protein abundance observed against both negative controls. The isolation of the CA2 hit is remarkable when juxtaposed with the results of diazirine analog 21′b (FIG. 25C and FIG. 25D).

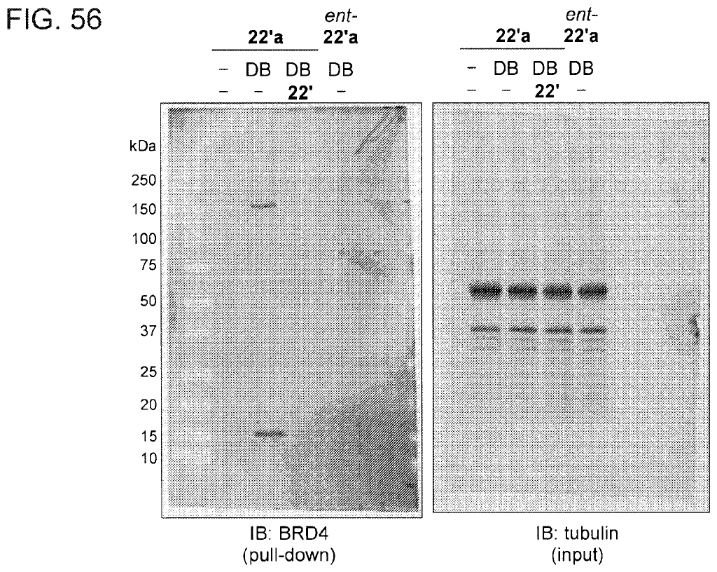

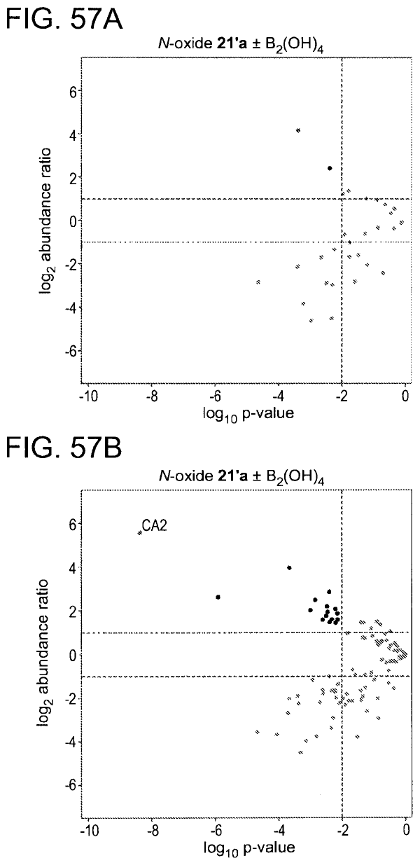

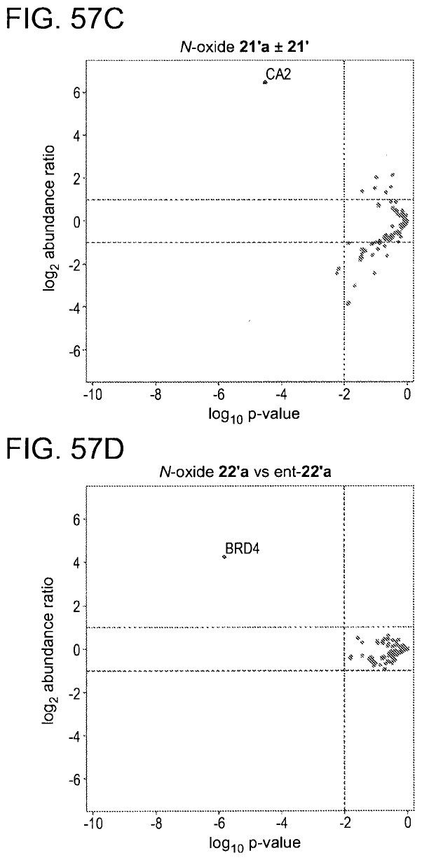

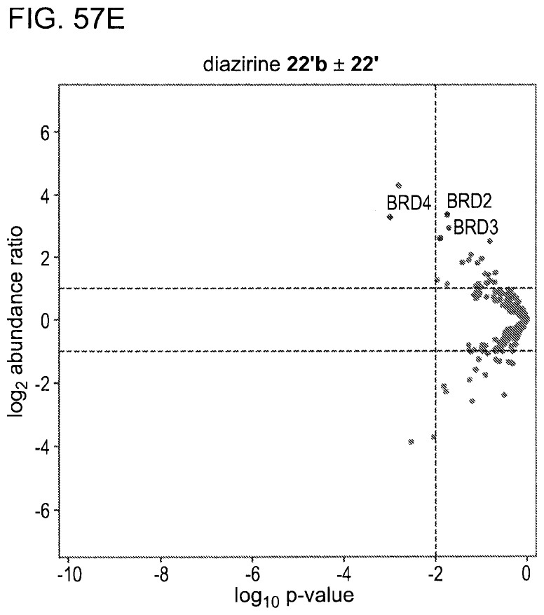

To further substantiate the described method for unbiased chemoproteomics, target identification with (+)-JQ1 (22′) was performed. Enamine N-oxide 22′a was employed in competition with parent drug (+)-JQ1 and in conjunction with the inactive (−)-JQ1 analog ent-22′a. When the proteomics data were processed identically to the CA2 samples with a protein identification threshold of 3 quantified peptides in 3 replicates, BRD4 was identified (FIG. 25E, FIG. 56). The stringency of the filter was reduced to a minimum of 1 quantified peptide in 3 replicates without introduction of appreciable background, enabling identification of proteins more than 200-fold less abundant than CA2 (Wang et al., Proteomics, 15:3163-3168 (2015)). Against competitor or enantiomer controls, BRD2/3/4 were identified as hits with just three false positives in the former and one in the latter (FIG. 25G and FIG. 25H). The availability of at least two controls for any compound facilitated the curation of protein hits for validation; each of the false positives for (+)-JQ1-derived probe 22′a was eliminated in this fashion. The results for diazirine probe 22′b are presented in FIG. 25F and FIG. 57A-FIG. 57E.

### Example 8: In-Vitro Labeling of Nucleic Acids with Enamine N-Oxides

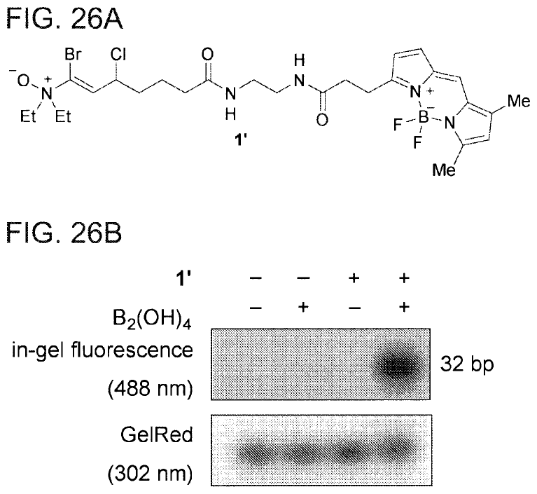

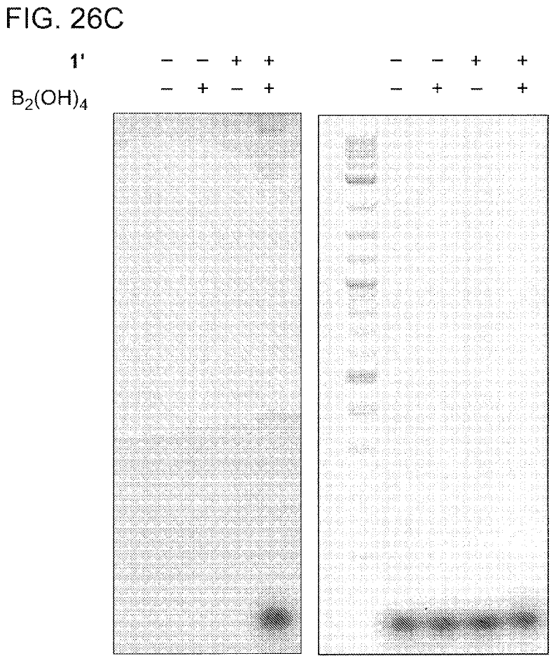

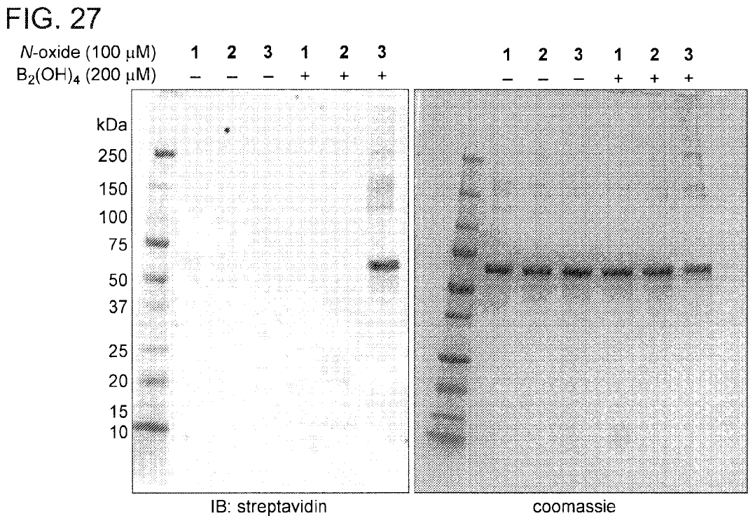

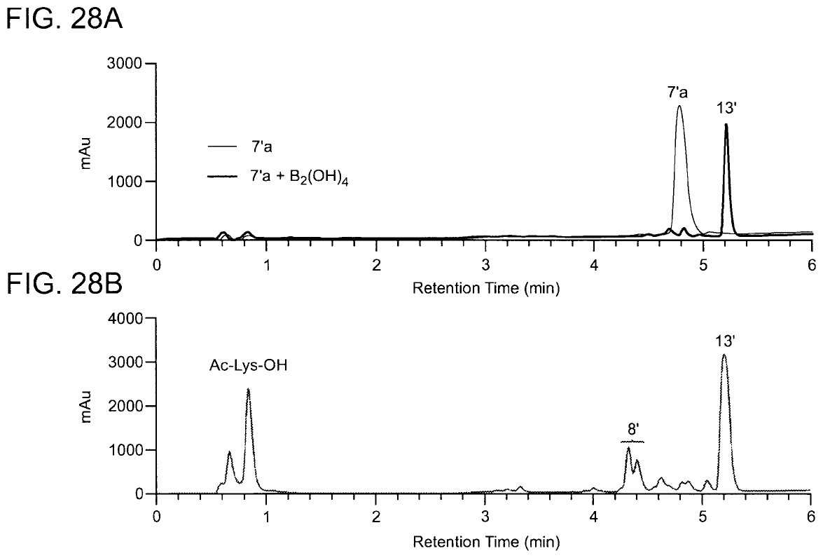

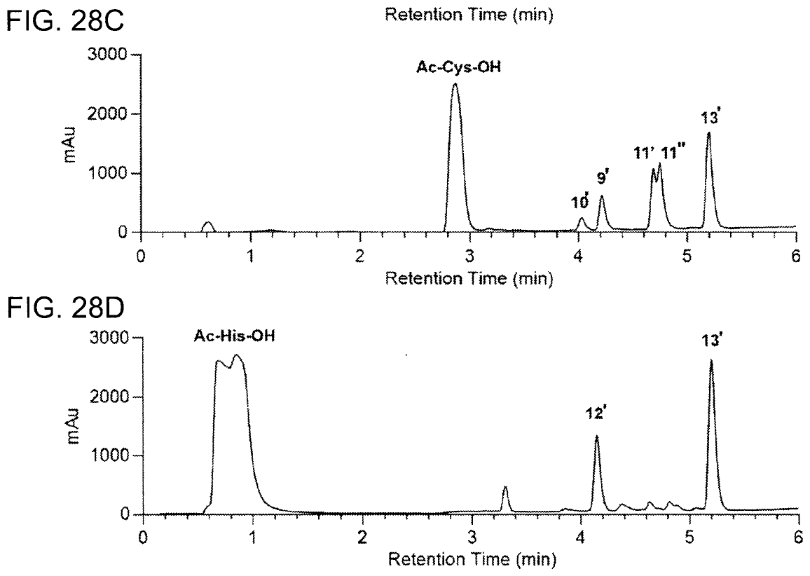

Enamine N-oxide compound 1′ (1 μL, 5 mM stock solution in 5% ethanol in water; 100 μM final concentration) or 5% ethanol in water (1 μL) vehicle control was added to microcentrifuge tubes containing a solution of single-stranded DNA (CATCATTGGGGTTACGGTAAACACAACGGTCC (SEQ ID NO: 6), 48 μL, 10 μM) in TE buffer (10 mM Tris, 1 mM EDTA, pH 8). Then tetrahydroxydiboron (1 μL, 10 mM solution in TE buffer; 200 M final concentration) or TE buffer, pH 8 (1 μL) vehicle control was added. The tubes were incubated at rt for 10 min. The samples were then diluted to 100 μL with TE buffer and each transferred to a desalting column (PD SpinTrap™ G-25, Cytiva) equilibrated with TE buffer. After all of the sample entered the packed gel matrix, 40 μL of TE buffer were additionally added to the column as a stacker volume. The columns were centrifuged once (800×g, 2 min) and the eluent was transferred to clean microcentrifuge tubes. 6×DNA loading buffer (20 μL) was added to 100 μL of the eluent and the samples were subjected to agarose gel electrophoresis. Ten (10) μL of each sample were loaded onto an unstained 1.5% agarose gel (50 mL) containing 15 wells alongside Quick-Load® Purple 1 kb Plus (New England Biolabs, 100 ng in 5 μL) as DNA ladder and run in freshly prepared TAE running buffer (130 V, 45 min) using the Mini-Sub Cell GT horizontal DNA electrophoresis system (Biorad). In-gel fluorescence imaging of the gel was performed on a Typhoon™ FLA 9500 (GE) at 488 nm with photomultiplier tube (PMT) setting of 500 V. After the image was taken, the gel was subsequently stained in GelRed® by incubating the gel in 70 mL DI water containing 3× GelRed® solution (Biotium) overnight at rt. The gel was then imaged on the Gel Doc XR+ gel imaging system (Biorad) using the UV-transilluminator (302 nm) setting and ethidium bromide filter (FIG. 26A-FIG. 26C).

### Example 9: Biological Procedures

### Cell Culture and Cell Lysate Preparation

Cells were cultured in RPMI (K562) or DMEM (HEK293T, HepG2) containing 10% FBS (Sigma®), 100 units/mL penicillin, and 0.1 mg/mL streptomycin (Sigma®) in a humidified chamber at 37° C. under an ambient atmosphere with 5% CO₂ unless otherwise stated. All cell lines were acquired from ATCC. Cells were passaged and dissociated with 0.25% trypsin, 0.1% EDTA in HBSS (Corning). All cells tested negative for mycobacteria with the MycoAlert™ PLUS Mycoplasma Detection Kit (Lonza) following the manufacturer's protocol.

For cell lysate preparation, cells were removed from the incubator and washed once with PBS, pH 7.4. An appropriate amount of cold NP40 lysis buffer containing 1 mM PMSF was added and the lysed cells were transferred to Eppendorf® tubes on ice. The lysate was centrifuged (14000×g, 10 min, 4° C.) and the supernatant was transferred to clean Eppendorf® tubes. The lysates were either used immediately or flash-frozen in liquid nitrogen and stored at −80° C. Concentrations were determined by BCA assay.

### Stock Solutions

Enamine N-oxide probes were prepared as a stock solution in ethanol (10-200 mM) and stored at −80° C. Upon removing from the freezer, the stock solutions were kept on ice during their duration of use. All other compounds were prepared as a stock solution in DMSO (10-500 mM) and stored at −80° C.

Diboron compounds are prepared as a stock solution of at least 100 mM in DMSO and always prepared fresh prior to use. Tetrahydroxydiboron was prepared as a stock solution in DMSO (200 mM) and always prepared fresh prior to use. Subsequent dilutions were then prepared with PBS, pH 7.4.

### Example 10: In Vitro Labeling of Endogenous Target Proteins in Cell Lysate Using Ligand-Directed Enamine N-Oxide Conjugates

### Probe Incubation, Activation, and Click Reaction

Cell lysate was diluted in lysis buffer and incubated with enamine N-oxide probe (1 pM-10 mM) with or without competitor ligand (at least 2-fold excess relative to probe concentration). The tubes were rotated end over end at rt. Diboron reagent (freshly made from a stock solution in DMSO) was subsequently added (at least 2-fold excess relative to probe concentration) and incubated. Subsequently, the reporter probe was then conjugated by click reaction then analyzed by in-gel fluorescence or Western blot. Alternatively, samples can be subjected to affinity purification and analyzed by mass spectrometry.

### Click-Cocktail Solution Preparation

Fifty (50) L of copper(II) sulfate pentahydrate (50 mM in water) were added to 15 μL of BTTAA or THPTA (20 mM in water) in an Eppendorf® tube. Subsequently, biotin picolyl azide (50 μL, 5 mM in DMSO, Click Chemistry Tools) or TAMRA azide (50 μL, 5 mM in DMSO, Click Chemistry Tools) was added, followed by sodium ascorbate (35 μL, 100 mM in water) and vortexed. For every 1 mL of cell lysate (2 mg/mL) or recombinant protein (0.1-1 mg/mL), 60 μL of this freshly prepared click-cocktail were added. The volume added was scaled up or down proportionally to the scale of the labeling reaction.

### Western Blot Analysis (No Pull-Down)

For sample preparation, 5× sample loading buffer was added directly at least 10 μg aliquot of the clicked cell lysate clicked with biotin (picolyl) azide. The samples were subsequently heated to 95° C. for 10 min and loaded onto a BioRad Mini-PROTEAN® TGX™ Precast Protein gel, 4-20% (15 μL) and run in freshly prepared Tris-glycine running buffer (160 V, 50 min). The gel was washed (3×DI water) and transferred to a 0.2 μm PVDF membrane. After transfer, the membrane was washed (3×TBST) and blocked with 5% fish skin gelatin (or any appropriate blocking buffer for Western blot) in TBST (rt, 1 h), and subsequently incubated with the appropriate primary antibody (1:1000 dilution in 5% fish skin gelatin in TBST, rt, 1 h). The membrane was washed (3×TBST) and incubated with the appropriate secondary antibody (1:10000 dilution in 5% fish gelatin in TBST, rt, 1 h). The membrane was again washed thoroughly with TBST and imaged using a chemiluminescence imager (Amersham™ Imager 600, GE).

### Streptavidin Pull-Down

The click-modified cell lysate (3 mg) was transferred to 15 mL centrifuge tubes and precipitated via methanol/chloroform precipitation using the following protocol. Methanol (6 mL) was added to the lysate and vortexed. Chloroform (1.5 mL) was subsequently added and vortexed. Finally, water (4 mL) was added and vortexed. The samples were centrifuged (3000×g, 5 min, 4° C.). The top aqueous layer was removed, and methanol (8 mL) was subsequently added and vortexed. The samples were centrifuged (3000×g, 5 min, 4° C.), the supernatant was removed, and the methanol wash was repeated once more. After the second wash, the supernatant was partially removed, and the protein suspension was transferred back to microcentrifuge tubes. The tubes were centrifuged once more (10000×g, 5 min, 4° C.) and the supernatant was removed completely. The pellet was allowed to air dry for 20 min and subsequently resuspended in resuspension buffer (300 μL, 50 mM Tris, pH 8.0 with 8 M urea) and passed through a micropipette tip several times. The resuspended lysate was transferred to new 15 mL centrifuge tubes and diluted with NP40 lysis buffer (3 mL). Avidin agarose bead slurry (100 μL, Thermo Fisher Scientific™) washed with NP40 lysis buffer (3×2 mL) was subsequently added to the lysate and incubated at rt for 1 h or overnight at 4° C. on the rotisserie. After the enrichment, the beads were pelleted (3000×g, 5 min, 4° C.) and the supernatant was removed. The beads were subsequently washed with 1% SDS in PBS, pH 7.4 (5×10 mL) and PBS, pH 7.4 (5×10 mL). After each wash, the supernatant above the pelleted beads was completely removed. The beads were then transferred to clean microcentrifuge tubes and centrifuged (5000×g, 5 min, 4° C.). The residual supernatant was completely removed and elution buffer (30 mM biotin, 6 M urea, 2 M thiourea, 2% w/v SDS in PBS, pH 11.5, 24 μL) and 5× sample loading buffer (6 μL) were added with gentle mixing. The beads were heated to 95° C. for 10 min, pelleted, and the supernatant was removed while hot. The samples were cooled to rt and subjected directly to SDS-PAGE. The input control samples (obtained from a 10 μL aliquot of the resuspended lysate before addition of the avidin agarose beads) were also prepared and were run alongside the pull-down samples.

### SDS-PAGE and Western Blot Analysis

Samples were loaded onto a Bio-Rad Mini-PROTEAN® TGX™ Precast Protein 15-well gel, 4-20% (15 μL) alongside All Blue Prestained Protein Standard (Bio-Rad, 5 μL) as a ladder, and run in freshly prepared Tris-glycine running buffer (180 V, 45 min). The gel was washed (3×50 mL deionized water) and transferred to a 0.2 m low fluorescence PVDF membrane (Bio-Rad) using a semi-dry transfer apparatus (Bio-Rad Trans-Blot® Turbo™ Transfer System) and preprogrammed transfer setting (1.3 A, 25 V, 10 min). After transfer, the membrane was washed (3×20 mL TBST) and blocked with 5% fish skin gelatin (Sigma-Aldrich®) in TBST (15 mL, rt, 1 h), and subsequently incubated with the appropriate primary antibody (1:1000 dilution in 5% fish skin gelatin in TBST, 5 mL, rt, 1 h) and/or streptavidin-fluorophore conjugate (1:2500 dilution in 5% fish skin gelatin in TBST, 5 mL, rt, 1 h). The membrane was washed (3×20 mL TBST) and incubated with the appropriate secondary antibody (1:10000 dilution in 5% fish gelatin in TBST, 5 mL, rt, 1 h). The membrane was again washed thoroughly with TBST (3×20 mL) and imaged using a chemiluminescence imager (Amersham™ Imager 600, GE). Pixel densitometry was performed using Image Studio™ Lite V. 5.2.5 (Licor) and normalized to the band with the highest density within each blot.

### Sample Preparation for LFQ-Based Chemoproteomic Analysis

The same procedure as the “Streptavidin pull-down” was followed up to the wash steps. After the final wash, the supernatant was completely removed. Formamide elution buffer (95% v/v formamide, 5% v/v 100 mM sodium acetate, pH 10, 50 μL) was then added to the beads with gentle mixing. The beads were heated to 95° C. for 10 min, pelleted, and the supernatant was removed while hot and then allowed to cool to rt. The formamide elution was repeated a second time for a combined eluate volume of approximately 100 μL. The eluate samples were then submitted to the Dana-Farber TPD Core facility for further processing.

### Sample Preparation for TMT-Based Chemoproteomic Analysis

The same procedure as the “Streptavidin pull-down and Western blot analysis” was followed up to the elution step. Additionally, at least 3 mg of clicked cell lysate was used for each sample, and the procedure for the labeling, activation, click reaction, and pull-down were scaled up proportionally. Samples were not eluted from the avidin agarose beads and instead, the washed beads were resuspended in PBS (sufficient volume to cover the beads) and submitted to the TCMP Proteomics Core (Harvard Medical School) for on-bead digestion and further processing.

### Sample Preparation for Liquid Chromatography Tandem Mass Spectrometry (LC-MS/MS)

Eluates from enrichments were reduced in 10 mM TCEP for 30 min at rt and then treated with 15 mM iodoacetamide for 45 min in the dark to alkylate cysteine resides. Alkylation reactions were quenched with 10 mM dithiothreitol. Proteins were separated by methanol/chloroform precipitation. The protein pellets were resuspended in 50 μL of 200 mM EPPS, pH 8. LysC (2 μg) and trypsin (1 μg) were added and digests were incubated at 37° C. overnight with shaking. Digests were quenched to 1% formic acid and desalted over SOLA™ HRP elution plates (Thermo Fisher Scientific™) prior to LC-MS/MS analysis.

### LC-MS/MS Acquisition

Peptides were analyzed on an Ultimate™ 3000 RSLCnano system coupled to a Tribrid series mass spectrometer (Thermo Fisher Scientific™). Peptides were separated across a 70 min gradient of 6→30% acetonitrile in 1% formic acid over a 50 cm C₁₈ column (ES803A, Thermo Fisher Scientific™) and electrosprayed (2.05 kV, 300° C.) into the mass spectrometer with an EASY-Spray™ ion source (Thermo Fisher Scientific™). Precursor ion scans (375-1,325 m/z) were obtained in the orbitrap at 120,000 resolution in profile (RF lens %=30, Max IT=200 ms). MS² scans were acquired in the orbitrap following HCD fragmentation (35% NCE) in data dependent mode with the following parameters: 0.5 m/z isolation window, 30,000 resolution, standard AGC target, dynamic max IT, dynamic exclusion, 3 sec cycle time.

### LC-MS/MS Data Analysis

RAW file processing, false discovery rate filtering (<1%), and protein roll-up and MS1 intensity-based label-free quantification (Minora algorithm) were achieved using Proteome Discoverer 2.5 (Thermo Fisher Scientific™). Raw data were searched against a human protein database (Uniprot, 2021) using SEQUEST, permitting a mass tolerance of ±10 ppm, 2 missed cleavages, and the following modifications: methionine oxidation, serine/threonine/tyrosine phosphorylation, and cysteine carbamidomethylation. Peptides were quantified by precursor ion abundance and summed across proteins.

Proteins were filtered to include those with three or more unique peptides quantified across four replicate treatments, unless otherwise stated. Protein abundances were normalized to total abundances across samples. Changes in protein abundance across samples were assessed by moderated t-test using the limma package (Ritchie et al., Nucleic Acids Research, 43:e47 (2015)) in R with a threshold of p-value <0.01 and fold change >2. One replicate was removed from the comparison in FIG. 25A as CA2 abundance differed by more than a factor of 10 from the other replicates. A comparison including all four replicates of FIG. 25A is shown in FIG. 57A. All peptides of target proteins quantified at the single peptide level were manually inspected to ensure accuracy in quantification.

### Example 11: In Vitro Labeling of BSA with Excess Reactive Species Using Enamine N-Oxides 1-3

Enamine N-oxide compound (1 μL, 10 mM stock solution in ethanol; 100 M final concentration) was added to a suspension of BSA (10 μg) in PBS, pH 7.4 (98 μL) and incubated at rt for 30 min. Tetrahydroxydiboron (1 μL, 20 mM stock solution in DMSO; 200 μM final concentration) was subsequently added and gently mixed. After 10 min, CuAAC click-cocktail solution containing biotin picolyl azide (6 μL) was added and the sample was incubated at rt for an additional 1 h.

For sample preparation, 5× sample loading buffer (25 μL) was added directly to the biotin-modified BSA samples. The samples were subsequently heated to 95° C. for 10 min and then subjected to SDS-PAGE and Western blotting.

### Example 12: Nano-LCMS/MS-Based Evaluation of Amino Acid Labeling Preference on Recombinant Proteins with Compound 7′b

Enamine N-oxide 7′b (1 μL, 10 mM in ethanol; 100 μM final concentration) was added to four microcentrifuge tubes each containing one of the four recombinant proteins carbonic anhydrase, lysozyme, myoglobin, or BSA (50 μg in 98 μL PBS, pH 7.4) and incubated at rt for 30 min. Tetrahydroxydiboron (1 μL, 20 mM solution in ethanol; 200 M final concentration) was subsequently added and gently mixed. The tubes were incubated at rt for an additional 10 min. Subsequently, all four samples were each diluted with 400 μL PBS, pH 7.4 and concentrated via spin filtration (Amicon® Ultra-0.5 mL, UFC500324, 3 kDa MWCO for lysozyme and myoglobin, 10 kDa MWCO for CA and BSA) following the manufacturer's recommended protocol. PBS was added after each filtration cycle and the concentration was repeated for a total of five times. Proteins were reduced with dithiothreitol (10 mM final concentration, 56° C., 30 min), alkylated with iodoacetamide (22.5 mM final concentration, rt, 30 min protected from light), and digested overnight with trypsin (1:25) at 37° C. in 100 mM ammonium bicarbonate. Digests were acidified, and 300 ng peptide was loaded onto EVO tips. Each digest was analyzed in duplicate using an EVO SEP LC system (100 SPD method, column=100 μm I.D., 8 cm, 3 μm particle size, Reprosil C₁₈, solvent A=0.1% formic acid, B=0.1% formic acid in acetonitrile) coupled to a timsTOF Pro 2 mass spectrometer (Bruker). The mass spectrometer collected ion mobility MS spectra over a mass range of m/z 100-1700 and 1/ko of 0.6-1.6 and performed 10 cycles of PASEF MS/MS with a target intensity of 14.5 k and a threshold of 1750. Active exclusion was enabled with a release time of 0.4 min. MS/MS spectra were matched to peptide sequences from the model proteins using a custom database with PEAKS Studio 10.0 software. Search parameters specified semi-trypsin specificity, precursor and product ion tolerances of 25 ppm and 0.05 Da, variable oxidation of methionine, and variable acetylation of the protein N-terminus. Custom modifications for 1,2- and 1,4-addition were created to allow for probe modification of any residue (including double modification of the histidine imidazole). Results from replicate analyses of all four proteins were combined and filtered for high confidence identifications (−log₁₀ P >30) and A-score for probe modification >15 (<5% false localization rate).

### Example 13: Labeling Site Identification and Intact Mass Spectrometry of Recombinant Carbonic Anhydrase

### Sample Preparation

BARS probe 21′a: Enamine N-oxide 21′a (10 μL, 500 μM in PBS, pH 7.4; 10 μM final concentration) was added to an aliquot of recombinant CA from bovine erythrocytes (50 g, MP Biomedicals) suspended in 480 μL PBS, pH 7.4 in a microcentrifuge tube and incubated at rt for 30 min. Tetrahydroxydiboron (10 μL, 5 mM in PBS, pH 7.4; 100 μM final concentration) was subsequently added and gently mixed. The tubes were incubated at rt for an additional 10 min. Subsequently, the sample was concentrated via spin filtration (Amicon® Ultra-0.5 mL, UFC500324, 10 kDa MWCO) following the manufacturer's recommended protocol. PBS was added after each filtration cycle and the concentration was repeated for a total of five times. The eluate was collected in a clean microcentrifuge tube and its concentration determined by NanoDrop (Thermo Fisher Scientific™).

PAL probe 21′b: Diazirine 21′b (10 μL, 500 μM in PBS, pH 7.4; 10 μM final concentration) was added to an aliquot of recombinant CA from bovine erythrocytes suspended in 490 μL PBS, pH 7.4 in a microcentrifuge tube and incubated at rt for 30 min. The samples were then transferred onto a 24-well plate and UV irradiated (365 nm) for 10 min on ice. After every 2 min of irradiation, the plate was rotated 900 to ensure complete and equal UV exposure. After irradiation, the samples were transferred to a spin filtration column (Amicon® Ultra-0.5 mL, UFC500324, 10 kDa MWCO) and concentrated following the manufacturer's recommended protocol. PBS was added after each filtration cycle, and the concentration was repeated for a total of five times. The eluate was collected in a clean microcentrifuge tube and its concentration determined by NanoDrop (Thermo Fisher Scientific™).

### Identification of Enamine N-Oxide 21′a Modification Sites on Recombinant Carbonic Anhydrase

An aliquot of carbonic anhydrase labeled with enamine N-oxide 21′a (prepared from the above procedure) was reduced with dithiothreitol (10 mM final concentration, 56° C., 30 min), alkylated with iodoacetamide (22.5 mM final concentration, rt, 30 min protected from light), and digested overnight with Glu-C (1:20) at 37° C. in 100 mM ammonium bicarbonate. Peptides were desalted using SP3 beads and analyzed by nano-LC-MS/MS using a nanoAcquity UPLC system interfaced to a QExactive HF mass spectrometer (Ficarro et al., Anal. Chem., 9:3440-3447 (2009)). Peptides were loaded on a precolumn (5 cm Symmetry C₁₈, 100 μm ID.), resolved on an analytical column (50 cm Monitor C₁₈, 30 μm I.D.), and eluted to the mass spectrometer with an HPLC gradient (7→50% B in 59 minutes, A=0.1% formic acid in water, B=0.1% formic acid in acetonitrile). The mass spectrometer performed MS/MS on the 10 most abundant precursor ions (target=1e5, isolation window=1.4 Da, NCE=28%, resolution=15 k, max IT=100 ms) in each MS1 scan (120 k resolution, 1e6 target, m/z 300-2000). MS/MS spectra were matched to peptide sequences from a custom database of laboratory proteins containing carbonic anhydrase with Mascot version 2.6.2. Search parameters specified Glu-C specificity, precursor and product ion tolerances of 10 ppm and 0.025 Da, variable oxidation of methionine, variable acetylation of the protein N-terminus, and variable modification of H or K by probe 21′a. PSMs corresponding to the modified CA N-terminus were manually validated using mzStudio software (Ficarro et al., Proteomes, 3:20 (2017)). Probe related fragments were assigned according to Ficarro et al., Anal. Chem., 24:12248-12254 (2016).

### LC-ESI-MS Analysis of Proteins

Replicate samples of carbonic anhydrase (n=4; labeled with enamine N-oxide 21′a or diazirine 21′b) were desalted on a self-packed column (500 μm I.D. fused silica packed with 5 cm POROSR2 resin) and gradient eluted (0→100% B in 1 min, A=200 mM acetic acid in water, B=200 mM acetic acid in acetonitrile) to the mass spectrometer (LTQ XL mass spectrometer, Thermo Fisher Scientific; spray voltage=5 kV). The mass spectrometer recorded full scan profile spectra n z 300-2000. Mass spectra were deconvoluted using MagTran version 1.03b2 (Zhang et al., J. Am. Chem. Soc. Mass. Spectrom., 3:225-244 (1998)). Percent labeling was calculated as intensity labeled protein/(intensity unlabeled protein+intensity labeled protein)×100%.

### Example 14: In Vitro BARS Labeling of 1:1 Recombinant BSA/CA with Enamine N-Oxide 21′a

An equimolar suspension of recombinant BSA and recombinant CA was prepared by combining BSA (22 μg, 1 mg/mL in PBS, pH 7.4) and CA (10 pg, 1 mg/mL in PBS, pH 7.4) in PBS, pH 7.4 (1 mL). This was approximately 333 nM of each protein. For each reaction, an aliquot of this mixture (97 μL) was added to a microcentrifuge tube. Enamine N-oxide probe 21′a (1 μL, 5 μM solution; 50 nM final concentration) was added to this aliquot. Sulfonamide competitor (21′, 1 μL, 5 mM solution in 1% DMSO/PBS; 50 μM final concentration) or PBS, pH 7.4 (1 μL) vehicle control was added to the samples. The tubes were incubated at rt for 30 min. Tetrahydroxydiboron (1 μL, 10 mM solution; 100 μM final concentration) was subsequently added with gentle mixing. The tubes were incubated at rt for an additional 10 min. Subsequently, CuAAC click-cocktail solution containing TAMRA-azide (6 μL) was added and the tubes were incubated at rt in the dark for 1 h.

For sample preparation, 5× sample loading buffer (25 μL) was added directly to the TAMRA-modified BSA/CA samples. The samples were subsequently heated to 95° C. for 10 min and then subjected to SDS-PAGE and in-gel fluorescence imaging. In-gel fluorescence imaging was performed on a Typhoon™ FLA 9500 (GE) at 532 nm with photomultiplier tube (PMT) setting of 300 V.

### Example 15: In Vitro PAL Labeling Using Ligand-Directed Diazirine Conjugates (21′b-26′b)

Diazirine PAL probe (15 μL; 200 nM or 1 μM final concentration for probe 21′b, 1 μM final concentration for probes 22′b-26′b) was added to a microcentrifuge tube containing cell lysate (3 mg, as determined by BCA assay) suspended in NP40 lysis buffer (1.47 mL). Competitor (15 μL, 500 μM solution; 50 μM final concentration) or PBS, pH 7.4 (15 μL) vehicle control was then added. The tubes were rotated at rt for 30 min. The samples were then transferred onto a 12-well plate and UV irradiated (365 nm) for 10 min on ice. After every 2 min of irradiation, the plate was rotated 900 to ensure complete and equal UV exposure. After irradiation, the samples were transferred back to microcentrifuge tubes. Finally, CuAAC click-cocktail solution containing biotin picolyl azide (90 μL) was added and the tubes were rotated at rt for 1 h. After this point, the PAL-labeled samples were processed identically to the BARS-labeled samples.

### Example 16: In Vitro BARS Labeling of Endogenous Target Proteins in Cell Lysate Using Ligand-Directed Enamine N-Oxide Conjugates (21′a-26′a)

### Probe Incubation, Activation, and Click Reaction

Enamine N-oxide probe (15 μL; 200 nM or 1 μM final concentration for probe 21′a, 1 pM for probes 22′a-26′a) was added to a microcentrifuge tube containing cell lysate (3 mg, as determined by BCA assay) suspended in cold NP40 lysis buffer (1.455 mL). Competitor (15 μL, 500 μM solution; 50 μM final concentration) or PBS, pH 7.4 (15 μL) vehicle control was then added. The tubes were rotated at rt for 30 min. A freshly made solution of tetrahydroxydiboron (15 μL, 10 mM solution; 100 μM final concentration) was subsequently added. The tubes were rotated at rt for an additional 10 min. CuAAC click-cocktail solution containing biotin picolyl azide (90 μL) was added and the tubes were rotated at rt for 1 h.

### Example 17: In Cellulo BARS Labeling of Endogenous Target Proteins Using Ligand-Directed Enamine N-Oxide Conjugates

HEK293T cells were cultured in DMEM containing 10% FBS (Sigma), 100 units/mL penicillin, and 0.1 mg/mL streptomycin (Sigma) in a humidified chamber at 37° C. under an ambient atmosphere with 5% CO₂. After culturing to >80% confluency in 150 mm plates, the plates were removed from the incubator, the media was aspirated, and the cells were trypsinized at 37° C. with 0.25% trypsin, 0.1% EDTA in HBSS (3 mL). After 3 min, complete growth medium (7 mL) was added to the detached cells, and the cell suspension was transferred to a 50 mL centrifuge tube. Cells were pelleted (400×g, 4 min, 23° C.) and washed with 2×20 mL PBS, pH 7.4. At this point, the density of the cell suspension in PBS was determined by the trypan blue exclusion method. The cells were pelleted once more, and the volume of PBS was adjusted to produce a cell suspension with a final density of 2×10⁷ cells/mL. The cell suspension was aliquoted into 2 mL microcentrifuge tubes (3×1 mL, 2×10⁷ cells each). Enamine N-oxide probe 21′a (5 μM final concentration, from a 20 mM stock in EtOH) was added to all three tubes. Competitor 21′ (50 μM final concentration, from a 500 mM stock in DMSO) was also added to the third tube. The samples were incubated at 37° C. with end-over-end rotation. After 2 h of incubation, the cells were pelleted (400×g, 4 min, 23° C.) and washed once with PBS, pH 7.4. Then, a freshly made solution of tetrahydroxydiboron (1 mL, 100 μM final concentration in PBS, pH 7.4, from a 200 mM stock in DMSO) was added to tubes 2 and 3, while 1 mL PBS vehicle was added to tube 1. The samples were incubated at rt with end-over-end rotation for an additional 10 min. The cells were then pelleted (400×g, 4 min, 23° C.), washed twice with PBS, pH 7.4 and then lysed in cold NP40 lysis buffer (1.5 mL) containing 1 mM PMSF. The lysate was centrifuged (14000×g, 10 min, 4° C.) and the supernatant was transferred to clean microcentrifuge tubes on ice. Subsequent steps, including click reaction, streptavidin pull-down, SDS-PAGE, and Western blot analysis, were performed identically to the in vitro labeling procedure. For labeling with (+)-JQ1 enamine N-oxide 22′a, a suspension of 4×10⁷ cells/mL was generated and 4×10⁷ cells were used in each experiment.

TABLE 1
List of antibodies

| Target | Supplier | Identifier | Dilution |
|---|---|---|---|
| CA2 | Abclonal | A9148 | 1:1000 |
| BRD4 | Abclonal | A12677 | 1:1000 |
| c-SRC | Abclonal | A19119 | 1:1000 |
| p38 | Abclonal | A4771 | 1:1000 |
| c-ABL | Abclonal | A22082 | 1:1000 |
| MEK 1 | Abclonal | A19565 | 1:1000 |
| PKA (C-α) | Cell Signaling | 5842T | 1:1000 |
| | Technology | | |
| PKC α | Abclonal | A11107 | 1:1000 |
| CRBN | Cell Signaling | 71810 | 1:1000 |
| | Technology | | |
| Tubulin | Cell Signaling | 11H10 | 1:1000 |
| | Technology | | |
| IRDye ® 680 Streptavidin | Licor | 926-68079 | 1:2500 |
| IRDye ® 800CW Goat anti- | Licor | 925-32211 | 1:10000 |
| Rabbit IgG | | | |

TABLE 2
Diazirine Pal probes

| Probe Structure | Ref. |
|---|---|
| 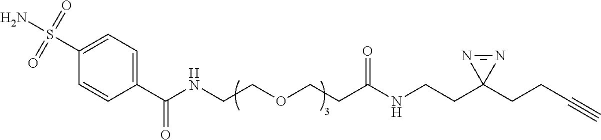 | Described above |
| sulfonamide diazirine | |
| 21′b | |
| 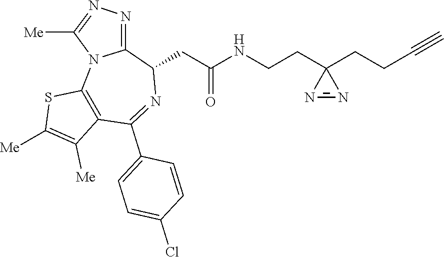 [SMILES: C#CCCC1(CCNC(=O)C[C@@H]2N=C(c3ccc(Cl)cc3)c3c(sc(C)c3C)-n3c(C)nnc32)N=N1] | Trowbridge et al., Proc. Natl. Acad. Sci., 119:e2208077119 (2022) |
| (+)-JQ1 diazirine | |
| 22′b | |
| 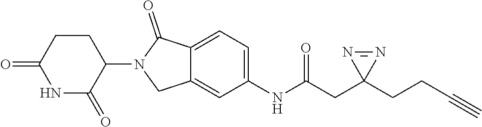 [SMILES: C#CCCC1(CC(=O)Nc2ccc3c(c2)CN(C2CCC(=O)NC2=O)C3=O)N=N1] | Lin et al., J. Am. Chem. Soc., 144:606-614 (2022) |
| lenalidomide diazirine | |
| 23′b | |
| 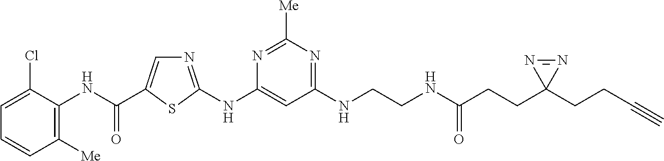 [SMILES: C#CCCC1(CCC(=O)NCCNc2cc(Nc3ncc(C(=O)Nc4c(C)cccc4Cl)s3)nc(C)n2)N=N1] | Li et al., Angew. Chem. Int. Ed., 52:8551-8556 (2013) |
| dasatinib diazirine | |
| 24′b | |
| 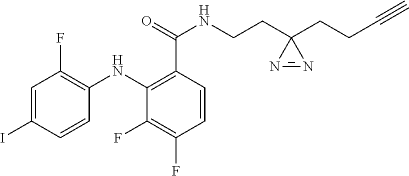 [SMILES: C#CCCC1(CCNC(=O)c2ccc(F)c(F)c2Nc2ccc(I)cc2F)N=N1] | Li et al., Angew. Chem. Int. Ed., 52:8551-8556 (2013) |
| mirdametinib diazirine | |
| 25′b | |
| 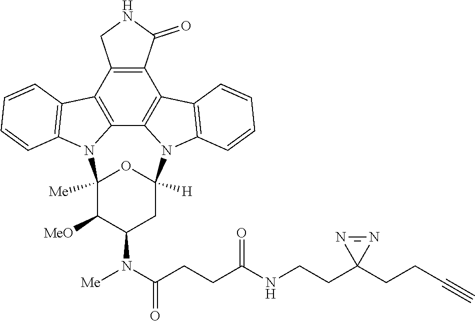 [SMILES: C#CCCC1(CCNC(=O)CCC(=O)N(C)[C@@H]2C[C@H]3O[C@@](C)([C@@H]2OC)n2c4ccccc4c4c5c(c6c7ccccc7n3c6c42)C(=O)NC5)N=N1] | Li et al., Angew. Chem. Int. Ed., 52:8551-8556 (2013) |
| staurosporine diazirine | |
| 26′b | |

All patent publications and non-patent publications are indicative of the level of skill of those skilled in the art to which this disclosure pertains. All these publications are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A compound having a structure represented by any one of formulas I-VI: 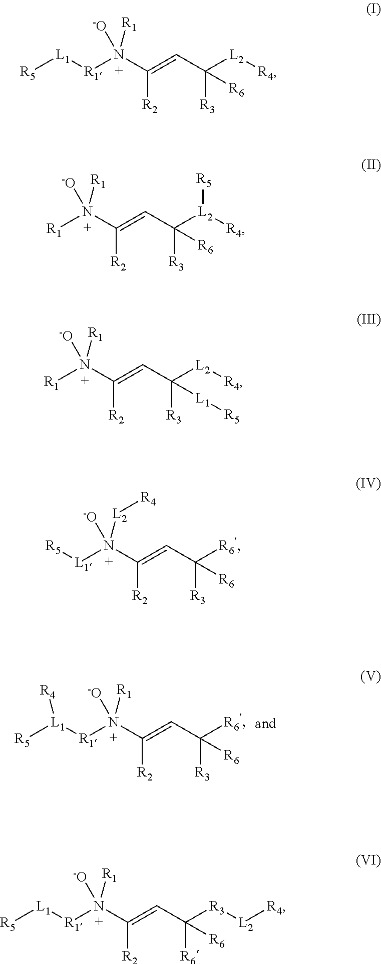
or a pharmaceutically acceptable salt or stereoisomer thereof,
wherein:
each of L₁ and L₂ is independently absent or a linking group, which may be the same or different;
R_{1′} is absent, or
R_{1′} and R₁, together with the nitrogen atom to which they are bound, form an optionally substituted 4- or 10-membered heterocyclyl comprising 1 to 3 heteroatoms selected from O, N, and S;
each R₁ is independently (C₁-C₈) alkyl, (C₃-C₁₀) carbocyclyl, or 4- or 10-membered heterocyclyl comprising 1 to 3 heteroatoms selected from O, N, and S, wherein said alkyl, carbocyclyl or heterocyclyl is further optionally substituted, or
two R₁ groups, together with the nitrogen atom to which they are bound, form an optionally substituted 4- or 10-membered heterocyclyl comprising 1 to 3 heteroatoms selected from O, N, and S;
R₂ is halo or alkoxy;
R₃ is a leaving group;
R₄ is a ligand that binds a cellular protein or a nucleic acid;
R₅ is a bioorthogonal handle, an affinity handle, or a reporter; and
R₆ and R_{6′} are independently hydrogen, (C₁-C₆) alkyl, or optionally substituted aryl, provided that P3 is not halo for the compound of formula VI.

2. The compound of claim 1, wherein R₁ is methyl.

3. The compound of claim 1, wherein R₂ is bromo.

4. The compound of claim 1, wherein R₂ is chloro.

5. The compound of claim 1, wherein R₂ is fluoro.

6. The compound of claim 1, wherein R₂ is iodo.

7. The compound of claim 1, wherein R₃ is halo or OC(O)NMe.

8. The compound of claim 7, wherein R₃ is bromo.

9. (canceled)

10. The compound of claim 1, wherein the linking group is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different, or
the linking group is a polyethylene glycol chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

11. The compound of claim 10, wherein each alkylene chain is a C₁-C₆ alkylene chain, or
wherein each polyethylene glycol chain has 1 to 6 —(CH₂CH₂—O)— units.

12.-13. (canceled)

14. The compound of claim 1, wherein R₄ is a small molecule, pharmacophore or a therapeutic moiety.

15. The compound of claim 14, wherein the pharmacophore is a substituted (C₆-C₁₀) aryl or substituted 5- to 10-membered heteroaryl.

16. The compound of claim 14, wherein R₄ is a therapeutic moiety.

17. The compound of claim 16, wherein the therapeutic moiety is an anti-cancer agent, an anti-bacterial agent, a non-steroidal anti-inflammatory drug (NSAID), a corticosteroid, or a disease-modifying antirheumatic drug (DMARD).

18. The compound of claim 17, wherein the anti-cancer agent is a kinase inhibitor.

19. The compound of claim 1, wherein R₅ is a bioorthogonal handle.

20. The compound of claim 19, wherein the bioorthogonal handle is CCH or N₃.

21. The compound of claim 1, wherein R₅ is an affinity handle.

22. The compound of claim 21, wherein the affinity handle is biotin or a derivative thereof.

23. The compound of claim 1, wherein R₅ is a reporter.

24. The compound of claim 23, wherein the reporter is a fluorophore.

25. The compound of claim 1, wherein:
each L₁ and L₂ is independently absent or (C₁-C₄) alkylene;
R₁ is Me, Et, iPr, or CH₂tBu;
R₂ is Cl, Br, or OMe;
R₃ is OC(O), C(O)O, OC(O)NH, NHC(O)O, OC(O)NMe, NMeC(O)O, OC(O)O, S(O)₂O, OS(O)₂, or OP(OR′)O₂;
R₅ is CCH, N₃, or biotin;
R₆ is hydrogen, Me, Et, iPr, phenyl, p-OMePh, p-CF₃Ph, or o-MePh.

26. A compound having a structure represented by formula VII: 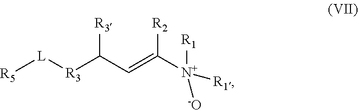
or a pharmaceutically acceptable salt or stereoisomer thereof,
wherein:
L is a linking group;
R₁ and R_{1′} are independently (C₁-C₈) alkyl, (C₃-C₁₀) carbocyclyl, or 4- or 10-membered heterocyclyl comprising 1 to 3 heteroatoms selected from O, N, and S, wherein said alkyl, carbocyclyl or heterocyclyl is further optionally substituted, or R₁ and R_{1′} together with the nitrogen atom to which they are attached, form a 4- to 7-membered heterocyclyl comprising 1 to 3 heteroatoms selected from O, N, and S;
R₂ is halo or alkoxy;
R₃ is absent, OC(O), C(O)O, OC(O)O, OC(O)NR′, NR′C(O)O, S(O), S(O)₂, OS(O)₂, S(O)₂O, OP(OR′)O₂, or a leaving group, wherein each R′ is independently hydrogen, (C₁-C₆) alkyl, (C₃-C₁₀) carbocyclyl, 4- or 7-membered heterocyclyl, and wherein said alkyl, carbocyclyl, or heterocyclyl is optionally substituted;
R_{3′} is hydrogen, (C₁-C₆) alkyl, (C₃-C₁₀) carbocyclyl, or 4- or 10-membered heterocyclyl comprising 1 to 3 heteroatoms selected from O, N, and S, or a leaving group, wherein said alkyl, carbocyclyl or heterocyclyl is further optionally substituted; and
R₅ is a bioorthogonal handle, an affinity handle, a reporter, or a ligand that binds a cellular protein or a nucleic acid,
provided that one of R₃ or R_{3′} is a leaving group.

27. The compound of claim 26, wherein R₁ is methyl or ethyl.

28. The compound of claim 26, wherein R_{1′} is methyl or ethyl.

29. The compound of claim 26, wherein R₂ is bromo.

30. The compound of claim 26, wherein R₂ is chloro.

31. The compound of claim 26, wherein R₂ is fluoro.

32. The compound of claim 26, wherein R₂ is iodo.

33. The compound of claim 26, wherein R_{3′} is a leaving group.

34. The compound of claim 33, wherein the leaving group is halo.

35. The compound of claim 26, wherein R₃ is OC(O)NMe.

36. The compound of claim 26, wherein L is an alkylene chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different, or
L is a polyethylene glycol chain, which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, —OP(O)O(R′)O—, —N(R′)P(O)N(R′R′)N(R′)—, C₃-C₁₂ carbocyclyl, 3- to 12-membered heterocyclyl, 5- to 12-membered heteroaryl or any combination thereof, wherein each R′ is independently H or optionally substituted C₁-C₂₄ alkyl, wherein the interrupting and the one or both terminating groups may be the same or different.

37. The compound of claim 36, wherein the alkylene chain is a C₁-C₆ alkylene chain, or
wherein the polyethylene glycol chain has 1 to 6 —(CH₂CH₂—O)— units.

38.-39. (canceled)

40. The compound of claim 26, wherein R₅ is a bioorthogonal handle.

41. The compound of claim 40, wherein the bioorthogonal handle is CCH or N₃.

42. The compound of claim 26, wherein R₅ is an affinity handle.

43. The compound of claim 42, wherein the affinity handle is biotin or a derivative thereof.

44. The compound of claim 26, wherein R₅ is a reporter.

45. The compound of claim 44, wherein the reporter is a fluorophore.

46. The compound of claim 26, wherein R₅ is a ligand that binds a cellular protein or a nucleic acid.

47. A diagnostic composition, comprising a diagnostically effective amount of the compound or pharmaceutically acceptable salt or stereoisomer of claim 1, and a pharmaceutically acceptable carrier.

48. A method of protein or nucleic acid labeling, comprising contacting a cell or lysate thereof with the compound or pharmaceutically acceptable salt or stereoisomer of claim 1, and a diboron reagent.

49. The method of claim 48, wherein the method comprises:
a) contacting a cell or lysate thereof with the diagnostically effective amount of a compound of formula I-VII or a pharmaceutically acceptable salt or stereoisomer thereof;
b) incubating the cells or lysate thereof, and the compound for a suitable period of time;
c) contacting the incubated cells or lysate thereof and the compound with a diboron reagent;
d) another period of incubation; and
e) analyzing the sample to detect binding of the binding ligand and the protein or nucleic acid.

50. The method of claim 48, wherein the diboron reagent is the diboron reagent is a symmetrical diboron reagent.

51. The method of claim 48, wherein the diboron reagent is the diboron reagent is an unsymmetrical diboron reagent.

52. The method of claim 48, wherein the contacting is conducted in vitro.

53. A process of preparing a compound of formula I: 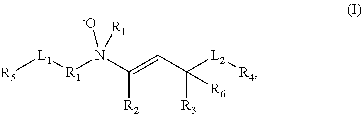
comprising reacting a compound of formula VIII: 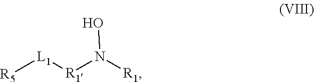
with a compound of formula IX: 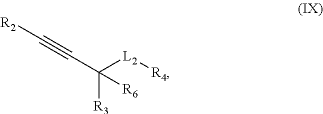
or
a compound of formula II: 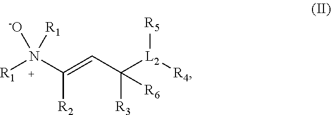
comprising reacting a compound of formula X: 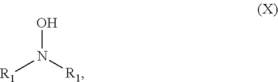
with a compound of formula XI: 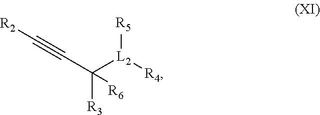
or
a compound of formula III: 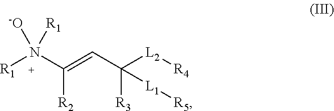
comprising reacting a compound of formula X: 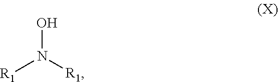
with a compound of formula XII: 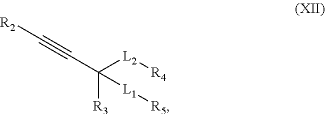
a compound of formula IV: 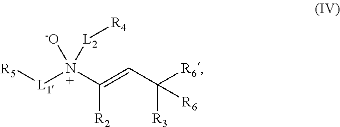
comprising reacting a compound of formula XIII: 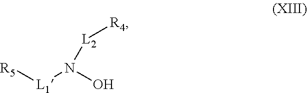
with a compound of formula XIV: 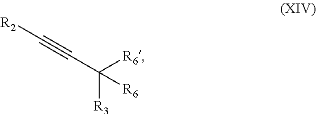
or
a compound of formula V: 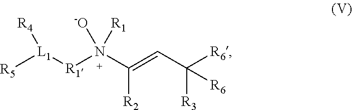
comprising reacting a compound of formula XV: 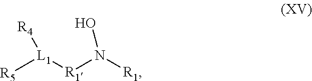
with a compound of formula XIV: 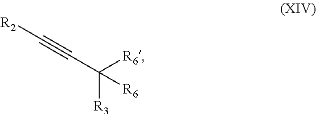
or
a compound of formula VI: 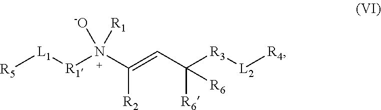
comprising reacting a compound of formula VIII: 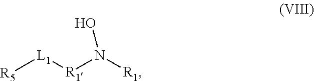
with a compound of formula XV:

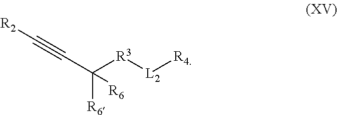

54.-65. (canceled)


SEQUENCE LISTING (9 sequences)

SEQ ID NO: 1 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLASSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 2 - protein, 260 aa, Bos taurus
MSHHWGYGKHNGPEHWHKDFPIANGERQSPVDIDTKAVVQDPALKPLALVYGEATSRRMVNNGHSFNVEYDDSQDKAVLKDGPLTGTYRLVQFHFHWGSSDDQGSEHTVDRKKYAAELHLVHWNTKYGDFGTAAQQPDGLAVVGVFLKVGDANPALQKVLDALDSIKTKGKSTDFPNFDPGSLLPNVLDYWTYPGSLTTPPLLESVTWIVLKEPISVSSQQMLKFRTLNFNAEGEPELLMLANWRPAQPLKNRQVRGFPK

SEQ ID NO: 3 - protein, 147 aa, Gallus gallus
MRSLLILVLCFLPLAALGKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRL

SEQ ID NO: 4 - protein, 161 aa, Homo sapiens
MGLSDGEWQLVLNVWGKVEADIPGHGQEVLIRLFKGHPETLEKFDKFKHLKSEDEMKASEDLKKHGATVLTALGGILKKKGHHEAEIKPLAQSHATKHKIPVKYLEFISECIIQVLQSKHPGDFGADAQGAMNKALELFRKDMASNYKELGFQGEHHHHHH

SEQ ID NO: 5 - protein, 13 aa, synthetic construct
  BINDING 2: note = His2 binding site
  BINDING 3: note = His3 binding site
SHHWGYGKHNGPE

SEQ ID NO: 6 - DNA, 32 bp, synthetic construct
  source 1..32: mol_type = other DNA
catcattggggttacggtaaacacaacggtcc

SEQ ID NO: 7 - protein, 19 aa, synthetic construct
  BINDING 2: note = His93 binding site
FHFHWGSSDDQGSEHTVDR

SEQ ID NO: 8 - protein, 19 aa, synthetic construct
  BINDING 4: note = His95 binding site
FHFHWGSSDDQGSEHTVDR

SEQ ID NO: 9 - protein, 13 aa, synthetic construct
  BINDING 9: note = Ly166 binding site
VLDALDSIKTKGK